(19) United States
(12) Patent Application Publication
(10) Pub. No.: US 2021/0163599 A1
(43) Pub. Date: Jun. 3, 2021

(54) PD-L1 BINDING AFFIMERS, AND USES RELATED THERETO

(71) Applicant: Avacta Life Sciences Limited, Wetherby (GB)

(72) Inventors: Amrik Basran, Cambridge (GB); Emma Jenkins, Stansted (GB); Estelle Adam, Hitchin (GB); Michelle Writer, Cambridge (GB); Emma Stanley, Hitchin (GB)

(73) Assignee: Avacta Life Sciences Limited, Wetherby (GB)

(21) Appl. No.: 17/046,646

(22) PCT Filed: Apr. 11, 2019
(86) PCT No.: PCT/EP2019/059354

§ 371 Date: Oct. 9, 2020

(30) Foreign Application Priority Data
Apr. 11, 2018 (GB) 1805963.4

(51) Int. Cl. C07K 16/28; C07K 14/705

(52) U.S. Cl. CPC C07K 16/2827; C07K 14/705; C07K 2319/30; C07K 2317/94; C07K 2317/565; C07K 2317/732; C07K 2317/92; C07K 2319/02; C07K 2317/734

(57) ABSTRACT

The present disclosure relates to proteins including PD-L1 binding affimer polypeptide sequences, gene expression constructs encoded those proteins, cells expressing those proteins, and pharmaceutical preparations of those proteins, gene expression constructs and cells and use in the treatment of various human conditions including cancer.

### RELATED APPLICATION

This application claims the benefit under 35 U.S.C. § 119(a) of Great Britain Application No. 1805963.4, filed Apr. 11, 2018, which is incorporated by reference herein in its entirety.

### BACKGROUND

Human cancers harbor numerous genetic and epigenetic alterations, generating neoantigens potentially recognizable by the immune system (Sjoblom et al., Science 314:268-74 (2006)). Although an endogenous immune response to cancer is observed in preclinical models and patients, this response is ineffective, and established cancers are viewed as “self” and tolerated by the immune system. Contributing to this state of tolerance, tumors may exploit several distinct mechanisms to actively suppress the host immune response (Topalian et al., J Clin Oncol 29:4828-36 (2011); Mellman et al., Nature 480:480-489 (2011)). Among these mechanisms, endogenous “immune checkpoints” that normally terminate immune responses to mitigate collateral tissue damage can be co-opted by tumors to evade immune destruction. Efforts to develop specific immune checkpoint pathway inhibitors have begun to provide new immunotherapeutic approaches for treating cancer, including the development of the anti-CTLA-4 antibody, ipilimumab, for the treatment of patients with advanced melanoma (Nodi et al., New Engl J Med 363:711-23 (2010)).

Programmed Death-1 (PD-1) is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1 (Freeman et al., J. Exp. Med. 192(7): 1027-34 (2000); Latchm an et al., Nat Immunol 2:261-8 (2001)).

PD-1 primarily functions in peripheral tissues where activated T-cells may encounter the immunosuppressive PD-L1 (also called B7-H1 or CD274) and PD-L2 (B7-DC) ligands expressed by tumor and/or stromal cells (Flies et al., Yale J Biol Med 84:409-21 (2011); Topalian et al., Curr Opin Immuno 24:1-6 (2012)).

Inhibition of the PD-1/PD-L1 interaction mediates potent antitumor activity in preclinical models (U.S. Pat. Nos. 8,008,449 and 7,943,743). It appears that upregulation of PD-L1 may allow cancers to evade the host immune system. An analysis of 196 tumor specimens from patients with renal cell carcinoma found that high tumor expression of PD-L1 was associated with increased tumor aggressiveness and a 4.5-fold increased risk of death (Thompson et al., Proc Natl Acad Sci USA 101 (49): 17174-9 (2004)). Ovarian cancer patients with higher expression of PD-L1 had a significantly poorer prognosis than those with lower expression. PD-L1 expression correlated inversely with intraepithelial CD8+ T-lymphocyte count, suggesting that PD-L1 on tumor cells may suppress antitumor CD8+ T cells (Hamanishi et al., Proc Natl Acad Sci USA 104 (9): 3360-3365 (2007)).

PD-L1 has also been implicated in infectious disease, in particular chronic infectious disease. Cytotoxic CD8 T lymphocytes (CTLs) play a pivotal role in the control of infection. Activated CTLs, however, often lose effector function during chronic infection. PD-1 receptor and its ligand PD-L1 of the B7/CD28 family function as a T cell co-inhibitory pathway and are emerging as major regulators converting effector CTLs into exhausted CTLs during chronic infection with human immunodeficiency virus, hepatitis B virus, hepatitis C virus, herpes virus, and other bacterial, protozoan, and viral pathogens capable of establishing chronic infections. Such bacterial and protozoal pathogens can include E. coli, Staphylococcus sp., Streptococcus sp., Mycobacterium tuberculosis, Giardia, Malaria, Leishmania, and Pseudomonas aeruginosa. Importantly, blockade of the PD-1/PD-L1 pathway is able to restore functional capabilities to exhausted CTLs. PD1/PD-L1 is thus a target for developing effective prophylactic and therapeutic vaccination against chronic bacterial and viral infections (see, e.g., Hofmeyer et al., Journal of Biomedicine and Biotechnology, vol. 2011, Article ID 451694, 9 pages, doi:10.1155/2011/451694).

Recent studies have also shown that systemic immune suppression may curtail the ability to mount the protective, cell-mediated immune responses that are needed for brain repair in neurodegenerative diseases. By using mouse models of Alzheimer's disease, immune checkpoint blockade directed against the programmed death-1 (PD-1) pathway was shown to evoke an interferon γ-dependent systemic immune response, which was followed by the recruitment of monocyte-derived macrophages to the brain. When induced in mice with established pathology, this immunological response led to clearance of cerebral amyloid-β (Aβ) plaques and improved cognitive performance. These findings suggest that immune checkpoints may be targeted therapeutically in neurodegenerative disease such as Alzheimer's disease using antibodies to PD-L1 (see, e.g., Baruch et al., Nature Medicine, January 2016, doi:10.1038/nm.4022).

Specific antibodies to PD-L1 have been developed as anti-cancer agents (see U.S. Pat. Nos. 9,212,224 and 8,008,449). The use of Ab inhibitors of the PD-1/PD-L1 interaction for treating cancer has entered clinical trials (Brahmer et al., J Clin Oncol 28:3167-75 (2010); Flies et al., Yale J Biol Med 84:409-21 (2011); Topalian et al., N Engl J Med 366:2443-54 (2012); Brahmer et al., N Engl J Med 366:2455-65 (2012)). There exists a need however, for additional PD-L1 inhibitory activities useful in the treatment of cancer, infectious disease, and neurodegenerative disease, e.g., Alzheimer's disease—such as PD-L1 inhibitors that can be readily formatted as part of fusion proteins with other polypeptide sequences providing, for instance, therapeutic activities or PK/ADME modifying activities. The present application fulfills this and other needs.

### SUMMARY

In some aspects, the present disclosure provides a protein comprising a PD-L1 binding affimer polypeptide sequence which binds to PD-L1 with a Kd of 1×10⁻⁶M or less and inhibits interaction of the PD-L1 to which it is bound with PD-1.

In some embodiments, the PD-L1 binding affimer polypeptide binds human PD-L1 and blocks interactions with human PD-1. In some embodiments, the PD-L1 binding affimer polypeptide binds human PD-L1 and blocks interactions with human CD80. In some embodiments, the PD-L1 binding affimer polypeptide bind PD-L1 with a Kd of 1×10⁻⁷M or less, Kd of 1×10⁻⁸M or less, Kd of 1×10⁻⁹M or less, or even a Kd of 1×10⁻¹⁰ M or less. In some embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In some embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In some embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In some embodiments, the PD-L1 binding affimer polypeptide binds PD-L1 in a competitive binding assay with human CD80 (B7-1) with an IC₅₀ of 1 μM or less, 100 nM or less, 40 nM or less, 20 nM or less, 10 nM or less, 1 nM or less, or 0.1 nM or less.

In some embodiments, the PD-L1 binding affimer polypeptide has an amino acid sequence represented in general formula (I)

FR1-(Xaa)ₙ-FR2-(Xaa)ₘ-FR3 (I)

wherein

FR1 is a polypeptide sequence represented by MIPGGLSEAK PATPEIQEIV DKVKPQLEEK TNETYGKLEA VQYKTQVLA (SEQ ID NO: 1) or a polypeptide sequence having at least 70% homology thereto;

FR2 is a polypeptide sequence represented by GTNYYIKVRA GDNKYMHLKV FKSL (SEQ ID NO: 2) or a polypeptide sequence having at least 70% homology thereto;

FR3 is a polypeptide sequence represented by EDLVLTGYQV DKNKDDELTG F (SEQ ID NO: 3) or a polypeptide sequence having at least 70% homology thereto; and

Xaa, individually for each occurrence, is an amino acid residue; and
n and m are each, independently, an integer from 3 to 20.

For some embodiments, the FR1 may a polypeptide sequence having at least 80%, 85%, 90%, 95% or even 98% homology with SEQ ID NO: 1. For some embodiments, FR2 is a polypeptide sequence having at least 80%, 85%, 90%, 95% or even 98% homology with SEQ ID NO: 2. For some embodiments, FR3 is a polypeptide sequence having at least 80%, 85%, 90%, 95% or even 98% homology with SEQ ID NO: 2.

In some embodiments, the PD-L1 binding affimer polypeptide has an amino acid sequence represented in the general formula:

(SEQ ID NO: 4)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF

wherein
Xaa, individually for each occurrence, is an amino acid residue;
n and m are each, independently, an integer from 3 to 20;
Xaa1 is Gly, Ala, Val, Arg, Lys, Asp, or Glu;
Xaa2 is Gly, Ala, Val, Ser or Thr;
Xaa3 is Arg, Lys, Asn, Gln, Ser, Thr;
Xaa4 is Gly, Ala, Val, Ser or Thr;
Xaa5 is Ala, Val, Ile, Leu, Gly or Pro;
Xaa6 is Gly, Ala, Val, Asp or Glu; and
Xaa5 is Ala, Val, Ile, Leu, Arg or Lys.

For some embodiments, Xaa1 is Gly, Ala, Arg or Lys, more even more preferably Gly or Arg. For some embodiments, Xaa2 is Gly or Ser. For some embodiments, Xaa3 is Arg Arg, Lys, Asn or Gln, more preferably Lys or Asn. For some embodiments, Xaa4 is Gly or Ser. For some embodiments, Xaa5 is Ala, Val, Ile, Leu, Gly or Pro, more preferably Ile, Leu or Pro, and even more preferably Leu or Pro. For some embodiments, Xaa6 is Ala, Val, Asp or Glu, even more preferably Ala or Glu. For some embodiments, Xaa5 is Ile, Leu or Arg, more preferably Leu or Arg.

In some embodiments, the PD-L1 binding affimer polypeptide has an amino acid sequence represented in the general formula:

(SEQ ID NO: 5)
MIPRGLSEAKPATPEIQEIVDKVKPQLEEKTNETYGKLEAVQYKTQVLA-
(Xaa)ₙ-STNYYIKVRAGDNKYMHLKVFNGP-(Xaa)ₘ-ADRVLTGYQVDK
NKDDELTGF

wherein Xaa, individually for each occurrence, is an amino acid residue; and n and m are each, independently, an integer from 3 to 20.

In some embodiments of the above sequences, (Xaa)ₙ (“loop 2”) is an amino acid sequence represented in the general formula (II)

-aa1-aa2-aa3-Gly-Pro-aa4-aa5-Trp-aa6- (II)

wherein

aa1 represents an amino acid residue with a basic sidechain;

aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain;

aa3 represents an amino acid residue with an aromatic or basic sidechain;

aa4 represents an amino acid residue with a neutral polar or non-polar sidechain or a charged (acidic or basic) sidechain; preferably a neutral polar sidechain or a charged (acidic or basic) sidechain;

aa5 represents an amino acid residue with a neutral polar or a charged (acidic or basic) or a small aliphatic or an aromatic sidechain; preferably a neutral polar sidechain or a charged sidechain; and

aa6 represents an amino acid residue with an aromatic or acid sidechain.

For some embodiments, aa1 represents Lys, Arg or His, more preferably Lys or Arg. For some embodiments, aa2 represents Ala, Pro, Ile, Gln, Thr, Asp, Glu, Lys, Arg or His, more preferably Ala, Gln, Asp or Glu. For some embodiments, aa3 represents Phe, Tyr, Trp, Lys, Arg or His, preferably Phe, Tyr, Trp, more preferably His or Tyr, Trp or His. For some embodiments, aa4 represents Ala, Pro, Ile, Gln, Thr, Asp, Glu, Lys, Arg or His, more preferably Gln, Lys, Arg, His, Asp or Glu. For some embodiments, aa5 represents Ser, Thr, Asn, Gln, Asp, Glu, Arg or His, more preferably Ser, Asn, Gln, Asp, Glu or Arg. For some embodiments, aa6 represents Phe, Tyr, Trp, Asp or Glu; preferably Trp or Asp; more preferably Trp.

In certain other embodiments of the above sequences, (Xaa)ₙ (“loop 2”) is an amino acid sequence represented in the general formula (III)

-aa1-aa2-aa3-Phe-Pro-aa4-aa5-Phe-Trp- (III)

wherein

aa1 represents an amino acid residue with a basic sidechain or aromatic sidechain;

aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain;

aa3 represents an amino acid residue with an aromatic or basic sidechain, preferably Phe, Tyr, Trp, Lys, Arg or His, more preferably Phe, Tyr, Trp or His, and even more preferably Tyr, Trp or His;

aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu; and

aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg.

For some embodiments, aa1 represents Lys, Arg, His, Ser, Thr, Asn or Gln, more preferably Lys, Arg, His, Asn or Gln, and even more preferably Lys or Asn. For some embodiments, aa2 represents Ala, Pro, Ile, Gln, Thr, Asp, Glu, Lys, Arg or His, more preferably Ala, Gln, Asp or Glu. For some embodiments, aa3 represents Phe, Tyr, Trp, Lys, Arg or His, more preferably Phe, Tyr, Trp or His, and even more preferably Tyr, Trp or His. For some embodiments, aa4 represents Ala, Pro, Ile, Gln, Thr, Asp, Glu, Lys, Arg or His, and even more preferably Gln, Lys, Arg, His, Asp or Glu. For some embodiments, aa5 represents Ser, Thr, Asn, Gln, Asp, Glu, Arg or His, and even more preferably Ser, Asn, Gln, Asp, Glu or Arg.

In some embodiments of the above sequences, (Xaa)ₙ (“loop 2”) is an amino acid sequence selected from SEQ ID NOS: 6 to 41, or an amino acid sequence having at least 80% homology thereto, and more preferably an amino acid sequence having at least 85%, 90%, 95% or even 98% homology thereto.

In some embodiments of the above sequences, (Xaa)ₙ (“loop 2”) is an amino acid sequence selected from SEQ ID NOS: 6 to 41, or an amino acid sequence having at least 80% identity thereto, and more preferably an amino acid sequence having at least 85%, 90%, 95% or even 98% identity thereto.

In some embodiments of the above sequences, (Xaa)ₘ (“loop 4”) is an amino acid sequence represented in the general formula (IV)

-aa7-aa8-aa9-aa10-aa11-aa12-aa13-aa14-aa15- (IV)

wherein

aa7 represents an amino acid residue with neutral polar or non-polar sidechain or an acidic sidechain;

aa8 represents an amino acid residue, preferably an amino acid residue with a neutral polar or non-polar sidechain or a charged (acidic or basic) sidechain or aromatic sidechain, more preferably a charged (acidic or basic) sidechain;

aa9 represents an amino acid residue, preferably an amino acid residue with a neutral polar or non-polar sidechain or a charged (acidic or basic) sidechain or aromatic sidechain, more preferably a neutral polar side chain or an acid side chain;

aa10 represents an amino acid residue, preferably an amino acid residue with a neutral polar or non-polar sidechain or a charged (acidic or basic) sidechain or aromatic sidechain, more preferably a neutral polar side chain or a basic or acid side chain;

aa11 represents an amino acid residue, preferably an amino acid residue with a neutral polar sidechain or a charged (acidic or basic) sidechain or a nonpolar aliphatic sidechain or an aromatic sidechain, more preferably a neutral polar side chain or a basic or acid side chain;

aa12 represents an amino acid residue, preferably an amino acid residue with a neutral polar sidechain or a charged (acidic or basic) sidechain or a nonpolar aliphatic sidechain or an aromatic sidechain, more preferably an acid side chain;

aa13 represents an amino acid residue, preferably an amino acid residue with a neutral polar sidechain or a charged (acidic or basic) sidechain or a nonpolar aliphatic sidechain or an aromatic sidechain, more preferably an acid side chain;

aa14 represents an amino acid residue, preferably an amino acid residue with a neutral polar sidechain or a charged (acidic or basic) sidechain; and

aa15 represents an amino acid residue, preferably an amino acid residue with a neutral polar or neutral non-polar sidechain or a charged (acidic or basic) sidechain.

For some embodiments, aa7 represents Gly, Ala, Val, Pro, Trp, Gln, Ser, Asp or Glu, and even more preferably Gly, Ala, Trp, Gln, Ser, Asp or Glu. For some embodiments, aa8 represents Asp, Glu, Lys, Arg, His, Gln, Ser, Thr, Asn, Ala, Val, Pro, Gly, Tyr or Phe, and even more preferably Asp, Glu, Lys, Arg, His or Gln. For some embodiments, aa9 represents Gln, Ser, Thr, Asn, Asp, Glu, Arg, Lys, Gly, Leu, Pro or Tyr, and even more preferably Gln, Thr or Asp. For some embodiments, aa10 represents Asp, Glu, Arg, His, Lys, Ser, Gln, Asn, Ala, Leu, Tyr, Trp, Pro or Gly, and even more preferably Asp, Glu, His, Gln, Asn, Leu, Trp or Gly. For some embodiments, aa11 represents Asp, Glu, Ser, Thr, Gln, Arg, Lys, His, Val, Ile, Tyr or Gly and even more preferably Asp, Glu, Ser, Thr, Gln, Lys or His. For some embodiments, aa12 represents Asp, Glu, Ser, Thr, Gln, Asn, Lys, Arg, Val, Leu, Ile, Trp, Tyr, Phe or Gly and even more preferably Asp, Glu, Ser, Tyr, Trp, Arg or Lys. For some embodiments, aa13 represents Ser, Thr, Gln, Asn, Val, Ile, Leu, Gly, Pro, Asp, Glu, His, Arg, Trp, Tyr or Phe and even more preferably Ser, Thr, Gln, Asn, Val, Ile, Leu, Gly, Asp or Glu. For some embodiments, aa14 represents Ala, Ile, Trp, Pro, Asp, Glu, Arg, Lys, His, Ser, Thr, Gln or Asn and even more preferably Ala, Pro, Asp, Glu, Arg, Lys, Ser, Gln or Asn. For some embodiments, aa15 represents His, Arg, Lys, Asp, Ser, Thr, Gln, Asn, Ala, Val, Leu, Gly or Phe and even more preferably His, Arg, Lys, Asp, Ser, Thr, Gln or Asn.

In some embodiments of the above sequences, (Xaa)ₙ (“loop 4”) is an amino acid sequence selected from SEQ ID NOS: 42 to 77, or an amino acid sequence having at least 80% homology thereto, and more preferably an amino acid sequence having at least 85%, 90%, 95% or even 98% homology thereto.

In some embodiments of the above sequences, (Xaa)ₙ (“loop 4”) is an amino acid sequence selected from SEQ ID NOS: 42 to 77, or an amino acid sequence having at least 80% identity thereto, and more preferably an amino acid sequence having at least 85%, 90%, 95% or even 98% identity thereto.

In some embodiments, the PD-L1 binding affimer polypeptide has an amino acid sequence selected from SEQ ID NOS: 78 to 86, or an amino acid sequence having at least 70% homology thereto, and even more preferably at least 75%, 80%, 85%, 90%, 95% or even 98% homology thereto.

In some embodiments, the PD-L1 binding affimer polypeptide has an amino acid sequence selected from SEQ ID NOS: 78 to 86, or an amino acid sequence having at least 70% identity thereto, and even more preferably at least 75%, 80%, 85%, 90%, 95% or even 98% identity thereto.

In some embodiments, the PD-L1 binding affimer polypeptide has an amino acid sequence can be encoded by a nucleic acid having a coding sequence corresponding to nucleotides 1-336 of one of SEQ ID NOS: 87 to 94, or a coding sequence at least 70% identical thereto, and even more preferably at least 75%, 80%, 85%, 90%, 95% or even 98% identity thereto.

In some embodiments, the PD-L1 binding affimer polypeptide has an amino acid sequence can be encoded by a nucleic acid having a coding sequence that hybridizes to any one of SEQ ID NOS: 87 to 94 under stringent conditions of 6× sodium chloride/sodium citrate (SSC) at 45° C. followed by a wash in 0.2×SSC at 65° C.

In some embodiments, the Affimer Agent proteins described herein bind PD-L1 through the PD-L1 binding affimer polypeptide in a manner competitive with PD-L1 binding by anti-PD-L1 antibodies Atezolizumab, Avelumab and/or Durvalumab.

In some embodiments, the Affimer Agent proteins described herein include a PD-L1 binding affimer polypeptide that forms a crystal structure with PD-L1 comprising an interface involving at least 10 residues of PD-L1 selected from Ile-54, Tyr-56, Glu-58, Glu-60, Asp-61, Lys-62, Asn-63, Gln 66, Val-68, Val-76, Val-111, Arg-113, Met-115, Ile-116, Ser-117, Gly-120, Ala-121, Asp-122, Tyr-123, and Arg-125.

In some embodiments, the Affimer Agent proteins described herein, in a manner dependent on the PD-L1 binding affimer polypeptide binding to PD-L1, (a) increases T-cell receptor signaling in subset of T cell bearing certain Vfi chains, for example, VB3, VB12, VB14, and VB17 in human PBMCs, when treated with staphylococcus enterotoxin B (SEB); (b) increases interferon-γ production in an SEB assay; and/or (c) increases interleukin-2 (IL-2) production in an SEB assay in a dose dependant manner.

In some embodiments, the Affimer Agent proteins described herein, in a manner dependent on the PD-L1 binding affimer polypeptide binding to PD-L1 (a) increases T-cell proliferation in a mixed lymphocyte reaction (MLR) assay; (b) increases interferon-γ production in an MLR assay; and/or (c) increases interleukin-2 (IL-2) secretion in an MLR assay.

In some embodiments, the Affimer Agent is a fusion protein which may include in addition to the PD-L1 binding affimer polypeptide, to illustrate, one or more additional amino acid sequences selected from the group consisting of: secretion signal sequences, peptide linker sequences, affinity tags, transmembrane domains, cell surface retention sequence, substrate recognition sequences for post-translational modifications, multimerization domains to create multimeric structures of the protein aggregating through protein-protein interactions, half-life extending polypeptide moieties, polypeptide sequences for altering tissue localization and antigen binding site of an antibody, and one or more additional affimer polypeptide sequences binding the PD-L1 or a different target.

In some embodiments, the fusion protein includes a half-life extending polypeptide moiety such as selected from the group consisting of an Fc domain or portion thereof, an albumin protein or portion thereof, an albumin-binding polypeptide moiety, transferrin or portion thereof, a transferrin-binding polypeptide moiety, fibronectin or portion thereof, or a fibronectin-binding polypeptide moiety.

Where the fusion protein includes an Fc domain or a portion thereof, in some embodiments it is an Fc domain that retains FcRn binding.

Where the fusion protein includes an Fc domain or a portion thereof, in some embodiments the Fc domain or a portion thereof is from IgA, IgD, IgE, IgG, and IgM or a subclass (isotype) thereof such as IgG1, IgG2, IgG3, IgG4, IgA1 or IgA2.

In some embodiments, the fusion protein has an amino acid sequence of SEQ ID NO: 111 or SEQ ID NO: 112 or a sequence having at least 70% homology thereto, and even more preferably at least 75%, 80%, 85%, 90%, 95% or even 98% identity thereto.

Where the fusion protein includes an Fc domain or a portion thereof, in some embodiments the Fc domain or a portion thereof retains effector function selected from C1q binding, complement dependent cytotoxicity (CDC), antibody-dependent cell-mediated cytotoxicity (ADCC); phagocytosis; down regulation of B cell receptor, or a combination thereof.

In some embodiments, where the fusion protein includes a half-life extending polypeptide moiety, that moiety increases the serum half-life of the protein by at least 5-fold relative to its absence from the protein, for example, 10-fold, 20-fold, 30-fold, 40-fold, 50-fold, 60-fold, 70-fold, 80-fold, 90-fold, 100-fold, 200-fold, 500-fold or even 1000-fold.

In some embodiments, the fusion protein of the disclosure is provided as a pharmaceutical preparation suitable for therapeutic use in a human patient, further comprising one or more pharmaceutically acceptable excipients, buffers, salts or the like.

In another aspect of the disclosure, there is provided a recombinant antibody comprising one or more V_{H} and/or V_{L} chains forming one or more antigen binding sites that bind to a target antigen, wherein at least one of the V_{H} and/or V_{L} chains is a fusion protein also including at least one PD-L1 binding affimer polypeptide sequences that binds PD-L1 with a Kd of 1×10⁶M or less and inhibits interaction of PD-1 with the PD-L1 to which it is bound. In some embodiments, the PD-L1 binding affimer polypeptide binds human PD-L1 and blocks interactions with human PD-1. In some embodiments, the PD-L1 binding affimer polypeptide bind PD-L1 with a Kd of 1×10⁺⁷M or less, Kd of 1×10⁻⁸M or less, Kd of 1×10⁻⁹M or less, or even a Kd of 1×10⁻¹⁰M or less. In some embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In some embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In some embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In some embodiments, the V_{H} chain includes an Fc domain.

In some embodiments, the target antigen is an immune checkpoint.

In some embodiments, the target antigen is an immune costimulatory receptor and the chimeric antibody agonizes the costimulatory receptor on binding.

In some embodiments, the target antigen is an angiogenic factor or a receptor therefore and the chimeric antibody antagonizes the angiogenic factor or receptor therefore.

In some embodiments, the target antigen is a tumor antigen.

In some embodiments, the target antigen is a soluble immunosuppressive factor or a receptor therefore, and the chimeric antibody inhibitors the immunosuppressive activity of the immunosuppressive factor to act as an immunostimulatory signal.

In some embodiments, wherein the target antigen is selected from the group consisting of PD-1, PD-L2, CTLA-4, NKG2A, KIR, LAG-3, TIM-3, CD96, VISTA, TIGIT, CD28, ICOS, CD137, OX40, GITR, CD27, CD30, HVEM, DNAM-1 or CD28H, CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX4OL, PVR, SIRPα, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DL5A, KIR2DL5B, KIR3DL1, KIR3DL2, KIR3DL3, NKG2A, NKG2C, NKG2E or TSLP.

In some embodiments, there is provided a recombinant Affimer-Ipilimumab antibody fusion protein comprising

an affimer-heavy chain fusion protein (wherein the secretion signal sequence MPLLLLLPLLWAGALA (SEQ ID NO: 136) is optionally removed) with an amino acid sequence of SEQ ID NO: 113 or a sequence having at least 70% homology thereto (for example, at least 75%, 80%, 85%, 90%, 95% or even 98% identity thereto), and

a light chain protein (wherein the secretion signal sequence MPLLLLLPLLWAGALA (SEQ ID NO: 136) is optionally removed) with an amino acid sequence of SEQ ID NO: 114 or a sequence having at least 70% homology thereto (for example, at least 75%, 80%, 85%, 90%, 95% or even 98% identity thereto).

In some embodiments, there is provided a recombinant Affimer-Bevacizumab antibody fusion protein comprising

an affimer-heavy chain fusion protein (wherein the secretion signal sequence MPLLLLLPLLWAGALA (SEQ ID NO: 136) is optionally removed) with an amino acid sequence of SEQ ID NO: 115 or 117 or a sequence having at least 70% homology thereto (for example, at least 75%, 80%, 85%, 90%, 95% or even 98% identity thereto), and

a light chain protein (wherein the secretion signal sequence MPLLLLLPLLWAGALA (SEQ ID NO: 136) is optionally removed) with an amino acid sequence of SEQ ID NO: 116 or a sequence having at least 70% homology thereto (for example, at least 75%, 80%, 85%, 90%, 95% or even 98% identity thereto).

In some embodiments, the recombinant antibodies of the disclosure are provided as a pharmaceutical preparation suitable for therapeutic use in a human patient, further comprising one or more pharmaceutically acceptable excipients, buffers, salts or the like.

In another aspect of the disclosure, there is provided a recombinant receptor trap fusion protein comprising (i) a ligand binding domain of a receptor, and (ii) a PD-L1 binding affimer polypeptide sequence(s) which binds to PD-L1 with a Kd of 1×10⁻⁶M or less and inhibits interaction of PD-1 to the PD-L1 to which it is bound. In some embodiments, the PD-L1 binding affimer polypeptide bind PD-L1 with a Kd of 1×10⁻⁷M or less, Kd of 1×10⁻⁸M or less, Kd of 1×10⁻⁹M or less, or even a Kd of 1×10⁻¹⁰M or less. In some embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In some embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In some embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In some embodiments, the binding domain binds to PGE2, TGF-β, VEGF, CCL2, IDO, CSF1, IL-10, IL-13, IL-23, or adenosine.

In some embodiments, the recombinant receptor trap fusion protein includes one or more multimerization domains that induces multimerization of the recombinant receptor trap fusion protein, i.e., complexes including 2, 3, 4, 5, 6, 7, 8, 9 or even 10 recombinant receptor trap fusion proteins in a multimeric complex.

In some embodiments, the recombinant receptor trap fusion proteins of the disclosure are provided as a pharmaceutical preparation suitable for therapeutic use in a human patient, further comprising one or more pharmaceutically acceptable excipients, buffers, salts or the like.

In another aspect of the disclosure, there is provided a recombinant receptor ligand fusion protein comprising (i) a polypeptide ligand sequence that binds to an agonizes or antagonizes its cognate receptor, and (ii) a PD-L1 binding affimer polypeptide sequence(s) which binds to PD-L1 with a Kd of 1×10⁻⁶M or less and inhibits interaction of PD-1 to the PD-L1 to which it is bound. In some embodiments, the PD-L1 binding affimer polypeptide bind PD-L1 with a Kd of 1×10⁻⁷M or less, Kd of 1×10⁻⁸M or less, Kd of 1×10⁻⁹M or less, or even a Kd of 1×10⁻¹⁰ M or less. In some embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In some embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In some embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In some embodiments of the recombinant receptor ligand fusion protein, the polypeptide ligand is a ligand for a co-stimulatory receptor and agonizes the co-stimulatory receptor upon binding.

For instance, the polypeptide ligand can be selected from B7.1, 4-1BBL, OX40L, GITRL or LIGHT.

For instance, the polypeptide ligand can be an immunostimulatory cytokine that promotes antitumor immunity, such as IFN-α2, IL-2, IL-15, IL-21, and IL-12.

In some embodiments, the recombinant receptor ligand fusion protein includes one or more multimerization domains that induces multimermization of the recombinant receptor ligand fusion protein, i.e., complexes including 2, 3, 4, 5, 6, 7, 8, 9 or even 10 recombinant receptor ligand fusion proteins in a multimeric complex.

In some embodiments, the recombinant receptor ligand fusion proteins of the disclosure are provided as a pharmaceutical preparation suitable for therapeutic use in a human patient, further comprising one or more pharmaceutically acceptable excipients, buffers, salts or the like.

In another aspect of the disclosure, there is provided a multispecific T-cell engaging fusion protein comprising (i) a CD3 binding polypeptide binds to CD3 on the surface of T-cells, and (ii) a PD-L1 binding affimer polypeptide sequence(s) which binds to PD-L1 with a Kd of 1×10⁻⁶M or less and inhibits interaction of PD-1 to the PD-L1 to which it is bound. In some embodiments, the PD-L1 binding affimer polypeptide bind PD-L1 with a Kd of 1×10⁻⁷M or less, Kd of 1×10⁻⁸M or less, Kd of 1×10⁻⁹M or less, or even a Kd of 1×10⁻¹⁰ M or less. In some embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In some embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In some embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In some embodiments, the multispecific T-cell engaging fusion proteins of the disclosure are provided as a pharmaceutical preparation suitable for therapeutic use in a human patient, further comprising one or more pharmaceutically acceptable excipients, buffers, salts or the like.

In another aspect of the disclosure, there is provided a chimeric receptor fusion protein comprising (i) an extracellular portion including a PD-L1 binding affimer polypeptide sequence(s) which binds to PD-L1 with a Kd of 1×10⁻⁶M or less and inhibits interaction of PD-1 to the PD-L1 to which it is bound; (ii) a transmembrane domain; and (c) a cytoplasmic domain comprising a 4-1BB signaling domain and a CDR signaling domain, and optional a costimulatory signaling region. In some embodiments, the PD-L1 binding affimer polypeptide bind PD-L1 with a Kd of 1×10⁺⁷M or less, Kd of 1×10⁻⁸M or less, Kd of 1×10⁻⁹M or less, or even a Kd of 1×10⁻¹⁰ M or less. In some embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In some embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In some embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In certain embodiments, the present disclosure also provides a cell, preferably a lymphocyte and even more preferably a T-lymphocyte, that has been engineered with a gene encoding the chimeric receptor fusion protein, which gene when expressed results in the presentation of the chimeric receptor fusion protein on the cell surface.

In another aspect of the disclosure, there is provided nucleic acids comprising a coding sequence encoding a Affimer Agent, such as protein described above (and herein).

In some embodiments, the coding sequence is operably linked to one or more transcriptional regulatory sequences, such as a promoter and/or enhancer.

In some embodiments, the nucleic acid includes one or more origins of replication, minichromosome maintenance elements (MME) and/or nuclear localization elements.

In some embodiments, the nucleic acid includes a polyadenylation signal sequence which is operably linked and transcribed with the coding sequence.

In some embodiments, the coding sequence includes one or more intronic sequences

In some embodiments, the nucleic acid includes one or more ribosome binding sites which are transcribed with the coding sequence.

In some embodiments, the nucleic acid is DNA.

In some embodiments, the nucleic acid is RNA, such as an mRNA.

In another aspect of the disclosure, there is provided viral vectors including a coding sequence encoding a Affimer Agent, such as protein described above (and herein).

In another aspect of the disclosure, there is provided plasmid DNA, plasmid Vectors or minicircles including a coding sequence encoding an Affimer Agent, such as protein described above (and herein).

In another aspect of the disclosure, the disclosure provides an antibody or antigen binding fragment thereof further comprising a PD-L1 binding affimer polypeptide conjugated thereto.

In another aspect of the disclosure, the disclosure provides a soluble receptor or ligand binding domain thereof further comprising a PD-L1 binding affimer polypeptide conjugated thereto.

In another aspect of the disclosure, the disclosure provides a growth factor, cytokine or chemokine biologically active polypeptide fragment thereof further comprising a PD-L1 binding affimer polypeptide conjugated thereto.

In another aspect of the disclosure, the disclosure provides a costimulatory agonist polypeptide further comprising a PD-L1 binding affimer polypeptide conjugated thereto.

In another aspect of the disclosure, the disclosure provides a checkpoint inhibitory polypeptide further comprising a PD-L1 binding affimer polypeptide conjugated thereto.

In another aspect of the disclosure, the disclosure provides an Affimer Agent comprising a PD-L1 binding affimer polypeptide and a detectable label, a toxin or one or more therapeutic agents conjugated thereto.

Also provided herein is a pharmaceutical preparation suitable for therapeutic gene delivery in a human patient, comprising a nucleic acid, a viral vector, a plasmid DNA, plasmid Vector or minicircle of the present disclosure, and (ii) one or more pharmaceutically acceptable excipients, buffers, salts, transfection enhancers, electroporation enhancers or the like.

Further provided herein are methods comprising administering to a subject the protein, recombinant antibody, or nucleic acid described herein (comprising an Affimer that binds to PD-L1).

In some embodiments, the subject comprises cancer cells that express PD-L1, optionally wherein the cancer cells are melanoma cells.

In some embodiments, the protein, recombinant antibody, or nucleic acid is administered in an effective amount to elicit increase of IFNγ production by T cells in a mixed lymphocyte reaction.

In some embodiments, the protein, recombinant antibody, or nucleic acid is administered in an effective amount to increase IFNγ production by T cells in the subject by at least 2-fold, relative to a vehicle-only control.

In some embodiments, the subject has a tumor comprising the cancer cells that express PD-L1, and the level of PD-L1 binding affimer polypeptide accumulation in the tumor is at least 5 times the level in plasma at 96 hours post-administration.

In some embodiments, the subject has a tumor comprising the cancer cells that express PD-L1, and the protein, recombinant antibody, or nucleic acid is administered in an effective amount to inhibit growth of the tumor in the subject by at least 10%.

In some embodiments, the subject has a melanoma.

### BRIEF DESCRIPTION OF THE DRAWINGS

FIG. 1. Generating Affimer Libraries: Variabilized Binding Loops Give Rise to Unique Binding Surfaces and Selectable Affimer Binders

FIG. 2. Monomeric affimer binding. Affimer binding by flow cytometry on human lung adenocarcinoma cancer cell lines.

FIG. 3. Affimer multimers are expressed easily in E. coli to high yield and purity of multiple formats (even in shaker flasks production)

FIGS. 4A and 4B. Affimer multimers bind to PD-L1 with kinetics demonstrating avidity beyond the monomeric binding domain.

FIGS. 5A and 5B and 5C. Affimer Fc fusions provide effector function, half-life extension and enhanced affinity.

FIGS. 6A-6C. Competiton ELISA to PD-1 and CD80 and against PD-L1 antibody benchmark.

FIG. 7. Affimer-Fc fusions demonstrate increased serum half-life.

FIG. 8. Immunogenicity Testing by human PBMC Stimulation assay indicates that there is a low risk that the core affimer sequence is immunogenic in humans.

FIG. 9. Data demonstrates that Affimers can be formatted at various sites on a human Fc, and so should translate to IgG-Affimer fusions. Typical expression yields in the range 400-800 mg/l. Analytical SEC-HPLC used to assess purity.

FIG. 10. Illustrates the K_{D} of several PD-L1 Affimer Fc formats determined using Biacore, showing highly flexible formatting for fine tuning of binding kinetics to suit therapeutic target. Avidity effects with the divalent Fc format clearly observed.

FIGS. 11A-11B. Ipilimumab (biosimilar)/AVA04-141 transiently expressed in Expi293F cells, purified yield of ˜160 mg/L post Protein A purification.

FIGS. 12A-12C. Unoptimized Biacore demonstrates that the bi-specific antibody-Affimer fusion is able to engage both targets

FIGS. 13A-13D. Bevacizumab (biosimilar)/AVA04-251 transiently expressed in Expi293F cells could be purified to greater than 97% yield, and Biacore demonstrates that the bi-specific antibody-Affimer fusions are able to engage both targets whether the constructed included a flexible linker [(G4S)3] or rigid linker [A(EAAAK)3]. The construct with a rigid linker has been then tested in a pharmacokinetic study in mouse (FIG. 13D)

FIG. 14. Shows the calculated 3-dimensional structures of the anti-PD-L1 affimer AVA04-261 and human PD-L1 derived from the crystallization of the protein complex.

FIG. 15. From the crystal-derived structure of anti-PD-L1 affimer AVA04-261 bound to human PD-L1 derived, FIG. 15 provides the hydrogen bonding interactions between amino acid residues at the interface of contact between the two proteins.

FIG. 16. From the crystal-derived structure of anti-PD-L1 affimer AVA04-261 bound to human PD-L1 derived, FIG. 16 provides a list of amino acid residues involved in the interface of contact between the two proteins.

FIGS. 17A-17B. Illustrative examples of anti-PD-L1 affimer formatting, including Fc fusions (showing a divalent PD-L1 binder format and a bispecific, divalent PD-L1 binder and Target X binder format), various formats of inline antibody fusions, a BiTE format and an inline fusion of the anti-PD-L1 affimer with a receptor trap domain. Flexible linker (G4S)₆ corresponds to SEQ ID NO: 106. Rigid linker A(EAAAK)₆ corresponds to SEQ ID NO: 196.

FIGS. 18A-18B. FIG. 18A shows various clone compositions combining an AVA04 Affimer, rigid or flexible linker, and Affimer XT (AVA03-42). Analytical SEC-HPLC was used to assess purity of each clone. FIG. 18B. Shows results of kinetics analyses for various affimers XT on rhPD-1-Fc or huSA.

FIG. 19. An SDS-PAGE run of 1 μl of purified variant proteins. Table summarizes E. coli production yield, AVA04-251 alanine scanning across amino acid positions in loop 4, and Biacore binding results for each affimer. Loop 2 sequence corresponds to SEQ ID NO: 39. Loop 4 sequences correspond, from top to bottom, to SEQ ID NOs: 187-195.

FIG. 20. The stability of AVA04-251 V.2 over a nine-month period when stored in PBS 1× buffer at +4° C. conditions. SEC-HPLC analysis on a Yarra-3000 (Phenomenex) column run in PBS 1× buffer at 1 ml/min.

FIG. 21. The structure and SEC-HPLC/SDS-PAGE results for AVA04-251 CG. Final AVA04-251 CG was >98% pure on SEC-HPLC Yarra-3000 column run on an Ultimate 3000 HPLC (Thermo) at 1 ml/min in PBS 1× buffer. SDS-PAGE image shows kDa difference between reduced and non-reduced AVA04-251 CG.

FIG. 22. SEC-HPLC and SDS-PAGE results for AVA04-251 CF. Final AVA04-251 CF was >99% pure on SEC-HPLC Yarra-3000 column run on an Ultimate 3000 HPLC (Thermo) at 1 ml/min in PBS 1× buffer. SDS-PAGE image shows kDa difference between reduced and non-reduced AVA04-251 CF.

FIG. 23. Results of purification and kinetics analysis for AVA04-261 BN format. SDS-PAGE image shows dimeric species dimerising through cysteines in the hinge under non-reducing conditions and reduced to monomer in reducing buffer AVA04-261 BN format has avidity in the PD-1 PD-L1 blockade Bioassay (Promega) compared to monomeric AVA04-261 and 59.9 pM K_{D} on Biacore.

FIG. 24. Results of purification and kinetics analysis for AVA04-251 AZ human (AVA04-251 formatted on hFc1 with no linker). Final protein was >99% pure on SEC-HPLC Yarra-3000 column run on an Ultimate 3000 HPLC (Thermo) at 1 ml/min in PBS 1× buffer. Biacore single cycle kinetics data blank subtracted and fitted to a 1:1 binding model demonstrates a K_{D} of 31.5 pM. PD-1 PD-L1 blockade Bioassay (Promega) demonstrates the same activity as V.2 format of (G4S)4 (SEQ ID NO: 197) linker between Fc hinge and Affimer.

FIG. 25. Results of purification and kinetics analysis for AVA04-251 AG.3 and AVA04-251 BS format. Analytical SEC-HPLC was used to assess purity of each protein. Biacore single cycle kinetics data blank subtracted and fitted to a 1:1 binding model demonstrates a K_{D} of 36.2 pM and 25.7 pM for AVA04-251 AG.3 and AVA04-251 BS, respectively.

FIG. 26. Cross linking mass spectrometry of PD-L1 binding domain (14 kDa) with Fc fusion AVA04-251 V.2 or AVA04-236 V (82 kDa) to analyse stochiometry of non-covalent binding complexes.

FIGS. 27 and 28. Cross reactivity of binding to cynomologus PD-L1 of AVA04-251 Fc formatted.

FIGS. 29A-29B. Level of IFNγ in a Mixed Lymphocyte reaction that increase after AVA04-251_V.2 treatment.

FIG. 30. Increase of IL2 after treatment with AVA04-251_V.2 in a Staphylococcal enterotoxin B stimulation assay.

FIG. 31. The concentration in function of time dosed by ELISA in serum from mice injected with AVA04-251 XT format in C57/B16 mice, allowing to calculate half-life.

FIG. 32A-32B. In vivo Characterization of AVA04-251_V.2 in a tissue distribution experiment in humanized NOG mice bearing orthotopic MDA-MB-231 tumor cells.

FIGS. 33A-33D. In vivo efficacy of AVA04-251_V.2 that slow down the Tumor growth in a humanized MC38 syngeneic model.

FIGS. 34A-34D. In vivo effiacy of AVA04-251_V.2 in A375 Xenograft Model.

FIGS. 35A-35B. Activity of mouse surrogate AVA04-182 V.2 in a mouse allogenic Mixed Lymphocyte reaction (MLR) Assay. Data presented as individual reaction and mean+/−SEM*P<0.05,**p<0.01 using a paired t-test comparing reference substance to isotype control.

FIGS. 36A-36B. Tumor growth inhibition in the MB49 mouse syngeneic model (FIG. 36A); Tumor size at Day 21 post-treatment in the MB49 model (FIG. 36B).

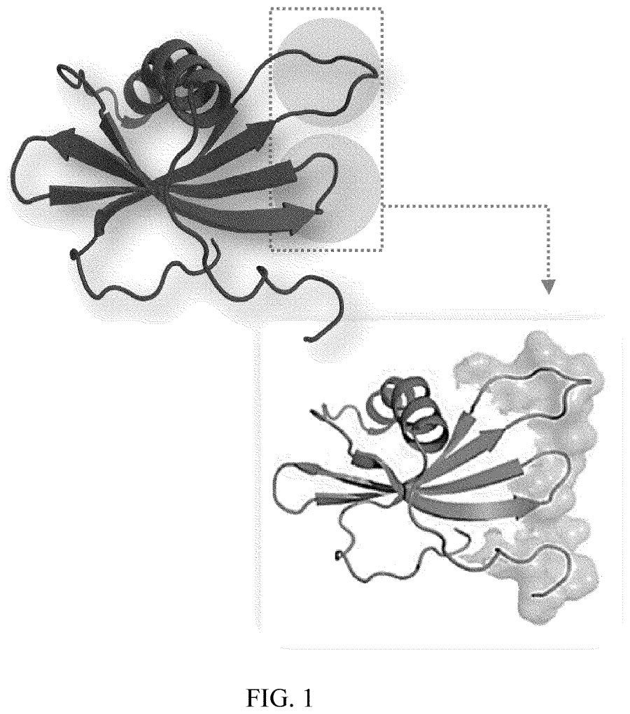

### DETAILED DESCRIPTION

### I. Overview

The present disclosure is based on the generation of affimers that bind to PD-L1 and inhibit the interation of that molecule with PD-1, and consequently represent checkpoint inhibitors that have utility in the treatment of cancers, metaplasias, neoplasias and certain viral and paracytic infections.

Based on naturally occurring proteins (cystatins) and engineered to stably display two loops which create a binding surface, the PD-L1 binding affimer polypeptides of the present disclosure provide a number of advantages over antibodies, antibody fragments and other non-antibody binding proteins.

One is the small size of the affimer polypeptide itself. In its monomeric form it is about 14 kDa, or 1/10th the size of an antibody. This small size gives greater potential for increased tissue penetration, particularly in poorly vascularized and/or fibrotic target tissues (like tumors).

Affimers have a simple protein structures (versus multi-domain antibodies), and as the affimers do not require disulfide bonds or other post-translational modifications for function, many of the format embodiments including these polypeptides can be manufactured in prokaryotic and eukaryotic systems.

The ability to utilize libraries of affimers (such as the phage display techniques described in the appended examples) as well as site directed mutagenesis, the affimers can be generated with tuneable binding kinetics with ideal ranges for therapeutic uses. For instance, the affimers can have high affinity for PD-L1, such as single digit nanomolar or lower K_{D} for monomeric affimers and picomolar K_{D} and avidity in multi-valent formats. The affimers can be generated with tight binding kinetics for PD-L1, such as slow Koff rates in the 10⁻⁴ to 10⁻⁵ (s-1) range which benefits target tissue localization.

The PD-L1 binding affimers of the present disclosure include affimers with exquisite selectivity.

Moreover, the PD-L1 binding affimers can be readily formatted, allowing formats such as Fc fusions, whole antibody fusions and in-line multimers to be generated and manufactured with ease.

The lack of need for disulfide bonds and post-translational modifications also permit many embodiments of proteins including the PD-L1 binding affimers (or monomeric affimer) to be delivered therapeutically by expression of gene delivery constructs that are introduced into the tissues of the patient, including formats where the protein is delivered systemically (such as expression from muscle tissue) or delivered locally (such as through intraturmoral gene delivery).

### II. Definitions

To facilitate an understanding of the present disclosure, a number of terms and phrases are defined below.

a. Affimer

The term “Stefin Polypeptide” refers to a sub-group of proteins in the cystatin superfamily, a family which encompasses proteins that contain multiple cystatin-like sequences.

The stefin sub-group of the cystatin family is relatively small (around 100 amino acids) single domain proteins. They receive no known post-translational modification, and lack disulphide bonds, suggesting that they will be able to fold identically in a wide range of extra- and intracellular environments. Stefin A itself is a monomeric, single chain, single domain protein of 98 amino acids. The structure of Stefin A has been solved, facilitating the rational mutation of Stefin A into the Affimer Scaffold. The only known biological activity of cystatins is the inhibition of cathepsin activity, which allowed us to exhaustively test for residual biological activity of our engineered proteins.

The term “Affimer” (or “Affimer Scaffold” or “Affimer Polypeptide”) refers to small, highly stable proteins that are a recombinantly engineered variants of Stefin Polypeptides. Affimer proteins display two peptide loops and an N-terminal sequence that can all be randomised to bind to desired target proteins with high affinity and specificity, in a similar manner to monoclonal antibodies. Stabilisation of the two peptides by the Stefin protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the Stefin polypeptide sequence can be carried out, with such variations improving the properties of these affinity reagents, such as increase stability, make them robust across a range of temperatures and pH and the like. Preferably the Affimer includes a sequence derived from stefin A, sharing substantial identify with a stefin A wild type sequence, such as human Stefin A. It will be apparent to a person skilled in the art that modifications may be made to the scaffold sequence without departing from the disclosure. In particular, an Affimer Scaffold can have an amino acid sequences that is at least 25%, 35%, 45%, 55% or 60% identity to the corresponding sequences to human Stefin A, preferably at least 70%, at least 80%, at least 85%, at least 90%, at least 92%, at least 94%, at least 95% identical, e.g., where the sequence variations do not adversely affect the ability of the scaffold to bind to the desired target (such as PD-L1), and e.g., which do not restore or generate biological functions such as those which are possessed by wild type stefin A but which are abolished in mutational changes described herein.

An “Affimer Agent” refers to a polypeptide including an Affimer Polypeptide sequence and having any other modifications (e.g., conjugation, post-translational modifications, etc) so as to represent the therapeutically active protein intended for delivery to a patient.

“Programmed death-ligand 1”, also known as “PD-L1”, “cluster of differentiation 274”, “CD274”, “B7 homolog 1” or “B7-H1”, refers a protein that, in the case of humans, is encoded by the CD274 gene. The human PD-L1 is a 40 kDa type 1 transmembrane protein that plays a major role in suppressing the immune system under different circumstances. A representative human PD-L1 sequence is provided by UniProtKB Primary accession number Q9NZQ7, and will include other human isoforms thereof. PD-L1 binds to its receptor, PD-1, found on activated T cells, B cells, and myeloid cells, to modulate activation or inhibition. PD-L1 also has an appreciable affinity for the costimulatory molecule CD80 (B7-1). Engagement of PD-L1 with its receptor PD-1 (“Programmed cell death protein 1” or “CD279”) on T cells delivers a signal that inhibits TCR-mediated activation of IL-2 production and T cell proliferation. In this regard, PD-L1 is considered a checkpoint, and its upregulated expression in tumors contributes to inhibition of T-cell mediated antitumor responses. While PD-L1 will be used generally with reference to PD-L1 from various mammalian species, it will be understood throughout the application that any reference to PD-L1 includes human PD-L1 and is, preferably, referring to human PD-L1 per se.

A “PD-L1 Affimer Agent” refers to an Affimer Agent having at least one Affimer Polypeptide that binds to PD-L1, particularly human PD-L1, with a dissociation constant (Kd) of at least 10⁻⁶M.

An “Encoded Affimer” refers to a nucleic acid construct which, when expressed by cells in a patient's body through a gene delivery process, produces an intended Affimer Agent in vivo.

An “Affimer-Linked Conjugate” refers to an Affimer Agent having one or more moieties conjugated thereto through a chemical conjugation other than through the formation of a contiguous peptide bond through the C-terminus or N-terminus of the polypeptide portion of the Affimer Agent containing Affimer Polypeptide sequence. An Affimer-linked Conjugate may be an “Affimer-Drug Conjugate”, which refers to an Affimer Agent including one or more pharmacologically active moieties conjugated thereto. An Affimer-linked Conjugate may also be an “Affimer-Tag Conjugate”, which refers to an Affimer Agent including one or more detectable moieties (i.e., detectable labels) conjugated thereto.

b. Polypeptides

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art.

The terms “amino acid residue” and “amino acid” are used interchangeably and means, in the context of a polypeptide, an amino acid that is participating in one more peptide bonds of the polypeptide. In general, the abbreviations used herein for designating the amino acids are based on recommendations of the IUPAC-IUB Commission on Biochemical Nomenclature (see Biochemistry (1972) 11:1726-1732). For instance, Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group. The term “amino acid side chain” is that part of an amino acid exclusive of the —CH(NH2)COOH portion, as defined by K. D. Kopple, “Peptides and Amino Acids”, W. A. Benjamin Inc., New York and Amsterdam, 1966, pages 2 and 33.

For the most part, the amino acids used in the application of this disclosure are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs which have been identified as constituents of peptidylglycan bacterial cell walls.

Amino acid residues having “basic sidechains” include Arg, Lys and His. Amino acid residues having “acidic sidechains” include Glu and Asp. Amino acid residues having “neutral polar sidechains” include Ser, Thr, Asn, Gln, Cys and Tyr. Amino acid residues having “neutral non-polar sidechains” include Gly, Ala, Val, Ile, Leu, Met, Pro, Trp and Phe. Amino acid residues having “non-polar aliphatic sidechains” include Gly, Ala, Val, Ile and Leu. Amino acid residues having “hydrophobic sidechains” include Ala, Val, Ile, Leu, Met, Phe, Tyr and Trp. Amino acid residues having “small hydrophobic sidechains” include Ala and Val. Amino acid residues having “aromatic sidechains” include Tyr, Trp and Phe.

The term amino acid residue further includes analogs, derivatives and congeners of any specific amino acid referred to herein, as for instance, the subject affimers (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally occurring amino acid metabolites or precursors having side chains which are suitable herein will be recognized by those skilled in the art and are included in the scope of the present disclosure.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this disclosure includes asymmetric carbon atoms. It is to be understood accordingly that the isomers arising from such asymmetry are included within the scope of this disclosure. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “fusion protein” or “fusion polypeptide” as used herein refers to a hybrid protein expressed by a nucleic acid molecule comprising nucleotide sequences of at least two genes.

The term “linker” or “linker region” as used herein refers to a linker inserted between a first polypeptide (e.g., copies of an affimer) and a second polypeptide (e.g., another affimer, an Fc domain, a ligand binding domain, etc). In some embodiments, the linker is a peptide linker. Linkers should not adversely affect the expression, secretion, or bioactivity of the polypeptides. Preferably, linkers are not antigenic and do not elicit an immune response.

An “Affimer-Antibody fusion” refers to a fusion protein including an affimer polypeptide portion and a variable region of an antibody. Affimer-Antibody fusions include full length antibodies having, for example, one or more affimer polypeptide sequences appended to the C-terminus or N-terminus of one or more of its VH and/or VL chains, i.e., at least one chain of the assembled antibody is a fusion protein with an affimer polypeptide. Affimer-Antibody fusions also include embodiments wherein one or more affimer polypeptide sequences are provided as part of a fusion protein with an antigen binding site or variable region of an antibody fragment.

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies provided those fragments have been formatted to include an Fc or other FcγRIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγRIII binding domain), and any other modified immunoglobulin molecule comprising an antigen-binding site as long as the antibodies exhibit the desired biological activity.

While the antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu.

The term “variable region” of an antibody refers to the variable region of an antibody light chain, or the variable region of an antibody heavy chain, either alone or in combination. Generally, the variable region of heavy and light chains each consist of four framework regions (FR) and three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

The terms “epitope” and “antigenic determinant” are used interchangeably herein and refer to that portion of an antigen capable of being recognized and specifically bound by a particular antibody, a particular affimer or other particular binding domain. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

As use herein, the term “specifically binds to” or is “specific for” refers to measurable and reproducible interactions such as binding between a target and an affimer, antibody or other binding partner, which is determinative of the presence of the target in the presence of a heterogeneous population of molecules including biological molecules. For example, an affimer that specifically binds to a target is an affimer that binds this target with greater affinity, avidity (if multimeric formatted), more readily, and/or with greater duration than it binds to other targets.

As used herein, “conjugate,” “conjugation” or grammatical variations thereof refers the joining or linking together of two or more compounds resulting in the formation of another compound, by any joining or linking methods known in the art. It can also refer to a compound which is generated by the joining or linking together two or more compounds. For example, an anti-PD-L1 affimer linked directly or indirectly to one or more chemical moieties or polypeptide is an exemplary conjugate. Such conjugates include fusion proteins, those produced by chemical conjugates and those produced by any other methods.

c. Nucleic Acids

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

As used herein, the term “nucleic acid molecule encoding”, “DNA sequence encoding,” and “DNA encoding” refer to the order or sequence of nucleotides along a strand of deoxyribonucleic acid deoxyribonucleotides. The order of these deoxyribonucleotides determines the order of amino acids along the polypeptide (protein) chain. Thus, a nucleic acid sequence encoding the amino acid sequence.

When used in reference to nucleotide sequences, “sequence” as used herein, the term grammatical and other forms may comprise DNA or RNA, and may be single or double stranded. Nucleic acid sequences may be mutated. Nucleic acid sequence may have any length, for example 2 to 000,000 or more nucleotides (or any integral value above or between) a nucleic acid, for example a length of from about 100 to about 10,000, or from about 200 nucleotides to about 500 nucleotides.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein, the term “transfection” refers to an exogenous nucleic acid into a eukaryotic cell. Transfection can be achieved by various means known in the art, including calcium phosphate-DNA co-precipitation, DEAE-dextran-mediated transfection, polybrene-mediated transfection, electroporation, microinjection, liposome fusion, lipofection, protoplast fusion, retroviral infection, and biolistics technology (biolistics).

The term “carrier” as used herein is an isolated nucleic acid comprising the isolated nucleic acid can be used to deliver a composition to the interior of the cell. It is known in the art a number of carriers including, but not limited to the linear polynucleotides, polynucleotides associated with ionic or amphiphilic compounds, plasmids, and viruses. Thus, the term “vector” includes an autonomously replicating plasmid or virus. The term should also be construed to include facilitate transfer of nucleic acid into cells of the non-plasmid and non-viral compounds, for example, polylysine compounds, liposomes, and the like. Examples of viral vectors include, but are not limited to adenoviral vectors, adeno-associated virus vectors, retroviral vectors and the like.

As used herein, the term “expression vector” refers to a vector comprising a recombinant polynucleotide comprising expression control sequence and a nucleotide sequence to be expressed operably linked. The expression vector comprises sufficient cis-acting elements (cis-acting elements) used for expression; other elements for expression can be supplied by the host cell or in vitro expression system. Expression vectors include all those known in the art, such as cosmids, plasmids (e.g., naked or contained in liposomes) and viruses (e.g., lentivirus, retroviruses, adenoviruses and adeno-associated viruses).

As used herein, the term “operably linked” refers to functional linkage between the regulatory sequence and a heterologous nucleic acid sequence is connected to a connection results in the expression of the latter. For example, when the first nucleic acid sequence and a second nucleic acid sequence is a functional relationship between the first nucleic acid sequence and the second nucleic acid sequence is operably linked. For example, if the promoter affects the transcription or expression of the coding sequence, the promoter is operably linked to a coding sequence. Typically, DNA sequencing operably linked are contiguous, and to join two protein coding regions in the same reading frame as necessary.

As used herein, the term “promoter” is defined as a promoter DNA sequence recognized by the synthetic machinery required for the synthesis machinery of the cell specific transcription of a polynucleotide sequence or introduced.

The term “constitutive expression” as used herein refers to all expressed under physiological conditions.

The term “inducible expression” as used herein refers to expression under certain conditions, such as activation (or inactivation) of an intracellular signaling pathway or the contacting of the cells harboring the expression construct with a small molecule that regulates the expression (or degree of expression) of a gene operably linked to an inducible promoter sensitive to the concentration of the small molecule.

The term “electroporation” refers to the use of a transmembrane electric field pulse to induce microscopic pathways (pores) in a bio-membrane; their presence allows biomolecules such as plasmids or other oligonucleotide to pass from one side of the cellular membrane to the other.

d. Checkpoint Inhibitors, Co-Stimulatory Agonists and Chemotherapeutics

A “checkpoint molecule” refers to proteins that are expressed by tissues and/or immune cells and reduce the efficacy of an immune response in a manner dependent on the level of expression of the checkpoint molecule. When these proteins are blocked, the “brakes” on the immune system are released and, for example, T cells are able to kill cancer cells more effectively. Examples of checkpoint proteins found on T cells or cancer cells include PD-1/PD-L1 and CTLA-4/B7-1/B7-2, PD-L2, NKG2A, KIR, LAG-3, TIM-3, CD96, VISTA and TIGIT.

A “checkpoint inhibitor” refers to a drug entity that reverses the immunosuppressive signaling from a checkpoint molecule.

A “costimulatory molecule” refers to an immune cell such as a T cell cognate binding partner which specifically binds to costimulatory ligands thereby mediating co-stimulation, such as, but not limited to proliferation. Costimulatory molecules are cell surface molecules other than the antigen receptor or ligand which facilitate an effective immune response. Co-stimulatory molecules include, but are not limited to MHCI molecules, BTLA receptor and Toll ligands, and OX40, CD27, CD28, CDS, ICAM-1, LFA-1 (CD11a/CD18), ICOS (CD278) and 4-1BB (CD137). Examples of costimulatory molecules include but are not limited to: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244,2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and CD83 ligand.

A “costimulatory agonists” refers to a drug entity that activates (agonizes) the costimulatory molecule, such as costimulatory ligand would do, and produces an immunostimulatory signal or otherwise increases the potency or efficacy of an immune response.

A “chemotherapeutic agent” is a chemical compound useful in the treatment of cancer. Examples of chemotherapeutic agents include alkylating agents such as thiotepa and cyclophosphamide (CYTOXAN); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN, morpholino-doxorubicin, cyanomorpholino-doxorubicin, 2-pyrrolino-doxorubicin, doxorubicin HCl liposome injection (DOXIL) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR), tegafur (UFTORAL), capecitabine (XELODA), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE, FILDESIN); dacarbazine; mannomustine; mitobronitol; mitolactol; pipobroman; gacytosine; arabinoside (“Ara-C”); thiotepa; taxoids, e.g., paclitaxel (TAXOL), albumin-engineered nanoparticle formulation of paclitaxel (ABRAXANE), and doxetaxel (TAXOTERE); chloranbucil; 6-thioguanine; mercaptopurine; methotrexate; platinum analogs such as cisplatin and carboplatin; vinblastine (VELBAN); platinum; etoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), raloxifene (EVISTA), droloxifene, 4-hydroxytamoxifen, trioxifene, keoxifene, LY117018, onapristone, and toremifene (FARESTON); anti-progesterones; estrogen receptor down-regulators (ERDs); estrogen receptor antagonists such as fulvestrant (FASLODEX); agents that function to suppress or shut down the ovaries, for example, leutinizing hormone-releasing hormone (LHRH) agonists such as leuprolide acetate (LUPRON and ELIGARD), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE), exemestane (AROMASIN), formestanie, fadrozole, vorozole (RIVISOR), letrozole (FEMARA), and anastrozole (ARIMIDEX). In addition, such definition of chemotherapeutic agents includes bisphosphonates such as clodronate (for example, BONEFOS or OSTAC), etidronate (DIDROCAL), NE-58095, zoledronic acid/zoledronate (ZOMETA), alendronate (FOSAMAX), pamidronate (AREDIA), tiludronate (SKELID), or risedronate (ACTONEL); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; topoisomerase 1 inhibitor (e.g., LURTOTECAN); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

e. Treatments

The term “dysfunctional”, as used herein, also includes refractory or unresponsive to antigen recognition, specifically, impaired capacity to translate antigen recognition into downstream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g. increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overridden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors.

“Enhancing T-cell function” means to induce, cause or stimulate a T-cell to have a sustained or amplified biological function, or renew or reactivate exhausted or inactive T-cells. Examples of enhancing T-cell function include: increased secretion of □-interferon from CD8+ T-cells, increased proliferation, increased antigen responsiveness (e.g., viral, pathogen, or tumor clearance) relative to such levels before the intervention. In some embodiments, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation. In a particular embodiment, a T-cell dysfunctional disorder is a disorder that is specifically associated with inappropriate increased levels of PD-1. A T-cell dysfunctional disorder can also be associated with inappropriate increased levels of PD-L1 in the tumor which gives rise to suppression of T-cell antitumor function(s). In another embodiment, a T-cell dysfunctional disorder is one in which T-cells are anergic or have decreased ability to secrete cytokines, proliferate, or execute cytolytic activity. In a specific aspect, the decreased responsiveness results in ineffective control of a pathogen or tumor expressing an immunogen. Examples of T cell dysfunctional disorders characterized by T-cell dysfunction include unresolved acute infection, chronic infection and tumor immunity.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0×length of the treatment duration.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions. Tumor growth is generally uncontrolled and progressive, does not induce or inhibit the proliferation of normal cells. Tumor can affect a variety of cells, tissues or organs, including but not limited to selected from bladder, bone, brain, breast, cartilage, glial cells, esophagus, fallopian tube, gall bladder, heart, intestine, kidney, liver, lung, lymph node, neural tissue, ovary, pancreas, prostate, skeletal muscle, skin, spinal cord, spleen, stomach, testis, thymus, thyroid, trachea, urethra, ureter, urethra, uterus, vagina organ or tissue or the corresponding cells. Tumors include cancers, such as sarcoma, carcinoma, plasmacytoma or (malignant plasma cells). Tumors of the present disclosure, may include, but are not limited to leukemias (e.g., acute leukemia, acute lymphoblastic leukemia, acute myeloid leukemia, acute myeloid leukemia, acute promyelocytic leukemia, acute myeloid-monocytic leukemia, acute monocytic leukemia, acute leukemia, chronic leukemia, chronic myeloid leukemia, chronic lymphocytic leukemia, polycythemia vera), lymphomas (Hodgkin's disease, non-Hodgkin's disease), primary macroglobulinemia disease, heavy chain disease, and solid tumors such as sarcomas cancer (e.g., fibrosarcoma, myxosarcoma, liposarcoma, chondrosarcoma, osteosarcoma, chordoma, endothelium sarcoma, lymphangiosarcoma, angiosarcoma, lymphangioendothelio sarcoma, synovioma vioma, mesothelioma, Ewing's tumor, leiomyosarcoma, rhabdomyosarcoma, colon carcinoma, pancreatic cancer, breast cancer, (including triple negative breast cancer), ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, carcinoma, bronchogenic carcinoma, medullary carcinoma, renal cell carcinoma, hepatoma, Nile duct carcinoma, choriocarcinoma, spermatogonia Tumor, embryonal carcinoma, Wilms' tumor, cervical cancer, uterine cancer, testicular cancer, lung carcinoma (including small cell lung carcinoma and non-small cell lung carcinoma or NSCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma), esophageal cancer, gallbladder, kidney cancer, multiple myeloma. Preferably, a “tumor” includes, but is not limited to: pancreatic cancer, liver cancer, lung cancer (including NSCLC), stomach cancer, esophageal cancer, head and neck squamous cell carcinoma, prostate cancer, colon cancer, breast cancer (including triple negative breast cancer), lymphoma, gallbladder cancer, renal cancer, leukemia, multiple myeloma, ovarian cancer, cervical cancer and glioma.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “effective amount” as used herein refers to an amount to provide therapeutic or prophylactic benefit.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

The term “treatment” as used herein refers to the individual trying to change the process or treatment of a clinical disease caused by intervention of a cell, may be either preventive intervention course of clinical pathology. Including but not limited to treatment to prevent the occurrence or recurrence of disease, alleviation of symptoms, reducing the direct or indirect pathological consequences of any disease, preventing metastasis, slow the rate of disease progression, amelioration or remission of disease remission or improved prognosis.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “agonist” and “agonistic” as used herein refer agents that are capable of, directly or indirectly, substantially inducing, activating, promoting, increasing, or enhancing the biological activity of a target or target pathway. The term “agonist” is used herein to include any agent that partially or fully induces, activates, promotes, increases, or enhances the activity of a protein or other target of interest.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein or other target of interest.

The terms “modulation” and “modulate” as used herein refer to a change or an alteration in a biological activity. Modulation includes, but is not limited to, stimulating an activity or inhibiting an activity. Modulation may be an increase in activity or a decrease in activity, a change in binding characteristics, or any other change in the biological, functional, or immunological properties associated with the activity of a protein, a pathway, a system, or other biological targets of interest.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one agent of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of an Affimer Agent described herein effective to “treat” a disease or disorder in a subject such as, a mammal. In the case of cancer or a tumor, the therapeutically effective amount of an PD-L1 binding Affimer Agent has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present disclosure if the patient shows one or more of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

f. Miscellaneous

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### III. PD-L1 Binding Affimers

An affimer is a scaffold based on stefin A, meaning that it has a sequence which is derived from stefin A, for example, a mammalian stefin A, for example, a human stefin A. Some aspects of the application provides affimers which bind PD-L1 (also referred to as “anti-PD-L1 affimers”) comprising an affimer in which one or more of the solvent accessible loops from the wild-type stefin A protein with amino acid sequences to provide an affimer having the ability to bind PD-L1, preferably selectively, and preferably with Kd of 10⁻⁶M or less.

In some embodiments, the anti-PD-L1 affimer is derived from the wild-type human stefin A protein having a backbone sequence and in which one or both of loop 2 [designated (Xaa] and loop 4 [designated (Xaa)ₘ] are replaced with alternative loop sequences (Xaa)ₙ and (Xaa)ₘ, to have the general formula (i)

FR1-(Xaa)ₙ-FR2-(Xaa)ₘ-FR3 (I)

wherein

FR1 is a polypeptide sequence represented by MIPGGLSEAK PATPEIQEIV DKVKPQLEEK TNETYGKLEA VQYKTQVLA (SEQ ID NO: 1) or a polypeptide sequence having at least 70% homology thereto;

FR2 is a polypeptide sequence represented by GTNYYIKVRA GDNKYMHLKV FKSL (SEQ ID NO: 2) or a polypeptide sequence having at least 70% homology thereto;

FR3 is a polypeptide sequence represented by EDLVLTGYQV DKNKDDELTG F (SEQ ID NO: 3) or a polypeptide sequence having at least 70% homology thereto; and

Xaa, individually for each occurrence, is an amino acid residue, n and m are each, independently, an integer from 3 to 20.

In some embodiments, FR1 is a polypeptide sequence having at least 80%, 85%, 90%, 95% or even 98% homology with SEQ ID NO: 1. In some embodiments, FR1 is a polypeptide sequence having at least 80%, 85%, 90%, 95% or even 98% identity with SEQ ID NO: 1; In some embodiments, FR2 is a polypeptide sequence having at least 80%, 85%, 90%, 95% or even 98% homology with SEQ ID NO: 2. In some embodiments, FR2 is a polypeptide sequence having at least 80%, 85%, 90%, 95% or even 98% identity with SEQ ID NO: 2; In some embodiments, FR3 is a polypeptide sequence having at least 80%, 85%, 90%, 95% or even 98% homology with SEQ ID NO: 3. In some embodiments, FR3 is a polypeptide sequence having at least 80%, 85%, 90%, 95% or even 98% identity with SEQ ID NO: 3.

In some embodiments, the anti-PD-L1 affimer has an amino acid sequence represented in the general formula:

(SEQ ID NO: 4)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF

wherein

Xaa, individually for each occurrence, is an amino acid residue; n and m are each, independently, an integer from 3 to 20; Xaa1 is Gly, Ala, Val, Arg, Lys, Asp, or Glu, more preferably Gly, Ala, Arg or Lys, and more even more preferably Gly or Arg; Xaa2 is Gly, Ala, Val, Ser or Thr, more preferably Gly or Ser; Xaa3 is Arg, Lys, Asn, Gln, Ser, Thr, more preferably Arg, Lys, Asn or Gln, and even more preferably Lys or Asn; Xaa4 is Gly, Ala, Val, Ser or Thr, more preferably Gly or Ser; Xaa5 is Ala, Val, Ile, Leu, Gly or Pro, more preferably Ile, Leu or Pro, and even more preferably Leu or Pro; Xaa6 is Gly, Ala, Val, Asp or Glu, more preferably Ala, Val, Asp or Glu, and even more preferably Ala or Glu; and Xaa5 is Ala, Val, Ile, Leu, Arg or Lys, more preferably Ile, Leu or Arg, and even more preferably Leu or Arg.

For instance, the anti-PD-L1 affimer can have an amino acid sequence represented in the general formula:
MIPRGLSEAKPATPEIQEIVDKVKPQLEEKTNETYGKLEAVQYKTQVLA-(Xaa)ₙ-STNYYIKVRAGDNKYMHLKVFNGP-(Xaa)ₘ-ADRVLTGYQVDKNKDDELTGF (SEQ ID NO: 5), wherein Xaa, individually for each occurrence, is an amino acid residue; n and m are each, independently, an integer from 3 to 20.

In some embodiments, n is 3 to 15, 3 to 12, 3 to 9, 3 to 7, 5 to 7, 5 to 9, 5 to 12, 5 to 15, 7 to 12 or 7 to 9.

In some embodiments, m is 3 to 15, 3 to 12, 3 to 9, 3 to 7, 5 to 7, 5 to 9, 5 to 12, 5 to 15, 7 to 12 or 7 to 9.

In some embodiments, Xaa, independently for each occurrence, is an amino acid that can be added to a polypeptide by recombinant expression in a prokaryotic or eukaryotic cell, and even more preferably one of the 20 naturally occurring amino acids.

In some embodiments of the above sequences and formulas, (Xaa)ₙ is an amino acid sequence represented in the general formula (II)

-aa1-aa2-aa3-Gly-Pro-aa4-aa5-Trp-aa6- (II)

wherein

aa1 represents an amino acid residue with a basic sidechain, more preferably Lys, Arg or His, and even more preferably Lys or Arg;

aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain, even more preferably Ala, Pro, Ile, Gln, Thr, Asp, Glu, Lys, Arg or His, and even more preferably Ala, Gln, Asp or Glu;

aa3 represents an amino acid residue with an aromatic or basic sidechain, preferably Phe, Tyr, Trp, Lys, Arg or His, more preferably Phe, Tyr, Trp, and even more preferably His or Tyr, Trp or His;

aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu;

aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg; and

aa6 represents an amino acid residue with an aromatic or acid sidechain, preferably Phe, Tyr, Trp, Asp or Glu; more preferably Trp or Asp; and even more preferably Trp.

In some embodiments of the above sequences and formulas, (Xaa)ₙ is an amino acid sequence represented in the general formula (III)

-aa1-aa2-aa3-Phe-Pro-aa4-aa5-Phe-Trp- (III)

wherein

aa1 represents an amino acid residue with a basic sidechain or aromatic sidechain, preferably Lys, Arg, His, Ser, Thr, Asn or Gln, more preferably Lys, Arg, His, Asn or Gln, and even more preferably Lys or Asn;

aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain, even more preferably Ala, Pro, Ile, Gln, Thr, Asp, Glu, Lys, Arg or His, and even more preferably Ala, Gln, Asp or Glu;

aa3 represents an amino acid residue with an aromatic or basic sidechain, preferably Phe, Tyr, Trp, Lys, Arg or His, more preferably Phe, Tyr, Trp or His, and even more preferably Tyr, Trp or His;

aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu; and

aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg.

In some embodiments of the above sequences and formulas, (Xaa)ₙ is an amino acid sequence selected from SEQ ID NOS: 6 to 40, or an amino acid sequence having at least 80%, 85%, 90%, 95% or even 98% homology with a sequence selected from SEQ ID NOS: 6 to 41. In some embodiments, (Xaa)ₙ is an amino acid sequence having at least 80%, 85%, 90%, 95% or even 98% identity with a sequence selected from SEQ ID NO: 6 to 41.

| Loop 2 sequences | SEQ ID NO. |
|---|---|
| KAWGPKQWW | 6 |
| KPYGPRDWD | 7 |
| KEYGPEEWW | 8 |
| HAYGPRDWD | 9 |
| KDHGPIAWW | 10 |
| NKHFHQRFW | 11 |
| NKHFPIHFW | 12 |
| HEFGPAEWD | 13 |
| NAHFPQSFW | 14 |
| KEHGPDSWW | 15 |
| NQHFPHSFW | 16 |
| NAHFGPRFW | 17 |
| NTWFPESFW | 18 |
| NQHFPQSFW | 19 |
| KQYGPDDWW | 20 |
| KDWGPSNWW | 21 |
| KQFGPKDWW | 22 |
| NHHFPKRFW | 23 |
| YRHFPQWH | 24 |
| NIHFPPNFW | 25 |
| YTHFPQWT | 26 |
| NDHFPHTFW | 27 |
| NQHFPSYFW | 28 |
| NQYFPPHFW | 29 |
| KKHFPASFW | 30 |
| KKFFPKHFW | 31 |
| KLHFPRSFW | 32 |
| YKHFPPNFW | 33 |
| EEHFPFQFW | 34 |
| KPHFPDNFW | 35 |
| YQYFPDQFN | 36 |
| VQWFPRSFW | 37 |
| AAHFPEHFW | 38 |
| REGRQDWVL | 39 |
| WVPFPHQQL | 40 |
| YNHFPEYMW | 41 |

In some embodiments of the above sequences and formulas, (Xaa)ₘ is an amino acid sequence represented in the general formula (IV)

-aa7-aa8-aa9-aa10-aa11-aa12-aa13-aa14-aa15- (IV)

wherein

aa7 represents an amino acid residue with neutral polar or non-polar sidechain or an acidic sidechain; preferably Gly, Ala, Val, Pro, Trp, Gln, Ser, Asp or Glu, and even more preferably Gly, Ala, Trp, Gln, Ser, Asp or Glu;

aa8 represents an amino acid residue, preferably an amino acid residue with a neutral polar or non-polar sidechain or a charged (acidic or basic) sidechain or aromatic sidechain, more preferably a charged (acidic or basic) sidechain, more preferably Asp, Glu, Lys, Arg, His, Gln, Ser, Thr, Asn, Ala, Val, Pro, Gly, Tyr or Phe, and even more preferably Asp, Glu, Lys, Arg, His or Gln;

aa9 represents an amino acid residue, preferably an amino acid residue with a neutral polar or non-polar sidechain or a charged (acidic or basic) sidechain or aromatic sidechain, more preferably a neutral polar side chain or an acid side chain, more preferably Gln, Ser, Thr, Asn, Asp, Glu, Arg, Lys, Gly, Leu, Pro or Tyr, and even more preferably Gln, Thr or Asp;

aa10 represents an amino acid residue, preferably an amino acid residue with a neutral polar or non-polar sidechain or a charged (acidic or basic) sidechain or aromatic sidechain, more preferably a neutral polar side chain or a basic or acid side chain, more preferably Asp, Glu, Arg, His, Lys, Ser, Gln, Asn, Ala, Leu, Tyr, Trp, Pro or Gly, and even more preferably Asp, Glu, His, Gln, Asn, Leu, Trp or Gly;

aa11 represents an amino acid residue, preferably an amino acid residue with a neutral polar sidechain or a charged (acidic or basic) sidechain or a nonpolar aliphatic sidechain or an aromatic sidechain, more preferably a neutral polar side chain or a basic or acid side chain, more preferably Asp, Glu, Ser, Thr, Gln, Arg, Lys, His, Val, Ile, Tyr or Gly and even more preferably Asp, Glu, Ser, Thr, Gln, Lys or His;

aa12 represents an amino acid residue, preferably an amino acid residue with a neutral polar sidechain or a charged (acidic or basic) sidechain or a nonpolar aliphatic sidechain or an aromatic sidechain, more preferably a an acid side chain, more preferably Asp, Glu, Ser, Thr, Gln, Asn, Lys, Arg, Val, Leu, Ile, Trp, Tyr, Phe or Gly and even more preferably Asp, Glu, Ser, Tyr, Trp, Arg or Lys;

aa13 represents an amino acid residue, preferably an amino acid residue with a neutral polar sidechain or a charged (acidic or basic) sidechain or a nonpolar aliphatic sidechain or an aromatic sidechain, more preferably a an acid side chain, more preferably Ser, Thr, Gln, Asn, Val, Ile, Leu, Gly, Pro, Asp, Glu, His, Arg, Trp, Tyr or Phe and even more preferably Ser, Thr, Gln, Asn, Val, Ile, Leu, Gly, Asp or Glu;

aa14 represents an amino acid residue, preferably an amino acid residue with a neutral polar sidechain or a charged (acidic or basic) sidechain, more preferably Ala, Ile, Trp, Pro, Asp, Glu, Arg, Lys, His, Ser, Thr, Gln or Asn and even more preferably Ala, Pro, Asp, Glu, Arg, Lys, Ser, Gln or Asn; and

aa15 represents an amino acid residue, preferably an amino acid residue with a neutral polar or neutral non-polar sidechain or a charged (acidic or basic) sidechain, more preferably His, Arg, Lys, Asp, Ser, Thr, Gln, Asn, Ala, Val, Leu, Gly or Phe and even more preferably His, Arg, Lys, Asp, Ser, Thr, Gln or Asn.

In some embodiments of the above sequences and formulas, (Xaa)ₘ is an amino acid sequence selected from SEQ ID NOS: 42 to 77, or an amino acid sequence having at least 80%, 85%, 90%, 95% or even 98% homology with a sequence selected from SEQ ID NOS: 42 to 77. In some embodiments, (Xaa)ₘ is an amino acid sequence having at least 80%, 85%, 90%, 95% or even 98% identity with a sequence selected from SEQ ID NO: 42 to 77.

| Loop 4 sequences | SEQ ID No. |
|---|---|
| GRTIQ | 42 |
| EPQLDTSPI | 43 |
| GDYEQVLIH | 44 |
| PADHVLEEA | 45 |
| EDTNTDGAL | 46 |
| GQSWDQRRQ | 47 |
| SKSPIDLPF | 48 |
| DPQDVYLNQ | 49 |
| GSLHSFGST | 50 |
| QEKNQWVEE | 51 |
| QKNYEEDPH | 52 |
| WDGHKRFAD | 53 |
| DDNQERQEH | 54 |
| AVTQEDQAV | 55 |
| EVDWKYQDH | 56 |
| VDDKTLSKD | 57 |
| QGQGKDPSQ | 58 |
| GHQSEVQHS | 59 |
| TGTSIWNQD | 60 |
| GVHDSLQGYDA | 61 |
| QKGQKIDKF | 62 |
| DDELHDTRH | 63 |
| ATTGDEWDR | 64 |
| SHPHSNHTS | 65 |
| WRTDYKYEE | 66 |
| NDPHDSVPH | 67 |
| GQQRENEQE | 68 |
| GERQQDDAN | 69 |
| AYREGSQWT | 70 |
| EFYDHGIIQ | 71 |
| ENEATRDQH | 72 |
| GYDHEDNRG | 73 |
| QPADMSAEF | 74 |
| WVPFPHQQL | 75 |
| REGRQDWVL | 76 |
| PPRFPHEPF | 77 |

In some embodiments, the anti-PD-L1 affimer has an amino acid sequence selected from SEQ ID NOS: 78 to 86, or an amino acid sequence having at least 70%, 75% 80%, 85%, 90%, 95% or even 98% homology with a sequence selected from SEQ ID NOS: 78 to 86. In some embodiments, the anti-PD-L1 affimer has an amino acid sequence having at least 70%, 75% 80%, 85%, 90%, 95% or even 98% identity with a sequence selected from SEQ ID NO: 78 to 86.

| | SEQ |
|---|---|
| | ID |
| Exemplary anti-PD-L1 Affimer Sequences | NO. |
| MIPRGLSEAK PATPEIQEIV DKVKPQLEEK TNETYGKLEA | 78 |
| VQYKTQVLAK EHGPDSWWST NYYIKVRAGD NKYMHLKVFN | |
| GPQEKNQWVE EADRVLTGYQ VDKNKDDELT GF | |
| MIPRGLSEAK PATPEIQEIV DKVKPQLEEK TNETYGKLEA | 79 |
| VQYKTQVLAK EYGPEEWWST NYYIKVRAGD NKYMHLKVFN | |
| GPGDYEQVLI HADRVLTGYQ VDKNKDDELT GF | |
| MIPRGLSEAK PATPEIQEIV DKVKPQLEEK TNETYGKLEA | 80 |
| VQYKTQVLAK DHGPIAWWST NYYIKVRAGD NKYMHLKVFN | |
| GPEDTNTDGA LADRVLTGYQ VDKNKDDELT GF | |
| MIPRGLSEAK PATPEIQEIV DKVKPQLEEK TNETYGKLEA | 81 |
| VQYKTQVLAK DWGPSNWWST NYYIKVRAGD NKYMHLKVFN | |
| GPVDDKTLSK DADRVLTGYQ VDKNKDDELT GF | |
| MIPRGLSEAK PATPEIQEIV DKVKPQLEEK TNETYGKLEA | 82 |
| VQYKTQVLAN TWFPESFWST NYYIKVRAGD NKYMHLKVFN | |
| GPDDNQERQE HADRVLTGYQ VDKNKDDELT GF | |
| MIPRGLSEAK PATPEIQEIV DKVKPQLEEK TNETYGKLEA | 83 |
| VQYKTQVLAK PYGPRDWDST NYYIKVRAGD NKYMHLKVFN | |
| GPEPQLDTSP IADRVLTGYQ VDKNKDDELT GF | |
| MIPRGLSEAK PATPEIQEIV DKVKPQLEEK TNETYGKLEA | 84 |
| VQYKTQVLAH AYGPRDWDST NYYIKVRAGD NKYMHLKVFN | |
| GPPADHVLEE AADRVLTGYQ VDKNKDDELT GF | |
| MIPRGLSEAK PATPEIQEIV DKVKPQLEEK TNETYGKLEA | 85 |
| VQYKTQVLAA AHFPEHFWST NYYIKVRAGD NKYMHLKVFN | |
| GPQPADMSAE FADRVLTGYQ VDKNKDDELT GF | |
| MIPRGLSEAK PATPEIQEIV DKVKPQLEEK TNETYGKLEA | 86 |
| VQYKTQVLAR EGRQDWVLST NYYIKVRAGD NKYMHLKVFN | |
| GPWVPFPHQQ LADRVLTGYQ VDKNKDDELT GF | |

In some embodiments, the anti-PD-L1 affimer has an amino acid sequence that is encoded by a nucleic acid having a coding sequence corresponding to nucleotides 1-336 of one of SEQ ID NOS: 87 to 94, or an amino acid sequence that can be encoded by a nucleic acid having a coding sequence at least 70%, 75% 80%, 85%, 90%, 95% or even 98% identical with nucleotides 1-336 of one of SEQ ID NOS: 87 to 94, or an amino acid sequence that can be encoded by a nucleic acid having a coding sequence that hybridizes nucleotides 1-336 of one of SEQ ID NOS: 87 to 94 under stringent conditions (such as in the presence of 6× sodium chloride/sodium citrate (SSC) at 45° C. followed by a wash in 0.2×SSC at 65° C.

| | SEQ |
|---|---|
| Exemplary anti-PD-L1 Affimer | ID |
| Coding Sequences | NO. |
| ATGATCCCGCGTGGCCTGTCTGAAGCTAAACCAGCAACTCCGG | 87 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCAAAAGATTGGGGTCCATCTAACTGGT | |
| GGTCCACCAACTATTACATTAAGGTTCGTGCCGGTGACAATAA | |
| GTATATGCACCTGAAAGTGTTCAACGGCCCGGTTGATGATAAA | |
| ACCCTGTCTAAAGATGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 88 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCAAAAGATCATGGTCCAATCGCATGGT | |
| GGTCCACCAACTATTACATTAAGGTTCGTGCCGGTGACAATAA | |
| GTATATGCACCTGAAAGTGTTCAACGGCCCGGAAGATACCAAC | |
| ACCGATGGTGCACTGGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 89 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCAAAACCATACGGTCCACGTGATTGGG | |
| ATTCCACCAACTATTACATTAAGGTTCGTGCCGGTGACAATAA | |
| GTATATGCACCTGAAAGTGTTCAACGGCCCGGAACCACAGCTG | |
| GATACCTCTCCAATCGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 90 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCAAACACCTGGTTTCCAGAATCTTTTT | |
| GGTCCACCAACTATTACATTAAGGTTCGTGCCGGTGACAATAA | |
| GTATATGCACCTGAAAGTGTTCAACGGCCCGGATGATAACCAG | |
| GAACGTCAGGAACATGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 91 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCACGTGAAGGTCGTCAGGATTGGGTTC | |
| TGTCCACCAACTATTACATTAAGGTTCGTGCCGGTGACAATAA | |
| GTATATGCACCTGAAAGTGTTCAACGGCCCGTGGGTTCCATTT | |
| CCACATCAGCAGCTGGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 92 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCACATGCATACGGTCCACGTGATTGGG | |
| ATTCCACCAACTATTACATTAAGGTTCGTGCCGGTGACAATAA | |
| GTATATGCACCTGAAAGTGTTCAACGGCCCGCCAGCAGATCAT | |
| GTTCTGGAAGAAGCAGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 93 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCAAAAGAATACGGTCCAGAAGAATGGT | |
| GGTCCACCAACTATTACATTAAGGTTCGTGCCGGTGACAATAA | |
| GTATATGCACCTGAAAGTGTTCAACGGCCCGGGTGATTACGAA | |
| CAGGTTCTGATCCATGCGGACCGTGTTCTGACCGGTTACCAGG | |
| TTGACAAGAACAAAGATGACGAGCTGACGGGTTTCGCGGCCGC | |
| GGGTCATCACCACCACCACCATTAG | |
| ATGATCCCGCGTGGCCTGTCTGAAGCTAAACCAGCAACTCCGG | 94 |
| AAATTCAAGAGATCGTCGATAAGGTGAAACCGCAGCTGGAAGA | |
| GAAAACGAACGAAACCTACGGTAAGCTGGAAGCGGTCCAGTAC | |
| AAAACCCAAGTGCTAGCAGCTGCTCATTTCCCGGAACATTTCT | |
| GGTCCACCAACTATTACATTAAGGTTCGTGCCGGTGACAATAA | |
| GTATATGCACCTGAAAGTGTTCAACGGCCCGCAGCCGGCTGAT | |
| ATGTCTGCTGAATTCGCGGACCGTGTTCTGACCGGTTACCAGG | |
| TTGACAAGAACAAAGATGACGAGCTGACGGGTTTCCTGCAGGC | |
| GGCCGCGCACCACCACCACCACCACTG | |

Furthermore, minor modifications may also include small deletions or additions—beyond the loop 2 and loop 4 inserts described above—to the stefin A or stefin A derived sequences disclosed herein, such as addition or deletion of up to 10 amino acids relative to stefin A or the stefin A derived Affimer polypeptide.

In some embodiments, the Affimer Agent is a PD-L1 binding Affimer Agent having an Affimer polypeptide portion that binds human PD-L1 as a monomer with a dissociation constant (K_{D}) of about 1 μM or less, about 100 nM or less, about 40 nM or less, about 20 nM or less, about 10 nM or less, about 1 nM or less, or about 0.1 nM or less.

In some embodiments, the Affimer Agent is a PD-L1 binding Affimer Agent having an Affimer polypeptide portion that binds human PD-L1 as a monomer with an off rate constant (K_{off}), such as measured by Biacore, of about 10⁻³ s⁻¹ (i.e., unit of 1/second) or slower; of about 10⁻⁴ s⁻¹ or slower or even of about 10⁻⁵ s⁻¹ or slower.

In some embodiments, the Affimer Agent is a PD-L1 binding Affimer Agent having an Affimer polypeptide portion that binds human PD-L1 as a monomer with an association constant (Kₒₙ), such as measured by Biacore, of at least about 10³ M⁻¹ s⁻¹ or faster; at least about 10⁴ M⁻¹ s⁻¹ or faster; at least about 10⁵ M⁻¹ s⁻¹ or faster; or even at least about 10⁶ M⁻¹ s⁻¹ or faster.

In some embodiments, the Affimer Agent is a PD-L1 binding Affimer Agent having an Affimer polypeptide portion that binds human PD-L1 as a monomer with an IC50 in a competitive binding assay with human PD-1 of 1 μM or less, about 100 nM or less, about 40 nM or less, about 20 nM or less, about 10 nM or less, about 1 nM or less, or about 0.1 nM or less.

In some embodiments, the Affimer Agent has a melting temperature (Tm, i.e., temperature at which both the folded and unfolded states are equally populated) of 65° C. or higher, and preferably at least 70° C., 75° C., 80° C. or even 85° C. or higher. Melting temperature is a particularly useful indicator of protein stability. The relative proportions of folded and unfolded proteins can be determined by many techniques known to the skilled person, including differential scanning calorimetry, UV difference spectroscopy, fluorescence, circular dichroism (CD), and NMR (Pace et al. (1997) “Measuring the conformational stability of a protein” in Protein structure: A practical approach 2: 299-321).

a. Fusions Proteins—General

In some embodiments, the affimer polypeptides may further comprise an additional insertion, substitution or deletion that modulates biological activity of the affimer polypeptide. For example, the additions, substitutions or deletions may modulate one or more properties or activities of modified affimer. For example, the additions, substitutions or deletions may modulate affinity for the affimer polypeptide, e.g., for binding to and inhibiting PD-1, modulate the circulating half-life, modulate the therapeutic half-life, modulate the stability of the affimer polypeptide, modulate cleavage by proteases, modulate dose, modulate release or bioavailability, facilitate purification, decrease deamidation, improve shelf-life, or improve or alter a particular route of administration. Similarly, affimer polypeptides may comprise protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection, purification or other traits of the polypeptide.

In some instances, these additional sequences are added to one end and/or the other of the affimer polypeptide in the form of a fusion protein. Accordingly, in certain aspects of the disclosure the Affimer Agent is a fusion protein having at least one affimer polypeptide sequence and one or more heterologous polypeptide sequences (“fusion domain” herein). A fusion domain may be selected so as to confer a desired property, such as secretion from a cell or retention on the cell surface (i.e., for Encoded Affimers), to serve as substrate or other recognition sequences for post-translational modifications, to create multimeric structures aggregating through protein-protein interactions, to alter (often to extend) serum half-life, or to alter tissue localization or tissue exclusion and other ADME properties—merely as examples.

For example, some fusion domains are particularly useful for isolation and/or purification of the fusion proteins, such as by affinity chromatography. Well known examples of such fusion domains that facilitate expression or purification include, merely to illustrate, affinity tags such as polyhistidine (i.e., a His₆ tag), Strep II tag, streptavidin-binding peptide (SBP) tag, calmodulin-binding peptide (CBP), glutathione S-transferase (GST), maltose-binding protein (MBP), S-tag, HA tag, c-Myc tag, thioredoxin, protein A and protein G.

In order for the Affimer Agent to be secreted, it will generally contain a signal sequence that directs the transport of the protein to the lumen of the endoplasmic reticulum and ultimately to be secreted (or retained on the cell surface if a transmembrane domain or other cell surface retention signal). Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence.

In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments, the signal peptide is a native signal peptide from a human protein. In other embodiments, the signal peptide is a non-native signal peptide. For example, in some embodiments, the non-native signal peptide is a mutant native signal peptide from the corresponding native secreted human protein, and can include one or more (such as 2, 3, 4, 5, 6, 7, 8, 9, or 10 or more) substitutions insertions or deletions.

In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g. chymotrypsinogen or trypsinogen) or other signal peptide able to efficiently secrete a protein from a cell. Exemplary signal peptides include, but are not limited to:

| Native Protein | Signal Sequence |
|---|---|
| Human Serum | MKWVTFISLLFLFSSAYS |
| Albumin (HSA) | (SEQ ID NO: 137) |
| Ig kappa light | MDMRAPAGIFGFLLVLFPGYRS |
| chain | (SEQ ID NO: 138) |
| Human azurocidin | MTRLTVLALLAGLLASSRA |
| preprotein | (SEQ ID NO: 139) |
| IgG heavy chain | MELGLSWIFLLAILKGVQC |
| | (SEQ ID NO: 140) |
| IgG heavy chain | MELGLRWVFLVAILEGVQC |
| | (SEQ ID NO: 141) |
| IgG heavy chain | MKHLWFFLLLVAAPRWVLS |
| | (SEQ ID NO: 142) |
| IgG heavy chain | MDWTWRILFLVAAATGAHS |
| | (SEQ ID NO: 143) |
| IgG heavy chain | MDWTWRFLFVVAAATGVQS |
| | (SEQ ID NO: 144) |
| IgG heavy chain | MEFGLSWLFLVAILKGVQC |
| | (SEQ ID NO: 145) |
| IgG heavy chain | MEFGLSWVFLVALFRGVQC |
| | (SEQ ID NO: 146) |
| IgG heavy chain | MDLLHKNMKHLWFFLLLVAAPRWVLS |
| | (SEQ ID NO: 147) |
| IgG Kappa light | MDMRVPAQLLGLLLLWLSGARC |
| | (SEQ ID NO: 148) |
| IgG Kappa light | MKYLLPTAAAGLLLLAAQPAMA |
| | (SEQ ID NO: 149) |
| Gaussia luciferase | MGVKVLFALICIAVAEA |
| | (SEQ ID NO: 150) |
| Human | MAFLWLLSCWALLGTTFG |
| chymotrypsinogen | (SEQ ID NO: 152) |
| Human inter- | MQLLSCIALILALV |
| leukin-2 | (SEQ ID NO: 153) |
| Human trypsin- | MNLLLILTFVAAAVA |
| ogen-2 | (SEQ ID NO: 154) |
| Human CD33 | MPLLLLLPLLWAGALA |
| | (SEQ ID NO: 155) |
| Prolactin | MDSKGSSQKGSRLLLLLVVSNLLLCQGVVS |
| | (SEQ ID NO: 156) |
| Human tPA | MDAMKRGLCCVLLLCGAVFVSPS |
| | (SEQ ID NO: 157) |
| Synthetic/ | MLLLLLLLLLLALALA |
| Consensus | (SEQ ID NO: 158) |
| Synthetic/ | MWWRLWWLLLLLLLLWPMVWA |
| Consensus | (SEQ ID NO: 159) |

In some embodiments of a secreted Affimer Agent, the recombinant polypeptide comprises a signal peptide when expressed, and the signal peptide (or a portion thereof) is cleaved from the Affimer Agent upon secretion.

The subject fusion proteins may also include one or more linkers separating heterologous protein sequences or domains. As used herein, the term “linker” refers to a linker amino acid sequence inserted between a first polypeptide (e.g., an affimer) and a second polypeptide (e.g., a second affimer, an Fc region, a receptor trap, albumin, etc). Empirical linkers designed by researchers are generally classified into 3 categories according to their structures: flexible linkers, rigid linkers, and in vivo cleavable linkers. Besides the basic role in linking the functional domains together (as in flexible and rigid linkers) or releasing free functional domain in vivo (as in in vivo cleavable linkers), linkers may offer many other advantages for the production of fusion proteins, such as improving biological activity, increasing expression yield, and achieving desirable pharmacokinetic profiles. Linkers should not adversely affect the expression, secretion, or bioactivity of the fusion protein. Linkers should not be antigenic and should not elicit an immune response.

Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. In some embodiments, the linker may comprise a cleavage site. In some embodiments, the linker may comprise an enzyme cleavage site, so that the second polypeptide may be separated from the first polypeptide.

In some embodiments, the linker can be characterized as flexible. Flexible linkers are usually applied when the joined domains require a certain degree of movement or interaction. They are generally composed of small, non-polar (e.g. Gly) or polar (e.g. Ser or Thr) amino acids. See, for example, Argos P. (1990) “An investigation of oligopeptides linking domains in protein tertiary structures and possible candidates for general gene fusion” J Mol Biol. 211:943-958. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). An example of the most widely used flexible linker has the sequence of (Gly-Gly-Gly-Gly-Ser)n. By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. As These flexible linkers are also rich in small or polar amino acids such as Gly and Ser, but can contain additional amino acids such as Thr and Ala to maintain flexibility, as well as polar amino acids such as Lys and Glu to improve solubility.

In some embodiments, the linker can be characterized as rigid. While flexible linkers have the advantage to connect the functional domains passively and permitting certain degree of movements, the lack of rigidity of these linkers can be a limitation in certain fusion protein embodiments, such as in expression yield or biological activity. The ineffectiveness of flexible linkers in these instances was attributed to an inefficient separation of the protein domains or insufficient reduction of their interference with each other. Under these situations, rigid linkers have been successfully applied to keep a fixed distance between the domains and to maintain their independent functions.

Many natural linkers exhibited α-helical structures. The α-helical structure was rigid and stable, with intra-segment hydrogen bonds and a closely packed backbone. Therefore, the stiff α-helical linkers can act as rigid spacers between protein domains. George et al. (2002) “An analysis of protein domain linkers: their classification and role in protein folding” Protein Eng. 15(11):871-9. In general, rigid linkers exhibit relatively stiff structures by adopting α-helical structures or by containing multiple Pro residues. Under many circumstances, they separate the functional domains more efficiently than the flexible linkers. The length of the linkers can be easily adjusted by changing the copy number to achieve an optimal distance between domains. As a result, rigid linkers are chosen when the spatial separation of the domains is critical to preserve the stability or bioactivity of the fusion proteins. In this regard, alpha helix-forming linkers with the sequence of (EAAAK)n (SEQ ID NO: 201) have been applied to the construction of many recombinant fusion proteins. Another type of rigid linkers has a Pro-rich sequence, (XP)n, with X designating any amino acid, preferably Ala, Lys, or Glu.

Merely to illustrate, exemplary linkers include:

Flexible
(SEQ ID NO: 198)
(GGGGS)ₙ (i.e., n = 1-6)
Flexible
(SEQ ID NO: 199)
(Gly)₈
Flexible
(SEQ ID NO: 200)
(Gly)₆
Flexible
(SEQ ID NO: 160)
KESGSVSSEQLAQFRSLD
Flexible
(SEQ ID NO: 161)
EGKSSGSGSESKST
Flexible
(SEQ ID NO: 162)
GSAGSAAGSGEF
Rigid
(SEQ ID NO: 201)
(EAAAK)ₙ (i.e., n = 1-6)
Rigid
(SEQ ID NO: 163)
A(EAAAK)₄ALEA(EAAAK)₄A
Rigid
(SEQ ID NO: 164)
PAPAP
Rigid
(SEQ ID NO: 165)
AEAAAKEAAAKA
Rigid
(SEQ ID NO: 202)
(Ala-Pro)ₙ (10 to 34 aa)

Other linkers that may be used in the subject fusion proteins include, but are not limited to, SerGly, GGSG (SEQ ID NO: 203), GSGS (SEQ ID NO: 204), GGGS (SEQ ID NO: 205), S(GGS)n (SEQ ID NO: 206) where n is 1-7, GRA, poly(Gly), poly(Ala), GGGSGGG (SEQ ID NO: 166), ESGGGGVT (SEQ ID NO: 167), LESGGGGVT (SEQ ID NO: 168), GRAQVT (SEQ ID NO: 169), WRAQVT (SEQ ID NO: 170), and ARGRAQVT (SEQ ID NO: 171). The hinge regions of the Fc fusions described below may also be considered linkers.

Various elements can be employed to anchor proteins on the plasma membrane of cells. For example, the transmembrane domains (TM) of type-I (oriented with the N-terminus outside the cell) and type-II (oriented with the N-terminus in the cytosol) integral membrane proteins can be used to target chimeric proteins to the plasma membrane. Proteins can also be attached to the cell surface by fusion of a GPI (glycophosphatidylinositol lipid) signal to the 3′ end of genes. Cleavage of the short carboxy-terminal peptide allows attachment of a glycolipid to the newly exposed C-terminus through an amide linkage. See Udenfriend et al. (1995) “How Glycosylphosphatidylinositol Anchored Membrane Proteins are Made” Annu Rev Biochem 64:563-591.

In some embodiments, the fusion protein includes a transmembrane polypeptide sequence (a transmembrane domain). The distinguishing features of appropriate transmembrane polypeptides comprise the ability to be expressed at the surface of the cell on which the Affimer Agent is to be displayed. In some embodiments, that may be an immune cell, in particular lymphocyte cells or Natural killer (NK) cells, and once there to interact with PD-L1 so as to directing cellular response of the immune cell against a predefined target Tumor cell on which PD-L1 is upregulated. The transmembrane domain can be derived either from a natural or from a synthetic source. The transmembrane domain can be derived from any membrane-bound or transmembrane protein. As non limiting examples, the transmembrane polypeptide can be a subunit of the T cell receptor such as α, β, γ or δ, polypeptide constituting CD3 complex, IL2 receptor p55 (a chain), p75 (β chain) or γ chain, subunit chain of Fc receptors, in particular Fey receptor III or CD proteins. Alternatively the transmembrane domain can be synthetic and can comprise predominantly hydrophobic residues such as leucine and valine.

In certain other embodiments, the Affimer Agent is a fusion protein including, in addition to an affimer polypeptide, a sequence that signals for the posttranslational addition of a glycosylphosphatidylinositol (GPI) anchor. GPI anchors are glycolipid structures that are added post-translationally to the C-terminus of many eukaryotic proteins. This modification to the Affimer Agent will cause it to be anchored (attached) on the extracellular surface of the cell membrane of the cell in which the Affimer Agent is re-expressed as a recombinant protein (i.e., an Encoded Affimer as described below). In these embodiments, the GPI anchor domain is C-terminal to the affimer polypeptide sequence, and preferably occurs at the C-terminus of the fusion protein.

In some embodiments, the GPI anchor domain is a polypeptide that signals for the posttranslational addition of a GPI anchor when the fusion protein of which it is a part is expressed in a eukaryotic system. The GPI anchor signal sequence consists of a set of small amino acids at the site of anchor addition (the w site) followed by a hydrophilic spacer and ending in a hydrophobic stretch (Low, (1989) FASEB J. 3:1600-1608). Cleavage of this signal sequence occurs in the ER before the addition of an anchor with conserved central components but with variable peripheral moieties (Homans et al., Nature, 333:269-272 (1988)). The C-terminus of a GPI-anchored protein is linked through a phosphoethanolamine bridge to the highly conserved core glycan, mannose(α1-2)mannose(α1-6)mannose(α1-4)glucosamine(α1-6)myo-inositol. A phospholipid tail attaches the GPI anchor to the cell membrane.

Exemplary GPI anchor domains that can be used in the subject affimer-containing fusion proteins include:

(SEQ ID NO: 172)
SGTTSGTTRLLSGHTCFTLTGLLGTLVTMGLLT
(SEQ ID NO: 173)
SGTSPGLSAGATVGIMIGVLVGVALI
(SEQ ID NO: 174)
SAPVLSAVATVGITIGVLARVALI
(SEQ ID NO: 175)
SSPDLSAGTAVSIMIGVLAGMALI
(SEQ ID NO: 176)
TLGGNSASYTFVSLLFSAVTLLLLC
(SEQ ID NO: 177)
SGTSPGLSAGATVGIMIGVLVGVALI

GPI anchor attachment can be achieved by expression of the affimer fusion protein containing the GPI anchor domain in a eukaryotic system capable of carrying out GPI posttranslational modifications. As with the transmembrane domain fusion proteins, human cells, including lymphocytes and other cells involved in initiating or promoting an antitumor are so capable and can be engineered to express and Encoded Affimer including a GPI anchor domain in order retain the expressed affimer containing fusion on the surface of the engineered cell.

Still other modifications that can be made to the affimer polypeptide sequence itself or to a flanking polypeptide moiety provided as part of a fusion protein is one or more sequences that are sites for post-translational modifications by enzymes. These can include, but are not limited to, glycosylation, acetylation, acylation, lipid-modification, palmitoylation, palmitate addition, phosphorylation, glycolipid-linkage modification, and the like.

b. Engineering PK and ADME Properties

In some embodiment, the Affimer Agent may not have a half-life and/or PK profile that is optimal for the route of administration, such as parenteral therapeutic dosing. The term “half-life” refers to the amount of time it takes for a substance, such as an Affimer Agent of the present disclosure, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). To address this shortcoming, there are a variety of general strategies for prolongation of half-life that have been used in the case of other protein therapeutics, including the incorporation of half-life extending moieties as part of the Affimer Agent.

The term “half-life extending moiety” refers to a pharmaceutically acceptable moiety, domain, or molecule covalently linked (“conjugated” or “fused”) to the affimer polypeptide to form the Affimer Agents described herein, optionally via a non-naturally encoded amino acid, directly or via a linker, that prevents or mitigates in vivo proteolytic degradation or other activity-diminishing modification of the affimer polypeptide, increases half-life, and/or improves or alters other pharmacokinetic or biophysical properties including but not limited to increasing the rate of absorption, reducing toxicity, improving solubility, reducing protein aggregation, increasing biological activity and/or target selectivity of the modified affimer polypeptide, increasing manufacturability, and/or reducing immunogenicity of the modified affimer polypeptide, compared to a comparator such as an unconjugated form of the modified affimer polypeptide. The term “half-life extending moiety” includes non-proteinaceous, half-life extending moieties, such as a water soluble polymer such as polyethylene glycol (PEG) or discrete PEG, hydroxyethyl starch (HES), a lipid, a branched or unbranched acyl group, a branched or unbranched C8-C30 acyl group, a branched or unbranched alkyl group, and a branched or unbranched C8-C30 alkyl group; and proteinaceous half-life extending moieties, such as serum albumin, transferrin, adnectins (e.g., albumin-binding or pharmacokinetics extending (PKE) adnectins), Fc domain, and unstructured polypeptide, such as XTEN and PAS polypeptide (e.g. conformationally disordered polypeptide sequences composed of the amino acids Pro, Ala, and/or Ser), and a fragment of any of the foregoing. An examination of the crystal structure of an affimer and its interaction with its target, such as the anti-PD-L1 affimer complex with PD-1 shown in the FIGS., can indicate which certain amino acid residues have side chains that are fully or partially accessible to solvent.

In some embodiments, the half-life extending moiety extends the half-life of the resulting Affimer Agent circulating in mammalian blood serum compared to the half-life of the protein that is not so conjugated to the moiety (such as relative to the Affimer polypeptide alone). In some embodiments, half-life is extended by greater than or greater than about 1.2-fold, 1.5-fold, 2.0-fold, 3.0-fold, 4.0-fold, 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety.

As means for further exemplification, half-life extending moieties that can be used in the generation of Affimer Agents of the disclosure include:
Genetic fusion of the pharmacologically affimer sequence to a naturally long-half-life protein or protein domain (e.g., Fc fusion, transferrin [Tf] fusion, or albumin fusion. See, for example, Beck et al. (2011) “Therapeutic Fc-fusion proteins and peptides as successful alternatives to antibodies. MAbs. 3:1-2; Czajkowsky et al. (2012) “Fc-fusion proteins: new developments and future perspectives. EMBO Mol Med. 4:1015-28; Huang et al. (2009) “Receptor-Fc fusion therapeutics, traps, and Mimetibody technology” Curr Opin Biotechnol. 2009; 20:692-9; Keefe et al. (2013) “Transferrin fusion protein therapies: acetylcholine receptor-transferrin fusion protein as a model. In: Schmidt S, editor. Fusion protein technologies for biopharmaceuticals: applications and challenges. Hoboken: Wiley; p. 345-56; Weimer et al. (2013) “Recombinant albumin fusion proteins. In: Schmidt S, editor. Fusion protein technologies for biopharmaceuticals: applications and challenges. Hoboken: Wiley; 2013. p. 297-323; Walker et al. (2013) “Albumin-binding fusion proteins in the development of novel long-acting therapeutics. In: Schmidt S, editor. Fusion protein technologies for biopharmaceuticals: applications and challenges. Hoboken: Wiley; 2013. p. 325-43.
Genetic fusion of the pharmacologically affimer sequence to an inert polypeptide, e.g., XTEN (also known as recombinant PEG or “rPEG”), a homoamino acid polymer (HAP; HAPylation), a proline-alanine-serine polymer (PAS; PASylation), or an elastin-like peptide (ELP; ELPylation). See, for example, Schellenberger et al. (2009) “A recombinant polypeptide extends the in vivo half-life of peptides and proteins in a tunable manner. Nat Biotechnol. 2009; 27:1186-90; Schlapschy et al. Fusion of a recombinant antibody fragment with a homo-amino-acid polymer: effects on biophysical properties and prolonged plasma half-life. Protein Eng Des Sel. 2007; 20:273-84; Schlapschy (2013) PASylation: a biological alternative to PEGylation for extending the plasma halflife of pharmaceutically active proteins. Protein Eng Des Sel. 26:489-501. Floss et al. (2012) “Elastin-like polypeptides revolutionize recombinant protein expression and their biomedical application. Trends Biotechnol. 28:37-45. Floss et al. “ELP-fusion technology for biopharmaceuticals. In: Schmidt S, editor. Fusion protein technologies for biopharmaceuticals: application and challenges. Hoboken: Wiley; 2013. p. 372-98.
Increasing the hydrodynamic radius by chemical conjugation of the pharmacologically active peptide or protein to repeat chemical moieties, e.g., to PEG (PEGylation) or hyaluronic acid. See, for example, Caliceti et al. (2003) “Pharmacokinetic and biodistribution properties of poly(ethylene glycol)-protein conjugates” Adv Drug Delivery Rev. 55:1261-77; Jevsevar et al. (2010) PEGylation of therapeutic proteins. Biotechnol J 5:113-28; Kontermann (2009) “Strategies to extend plasma half-lives of recombinant antibodies” BioDrugs. 23:93-109; Kang et al. (2009) “Emerging PEGylated drugs” Expert Opin Emerg Drugs. 14:363-80; and Mero et al. (2013) “Conjugation of hyaluronan to proteins” Carb Polymers. 92:2163-70.
Significantly increasing the negative charge of fusing the pharmacologically active peptide or protein by polysialylation; or, alternatively, (b) fusing a negatively charged, highly sialylated peptide (e.g., carboxy-terminal peptide [CTP; of chorionic gonadotropin (CG) b-chain]), known to extend the half-life of natural proteins such as human CG b-subunit, to the biological drug candidate. See, for example, Gregoriadis et al. (2005) “Improving the therapeutic efficacy of peptides and proteins: a role for polysialic acids” Int J Pharm. 2005; 300:125-30; Duijkers et al. “Single dose pharmacokinetics and effects on follicular growth and serum hormones of a long-acting recombinant FSH preparation (FSHCTP) in healthy pituitary-suppressed females” (2002) Hum Reprod. 17:1987-93; and Fares et al. “Design of a longacting follitropin agonist by fusing the C-terminal sequence of the chorionic gonadotropin beta subunit to the follitropin beta subunit” (1992) Proc Natl Acad Sci USA. 89:4304-8. 35; and Fares “Half-life extension through O-glycosylation.
Binding non-covalently, via attachment of a peptide or protein-binding domain to the bioactive protein, to normally long-half-life proteins such as HSA, human IgG, transferrin or fibronectin. See, for example, Andersen et al. (2011) “Extending half-life by indirect targeting of the neonatal Fc receptor (FcRn) using a minimal albumin binding domain” J Biol Chem. 286:5234-41; O'Connor-Semmes et al. (2014) “GSK2374697, a novel albumin-binding domain antibody (albudAb), extends systemic exposure of extendin-4: first study in humans—PK/PD and safety” Clin Pharmacol Ther. 2014; 96:704-12. Sockolosky et al. (2014) “Fusion of a short peptide that binds immunoglobulin G to a recombinant protein substantially increases its plasma half-life in mice” PLoS One. 2014; 9:e102566.

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HSA). Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism: the neonatal Fc receptor, FcRn. The pH-dependent binding of these proteins to FcRn prevents degradation of the fusion protein in the endosome. Fusions based on these proteins can have half-lives in the range of 3-16 days, much longer than typical PEGylated or lipidated peptides. Fusion to antibody Fc domains can improve the solubility and stability of the peptide or protein drug. An example of a peptide Fc fusion is dulaglutide, a GLP-1 receptor agonist currently in late-stage clinical trials. Human serum albumin, the same protein exploited by the fatty acylated peptides is the other popular fusion partner. Albiglutide is a GLP-1 receptor agonist based on this platform. A major difference between Fc and albumin is the dimeric nature of Fc versus the monomeric structure of HSA leading to presentation of a fused peptide as a dimer or a monomer depending on the choice of fusion partner. The dimeric nature of an Affimer-Fc fusion can produce an avidity effect if the Affimer target, such as PD-L1 on Tumor cells, are spaced closely enough together or are themselves dimers. This may be desirable or not depending on the target.

(i) Fc Fusions

In some embodiments, the affimer polypeptide may be part of a fusion protein with an immunoglobulin Fc domain (“Fc domain”), or a fragment or variant thereof, such as a functional Fc region. In this context, an Fc fusion (“Fc-fusion”), such as an Affimer Agent created as an Affimer-Fc fusion protein, is a polypeptide comprising one or more affimer sequences covalently linked through a peptide backbone (directly or indirectly) to an Fc region of an immunoglobulin. An Fc-fusion may comprise, for example, the Fc region of an antibody (which facilitates effector functions and pharmacokinetics) and an affimer sequence as part of the same polypeptide. An immunoglobulin Fc region may also be linked indirectly to one or more affimers. Various linkers are known in the art and can optionally be used to link an Fc to a polypeptide including an affimer sequence to generate an Fc-fusion. In some embodiments, Fc-fusions can be dimerized to form Fc-fusion homodimers, or using non-identical Fc domains, to form Fc-fusion heterodimers.

There are several reasons for choosing the Fc region of human antibodies for use in generating the subject Affimer Agents as affimer fusion proteins. The principle rationale is to produce a stable protein, large enough to demonstrate a similar pharmacokinetic profile compared with those of antibodies, and to take advantage of the properties imparted by the Fc region; this includes the salvage neonatal FcRn receptor pathway involving FcRn-mediated recycling of the fusion protein to the cell surface post endocytosis, avoiding lysosomal degradation and resulting in release back into the bloodstream, thus contributing to an extended serum half-life. Another obvious advantage is the Fc domain's binding to Protein A, which can simplify downstream processing during production of the Affimer Agent and permit generation of highly pure preparation of the Affimer Agent.

In general, an Fc domain will include the constant region of an antibody excluding the first constant region immunoglobulin domain. Thus, Fc domain refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. Although the boundaries of the Fc domain may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, NIH, Bethesda, Md. (1991)). Fc may refer to this region in isolation, or this region in the context of a whole antibody, antibody fragment, or Fc fusion protein. Polymorphisms have been observed at a number of different Fc positions and are also included as Fc domains as used herein.

In some embodiments, the Fc As used herein, a “functional Fc region” refers to an Fc domain or fragment thereof which retains the ability to bind FcRn. A functional Fc region binds to FcRn, but does not possess effector function. The ability of the Fc region or fragment thereof to bind to FcRn can be determined by standard binding assays known in the art. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions can be assessed using various assays known in the art for evaluating such antibody effector functions.

In an exemplary embodiment, the Fc domain is derived from an IgG1 subclass, however, other subclasses (e.g., IgG2, IgG3, and IgG4) may also be used. An exemplary sequence of a human IgG1 immunoglobulin Fc domain which can be used is:

(SEQ ID NO: 95)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

In some embodiments, the Fc region used in the fusion protein may comprise the hinge region of an Fc molecule. An exemplary hinge region comprises the core hinge residues spanning positions 1-16 (i.e., DKTHTCPPCPAPELLG (SEQ ID NO: 178)) of the exemplary human IgG1 immunoglobulin Fc domain sequence provided above. In some embodiments, the affimer-containing fusion protein may adopt a multimeric structure (e.g., dimer) owing, in part, to the cysteine residues at positions 6 and 9 within the hinge region of the exemplary human IgG1 immunoglobulin Fc domain sequence provided above. In other embodiments, the hinge region as used herein, may further include residues derived from the CH1 and CH2 regions that flank the core hinge sequence of the exemplary human IgG1 immunoglobulin Fc domain sequence provided above. In yet other embodiments, the hinge sequence may comprise or consist of GSTHTCPPCPAPELLG (SEQ ID NO: 179) or EPKSCDKTHTCPPCPAPELLG (SEQ ID NO: 180).

In some embodiments, the hinge sequence may include one or more substitutions that confer desirable pharmacokinetic, biophysical, and/or biological properties. Some exemplary hinge sequences include:

(SEQ ID NO: 181)
EPKSCDKTHTCPPCPAPELLGGPS;
(SEQ ID NO: 182)
EPKSSDKTHTCPPCPAPELLGGPS;
(SEQ ID NO: 183)
EPKSSDKTHTCPPCPAPELLGGSS;
(SEQ ID NO: 184)
EPKSSGSTHTCPPCPAPELLGGSS;
(SEQ ID NO: 185)
DKTHTCPPCPAPELLGGPS;
and
(SEQ ID NO: 151)
DKTHTCPPCPAPELLGGSS.

In some embodiments, the residue P at position 18 of the exemplary human IgG1 immunoglobulin Fc domain sequence provided above may be replaced with S to ablate Fc effector function; this replacement is exemplified in hinges having the sequences EPKSSDKTHTCPPCPAPELLGGSS (SEQ ID NO: 183), EPKSSGSTHTCPPCPAPELLGGSS (SEQ ID NO: 184), and DKTHTCPPCPAPELLGGSS (SEQ ID NO: 186). In another embodiment, the residues DK at positions 1-2 of the exemplary human IgG1 immunoglobulin Fc domain sequence provided above may be replaced with GS to remove a potential clip site; this replacement is exemplified in the sequence EPKSSGSTHTCPPCPAPELLGGSS (SEQ ID NO: 184). In another embodiment, the C at the position 103 of the heavy chain constant region of human IgG1 (i.e., domains CH₁—CH₃), may be replaced with S to prevent improper cysteine bond formation in the absence of a light chain; this replacement is exemplified by EPKSSDKTHTCPPCPAPELLGGPS (SEQ ID NO: 182), EPKSSDKTHTCPPCPAPELLGGSS (SEQ ID NO: 183), and EPKSSGSTHTCPPCPAPELLGGSS (SEQ ID NO: 184).

In some embodiments, the Fc is a mammalian Fc such as a human Fc, including Fc domains derived from IgG1, IgG2, IgG3 or IgG4. The Fc region may possess at least about 80%, 85%, 90%, 95%, 96%, 97%, 98%, or 99% sequence identity with a native Fc region and/or with an Fc region of a parent polypeptide. In some embodiments, the Fc region may have at least about 90% sequence identity with a native Fc region and/or with an Fc region of a parent polypeptide.

In some embodiments, the Fc domain comprises an amino acid sequence selected from SEQ ID NOs: 95, or an Fc sequence from the examples provided by SEQ ID NOS: 96-108. It should be understood that the C-terminal lysine of an Fc domain is an optional component of a fusion protein comprising an Fc domain. In some embodiments, the Fc domain comprises an amino acid sequence selected from SEQ ID NOs: 95-108, except that the C-terminal lysine thereof is omitted. In some embodiments, the Fc domain comprises the amino acid sequence of SEQ ID NO: 95. In some embodiments, the Fc domain comprises the amino acid sequence of SEQ ID NOs: 95 except the C-terminal lysine thereof is omitted.

| | |
|---|---|
| hIgG1a_191 | DKTHTCPPCPAPELLGGPSVFLFPPKPKDTLMISRTPEVTCV |
| [A subatype] | VVDVSHEDPEVKFNWYVDGVEVHNAKTKPREEQYNSTYR |
| (SEQ ID NO: 96) | VVSVLTVLHQDWLNGKEYKCKVSNKALPAPIEKTISKAKG |
| | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPGK |
| hIgG1a_189 | DKTHTCPPCPAPELLGGPSVFLFPPKPKDTLMISRTPEVTCV |
| [hIgG1a_191 sans ″GK″ | VVDVSHEDPEVKFNWYVDGVEVHNAKTKPREEQYNSTYR |
| on C term; A subtype] | VVSVLTVLHQDWLNGKEYKCKVSNKALPAPIEKTISKAKG |
| (SEQ ID NO: 97) | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSP |
| hIgG1a_191b | DKTHTCPPCPAPELLGGPSVFLFPPKPKDTLMISRTPEVTCV |
| [A/F subtype] | VVDVSHEDPEVKFNWYVDGVEVHNAKTKPREEQYNSTYR |
| (SEQ ID NO: 98) | VVSVLTVLHQDWLNGKEYKCKVSNKALPAPIEKTISKAKG |
| | QPREPQVYTLPPSRDEMTKNQVSLTCLVKGFYPSDIAVEWE |
| | SNGQPENNYKTTPPVLDSDGSFFLYSKLTVDKSRWQQGNV |
| | FSCSVMHEALHNHYTQKSLSLSPGK |
| hIgG1f_1.1_191 | DKTHTCPPCPAPEAEGAPSVFLFPPKPKDTLMISRTPEVTCV |
| [Contains 5 point | VVDVSHEDPEVKFNWYVDGVEVHNAKTKPREEQYNSTYR |
| mutations to alter | VVSVLTVLHQDWLNGKEYKCKVSNKALPSSIEKTISKAKGQ |
| ADCC function, F | PREPQVYTLPPSREEMTKNQVSLTCLVKGFYPSDIAVEWES |
| subtype] (SEQ ID NO: | NGQPENNYKTTPPVLDSDGSFFLYSKLTVDKSRWQQGNVF |
| 99) | SCSVMHEALHNHYTQKSLSLSPGK |
| hIgG1f_1.1_186 | EPKSSDKTHTCPPCPAPEAEGAPSVFLFPPKPKDTLMISRTPE |
| [Contains 5 point | VTCVVVDVSHEDPEVKFNWYVDGVEVHNAKTKPREEQYN |
| mutations to alter | STYRVVSVLTVLHQDWLNGKEYKCKVSNKALPSSIEKTISK |
| ADCC function and | AKGQPREPQVYTLPPSREEMTKNQVSLTCLVKGFYPSDIAV |
| C225S (Edlemen | EWESNGQPENNYKTTPPVLDSDGSFFLYSKLTVDKSRWQQ |
| numbering); F subtype] | GNVFSCSVMHEALHNHYTQKSLSLSPGK |
| (SEQ ID NO: 100) | |
| hIgG1a_(N297G)_191 | DKTHTCPPCPAPELLGGPSVFLFPPKPKDTLMISRTPEVTCV |
| [A subtype] | VVDVSHEDPEVKFNWYVDGVEVHNAKTKPREEQYGSTYR |
| (SEQ ID NO: 101) | VVSVLTVLHQDWLNGKEYKCKVSNKALPAPIEKTISKAKG |
| | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPGK |
| hIgG1a_190 | DKTHTCPPCPAPELLGGPSVFLFPPKPKDTLMISRTPEVTCV |
| [hIgG1a_190 sans ″K″ | VVDVSHEDPEVKFNWYVDGVEVHNAKTKPREEQYNSTYR |
| on C term; A subtype] | VVSVLTVLHQDWLNGKEYKCKVSNKALPAPIEKTISKAKG |
| (SEQ ID NO: 102) | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPG |
| hIgG1a_(N297Q)_191 | DKTHTCPPCPAPELLGGPSVFLFPPKPKDTLMISRTPEVTCV |
| [A subtype] | VVDVSHEDPEVKFNWYVDGVEVHNAKTKPREEQYQSTYR |
| (SEQ ID NO: 103) | VVSVLTVLHQDWLNGKEYKCKVSNKALPAPIEKTISKAKG |
| | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPGK |
| hIgG1a_(N297S)_191 | DKTHTCPPCPAPELLGGPSVFLFPPKPKDTLMISRTPEVTCV |
| [A subtype] | VVDVSHEDPEVKFNWYVDGVEVHNAKTKPREEQYSSTYR |
| (SEQ ID NO: 104) | VVSVLTVLHQDWLNGKEYKCKVSNKALPAPIEKTISKAKG |
| | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPGK |
| hIgG1a_(N297A)_191 | DKTHTCPPCPAPELLGGPSVFLFPPKPKDTLMISRTPEVTCV |
| [A subtype] | VVDVSHEDPEVKFNWYVDGVEVHNAKTKPREEQYASTYR |
| (SEQ ID NO: 105) | VVSVLTVLHQDWLNGKEYKCKVSNKALPAPIEKTISKAKG |
| | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPGK |
| hIgG1a_(N297H)_191 | DKTHTCPPCPAPELLGGPSVFLFPPKPKDTLMISRTPEVTCV |
| [A subtype] | VVDVSHEDPEVKFNWYVDGVEVHNAKTKPREEQYHSTYR |
| (SEQ ID NO: 107) | VVSVLTVLHQDWLNGKEYKCKVSNKALPAPIEKTISKAKG |
| | QPREPQVYTLPPSRDELTKNQVSLTCLVKGFYPSDIAVEWES |
| | NGQPENNYKTTPPVLDSDGSFFLYSKLTVDKSRWQQGNVF |
| | SCSVMHEALHNHYTQKSLSLSPGK |
| hIgG4 | DKRVESKYGPPCPSCPAPEFLGGPSVFLFPPKPKDTLMISRTP |
| (SEQ ID NO: 108) | EVTCVVVDVSQEDPEVQFNWYVDGVEVHNAKTKPREEQF |
| | NSTYRVVSVLTVLHQDWLNGKEYKCKVSNKGLPSSIEKTIS |
| | KAKGQPREPQVYTLPPSQEEMTKNQVSLTCLVKGFYPSDIA |
| | VEWESNGQPENNYKTTPPVLDSDGSFFLYSRLTVDKSRWQ |
| | EGNVFSCSVMHEALHNHYTQKSLSLSLGK |
| hIgG4_(S241P) | DKRVESKYGPPCPPCPAPEFLGGPSVFLFPPKPKDTLMISRTP |
| (SEQ ID NO: 109) | EVTCVVVDVSQEDPEVQFNWYVDGVEVHNAKTKPREEQF |
| | NSTYRVVSVLTVLHQDWLNGKEYKCKVSNKGLPSSIEKTIS |
| | KAKGQPREPQVYTLPPSQEEMTKNQVSLTCLVKGFYPSDIA |
| | VEWESNGQPENNYKTTPPVLDSDGSFFLYSRLTVDKSRWQ |
| | EGNVFSCSVMHEALHNHYTQKSLSLSLGK |
| hIgG1 (Contain 2 point | SEPKSCDKTHTCPPCPAPEAAGGPSVFLFPPKPKDTLMISRTP |
| mutations to alter | EVTCVVVDVSHEDPEVKFNWYVDGVEVHNAKTKPREEQY |
| ADCC function L20A, | NSTYRVVSVLTVLHQDWLNGKEYKCKVSNKALPAPIEKTIS |
| L21A) | KAKGQPREPQVYTLPPSREEMTKNQVSLTCLVKGFYPSDIA |
| (SEQ ID 110) | VEWESNGQPENNYKTTPPVLDSDGSFFLYSKLTVDKSRWQ |
| | QGNVFSCSVMHEALHNHYTQKSLSLSPGK |

Exemplary Fc fusions of a PD-L1 binding Affimer with an Fc are provided in the Examples and FIGS., demonstrating that the affimer sequence can be placed at either the N-terminal or C-terminal end of the Fc domain, and may be attached directly or the fusion protein may have other polypeptide sequences intervening between the Fc domain and the affimer polypeptide sequence. In the illustrated examples, an unstructured (flexible) linker, (Gly₄Ser)ₙ, is used with PD-L1 Binding Affimer “251” (SEQ ID NO: 86) and the Fc domain of human IgG1 (SEQ ID NO: 95) with the hinge region being EPKSCDKTHTCPPCPAPELLG. The constructs both included the CD33 secretion signal sequence MPLLLLLPLLWAGALA (SEQ ID NO: 136) which is cleaved from mature versions of the protein.

| | |
|---|---|
| PD-L1 251_Fc1 (V.2) | MPLLLLLPLLWAGALAIPRGLSEAKPATPEIQEIVDKVKPQL |
| (N-term Affimer) | EEKTGETYGKLEAVQYKTQVLAREGRQDWVLSTNYYIKVR |
| (SEQ ID NO: 111) | AGDNKYMHLKVFNGPWVPFPHQQLADRVLTGYQVDKNK |
| | DDELTGFAAAGGGGSGGGGSGGGGSGGGGSEPKSCDKTHT |
| | CPPCPAPELLGGPSVFLFPPKPKDTLMISRTPEVTCVVVDVS |
| | HEDPEVKFNWYVDGVEVHNAKTKPREEQYNSTYRVVSVL |
| | TVLHQDWLNGKEYKCKVSNKALPAPIEKTISKAKGQPREPQ |
| | VYTLPPSREEMTKNQVSLTCLVKGFYPSDIAVEWESNGQPE |
| | NNYKTTPPVLDSDGSFFLYSKLTVDKSRWQQGNVFSCSVM |
| | HEALHNHYTQKSLSLSPGK |
| PD-L1 251 Fc1 (AQ.2) | MPLLLLLPLLWAGALAEPKSCDKTHTCPPCPAPELLGGPSV |
| (C-term Affimer) | FLFPPKPKDTLMISRTPEVTCVVVDVSHEDPEVKFNWYVDG |
| (SEQ ID NO: 112) | VEVHNAKTKPREEQYNSTYRVVSVLTVLHQDWLNGKEYK |
| | CKVSNKALPAPIEKTISKAKGQPREPQVYTLPPSREEMTKNQ |
| | VSLTCLVKGFYPSDIAVEWESNGQPENNYKTTPPVLDSDGS |
| | FFLYSKLTVDKSRWQQGNVFSCSVMHEALHNHYTQKSLSL |
| | SPGKGGGGSGGGGSGGGGSGGGGSIPRGLSEAKPATPEIQEI |
| | VDKVKPQLEEKTGETYGKLEAVQYKTQVLAREGRQDWVL |
| | STNYYIKVRAGDNKYMHLKVFNGPWVPFPHQQLADRVLT |
| | GYQVDKNKDDELTGFAAA |

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins.

In some embodiments, the fusion protein includes an Fc domain sequence for which the resulting Affimer Agent has no (or reduced) ADCC and/or complement activation or effector functionality. For example, the Fc domain may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

In other embodiments, the fusion protein includes an Fc domain sequence for which the resulting Affimer Agent will retain some or all Fc functionality for example will be capable of one or both of ADCC and CDC activity, as for example if the fusion protein comprises the Fc domain from human IgG1 or IgG3. Levels of effector function can be varied according to known techniques, for example by mutations in the CH2 domain, for example wherein the IgG1 CH2 domain has one or more mutations at positions selected from 239 and 332 and 330, for example the mutations are selected from S239D and I332E and A330L such that the antibody has enhanced effector function, and/or for example altering the glycosylation profile of the antigen-binding protein of the disclosure such that there is a reduction in fucosylation of the Fc region.

(ii) Albumin Fusion

In other embodiments, the Affimer Agent is a fusion protein comprising, in addition to at least one affimer sequence, an albumin sequence or an albumin fragment. In other embodiments, the Affimer Agent is conjugated to the albumin sequence or an albumin fragment through chemical linkage other than incorporation into the polypeptide sequence including the affimer. In some embodiments, the albumin, albumin variant, or albumin fragment is human serum albumin (HSA), a human serum albumin variant, or a human serum albumin fragment. Albumin serum proteins comparable to HSA are found in, for example, cynomologus monkeys, cows, dogs, rabbits and rats. Of the non-human species, bovine serum albumin (BSA) is the most structurally similar to HSA. See, e.g., Kosa et al., (2007) J Pharm Sci. 96(11):3117-24. The present disclosure contemplates the use of albumin from non-human species, including, but not limited to, albumin sequence derived from cyno serum albumin or bovine serum albumin.

Mature HSA, a 585 amino acid polypeptide (approx. 67 kDa) having a serum half-life of about 20 days, is primarily responsible for the maintenance of colloidal osmotic blood pressure, blood pH, and transport and distribution of numerous endogenous and exogenous ligands. The protein has three structurally homologous domains (domains I, II and III), is almost entirely in the alpha-helical conformation, and is highly stabilized by 17 disulphide bridges. In some embodiments, the Affimer Agent can be an albumin fusion protein including one or more affimer polypeptide sequences and the sequence for mature human serum albumin (SEQ ID NO: 113) or a variant or fragment thereof which maintains the PK and/or biodistribution properties of mature albumin to the extent desired in the fusion protein.

(SEQ ID NO: 113)
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVEN
DEMPADLPSLAADFVESKDVCKNYAEAKDVFLGMFLYEYARRHPDYSV
VLLLRLAKTYETTLEKCCAAADPHECYAKVFDEFKPLVEEPQNLIKQNCE
LFEQLGEYKFQNALLVRYTKKVPQVSTPTLVEVSRNLGKVGSKCCKHPE
AKRMPCAEDYLSVVLNQLCVLHEKTPVSDRVTKCCTESLVNRRPCFSAL
EVDETYVPKEFNAETFTFHADICTLSEKERQIKKQTALVELVKHKPKATK
EQLKAVMDDFAAFVEKCCKADDKETCFAEEGKKLVAASQAALGL

The albumin sequence can be set off from the affimer polypeptide sequence or other flanking sequences in the Affimer Agent by use of linker sequences as described above.

While unless otherwise indicated, reference herein to “albumin” or to “mature albumin” is meant to refer to HSA. However, it is noted that full-length HSA has a signal peptide of 18 amino acids (MKWVTFISLLFLFSSAYS (SEQ ID NO: 137)) followed by a pro-domain of 6 amino acids (RGVFRR) (SEQ ID NO: 207); this 24 amino acid residue peptide may be referred to as the pre-pro domain. The Affimer-HSA fusion proteins can be expressed and secreted using the HSA pre-pro-domain in the recombinant proteins coding sequence. Alternatively, the affimer-HSA fusion can be expressed and secreted through inclusion of other secretion signal sequences, such as described above.

In alternative embodiments, rather than provided as part of a fusion protein with the affimer polypeptide, the serum albumin polypeptide can be covalently coupled to the affimer-containing polypeptide through a bond other than a backbone amide bond, such as cross-linked through chemical conjugation between amino acid sidechains on each of the albumin polypeptide and the affimer-containing polypeptide.

(iii) Albumin Binding Domain

In some embodiments, the Affimer Agent can include a serum-binding moiety—either as part of a fusion protein (if also a polypeptide) with the affimer polypeptide sequence or chemically conjugated through a site other than being part of a contiguous polypeptide chain.

In some embodiments, the serum-binding polypeptide is an albumin binding moiety. Albumin contains multiple hydrophobic binding pockets and naturally serves as a transporter of a variety of different ligands such as fatty acids and steroids as well as different drugs. Furthermore, the surface of albumin is negatively charged making it highly water-soluble.

The term “albumin binding moiety” as used herein refers to any chemical group capable of binding to albumin, i.e. has albumin binding affinity. Albumin binds to endogenous ligands such as fatty acids; however, it also interacts with exogenous ligands such as warfarin, penicillin and diazepam. As the binding of these drugs to albumin is reversible the albumin-drug complex serves as a drug reservoir that can enhance the drug biodistribution and bioavailability. Incorporation of components that mimic endogenous albumin-binding ligands, such as fatty acids, has been used to potentiate albumin association and increase drug efficacy.

In some embodiments, a chemical modification method that can be applied in the generation of the subject Affimer Agents to increase protein half-life is lipidation, which involves the covalent binding of fatty acids to peptide side chains. Originally conceived of and developed as a method for extending the half-life of insulin, lipidation shares the same basic mechanism of half-life extension as PEGylation, namely increasing the hydrodynamic radius to reduce renal filtration. However, the lipid moiety is itself relatively small and the effect is mediated indirectly through the non-covalent binding of the lipid moiety to circulating albumin. One consequence of lipidation is that it reduces the water-solubility of the peptide but engineering of the linker between the peptide and the fatty acid can modulate this, for example by the use of glutamate or mini PEGs within the linker. Linker engineering and variation of the lipid moeity can affect self-aggregation which can contribute to increased half-life by slowing down biodistribution, independent of albumin. See, for example, Jonassen et al. (2012) Pharm Res. 29(8):2104-14.

Other examples of albumin binding moieties for use in the generation of certain Affimer Agents include albumin-binding (PKE2) adnectins (See WO2011140086 “Serum Albumin Binding Molecules”, WO2015143199 “Serum albumin-binding Fibronectin Type III Domains” and WO2017053617 “Fast-off rate serum albumin binding fibronectin type iii domains”), the albumin binding domain 3 (ABD3) of protein G of Streptococcus strain G148, and the albumin binding domain antibody GSK2374697 (“AlbudAb”) or albumin binding nanobody portion of ATN-103 (Ozoralizumab).

(iv) PEGylation, XTEN, PAS and Other Polymers

A wide variety of macromolecular polymers and other molecules can be linked to the affimer containing polypeptides of the present disclosure to modulate biological properties of the resulting Affimer Agent, and/or provide new biological properties to the Affimer Agent. These macromolecular polymers can be linked to the affimer containing polypeptide via a naturally encoded amino acid, via a non-naturally encoded amino acid, or any functional substituent of a natural or non-natural amino acid, or any substituent or functional group added to a natural or non-natural amino acid. The molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

For this purpose, various methods including pegylation, polysialylation, HESylation, glycosylation, or recombinant PEG analogue fused to flexible and hydrophilic amino acid chain (500 to 600 amino acids) have been developed (See Chapman, (2002) Adv Drug Deliv Rev. 54. 531-545; Schlapschy et al., (2007) Prot Eng Des Sel. 20, 273-283; Contermann (2011) Curr Op Biotechnol. 22, 868-876; Jevsevar et al., (2012) Methods Mol Biol. 901, 233-246).

Examples of polymers include but are not limited to polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethylene glycol or PEG); discrete PEG (dPEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof; hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof; terpolymers thereof; mixtures thereof; and derivatives of the foregoing.

The polymer selected may be water soluble so that the Affimer Agent to which it is attached does not precipitate in an aqueous environment, such as a physiological environment. The water soluble polymer may be any structural form including but not limited to linear, forked or branched. Typically, the water soluble polymer is a poly(alkylene glycol), such as poly(ethylene glycol) (PEG), but other water soluble polymers can also be employed. By way of example, PEG is used to describe some embodiments of this disclosure. For therapeutic use of the Affimer Agent, the polymer may be pharmaceutically acceptable.

The term “PEG” is used broadly to encompass any polyethylene glycol molecule, without regard to size or to modification at an end of the PEG, and can be represented as linked to the affimer containing polypeptide by the formula:

XO—(CH₂CH₂O)ₙ—CH₂CH₂—

or

XO—(CH₂CH₂O)ₙ—

where n is 2 to 10,000 and X is H or a terminal modification, including but not limited to, a C1-4 alkyl, a protecting group, or a terminal functional group. In some cases, a PEG used in the polypeptides of the disclosure terminates on one end with hydroxy or methoxy, i.e., X is H or CH₃ (“methoxy PEG”).

It is noted that the other end of the PEG, which is shown in the above formulas by a terminal “-”, may attach to the affimer containing polypeptide via a naturally-occurring or non-naturally encoded amino acid. For instance, the attachment may be through an amide, carbamate or urea linkage to an amine group (including but not limited to, the epsilon amine of lysine or the N-terminus) of the polypeptide. Alternatively, the polymer is linked by a maleimide linkage to a thiol group (including but not limited to, the thiol group of cysteine)—which in the case of attachment to the affimer polypeptide sequence per se requires altering a residue in the affimer sequence to a cysteine.

The number of water soluble polymers linked to the affimer-containing polypeptide (i.e., the extent of PEGylation or glycosylation) can be adjusted to provide an altered (including but not limited to, increased or decreased) pharmacologic, pharmacokinetic or pharmacodynamic characteristic such as in vivo half-life in the resulting Affimer Agent. In some embodiments, the half-life of the resulting Affimer Agent is increased at least about 10, 20, 30, 40, 50, 60, 70, 80, 90 percent, 2-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 50-fold, or at least about 100-fold over an unmodified polypeptide.

Another variation of polymer system useful to modify the PK or other biological properties of the resulting Affimer Agent are the use of unstructured, hydrophilic amino acid polymers that are functional analogs of PEG, particularly as part of a fusion protein with the affimer polypeptide sequence. The inherent biodegradability of the polypeptide platform makes it attractive as a potentially more benign alternative to PEG. Another advantage is the precise molecular structure of the recombinant molecule in contrast to the polydispersity of PEG. Unlike HSA and Fc peptide fusions, in which the three-dimensional folding of the fusion partner needs to be maintained, the recombinant fusions to unstructured partners can, in many cases, be subjected to higher temperatures or harsh conditions such as HPLC purification.

One of the more advanced of this class of polypeptides is termed XTEN (Amunix) and is 864 amino acids long and comprised of six amino acids (A, E, G, P, S and T). See Schellenberger et al. “A recombinant polypeptide extends the in vivo half-life of peptides and proteins in a tunable manner” 2009 Nat Biotechnol. 27(12):1186-90. Enabled by the biodegradable nature of the polymer, this is much larger than the 40 KDa PEGS typically used and confers a concomitantly greater half-life extension. The fusion of XTEN to the affimer containing polypeptide should result in halflife extension of the final Affimer Agent by 60- to 130-fold over the unmodified polypeptide.

A second polymer based on similar conceptual considerations is PAS (XL-Protein GmbH). Schlapschy et al. “PASYlation: a biological alternative to PEGylation for extending the plasma half-life of pharmaceutically active proteins” 2013 Protein Eng Des Sel. 26(8):489-501. A random coil polymer comprised of an even more restricted set of only three small uncharged amino acids, proline, alanine and serine. AS with Fc, HAS and XTEN, the PAS modification can be genetically encoded with the affimer polypeptide sequence to produce an inline fusion protein when expressed.

c. Multispecific Fusion Proteins

In some embodiments, the Affimer Agent is a multi-specific polypeptide including, for example, a first anti-PD-L1 affimer polypeptide and at least one additional binding domain. The additional binding domain may be a polypeptide sequence selected from amongst, to illustrate, a second affimer polypeptide sequence (which may be the same or different than the first affimer polypeptide sequence), an antibody or fragment thereof or other antigen binding polypeptide, a ligand binding portion of a receptor (such as a receptor trap polypeptide), a receptor-binding ligand (such as a cytokine, growth factor or the like), engineered T-cell receptor, an enzyme or catalytic fragment thereof, or other polypeptide sequence that confers some

In some embodiments, the Affimer Agent includes one or more additional affimer polypeptide sequence that are also directed to PD-L1. The additional anti-PD-L1 affimers may be the same or different (or a mixture thereof) as the first anti-PD-L1 affimer polypeptide in order to create a multi-specific affimer fusion protein. The Affimer Agents can bind the same or overlapping sites on PD-L1, or can bind two different sites such that the Affimer Agent can simultaneously bind two sites on the same PD-L1 protein (biparatopic) or more than two sites (multiparatopic).

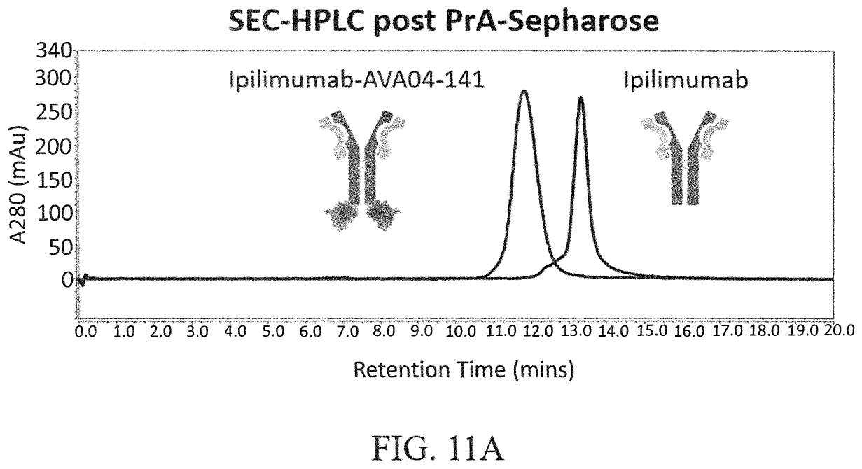

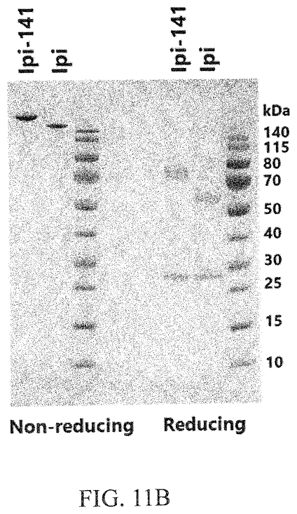

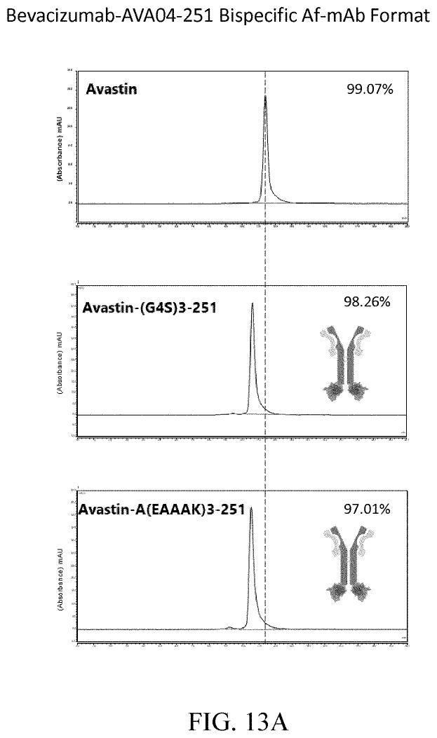

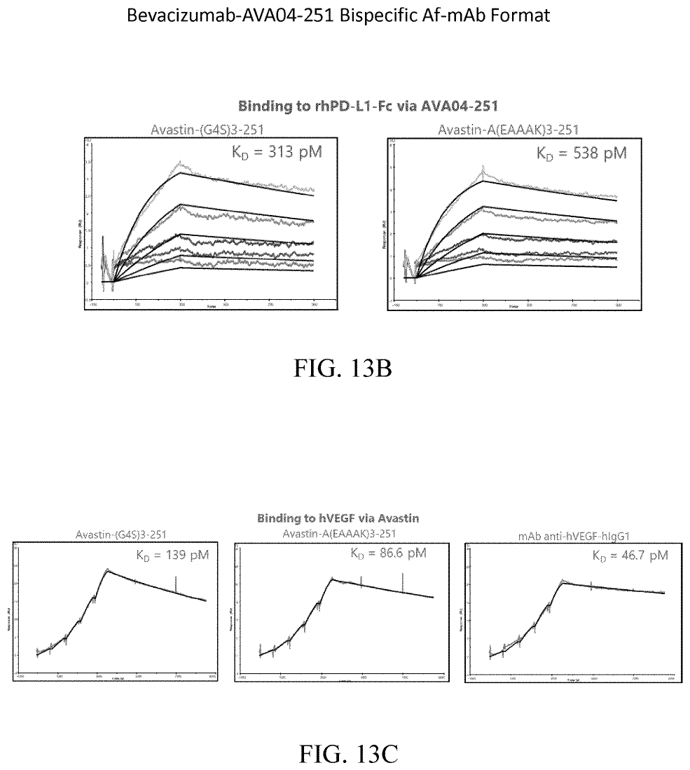

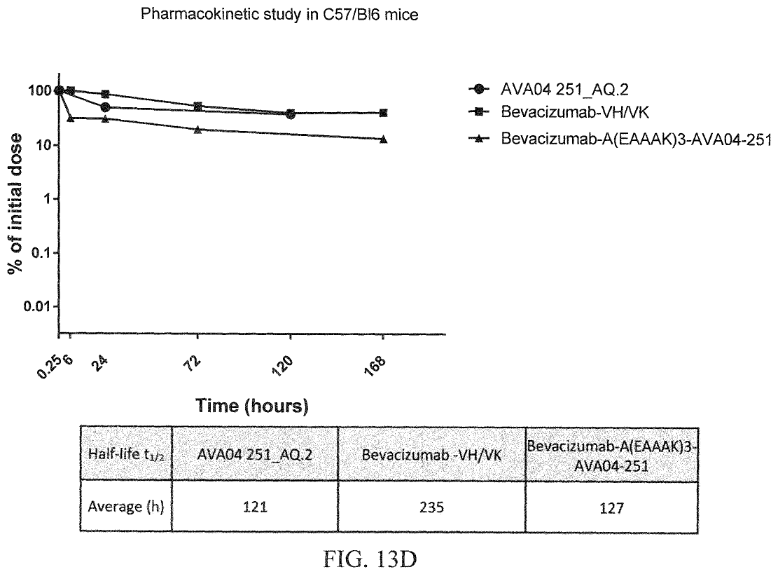

In some embodiments, the Affimer Agent includes one or more antigen binding sites from an antibody. The resulting Affimer Agent can be a single chain including both the anti-PD-L1 affimer and the antigen binding site (such as in the case of an scFV), or can be a multimeric protein complex such as in antibody assembled with heavy and/or light chains to which the sequence of the anti-PD-L1 antibody has also been fused. An exemplary affimer/antibody fusion of this format is the Ipilimumab-AVA04-141 bispecific antibody shown in FIG. 11A, which is divalent for each of CTLA-4 and PD-L1. Another is the Bevacizumab-AVA04-251 bispecific antibody shown in FIG. 13A, which is divalent for each of VEGF-A and PD-L1.

In the case of the illustrated Ipilimumab-AVA04-141 bispecific antibody, the anti-PD-L1 affimer polypeptide is provided as an in-line fusion at the C-terminal end of the heavy chain of the anti-CTLA-4 antibody, where the heavy chain (including the secretion signal sequence MPLLLLLPLLWAGALA (SEQ ID NO: 136) which can be removed, and a Gly₄-Ser repeat linker) has the affimer fusion sequence:

(SEQ ID NO: 114)
MPLLLLLPLLWAGALAQVQLVESGGGVVQPGRSLRLSCAASGFTFSSYTM
HWVRQAPGKGLEWVTFISYDGNNKYYADSVKGRFTISRDNSKNTLYLQM
NSLRAEDTAIYYCARTGWLGPFDYWGQGTLVTVSSASTKGPSVFPLAPSS
KSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGKGGGGSGGGGSGGGGSIPRGLSEAKPATPEIQEIVDKV
KPQLEEKTNETYGKLEAVQYKTQVLAAAHFPEHFWSTNYYIKVRAGDNK
YMHLKVFNGPQPADMSAEFADRVLTGYQVDKNKDDELTGF

And the light chain (including the secretion signal sequence MPLLLLLPLLWAGALA (SEQ ID NO: 136) which can be removed) has the sequence of the native Ipilimumab antibody:

(SEQ ID NO: 115)
MPLLLLLPLLWAGALAEIVLTQSPGTLSLSPGERATLSCRASQSVGSSYLA
WYQQKPGQAPRLLIYGAFSRATGIPDRFSGSGSGTDFTLTISRLEPEDFAV
YYCQQYGSSPWTFGQGTKVEIKRTVAAPSVFIFPPSDEQLKSGTASVVCLL
NNFYPREAKVQWKVDNALQSGNSQESVTEQDSKDSTYSLSSTLTLSKAD
YEKHKVYACEVTHQGLSSPVTKSFNRGEC

Likewise, in the case of the illustrated Bevacizumab-AVA04-251 bispecific antibody, the anti-PD-L1 affimer polypeptide is provided as an in-line fusion at the C-terminal end of the heavy chain of the anti-VEGF-A antibody, where the heavy chain (including the secretion signal sequence MPLLLLLPLLWAGALA (SEQ ID NO: 136) which can be removed and a flexible Gly₄-Ser repeat linker) has the affimer fusion sequence:

(SEQ ID NO: 116)
MPLLLLLPLLWAGALAEVQLVESGGGLVQPGGSLRLSCAASGYTFTNYG
MNWVRQAPGKGLEWVGWINTYTGEPTYAADFKRRFTFSLDTSKSTAYLQ
MNSLRAEDTAVYYCAKYPHYYGSSHWYFDVWGQGTLVTVSSASTKGPS
VFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSV
MHEALHNHYTQKSLSLSPGGGGGSGGGGSGGGGSIPRGLSEAKPATPEIQE
IVDKVKPQLEEKTGETYGKLEAVQYKTQVLAREGRQDWVLSTNYYIKVR
AGDNKYMHLKVFNGPWVPFPHQQLADRVLTGYQVDKNKDDELTGF

And the light chain (including the secretion signal sequence MPLLLLLPLLWAGALA (SEQ ID NO: 136) which can be removed) has the sequence of the native Bevacizumab antibody:

(SEQ ID NO: 117)
MPLLLLLPLLWAGALADIQMTQSPSSLSASVGDRVTITCSASQDISNYLN
WYQQKPGKAPKVLIYFTSSLHSGVPSRFSGSGSGTDFTLTISSLQPEDFAT
YYCQQYSTVPWTFGQGTKVEIKRTVAAPSVFIFPPSDEQLKSGTASVVCL
LNNFYPREAKVQWKVDNALQSGNSQESVTEQDSKDSTYSLSSTLTLSKA
DYEKHKVYACEVTHQGLSSPVTKSFNRGEC

To further illustrate the flexibility in formatting the affimers of the present disclosure provide, a version of the Bevacizumab-AVA04-251 bispecific antibody was also generated in which the light chain was the same as above but the heavy chain included a rigid linker between the antibody heavy chain and anti-PD-L1 affimer, where the heavy chain (including the secretion signal sequence MPLLLLLPLLWAGALA (SEQ ID NO: 136) which can be removed and a rigid A(EAAAK)₃ linker) has the affimer fusion sequence:

(SEQ ID NO: 118)
MPLLLLLPLLWAGALAEVQLVESGGGLVQPGGSLRLSCAASGYTFTNYG
MNWVRQAPGKGLEWVGWINTYTGEPTYAADFKRRFTFSLDTSKSTAYLQ
MNSLRAEDTAVYYCAKYPHYYGSSHWYFDVWGQGTLVTVSSASTKGPS
VFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSV
MHEALHNHYTQKSLSLSPGKAEAAAKEAAAKEAAAKIPRGLSEAKPATPE
IQEIVDKVKPQLEEKTGETYGKLEAVQYKTQVLAREGRQDWVLSTNYYIK
VRAGDNKYMHLKVFNGPWVPFPHQQLADRVLTGYQVDKNKDDELTGF

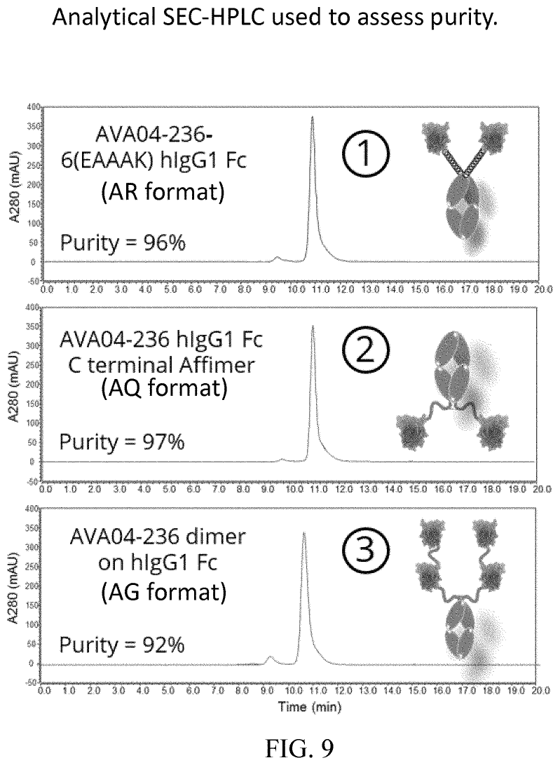

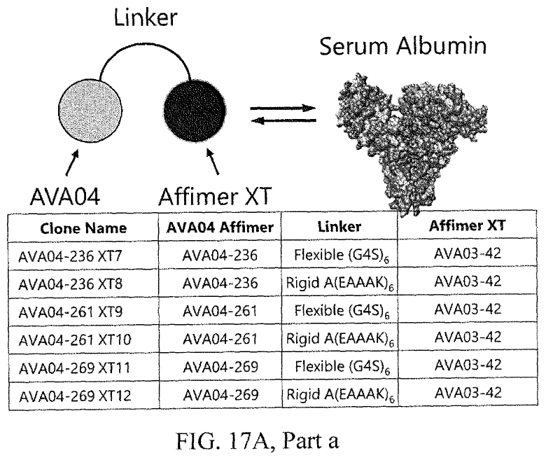

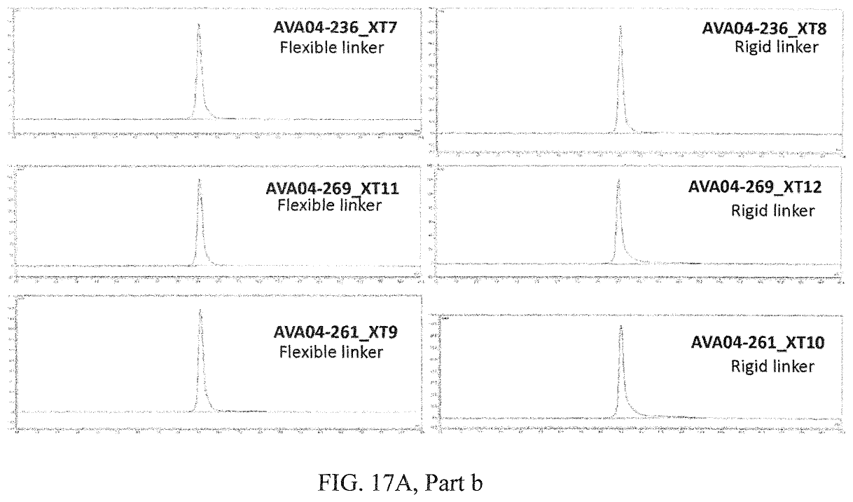

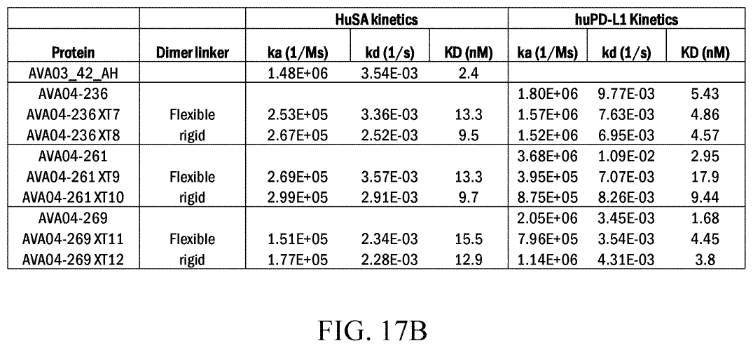

As will be apparent to those skilled in the art and illustrated in FIG. 17, the anti-PD-L1 affimer polypeptide sequence can be added at either of the N-terminal or C-terminal ends of the heavy or light chain of the antibody, or combinations/permutations thereof. Moreover, as shown in FIG. 9 in the context of multimeric affimers, more than one affimer sequence can be included to an any given antibody chain.

In some embodiments with respect to a multi-specific Affimer Agent comprising a full-length immunoglobulin, the fusion of the affimer polypeptide sequence to the antibody will preserve the Fc function of the Fc region of the immunoglobulin. For instance, in some embodiments, the Affimer Agent will be capable of binding, via its Fc portion, to the Fc receptor of Fc receptor-positive cells. In some further embodiments, the Affimer Agent may activate the Fc receptor-positive cell by binding to the Fc receptor-positive cell, thereby initiating or increasing the expression of cytokines and/or co-stimulatory antigens. Furthermore, the Affimer Agent may transfer at least a second activation signal required for physiological activation of the T cell to the T cell via the co-stimulatory antigens and/or cytokines.

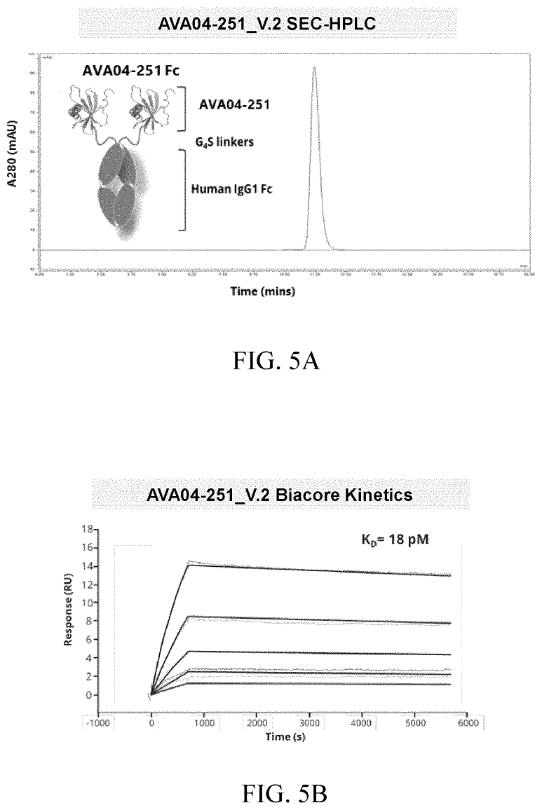

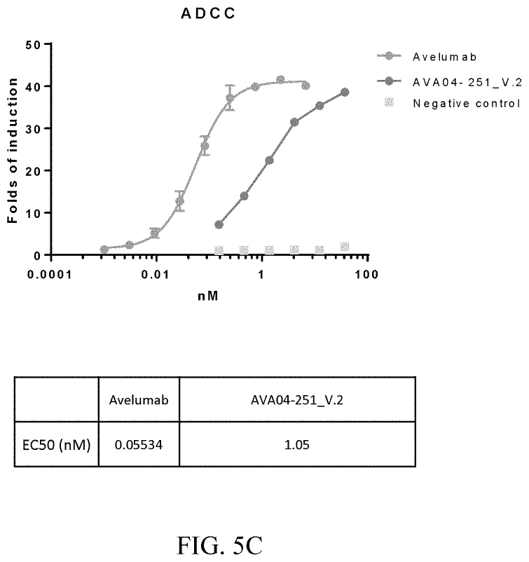

In some embodiments, resulted from the binding of its Fc portion to other cells that express Fc receptors present on the surface of effector cells from the immune system, such as immune cells, hepatocytes, and endothelial cells, the Affimer Agent may possess antibody-dependent cellular cytotoxicity (ADCC) function, a mechanism of cell-mediated immune defense whereby an effector cell of the immune system actively lyses a target cell, whose membrane-surface antigen has been bound by an antibody, and therefore, trigger tumor cell death via ADCC. In some further embodiments, the Affimer Agent is capable of demonstrating ADCC function (FIG. 5C).

As described above, apart from the Fc-mediated cytotoxicity, the Fc portion may contribute to maintaining the serum levels of the Affimer Agent, critical for its stability and persistence in the body. For example, when the Fc portion binds to Fc receptors on endothelial cells and on phagocytes, the Affimer Agent may become internalized and recycled back to the blood stream, enhancing its half-life within the body.

Exemplary targets of the additional affimer polypeptides include, but are not limited to, another immune checkpoint protein, and immune co-stimulatory receptor (particularly if the additional affimer(s) can agonize the co-stimulatory receptor), a receptor, a cytokine, a growth factor, or a tumor-associated antigen, mere to illustrate.

Where the Affimer Agent is an affimer/antibody fusion protein, the immunoglobulin portion, for example, may be an immunoglobulin is a monoclonal antibody against CD20, CD30, CD33, CD38, CD52, VEGF, VEGF receptors, EGFR or Her2/neu. A few illustrative examples for such immunoglobulins include an antibody comprised within any of the following: trastuzumab, panitumumab, cetuximab, obinutuzumab, rituximab, pertuzumab, alemtuzumab, bevacizumab, tositumomab, ibritumomab, ofatumumab, brentuximab and gemtuzumab.

In some embodiments, the anti-PD-L1 affimer polypeptide is part of an Affimer Agent that includes one more binding domains that inhibit an immune checkpoint molecule, such as expressed on a T-cell, including but not limited to PD-1, PD-L2, CTLA-4, NKG2A, KIR, LAG-3, TIM-3, CD96, VISTA, or TIGIT.

In some embodiments, the anti-PD-L1 affimer polypeptide is part of an Affimer Agent that includes one more binding domains that agonizes an immune co-stimulatory molecule, such as expressed on a T-cell, including but not limited to CD28, ICOS, CD137, OX40, GITR, CD27, CD30, HVEM, DNAM-1 or CD28H.

In some embodiments, the anti-PD-L1 affimer polypeptide is part of an Affimer Agent that includes one more ligand agonists of immune co-stimulatory molecules, such as an agonist ligand for CD28, ICOS, CD137, OX40, GITR, CD27, CD30, HVEM, DNAM-1 or CD28H.

By combining the PD-L1 inhibitory activity of the anti-PD-L1 affimer with binding domains that block of one or several of inhibitory immune checkpoints and/or activate one or more of immune costimulatory pathways, the multi-specific Affimer Agents can rescue otherwise exhausted anti-tumor T cells, enhance anti-tumor immunity and, thereby, enlists positive responses in cancer patients. In some further embodiments, dual blockade by the Affimer Agent of coordinately expressed immune-checkpoint proteins can produce additive or synergistic anti-tumor activities.

In some embodiments, the anti-PD-L1 affimer polypeptide is part of an Affimer Agent that includes one more binding domains that inhibit a soluble immune suppressing molecule, such as a binding domain that binds to the soluble immune suppressing molecules (such as a receptor trap) or a binding domain that binds to the corresponding cognate receptor and prevents ligand activation of the receptor, including but not limited to antagonists of PGE2, TGF-β, VEGF, CCL2, IDO, CSF1, IL-10, IL-13, IL-23, adenosine, or STAT3 activators. In certain instances, the Affimer Agent includes a VEGF Receptor Trap domain, such as the VEGF binding receptor domain of Aflibercept. In another example, the Affimer Agent includes a TGF-β Receptor Trap domain, such as the TGF-β binding receptor domain of MSB0011359C.

In some embodiments, the anti-PD-L1 affimer polypeptide is part of an Affimer Agent that includes one more binding domains that bind to a protein upregulated in the tumor microenvironment, i.e., a tumor associated antigen, such as upregulated on tumor cells in the tumor, or macrophage, fibroblasts, T-cells or other immune cells that infiltrate the tumor.

In some embodiments, the anti-PD-L1 affimer polypeptide is part of an Affimer Agent that includes one more binding domains that bind to a protein selected from the groups consisting of CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX4OL, PVR, SIRPα, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DL5A, KIR2DL5B, KIR3DL1, KIR3DL2, KIR3DL3, NKG2A, NKG2C, NKG2E or TSLP.

d. Conjugates

The subject Affimer Agents may also include one or more Functional Moieties intended to impart detectability or additional pharmacologic activity to the Affimer Agent. Functional Moieties for detection are those which can be employed to detect association of the Affimer Agent with a cell or tissue (such as a Tumor cell) in vivo. Functional Moieties with pharmacologic activity are those agents which are meant to be delivered to the tissue expressing the target of the Affimer Agent (PD-L1 in the case of the PDL-L1 Affimer Agents of the present disclosure) and in doing so have a pharmacologic consequence to the targeted tissues or cells.

The present disclosure provides Affimer Agents including conjugates of substances having a wide variety of functional groups, substituents or moieties, with those Functional Moieties including but not limited to a label; a dye; an immunoadhesion molecule; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance.

(i) Labels and Detectable Moieties

Where the moiety is a detectable label, it can be a fluorescent label, radioactive label, enzymatic label or any other label known to the skilled person. In some embodiments, the Functional Moiety is a detectable label that can be included as part of a conjugate to form certain Affimer Agents suitable for medical imaging. By “medical imaging” is meant any technique used to visualise an internal region of the human or animal body, for the purposes of diagnosis, research or therapeutic treatment. For instance, the Affimer Agent can be detected (and quantitated) by radioscintigraphy, magnetic resonance imaging (MRI), computed tomography (CT scan), nuclear imaging, positron emission comprising a metal tomography (PET) contrast agent, optical imaging (such as fluorescence imaging including near-infrared fluorescence (NIRF) imaging), bioluminescence imaging, or combinations thereof. The Functional Moiety is optionally a contrast agent for X-ray imaging. Agents useful in enhancing such techniques are those materials that enable visualization of a particular locus, organ or disease site within the body, and/or that lead to some improvement in the quality of the images generated by the imaging techniques, providing improved or easier interpretation of those images. Such agents are referred to herein as contrast agents, the use of which facilitates the differentiation of different parts of the image, by increasing the “contrast” between those different regions of the image. The term “contrast agents” thus encompasses agents that are used to enhance the quality of an image that may nonetheless be generated in the absence of such an agent (as is the case, for instance, in MRI), as well as agents that are prerequisites for the generation of an image (as is the case, for instance, in nuclear imaging).

In some embodiments, the detectable label includes a chelate moiety for chelating a metal, e.g., a chelator for a radiometal or paramagnetic ion. In some embodiments, the detectable label is a chelator for a radionuclide useful for radiotherapy or imaging procedures. Radionuclides useful within the present disclosure include gamma-emitters, positron-emitters, Auger electron-emitters, X-ray emitters and fluorescence-emitters, with beta- or alpha-emitters for therapeutic use. Examples of radionuclides useful as toxins in radiation therapy include: ⁴³K, ⁴⁷Sc, ⁵¹Cr, ⁵⁷Co, ⁵⁸Co, ⁵⁹Fe, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁸¹Rb, ⁹⁰Y, ⁹⁷Ru, ^{99m}TC, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Pb, ²¹²Bi and ²¹³Bi. Conditions under which a chelator will coordinate a metal are described, for example, by Gansow et al., U.S. Pat. Nos. 4,831,175, 4,454,106 and 4,472,509. Examples of chelators includes, merely to illustrate, 1,4,7-triazacyclononane-N,N′,N″-triacetic acid (NOTA) 1,4,7,10-tetraazacyclododecane-N,N′,N″,N′″-tetraacetic acid (DOTA) 1,4,8,11-tetraazacyclotetradecane-N,N′,N″,N′″-tetraacetic acid (TETA).

Other detectable isotopes that can be incorporated directly into the amino acid residues of the affimer polypeptide or which otherwise do not require a chelator, include ³H, ¹⁴C, ³²P, ³⁵S and ³⁶Cl.

Paramagnetic ions, useful for diagnostic procedures, may also be administered. Examples of paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), or combinations of these paramagnetic ions.

Examples of fluorescent labels include, but are not restricted to, organic dyes (e.g. cyanine, fluorescein, rhodamine, Alexa Fluors, Dylight fluors, ATTO Dyes, BODIPY Dyes, etc.), biological fluorophores (e.g. green fluorescent protein (GFP), R-Phycoerythrin, etc.), and quantum dots.

Non-limiting fluorescent compound that may be used in the present disclosure include, Cy5, Cy5.5 (also known as Cy5++), Cy2, fluorescein isothiocyanate (FITC), tetramethylrhodamine isothiocyanate (TRITC), phycoerythrin, Cy7, fluorescein (FAM), Cy3, Cy3.5 (also known as Cy3++), Texas Red, LightCycler-Red 640, LightCycler Red 705, tetramethylrhodamine (TMR), rhodamine, rhodamine derivative (ROX), hexachlorofluorescein (HEX), rhodamine 6G (R6G), the rhodamine derivative JA133, Alexa Fluorescent Dyes (such as Alexa Fluor 488, Alexa Fluor 546, Alexa Fluor 633, Alexa Fluor 555, and Alexa Fluor 647), 4′,6-diamidino-2-phenylindole (DAPI), Propidium iodide, AMCA, Spectrum Green, Spectrum Orange, Spectrum Aqua, Lissamine, and fluorescent transition metal complexes, such as europium. Fluorescent compound that can be used also include fluorescent proteins, such as GFP (green fluorescent protein), enhanced GFP (EGFP), blue fluorescent protein and derivatives (BFP, EBFP, EBFP2, Azurite, mKalamal), cyan fluorescent protein and derivatives (CFP, ECFP, Cerulean, CyPet) and yellow fluorescent protein and derivatives (YFP, Citrin, Venus, YPet). WO2008142571, WO2009056282, WO9922026.

Examples of enzymatic labels include, but are not restricted to, horseradish peroxidase (HRP), alkaline phosphatase (AP), glucose oxidase and β-galactosidase.

Another well-known label is biotin. Biotin labels are typically composed of the biotinyl group, a spacer arm and a reactive group that is responsible for attachment to target functional groups on proteins. Biotin can be useful for attaching the labelled protein to other moieties which comprise an avidin moiety.

(ii) Affimer-Drug Conjugates

In some embodiments, the Affimer Agent includes one or more therapeutic agents, e.g., to form an affimer-drug conjugate. As used herein, the term “therapeutic agent” refers to a substance that may be used in the cure, mitigation, treatment, or prevention of disease in a human or another animal. Such therapeutic agents include substances recognized in the official United States Pharmacopeia, official Homeopathic Pharmacopeia of the United States, official National Formulary, or any supplement thereof, and include but are not limited to small molecules, nucleotides, oligopeptides, polypeptides, etc. Therapeutic agents that may be attached to affimer-containing polypeptides include, but are not limited to, cytotoxic agents, anti-metabolites, alkylating agents, antibiotics, growth factor, cytokines, anti-angiogenic agents, antimitotic agents, toxins, apoptotic agents or the like, such as DNA alkylating agents, topoisomerase inhibitors, endoplasmic reticulum stress inducing agents, platinum compounds, antimetabolites, vincalkaloids, taxanes, epothilones, enzyme inhibitors, receptor antagonists, therapeutic antibodies, tyrosine kinase inhibitors, radiosensitizers, and chemotherapeutic combination therapies, such as illustrations.

Non-limiting examples of DNA alkylating agents are nitrogen mustards, such as Mechlorethamine, Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, ThioTEPA, Triaziquone, Triethylenemelamine; Hydrazines (Procarbazine); Triazenes such as Dacarbazine and Temozolomide; Altretamine and Mitobronitol.

Non-limiting examples of Topoisomerase I inhibitors include Campothecin derivatives including CPT-11 (irinotecan), SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier Y. (2006) Nat. Rev. Cancer 6(10):789-802 and U.S. Patent Publication No. 200510250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-]25, Crow et al. (1994) J. Med. Chem. 37(19):31913194, and Crespi et al. (1986) Biochem. Biophys. Res. Commun. 136(2):521-8. Topoisomerase II inhibitors include, but are not limited to Etoposide and Teniposide. Dual topoisomerase I and II inhibitors include, but are not limited to, Saintopin and other Naphthecenediones, DACA and other Acridine-4-Carboxamindes, Intoplicine and other Benzopyridoindoles, TAS-103 and other 7H-indeno[2,1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]Perimidines, and Anthracenyl-amino Acid Conjugates as described in Denny and Baguley (2003) Curr. Top. Med. Chem. 3(3):339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Examples of endoplasmic reticulum stress inducing agents include, but are not limited to, dimethyl-celecoxib (DMC), nelfinavir, celecoxib, and boron radiosensitizers (i.e. velcade (Bortezomib)).

Non-limiting examples of platinum-based compound include Carboplatin, Cisplatin, Nedaplatin, Oxaliplatin, Triplatin tetranitrate, Satraplatin, Aroplatin, Lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Non-limiting examples of antimetabolite agents include Folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-I (MBMS-247616, consisting of tegafur and two modulators, a 5-chloro-2,4-dihydroxypyridine and potassium oxonate), ralititrexed (tomudex), no latrexed (Thymitaq, AG337), LY231514 and ZD9331, as described for example in Papamicheal (1999) The Oncologist 4:478-487.

Examples of vincalkaloids, include, but are not limited to Vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (Tipifamib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include, but are not limited to ERA (Atrasentan); retinoid X receptor (Bexarotene); and a sex steroid (Testolactone).

Examples of therapeutic antibodies include, but are not limited to anti-HER1/EGFR (Cetuximab, Panitumumab); Anti-HER2/neu (erbB2) receptor (Trastuzumab); Anti-EpCAM (Catumaxomab, Edrecolomab) Anti-VEGF-A (Bevacizumab); Anti-CD20 (Rituximab, Tositumomab, Ibritumomab); Anti-CD52 (Alemtuzumab); and Anti-CD33 (Gemtuzumab). U.S. Pat. Nos. 5,776,427 and 7,601,355.

Examples of tyrosine kinase inhibitors include, but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

Chemotherapeutic agents that can be attached to the present affimer-containing polypeptides may also include amsacrine, Trabectedin, retinoids (Alitretinoin, Tretinoin), Arsenic trioxide, asparagine depleter Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

Examples of specific therapeutic agents that can be linked, ligated, or associated with the affimer-containing polypeptides of the disclosure are flomoxef; fortimicin(s); gentamicin(s); glucosulfone solasulfone; gramicidin S; gramicidin(s); grepafloxacin; guamecycline; hetacillin; isepamicin; josamycin; kanamycin(s); flomoxef; fortimicin(s); gentamicin(s); glucosulfone solasulfone; gramicidin S; gramicidin(s); grepafloxacin; guamecycline; hetacillin; isepamicin; josamycin; kanamycin(s); bacitracin; bambermycin(s); biapenem; brodimoprim; butirosin; capreomycin; carbenicillin; carbomycin; carumonam; cefadroxil; cefamandole; cefatrizine; cefbuperazone; cefclidin; cefdinir; cefditoren; cefepime; cefetamet; cefixime; cefmenoxime; cefininox; cladribine; apalcillin; apicycline; apramycin; arbekacin; aspoxicillin; azidamfenicol; aztreonam; cefodizime; cefonicid; cefoperazone; ceforamide; cefotaxime; cefotetan; cefotiam; cefozopran; cefpimizole; cefpiramide; cefpirome; cefprozil; cefroxadine; cefteram; ceftibuten; cefuzonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin; 6-mercaptopurine; thioguanine; capecitabine; docetaxel; etoposide; gemcitabine; topotecan; vinorelbine; vincristine; vinblastine; teniposide; melphalan; methotrexate; 2-p-sulfanilyanilinoethanol; 4,4′-sulfinyldianiline; 4-sulfanilamidosalicylic acid; butorphanol; nalbuphine. streptozocin; doxorubicin; daunorubicin; plicamycin; idarubicin; mitomycin C; pentostatin; mitoxantrone; cytarabine; fludarabine phosphate; butorphanol; nalbuphine. streptozocin; doxorubicin; daunorubicin; plicamycin; idarubicin; mitomycin C; pentostatin; mitoxantrone; cytarabine; fludarabine phosphate; acediasulfone; acetosulfone; amikacin; amphotericin B; ampicillin; atorvastatin; enalapril; ranitidine; ciprofloxacin; pravastatin; clarithromycin; cyclosporin; famotidine; leuprolide; acyclovir; paclitaxel; azithromycin; lamivudine; budesonide; albuterol; indinavir; metformin; alendronate; nizatidine; zidovudine; carboplatin; metoprolol; amoxicillin; diclofenac; lisinopril; ceftriaxone; captopril; salmeterol; xinafoate; imipenem; cilastatin; benazepril; cefaclor; ceftazidime; morphine; dopamine; bialamicol; fluvastatin; phenamidine; podophyllinic acid 2-ethylhydrazine; acriflavine; chloroazodin; arsphenamine; amicarbilide; aminoquinuride; quinapril; oxymorphone; buprenorphine; floxuridine; dirithromycin; doxycycline; enoxacin; enviomycin; epicillin; erythromycin; leucomycin(s); lincomycin; lomefloxacin; lucensomycin; lymecycline; meclocycline; meropenem; methacycline; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; paromomycin; pazufloxacin; penicillin N; pipacycline; pipemidic acid; polymyxin; primycin; quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fungichromin; mepartricin; nystatin; oligomycin(s); perimycin A; tubercidin; 6-azauridine; 6-diazo-5-oxo-L-norleucine; aclacinomycin(s); ancitabine; anthramycin; azacitadine; azaserine; bleomycin(s); ethyl biscoumacetate; ethylidene dicoumarol; iloprost; lamifiban; taprostene; tioclomarol; tirofiban; amiprilose; bucillamine; gusperimus; gentisic acid; glucamethacin; glycol salicylate; meclofenamic acid; mefenamic acid; mesalamine; niflumic acid; olsalazine; oxaceprol; S-enosylmethionine; salicylic acid; salsalate; sulfasalazine; tolfenamic acid; carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; mycophenolic acid; procodazole; romurtide; sirolimus (rapamycin); tacrolimus; butethamine; fenalcomine; hydroxytetracaine; naepaine; orthocaine; piridocaine; salicyl alcohol; 3-amino-4-hydroxybutyric acid; aceclofenac; alminoprofen; amfenac; bromfenac; bromosaligenin; bumadizon; carprofen; diclofenac; diflunisal; ditazol; enfenamic acid; etodolac; etofenamate; fendosal; fepradinol; flufenamic acid; Tomudex (N-[[5-[[(1,4-Dihydro-2-methyl-4-oxo-6-quinazolinyl)methyl]methylamino]-2-thienyl]carbonyl]-L-glutamic acid), trimetrexate, tubercidin, ubenimex, vindesine, zorubicin; argatroban; coumetarol or dicoumarol.

In some embodiments, the Affimer Agent includes a conjugated cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating to antibodies and other proteins may be employed in generating the conjugates of the present disclosure, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating peptide, polypeptide and organic and inorganic moieties to antibodies and other proteins are conventional and very well known in the art and readily adapted for generating those versions of the subject Affimer Agents.

Where the conjugated moiety is a peptide or polypeptide, that moiety can be chemically cross-linked to the affimer-containing polypeptide, or can be included as part of a fusion protein with the affimer-containing polypeptide. And illustrative example would be a diptheria toxin-affimer fusion protein. In the case of non-peptide entities, the addition to the affimer-containing polypeptide will generally be by way of chemical conjugation to the affimer-containing polypeptide—such as through a functional group on an amino acid side chain or the carboxyl group at the C-terminal or amino group at the N-terminal end of the polypeptide. In some embodiment, whether as a fusion protein or chemically cross-linked moiety, the conjugated moiety will include one or more sites that can be cleaved by an enzyme or are otherwise sensitive to an environmental condition (such as pH) that permits the conjugated moiety to be released from the affimer-containing polypeptide, such as in the Tumor or other diseased tissue (or tissue to be protected if the conjugated moiety functions to protect healthy tissue).

### IV. Expression Methods and Systems

Recombinant Affimer Agent proteins described herein can be produced by any suitable method known in the art. Such methods range from direct protein synthesis methods to constructing a DNA sequence encoding polypeptide sequences and expressing those sequences in a suitable host. For those recombinant Affimer Agent proteins including further modifications, such as a chemical modifications or conjugation, the recombinant Affimer Agent protein can be further manipulated chemically or enzymatically after isolation form the host cell or chemical synthesis.

The present disclosure includes recombinant methods and nucleic acids for recombinantly expressing the recombinant Affimer Agent proteins of the present disclosure comprising (i) introducing into a host cell a polynucleotide encoding the amino acid sequence of said Affimer Agent, for example, wherein the polynucleotide is in a vector and/or is operably linked to a promoter; (ii) culturing the host cell (e.g., eukaryotic or prokaryotic) under condition favorable to expression of the polynucleotide and, (iii) optionally, isolating the Affimer Agent from the host cell and/or medium in which the host cell is grown. See e.g., WO 04/041862, WO 2006/122786, WO 2008/020079, WO 2008/142164 or WO 2009/068627.

In some embodiments, a DNA sequence encoding a recombinant Affimer Agent protein of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once a nucleic acid sequence encoding a recombinant Affimer Agent protein of the disclosure has been obtained, the vector for the production of the recombinant Affimer Agent protein may be produced by recombinant DNA technology using techniques well known in the art. Methods which are well known to those skilled in the art can be used to construct expression vectors containing the recombinant Affimer Agent coding sequences and appropriate transcriptional and translational control signals. These methods include, for example, in vitro recombinant DNA techniques, synthetic techniques, and in vivo genetic recombination. (See, for example, the techniques described in Sambrook et al, 1990, MOLECULAR CLONING, A LABORATORY MANUAL, 2d Ed., Cold Spring Harbor Laboratory, Cold Spring Harbor, N.Y. and Ausubel et al. eds., 1998, CURRENT PROTOCOLS IN MOLECULAR BIOLOGY, John Wiley & Sons, NY).

An expression vector comprising the nucleotide sequence of a recombinant Affimer Agent protein can be transferred to a host cell by conventional techniques (e.g., electroporation, liposomal transfection, and calcium phosphate precipitation) and the transfected cells are then cultured by conventional techniques to produce the recombinant Affimer Agent protein of the disclosure. In specific embodiments, the expression of the recombinant Affimer Agent protein is regulated by a constitutive, an inducible or a tissue, specific promoter.

The expression vector may include an origin of replication, such as may be selected based upon the type of host cell being used for expression. By way of example, the origin of replication from the plasmid pBR322 (Product No. 303-3s, New England Biolabs, Beverly, Mass.) is useful for most Gram-negative bacteria while various origins from SV40, polyoma, adenovirus, vesicular stomatitus virus (VSV) or papillomaviruses (such as HPV or BPV) are useful for cloning vectors in mammalian cells. Generally, the origin of replication component is not needed for mammalian expression vectors (for example, the SV40 origin is often used because it contains the early promoter).

The vector may include one or more selectable marker genes, e.g., genetic elements that encode a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells. Other selection genes may be used to amplify the gene which will be expressed. Amplification is a process where genes which are in greater demand for the production of a protein critical for growth are reiterated in tandem within the chromosomes of successive generations of recombinant cells. Examples of selectable markers for mammalian cells include dihydrofolate reductase (DHFR) and thymidine kinase. The mammalian cell transformants are placed under selection pressure which only the transformants are uniquely adapted to survive by virtue of the marker present in the vector. Selection pressure is imposed by culturing the transformed cells under conditions in which the concentration of selection agent in the medium is successively changed, thereby leading to amplification of both the selection gene and the DNA that encodes the recombinant Affimer Agent protein. As a result, increased quantities of the recombinant Affimer Agent protein are synthesized from the amplified DNA.

The vector may also include one or more ribosome binding site, which will be transcribed into the mRNA including the coding sequence for the recombinant Affimer Agent protein. For example, such a site is characterized by a Shine-Dalgarno sequence (prokaryotes) or a Kozak sequence (eukaryotes). The element is typically located 3′ to the promoter and 5′ to the coding sequence of the polypeptide to be expressed. The Shine-Dalgarno sequence is varied but is typically a polypurine (having a high A-G content). Many Shine-Dalgarno sequences have been identified, each of which can be readily synthesized using methods set forth above and used in a prokaryotic vector.

The expression vectors will typically contain a promoter that is recognized by the host organism and operably linked to a nucleic acid molecule encoding the recombinant Affimer Agent protein. Either a native or heterologous promoter may be used depending the host cell used for expression and the yield desired.

Promoters for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, and they can be ligated to a desired nucleic acid sequence(s), using linkers or adapters as desired to supply restriction sites.

Promoters for use with yeast hosts are also known in the art. Yeast enhancers are advantageously used with yeast promoters. Suitable promoters for use with mammalian host cells are well known and include those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40). Other suitable mammalian promoters include heterologous mammalian promoters, e.g., heat-shock promoters and the actin promoter.

Additional promoters which may be used for expressing the selective binding agents of the disclosure include, but are not limited to: the SV40 early promoter region (Bernoist and Chambon, Nature, 290:304-310, 1981); the CMV promoter; the promoter contained in the 3′ long terminal repeat of Rous sarcoma virus (Yamamoto et al. (1980), Cell 22: 787-97); the herpes thymidine kinase promoter (Wagner et al. (1981), Proc. Natl. Acad. Sci. U.S.A. 78: 1444-5); the regulatory sequences of the metallothionine gene (Brinster et al, Nature, 296; 39-42, 1982); prokaryotic expression vectors such as the beta-lactamase promoter (Villa-Kamaroff, et al., Proc. Natl. Acad. Sci. U.S.A., 75; 3727-3731, 1978); or the tac promoter (DeBoer, et al. (1983), Proc. Natl. Acad. Sci. U.S.A., 80: 21-5). Also of interest are the following animal transcriptional control regions, which exhibit tissue specificity and have been utilized in transgenic animals: the elastase I gene control region which is active in pancreatic acinar cells (Swift et al. (1984), Cell 38: 639-46; Ornitz et al. (1986), Cold Spring Harbor Symp. Quant. Biol. 50: 399-409; MacDonald (1987), Hepatology 7: 425-515); the insulin gene control region which is active in pancreatic beta cells (Hanahan (1985), Nature 315: 115-22); the immunoglobulin gene control region which is active in lymphoid cells (Grosschedl et al. (1984), Cell 38; 647-58; Adames et al. (1985), Nature 318; 533-8; Alexander et al. (1987), Mol. Cell. Biol. 7: 1436-44); the mouse mammary tumor virus control region which is active in testicular, breast, lymphoid and mast cells (Leder et al. (1986), Cell 45: 485-95), albumin gene control region which is active in liver (Pinkert et al. (1987), Genes and Devel. 1: 268-76); the alphafetoprotein gene control region which is active in liver (Krumlauf et al. (1985), MoI. Cell. Biol. 5: 1639-48; Hammer et al. (1987), Science, 235: 53-8); the alpha 1-antitrypsin gene control region which is active in the liver (Kelsey et al. (1987), Genes and Devel. 1: 161-71); the beta-globin gene control region which is active in myeloid cells (Mogram et al., Nature, 315 338-340, 1985; Kollias et al. (1986), Cell 46: 89-94); the myelin basic protein gene control region which is active in oligodendrocyte cells in the brain (Readhead et al. (1987), Cell, 48: 703-12); the myosin light chain-2 gene control region which is active in skeletal muscle (Sani (1985), Nature, 314: 283-6); and the gonadotropic releasing hormone gene control region which is active in the hypothalamus (Mason et al. (1986), Science 234: 1372-8).

An enhancer sequence may be inserted into the vector to increase transcription in eukaryotic host cells. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters.

While an enhancer may be spliced into the vector at a position 5′ or 3′ to the polypeptide coding region, it is typically located at a site 5′ from the promoter.

Vectors for expressing nucleic acids include those which are compatible with bacterial, insect, and mammalian host cells. Such vectors include, inter alia, pCRII, pCR3, and pcDNA3.1 (Invitrogen Company, San Diego, Calif.), pBSII (Stratagene Company, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO90/14363) and pFastBacDual (Gibco/BRL, Grand Island, N.Y.).

Additional possible vectors include, but are not limited to, cosmids, plasmids or modified viruses, but the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™. TA Cloning® Kit, PCR2.1 plasmid derivatives, Invitrogen, Carlsbad, Calif.), and mammalian, yeast or virus vectors such as a baculovirus expression system (pBacPAK plasmid derivatives, Clontech, Palo Alto, Calif.). The recombinant molecules can be introduced into host cells via transformation, transfection, infection, electroporation, or other known techniques

Eukaryotic and prokaryotic host cells, including mammalian cells as hosts for expression of the recombinant Affimer Agent protein disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa.

A variety of host-expression vector systems may be utilized to express the recombinant Affimer Agent protein of the disclosure. Such host-expression systems represent vehicles by which the coding sequences of the recombinant Affimer Agent protein may be produced and subsequently purified, but also represent cells which may, when transformed or transfected with the appropriate nucleotide coding sequences, express the recombinant Affimer Agent protein of the disclosure in situ. These include, but are not limited to, microorganisms such as bacteria (e.g., E. coli and B. subtilis) transformed with recombinant bacteriophage DNA, plasmid DNA or cosmid DNA expression vectors containing Affimer Agent protein coding sequences; yeast (e.g., Saccharomyces pichia) transformed with recombinant yeast expression vectors containing Affimer Agent protein coding sequences; insect cell systems infected with recombinant virus expression vectors (e.g., baculovirus) containing the Affimer Agent protein coding sequences; plant cell systems infected with recombinant virus expression vectors (e.g., cauliflower mosaic virus (CμMV) and tobacco mosaic virus (TMV)) or transformed with recombinant plasmid expression vectors (e.g., Ti plasmid) containing Affimer Agent protein coding sequences; or mammalian cell systems (e.g., COS, CHO, BHK, 293, 293T, 3T3 cells, lymphotic cells (see U.S. Pat. No. 5,807,715), Per C.6 cells (rat retinal cells developed by Crucell)) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the recombinant Affimer Agent protein being expressed. For example, when a large quantity of such a protein is to be produced, for the generation of pharmaceutical compositions of the recombinant Affimer Agent protein, vectors which direct the expression of high levels of fusion protein products that are readily purified may be desirable. Such vectors include, but are not limited, to the E. coli expression vector pUR278 (Ruther et al. (1983) “Easy Identification Of cDNA Clones,” EMBO J. 2:1791-1794), in which the Affimer Agent protein coding sequence may be ligated individually into the vector in frame with the lac Z coding region so that a fusion protein is produced; pIN vectors (Inouye et al. (1985) “Up-Promoter Mutations In The Lpp Gene Of Escherichia coli,” Nucleic Acids Res. 13:3101-3110; Van Heeke et al. (1989) “Expression Of Human Asparagine Synthetase In Escherichia coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free gluta-thione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The Affimer Agent protein coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the Affimer Agent protein coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts. (see e.g., see Logan et al. (1984) “Adenovirus Tripartite Leader Sequence Enhances Translation Of mRNAs Late After Infection,” Proc. Natl. Acad. Sci. (U.S.A.) 81:3655-3659). Specific initiation signals may also be required for efficient translation of inserted Affimer Agent protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, WI38, BT483, Hs578T, HTB2, BT20 and T47D, CRL7030 and Hs578Bst.

For long-term, high-yield production of recombinant proteins, stable expression is contemplated. For example, cell lines which stably express an antibody of the disclosure may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the recombinant Affimer Agent proteins of the disclosure. Such engineered cell lines may be particularly useful in screening and evaluation of compounds that interact directly or indirectly with the recombinant Affimer Agent proteins.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11:223-232), hypoxanthine-guanine phosphoribosyltransferase (Szybalska et al. (1962) “Genetics Of Human Cess Line. IV. DNA-Mediated Heritable Transformation Of A Biochemical Trait,” Proc. Natl. Acad. Sci. (U.S.A.) 48:2026-2034), and adenine phosphoribosyltransferase (Lowy et al. (1980) “Isolation Of Transforming DNA: Cloning The Hamster Aprt Gene,” Cell 22:817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplfiable Dominant-Acting Gene,” Proc. Natl. Acad. Sci. (U.S.A.) 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. (U.S.A.) 78:1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. (U.S.A.) 78:2072-2076); neo, which confers resistance to the aminoglycoside G-418 (Tachibana et al. (1991) “Altered Reactivity Of Immunoglobutin Produced By Human-Human Hybridoma Cells Transfected By pSV.2-Neo Gene,” Cytotechnology 6(3):219-226; Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human gene therapy,” Ann. Rev. Biochem. 62:191-217). Methods commonly known in the art of recombinant DNA technology which can be used are described in Ausubel et al. (eds.), 1993, CURRENT PROTOCOLS IN MOLECULAR BIOLOGY, John Wiley & Sons, NY; Kriegler, 1990, GENE TRANSFER AND EXPRESSION, A LABORATORY MANUAL, Stockton Press, NY; and in Chapters 12 and 13, Dracopoli et al. (eds), 1994, CURRENT PROTOCOLS IN HUMAN GENETICS, John Wiley & Sons, NY.; Colbere-Garapin et al. (1981) “A New Dominant Hybrid Selective Marker For Higher Eukaryotic Cells,” J. Mol. Biol. 150:1-14; and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156).

The expression levels of a recombinant Affimer Agent protein can be increased by vector amplification (for a review, see Bebbington and Hentschel, “The Use Of Vectors Based On Gene Amplification For The Expression Of Cloned Genes In Mammaian Cells,” in DNA CLONING, Vol. 3. (Academic Press, New York, 1987)). When a marker in the vector system expressing a recombinant Affimer Agent protein is amplifiable, increase in the level of inhibitor present in culture of host cell will increase the number of copies of the marker gene. Since the amplified region is associated with the nucleotide sequence of the recombinant Affimer Agent protein, production of the recombinant Affimer Agent protein will also increase (Crouse et al. (1983) “Expression And Amplification Of Engineered Mouse Dihydrofolate Reductase Minigenes,” Mol. Cell. Biol. 3:257-266).

Where the Affimer Agent is an affimer antibody fusion or other multiprotein complex, the host cell may be co-transfected with two expression vectors, for instance the first vector encoding a heavy chain and the second vector encoding a light chain derived polypeptide, one or both of which includes an affimer polypeptide coding sequence. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot (1986) “Expression And Amplification Of Engineered Mouse Dihydrofolate Reductase Minigenes,” Nature 322:562-565; Kohler (1980) “Immunoglobulin Chain Loss In Hybridoma Lines,” Proc. Natl. Acad. Sci. (U.S.A.) 77:2197-2199). The coding sequences for the heavy and light chains may comprise cDNA or genomic DNA.

In general, glycoproteins produced in a particular cell line or transgenic animal will have a glycosylation pattern that is characteristic for glycoproteins produced in the cell line or transgenic animal. Therefore, the particular glycosylation pattern of the recombinant Affimer Agent protein will depend on the particular cell line or transgenic animal used to produce the protein. In some embodiments of affimer/antibody fusions, a glycosylation pattern comprising only non-fucosylated N-glycans may be advantageous, because in the case of antibodies this has been shown to typically exhibit more potent efficacy than fucosylated counterparts both in vitro and in vivo (See for example, Shinkawa et al., J. Biol. Chem. 278: 3466-3473 (2003); U.S. Pat. Nos. 6,946,292 and 7,214,775).

Further, expression of an Affimer Agent from production cell lines can be enhanced using a number of known techniques. For example, the glutamine synthetase gene expression system (the GS system) is a common approach for enhancing expression under certain conditions. The GS system is discussed in whole or part in connection with European Patent NOS: 0216846, 0256055, and 0323997 and European Patent Application No. 89303964.4. Thus, in some embodiments of the disclosure, the mammalian host cells (e.g., CHO) lack a glutamine synthetase gene and are grown in the absence of glutamine in the medium wherein, however, the polynucleotide encoding the immunoglobulin chain comprises a glutamine synthetase gene which complements the lack of the gene in the host cell. Such host cells containing the binder or polynucleotide or vector as discussed herein as well as expression methods, as discussed herein, for making the binder using such a host cell are part of the present disclosure.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The recombinant Affimer Agent proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, recombinant Affimer Agent proteins produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

### V. Encoded Affimers for In Vivo Delivery

An alternative approach to the delivery of therapeutic Affimer Agents protein, such as a PD-L1 Affimer Agent, would be to leave the production of the therapeutic polypeptide to the body itself. A multitude of clinical studies have illustrated the utility of in vivo gene transfer into cells using a variety of different delivery systems. In vivo gene transfer seeks to administer to patients the Encoded Affimer nucleotide sequence, rather than the Affimer Agent. This allows the patient's body to produce the therapeutic Affimer Agent of interest for a prolonged period of time, and secrete it either systemically or locally, depending on the production site. Gene-based Encoded Affimers can present a labor- and cost-effective alternative to the conventional production, purification and administration of the polypeptide version of the Affimer Agent. A number of antibody expression platforms have been pursued in vivo to which delivery of Encoded Affimers can be adapted: these include viral vectors, naked DNA and RNA. Encoded Affimer gene transfer can not only enable cost-savings by reducing the cost of goods and of production, but may also be able to reduce the frequency of drug administration. Overall, a prolonged in vivo production of the therapeutic Affimer Agent by expression of the Encoded Affimer can contribute to (i) a broader therapeutic or prophylactic application of Affimer Agents in price-sensitive conditions, (ii) an improved accessibility to therapy in both developed and developing countries, and (iii) more effective and affordable treatment modalities. In addition to in vivo gene transfer, cells can be harvested from the host (or a donor), engineered with Encoded Affimer sequences to produce Affimer Agents and re-administered to patients.

Intramuscular antibody gene administration has been most widely evaluated (reviewed in Deal et al. (2015) “Engineering humoral immunity as prophylaxis or therapy” Curr Opin Immunol. 35:113-22), and also carries the highest clinical translatability and application when applied to Encoded Affimers. Indeed, the inherent anatomical, cellular and physiological properties of skeletal muscle make it a stable environment for long-term Encoded Affimer expression and systemic circulation. Skeletal muscle is easily accessible, allowing multiple or repeated administrations. The abundant blood vascular supply provides an efficient transport system for secreted therapeutic Affimer Agents into the circulation. The syncytial nature of muscle fibers allows dispersal of nucleotides from a limited site of penetration to a large number of neighboring nuclei within the fiber. Skeletal muscle fibers are also terminally differentiated cells, and nuclei within the fibers are post-mitotic. Consequently, integration in the host genome is not a prerequisite to attain prolonged mAb expression. The liver is another site often used for pre-clinical antibody gene transfer, and is typically transfected via i.v. injection, and can also be a site of gene transfer for Encoded Affimers either for local delivery of Affimer Agents (such as in the treatment of liver cancer and/or metaplasias) or for the generation of Affimer Agents that are secreted into the vascular for systemic circulation. This organ has various physiological functions, including the synthesis of plasma proteins. This organ can be particularly well suited for in vivo Encoded Affimer expression.

The tumor presents another site for Encoded Affimer transfer, targeted either via i.v. or direct injection/electroporation. Indeed, intratumoral Encoded Affimer expression can allow for a local production of the therapeutic Affimer Agents, waiving the need for high systemic Affimer Agent levels that might otherwise be required to penetrate and impact solid tumors. A similar rationale applies for the brain, which is frequently targeted in the context of antibody gene transfer to avoid the difficulties with blood-brain barrier trafficking and would likewise be a target for delivery of Encoded Affimers. See, for example, Beckman et al. (2015) “Antibody constructs in cancer therapy: protein engineering strategies to improve exposure in solid tumors” Cancer 109(2):170-9; Dronca et al. (2015) “Immunomodulatory antibody therapy of cancer: the closer, the better” Clin Cancer Res. 21(5):944-6; and Neves et al. (2016) “Antibody approaches to treat brain diseases” Trends Biotechnol. 34(1):36-48.

The success of gene therapy has largely been driven by improvements in nonviral and viral gene transfer vectors. An array of physical and chemical nonviral methods have been used to transfer DNA and mRNA to mammalian cells and a substantial number of these have been developed as clinical stage technologies for gene therapy, both ex vivo and in vivo, and are readily adapted for delivery of the Encoded Affimers of the present disclosure. To illustrate, cationic liposome technology can be employed, which is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. See, for example, Felgner et al. (1987) Lipofection: a highly efficient, lipid-mediated DNA-transfection procedure. MNAS 84:7413-7417; San et al. (1983) “Safety and short term toxicity of a novel cationic lipid formulation for human gene therapy” Hum. Gene Ther. 4:781-788; Xu et al. (1996) “Mechanism of DNA release from cationic liposome/DNA complexes used in cell transfection” Biochemistry 35:5616-5623; and Legendre et al. (1992) “Delivery of plasmid DNA into mammalian cell lines using pH-sensitive liposomes: comparison with cationic liposomes” Pharm. Res. 9, 1235-1242.

Similarly, other polycations, such as poly-1-lysine and polyethylene-imine, can be used to deliver Encoded Affimers. These polycations complex with nucleic acids via charge interaction and aid in the condensation of DNA or RNA into nanoparticles, which are then substrates for endosome-mediated uptake. Several of these cationic nucleic acid complex technologies have been developed as potential clinical products, including complexes with plasmid DNA, oligodeoxynucleotides, and various forms of synthetic RNA. Modified (and unmodified or “naked”) DNA and RNA have also been shown to mediate successful gene transfer in a number of circumstances and can also be used as systems for delivery of Encoded Affimers. These include the use of plasmid DNA by direct intramuscular injection, the use of intratumoral injection of plasmid DNA. See, for example, Rodrigo et al. (2012) “De novo automated design of small RNA circuits for engineering synthetic riboregulation in living cells” PNAS 109:15271-15276; Oishi et al. (2005) “Smart polyion complex micelles for targeted intracellular delivery of PEGylated antisense oligonucleotides containing acid-labile linkages” Chembiochem. 6:718-725; Bhatt et al. (2015) “Microbeads mediated oral plasmid DNA delivery using polymethacrylate vectors: an effectual groundwork for colorectal cancer” Drug Deliv. 22:849-861; Ulmer et al. (1994) Protective immunity by intramuscular injection of low doses of influenza virus DNA vaccines” Vaccine 12: 1541-1544; and Heinzerling et al. (2005) “Intratumoral injection of DNA encoding human interleukin 12 into patients with metastatic melanoma: clinical efficacy” Hum. Gene Ther. 16:35-48.

Viral vectors are currently used as a delivery vehicle in the vast majority of pre-clinical and clinical gene therapy trials and in the first to be approved directed gene therapy. See Gene Therapy Clinical Trials Worldwide 2017 (abedia.com/wiley/). The main driver thereto is their exceptional gene delivery efficiency, which reflects a natural evolutionary development; viral vector systems are attractive for gene delivery, because viruses have evolved the ability to cross through cellular membranes by infection, thereby delivering nucleic acids such as Encoded Affimers to target cells. Pioneered by adenoviral systems, the field of viral vector-mediated antibody gene transfer made significant strides in the past decades. The myriad of successfully evaluated administration routes, pre-clinical models and disease indications puts the capabilities of antibody gene transfer at full display through which the skilled artisan would readily be able to identify and adapt antibody gene transfer systems and techniques for in vivo delivery of Encoded Affimer constructs. Muscle has emerged as the administration site of choice for prolonged mAb expression and would similarly be a suitable target tissue for prolonged Affimer Agent expression. In the context of vectored intratumoral Encoded Affimer gene transfer, oncolytic viruses have a distinct advantage, as they can specifically target tumor cells, boost Affimer Agent expression, and amplify therapeutic responses—such as to a PD-L1 Affimer Agent.

In vivo gene transfer of Encoded Affimers can also be accomplished by use of nonviral vectors, such as expression plasmids. Nonviral vectors are easily produced and do not seem to induce specific immune responses. Muscle tissue is most often used as target tissue for transfection, because muscle tissue is well vascularized and easily accessible, and myocytes are long-lived cells. Intramuscular injection of naked plasmid DNA results in transfection of a certain percentage of myocytes. Using this approach, plasmid DNA encoding cytokines and cytokine/IgG1 chimeric proteins has been introduced in vivo and has positively influenced (autoimmune) disease outcome.

In some instances, in order to increase transfection efficiency via so-called intravascular delivery in which increased gene delivery and expression levels are achieved by inducing a short-lived transient high pressure in the veins. Special blood-pressure cuffs that may facilitate localized uptake by temporarily increasing vascular pressure and can be adapted for use in human patients for this type of gene delivery. See, for example, Zhang et al. (2001) “Efficient expression of naked DNA delivered intraarterially to limb muscles of nonhuman primates” Hum. Gene Ther., 12:427-438

Increased efficiency can also be gained through other techniques, such as in which delivery of the nucleic acid is improved by use of chemical carriers—cationic polymers or lipids—or via a physical approach—gene gun delivery or electroporation. See Tranchant et al. (2004) “Physicochemical optimisation of plasmid delivery by cationic lipids” J. Gene Med., 6 (Suppl. 1):524-535; and Niidome et al. (2002) “Gene therapy progress and prospects: nonviral vectors” Gene Ther., 9:1647-1652. Electroporation is especially regarded as an interesting technique for nonviral gene delivery. Somiari, et al. (2000) “Theory and in vivo application of electroporative gene delivery” Mol. Ther. 2:178-187; and Jaroszeski et al. (1999) “In vivo gene delivery by electroporation” Adv. Drug Delivery Rev., 35:131-137. With electroporation, pulsed electrical currents are applied to a local tissue area to enhance cell permeability, resulting in gene transfer across the membrane. Research has shown that in vivo gene delivery can be at least 10-100 times more efficient with electroporation than without. See, for example, Aihara et al. (1998) “Gene transfer into muscle by electroporation in vivo” Nat. Biotechnol. 16:867-870; Mir, et al. (1999) “High-efficiency gene transfer into skeletal muscle mediated by electric pulses” PNAS 96:4262-4267; Rizzuto, et al. (1999) “Efficient and regulated erythropoietin production by naked DNA injection and muscle electroporation” PNAS 96: 6417-6422; and Mathiesen (1999) “Electropermeabilization of skeletal muscle enhances gene transfer in vivo” Gene Ther., 6:508-514.

Encoded PD-L1 binding affimers can be delivered by a wide range of gene delivery system commonly used for gene therapy including viral, non-viral, or physical. See, for example, Rosenberg et al., Science, 242:1575-1578, 1988, and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGraw-Hill, New York, (1996), Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions. The routes of delivery include, for example, systemic administration and administration in situ.

An effective Encoded Affimer gene transfer approach must be directed to the specific tissues/cells where it is needed, and the resulting transgene expression should be at a level that is appropriate to the specific application. Promoters are a major cis-acting element within the vector genome design that can dictate the overall strength of expression as well as cell-specificity.

TABLE 1
Exemplary Ubiquitous and Cell-specific Promoters.

| | | Relative | Size | |
|---|---|---|---|---|
| Promoter | Specificity | Strength | (bps) | Reference(s) |
| CMV | Ubiquitous | +++ | 750- | Xu et al. Gene Ther. 2001 8:1323- |
| | | | 800 | 1332; Gray et al., Hum Gene Ther. |
| | | | | 2011 22:1143-1153 |
| CBA (including | Ubiquitous | +++ | 248- | Klein et al. Exp Neurol. 2002 |
| derivatives: | | | 1,600 | 176(1):66-74; Ohlfest et al. Blood. |
| CAG, CBh, etc.) | | | | 2005 105:2691-2698; and Gray et |
| | | | | al. Hum Gene Ther. 2011 22:1143- |
| | | | | 1153. |
| EF-1α | Ubiquitous | ++ | 2,500 | Gill et al. Gene Ther. 2001 |
| | | | | 8(20):1539-1546; Xu et al. Gene |
| | | | | Ther. 2001 8:1323-1332; and |
| | | | | Gilham et al. J Gene Med. 2010 |
| | | | | 12(2): 129-136. |
| PGK | Ubiquitous | ++ | 426 | Gilham et al. J Gene Med. 2010 |
| | | | | 12(2): 129-136. |
| UBC | Ubiquitous | + | 403 | Gill et al. Gene Ther. 2001 |
| | | | | 8(20):1539-1546; Qin et al. PLoS |
| | | | | One. 2010 5(5):e10611. |
| GUSB (hGBp) | Ubiquitous | + | 378 | Husain et al. Gene Ther. 2009 |
| | | | | 16:927-932. |
| UCOE | Ubiquitous | ++ | 600- | Antoniou et al. Hum Gene Ther. |
| (Promoter of | | | 2,500 | 2013 24(4):363-374. |
| HNRPA2B1- | | | | |
| CBX3) | | | | |
| hAAT | Liver | ++ | 347- | Van Linthout et al. Hum Gene Ther. |
| | | | 1,500 | 2002 13(7):829- |
| | | | | 840; Cunningham et al. Mol Ther. |
| | | | | 2008 16(6):1081-1088 |
| TBG | Liver | ++ | 400 | Yan et al. Gene. 2012 506(2):289- |
| | | | | 294 |
| Desmin | Skeletal muscle | +++ | 1,700 | Talbot et al. Mol Ther. 2010 |
| | | | | 18:601-608 |
| MCK | Skeletal muscle | ++ | 595- | Talbot et al. Mol Ther. 2010 |
| | | | 1,089 | 18:601-608; Wang et al. Gene Ther. |
| | | | | 2008 15:1489-1499; Katwal et al. |
| | | | | Gene Ther. 2013 20(9):930-938. |
| C5-12 | Sketetal, cardiac, | ++ | 312 | Wang et al. Gene Ther. 2008 |
| | and diaphragm | | | 15:1489-1499 |
| NSE | Neuron | +++ | 300- | Xu et al. Gene Ther. 2001 8:1323- |
| | | | 2,200 | 1332 |
| Synapsin | Neuron | + | 470 | Kügler et al. Virology. 2003 |
| | | | | 311:89-95; Hiolki et al. Gene Ther. |
| | | | | 2007 14:872-882; Kuroda et al. J |
| | | | | Gene Med. 2008 10:1163-1175. |
| PDGF | Neuron | +++ | 1,400 | Patterna et al. Gene Ther. 2000 |
| | | | | 7(15):1304-1311; Hioki et al. Gene |
| | | | | Ther. 2007 14:872-882 |
| MecP2 | Neuron | + | 229 | Rastegar et al. LoS One. 2009 |
| | | | | 4:e6810; Gray et al., Hum Gene |
| | | | | Ther. 2011 22:1143-1153 |
| CaMKII | Neuron | ++ | 364- | Hioki et al. Gene Ther. 2007 |
| | | | 2,300 | 14:872-882; Kuroda et al. J Gene |
| | | | | Med. 2008 10:1163-1175 |
| mGluR2 | Neuron | + | 1,400 | Brené et al. Eur J Neyrosci. 2000 |
| | | | | 12:1525-1533; Kuroda et al. J Gene |
| | | | | Med. 2008 10:1163-1175 |
| NFL | Neuron | + | 650 | Xu et al. Gene Ther. 2001 8:1323- |
| | | | | 1332 |
| NFH | Neuron | + | 920 | Xu et al. Gene Ther. 2001 8:1323- |
| | | | | 1332 |
| nβ2 | Neuron | + | 650 | Xu et al. Gene Ther. 2001 8:1323- |
| | | | | 1332 |
| PPE | Neuron | + | 2700 | Xu et al. Gene Ther. 2001 8:1323- |
| | | | | 1332 |
| Enk | Neuron | + | 412 | Xu et al. Gene Ther. 2001 8:1323- |
| | | | | 1332 |
| EAAT2 | Neuron and astrocyte | ++ | 966 | Su et al. Proc Natl Acad Sci U S A. |
| | astocyte | | | 2003 100:1955-1960; Kuroda et al. |
| | | | | J Gene Med. 2008 10:1163-1175 |
| GFAP | Astrocyte | ++ | 681- | Brenner et al. J Neurosci. 1994 |
| | | | 2,200 | 14:1030-1037;Xu et al. Gene Ther. |
| | | | | 2001 8:1323-1332; Lee et al. Glia. |
| | | | | 2008 56:481-493; Dirren et al. Hum |
| | | | | Gene Ther. 2014 25:109-120 |
| MBP | Oligodendrocytes | ++ | 1,900 | Chen et al. Gene Ther. 1998 |
| | | | | 5(1):50-58 |

In some cases, ubiquitous expression of the Encoded Affimer construct in all cell types is desired. Constitutive promoters such as the human elongation factor 1α-subunit (EF1α), immediate-early cytomegalovirus (CMV), chicken β-actin (CBA) and its derivative CAG, the β glucuronidase (GUSB), or ubiquitin C (UBC) can be used to promote expression of the Encoded Affimer Construct in most tissues. Generally, CBA and CAG promote the larger expression among the constitutive promoters; however, their size of ˜1.7 kbs in comparison to CMV (˜0.8 kbs) or EF1α (˜1.2 kbs) may limit use in vectors with packaging constraints such as AAV, particularly where Affimer Agent produced by expression of the Encoded Affimer construct is large. The GUSB or UBC promoters can provide ubiquitous gene expression with a smaller size of 378 bps and 403 bps, respectively, but they are considerably weaker than the CMV or CBA promoter. Thus, modifications to constitutive promoters in order to reduce the size without affecting its expression have been pursued and examples such as the CBh (˜800 bps) and the miniCBA (˜800 bps) can promote expression comparable and even higher in selected tissues (Gray et al., Hum Gene Ther. 2011 22:1143-1153).

When expression of the Encoded Affimer construct should be restricted to certain cell types within an organ, promoters can be used to mediate this specificity. For example, within the nervous system promoters have been used to restrict expression to neurons, astrocytes, or oligodendrocytes. In neurons, the neuron-specific enolase (NSE) promoter drives stronger expression than ubiquitous promoters. Additionally, the platelet-derived growth factor B-chain (PDGF-β), the synapsin (Syn), and the methyl-CpG binding protein 2 (MeCP2) promoters can drive neuron-specific expression at lower levels than NSE. In astrocytes, the 680 bps-long shortened version [gfaABC(1)D] of the glial fibrillary acidic protein (GFAP, 2.2 kbs) promoter can confer higher levels of expression with the same astrocyte-specificity as the GFAP promoter. Targeting oligodendrocytes can also be accomplished by the selection of the myelin basic protein (MBP) promoter, whose expression is restricted to this glial cell; however, its size of 1.9 kbs and low expression levels limit its use.

In the case of expressing the Encoded Affimer constructs in skeletal muscle cells, exemplary promoters based on muscle creatine kinase (MCK) and desmin (1.7 kbs) have showed a high rate of specificity (with minimal expression in the liver if desired). The promoter of the α-myosin heavy chain (α-MHC; 1.2 kbs) has shown significant cardiac specificity in comparison with other muscle promoters (Lee et al., 2011 J Cardiol. 57(1):115-22). In hematopoietic stem cells the synthetic MND promoter (Li et al., 2010 J Neurosci Methods. 189(1):56-64) and the promoter contained in the 2AUCOE (ubiquitous chromatin opening element) have shown to drive a higher transgene expression in all cell lineages when compared to the EF1α and CMV promoters, respectively (Zhang et al., 2007 Blood. 110(5):1448-57; Koldej 2013 Hum Gene Ther Clin Dev. 24(2):77-85; Dighe et al., 2014 PLoS One. 9(8):e104805). Conversely, using promoters to restrict expression to only liver hepatocytes after vector-mediated gene transfer has been shown to reduce transgene-specific immune responses in systems where that is a risk, and to even induce immune tolerance to the expressed protein (Zhang et al., 2012 Hum Gene Ther. 23(5):460-72), which for certain Affimer Agents may be beneficial. The al-antitrypsin (hAAT; 347 bps) and the thyroxine binding globulin (TBG; ˜400 bps) promoters drive gene expression restricted to the liver with minimal invasion to other tissues (Yan et al., 2012 Gene. 506(2):289-94; Cunningham et al., 2008 Mol Ther. 16(6):1081-8).

In some embodiments, a mechanism to control the duration and amount of in vivo Encoded Affimer expression will typically be desired. There are a variety of inducible promoters which can be adapted for use with viral vectored- and plasmid DNA-based Encoded Affimer gene transfer. See Fang et al. (2007) “An antibody delivery system for regulated expression of therapeutic levels of monoclonal antibodies in vivo” Mol Ther. 5(6):1153-9; and Perez et al. (2004) “Regulatable systemic production of monoclonal antibodies by in vivo muscle electroporation” Genet Vaccines Ther. 2(1):2. An exemplary a regulatable mechanism currently under clinical evaluation is an ecdysone-based gene switch activated by a small molecule ligand. Cai et al. (2016) “Plasma pharmacokinetics of veledimex, a small-molecule activator ligand for a proprietary gene therapy promoter system, in healthy subjects” Clin Pharmacol Drug Dev. 2016.

In some embodiments of the Encoded Affimer constructs, viral post-transcriptional regulatory elements (PREs) may be used; these cis-acting elements are required for nuclear export of intronless viral RNA (Huang and Yen, 1994 J Virol. 68(5):3193-9; and 1995 Mol Cell Biol. 15(7):3864-9). Examples include HPRE (Hepatitis B Virus PRE, 533 bps) and WPRE (Woodchuck Hepatitis Virus PRE, 600 bps), which can increase the level of transgene expression by almost 10-fold in certain instances (Donello et al., 1998 J Virol. 72(6):5085-92). To further illustrate, using lentiviral and AAV vectors, WPRE was found to increase CMV promoter driven transgene expression, as well as increase PPE, PDGF and NSE promoter-driven transgene expression. Another effect of the WPRE can be to protect Encoded Affimer constructs transgenes from silencing (Paterna et al., 2000 Gene Ther. 7(15):1304-11; Xia et al., 2007 Stem Cells Dev. 2007 February; 16(1):167-76).

The polyadenylation of a transcribed Encoded Affimer transcript can also be important for nuclear export, translation, and mRNA stability. Therefore, in some embodiments, the Encoded Affimer construct will include a polyadenylation signal sequence. A variety of studies are available that have determined the effects of different polyA signals on gene expression and mRNA stability. Exemplary polyadenylation signal sequences include SV40 late or bovine growth hormone polyA (bGHpA) signal sequences, as well as minimal synthetic polyA (SPA) signal (Levitt et al., 1989 Genes Dev. 3(7):1019-25; Yew et al., 1997 Hum Gene Ther. 1997 8(5):575-84). The efficiency of polyadenylation is increased by the SV40 late polyA signal upstream enhancer (USE) placed upstream of other polyA signals (Schek et al., 1992 Mol Cell Biol. 12(12):5386-93). In some embodiments, merely to illustrate, the Encoded Affimer construct will include an SV40 late+2× USE polyA signal.

TABLE 2
Exemplary Polyadenylation Signals

| PolyA | Relative | Size | | |
|---|---|---|---|---|
| Signal and USE | Strength | (bps) | Source | Reference(s) |
| hGH | + | 624 | Human growth | Ostedgaard et al. Proc Natl Acad |
| | | | hormone | Sci U S A. 2005 102(8):2952-2957 |
| SV40 late | +++ | 135 | Simian virus 40 | Choi et al. Mol Brain. 2014 7:17 |
| SPA | + | 49 | Rabbit β-globin | Levitt et al. Genes Dev. 3(7):1019- |
| (synthetic | | | | 1025; Yew et al. Hum Gene Ther. |
| polyA) | | | | 1997 8(5):575-584; Ostedgaard et |
| | | | | al. Proc Natl Acad Sci U S A. 2005 |
| | | | | 102(8):2952-2957; Choi et al. Mol |
| | | | | Brain. 2014 7:17 |
| bGH | ++ | 250 | Bovine growth | Yew et al. Hum Gene Ther. 1997 |
| | | | hormone | 8(5):575-584; Xu et al. Gene Ther. |
| | | | | 2001 8:1323-1332; Wu et al. Mol |
| | | | | Ther. 2008 16(2):280-289; Gray et |
| | | | | al., Hum Gene Ther. 2011 22:1143- |
| | | | | 1153; Choi et al. Mol Brain. |
| | | | | 2014 7:17 |
| SV40 late | ++ | 100 | Simian virus 40 | Schambach et al. Mol Ther. 2007 |
| 2xUSE | | | | 15(6):1167-1173; Choi et al. Mol |
| | | | | Brain. 2014 7:17 |
| HIV-1 USE | + | 35 | Human | Schambach et al. Mol Ther. 2007 |
| | | | immunodeficiency | 15(6): 1167-1173 |
| | | | virus 1 | |
| GHV USE | + | 39 | Ground squirrel | Schambach et al. Mol Ther. 2007 |
| | | | hepatitis virus | 15(6):1167-1173 |
| Adenovirus | + | 21 | Adenovirus | Schambach et al. Mol Ther. 2007 |
| (L3) USE | | | | 15(6):1167-1173 |
| hTHGB | + | 21 | Human | Schambach et al. Mol Ther. 2007 |
| USE | | | prothrombin | 15(6):1167-1173 |
| hC2 USE | + | 53 | Human C2 | Schambach et al. Mol Ther. 2007 |
| | | | complement gene | 15(6):1167-1173 |

In some embodiments, it may be desirable for the Encoded Affimer construct to include one or more regulatory enhancers, i.e., in addition to any promoter sequences. The CMV enhancer is upstream of the CMV promoter at −598 to −68 (Boshart et al., 1985 Cell. 41(2):521-30) (˜600 bps) and contains transcription binding sites. In some embodiments, a CMV enhancer can be included in the construct to increase tissue-specific promoter-driven transgene expression, such as using the ANF (atrial natriuretic factor) promoter, the CC10 (club cell 10) promoter, SP-C (surfactant protein C) promoter, or the PDGF-β (platelet-derived growth factor-β) promoter (merely as examples). Altogether, the CMV enhancer increases transgene expression under different cell-specific promoters and different cell types making it a broadly applicable tool to increase transgene expression levels. In muscle, for example, in AAV expression systems transgene expression using the CMV enhancer with a muscle-specific promoter can increase expression levels of the protein encoded by the transgene, so would be particularly useful in the current disclosure for expressing Affimer Agents from Encoded Affimer constructs introduced into muscle cells of a patient.

The subject Encoded Affimer constructs may also include one or more intronic sequences. The presence of an intron or intervening sequence in mRNA was first described, in vitro, to be important for mRNA processing and increased transgene expression (Huang and Gorman, 1990 Mol Cell Biol. 10(4):1805-10; Niwa et al., 1990 Genes Dev. 4(9):1552-9). The intron(s) can be placed within the coding sequence for the Affimer Agent and/or can be placed between the promoter and transgene. A variety of introns (Table 3) placed between the promoter and transgene were compared, in mice using AAV2, for liver transgene expression (Wu et al., 2008). The MVM (minute virus of mice) intron increased transgene expression more than any other intron tested and more than 80-fold over no intron (Wu et al., 2008). However, in cultured neurons using AAV expression cassettes, transgene expression was less under a CaMPKII promoter with a chimeric intron (human β-globin donor and immunoglobulin heavy chain acceptor) between the transgene and polyA signal compared to a WPRE (Choi et al., 2014). Together, an intron can be a valuable element to include in an expression cassette to increase transgene expression.

TABLE 3
Exemplary Introns

| | Relative | Size | | |
|---|---|---|---|---|
| Itron | Strength | (bps) | Source | Reference(s) |
| MVM | +++ | 67-97 | Minute virus | Wu et al. Mol Ther. 2008 |
| | | | of mice | 16(2):280-289 |
| F.IX truncated | + | 300 | Human | Wu et al. Mol Ther. 2008 |
| intron 1 | | | factor IX | 16(2):280-289; Kurachi et al. J |
| | | | | Biol Chem. 1995 270(10):5276- |
| | | | | 5281 |
| β-globin SD/ | + | 250 | Human, | Wu et al. Mol Ther. 2008 |
| immunoglobin heavy | | | pZac2.1 | 16(2):280-289; Choi et al. Mol |
| chain SA | | | | Brain. 2014;7:17 |
| Adenovirus SD^{#}/ | ++ | 500 | pAdβ | Wong et al. Chromosoma. 1985 |
| immunoglobulin SA* | | | | 92(2):124-135; Yew et al. Hum |
| | | | | Gene Ther. 1997 8(5):575-584 |
| SV40 late SD^{#}/SA* | + | 180 | pCMVβ | Yew et al. Hum Gene Ther. 1997 |
| (19S/16S) | | | | 8(5):575-584 |
| Hybrid adenovirus | +++ | 230 | Adenovirus | Choi et al. Mol Brain. 2014;7:17; |
| SD^{#}/IgG SA* | | | | Huang et al. Mol Cell Biol. 1990 |
| | | | | 10(4):1805-1810 |

In the case of episomal vectors, the subject Encoded Affimer constructs may also include one or more origins of replication, minichromosome maintenance elements (MME) and/or nuclear localization elements. Episomal vectors of the disclosure comprise a portion of a virus genomic DNA that encodes an origin of replication (ori), which is required for such vectors to be self-replicating and, thus, to persist in a host cell over several generations. In addition, an episomal vector of the disclosure also may contain one or more genes encoding viral proteins that are required for replication, i.e., replicator protein (s). Optionally, the replicator protein(s) which help initiate replication may be expressed in trans on another DNA molecule, such as on another vector or on the host genomic DNA, in the host cell containing a self-replicating episomal expression vector of this disclosure. Preferred self-replicating episomal LCR-containing expression vectors of the disclosure do not contain viral sequences that are not required for long-term stable maintenance in a eukaryotic host cell such as regions of a viral genome DNA encoding core or capsid proteins that would produce infectious viral particles or viral oncogenic sequences which may be present in the full-length viral genomic DNA molecule. The term “stable maintenance” herein, refers to the ability of a self-replicating episomal expression vector of this disclosure to persist or be maintained in non-dividing cells or in progeny cells of dividing cells in the absence of continuous selection without a significant loss (e.g., >50%) in copy number of the vector for two, three, four, or five or more generations. In some embodiments, the vectors will be maintained over 10-15 or more cell generations. In contrast, “transient” or “short-term” persistence of a plasmid in a host cell refers to the inability of a vector to replicate and segregate in a host cell in a stable manner; that is, the vector will be lost after one or two generations, or will undergo a loss of >51% of its copy number between successive generations.

Several representative self-replicating, LCR-containing, episomal vectors useful in the context of the present disclosure are described further below. The self-replicating function may alternatively be provided by one or more mammalian sequences such as described by Wohlge uth et al., 1996, Gene Therapy 3:503; Vos et al., 1995, Jour. Cell. Biol., Supp. 21A, 433; and Sun et al., 1994, Nature Genetics 8:33, optionally in combination with one or more sequence which may be required for nuclear retention. The advantage of using mammalian, especially human sequences for providing the self-replicating function is that no extraneous activation factors are required which could have toxic or oncogenic properties. It will be understood by one of skill in the art that the disclosure is not limited to any one origin of replication or any one episomal vector, but encompasses the combination of the tissue-restricted control of an LCR in an episomal vector. See also WO1998007876 “Self-replicating episomal expression vectors conferring tissue-specific gene expression” and U.S. Pat. No. 7,790,446 “Vectors, cell lines and their use in obtaining extended episomal maintenance replication of hybrid plasmids and expression of gene products”

Epstein-Barr Virus-Based Self-Replicating Episomal Expression Vectors. The latent origin oriP from Epstein-Barr Virus (EBV) is described in Yates et. al., Proc. Natl. Acad. Sci. USA 81:3806-3810 (1984); Yates et al., Nature 313:812-815 (1985); Krysan et al., Mol. Cell. Biol. 9:1026-1033 (1989); James et al. Gene 86: 233-239 (1990), Peterson and Legerski, Gene 107:279-284 (1991); and Pan et al., Som. Cell Molec. Genet. 18:163-177 (1992)). An EBV-based episomal vector useful according to the disclosure can contain the oriP region of EBV which is carried on a 2.61 kb fragment of EBV and the EBNA-1 gene which is carried on a 2.18 kb fragment of EBV. The EBNA-1 protein, which is the only viral gene product required to support in trans episomal replication of vectors containing oriP, may be provided on the same episomal expression vector containing oriP. It is also understood, that as with any protein such as EBNA-1 known to be required to support replication of viral plasmid in trans, the gene also may be expressed on another DNA molecule, such as a different DNA vector.

Papilloma Virus-Based, Self-Replicating, Episomal Expression Vectors. The episomal expression vectors of the disclosure also may be based on replication functions of the papilloma family of virus, including but not limited to Bovine Papilloma Virus (BPV) and Human Papilloma Viruses (HPVs). BPV and HPVs persist as stably maintained plasmids in mammalian cells. -S trans-acting factors encoded by BPV and HPVs, namely E1 and E2, have also been identified which are necessary and sufficient for mediate replication in many cell types via minimal origin of replication (Ustav et al., EMBO J. 10: 449-457 (1991); Ustav et al., EMBO J. 10:4231-4329, (1991); Ustav et al., Proc. Natl. Acad. Sci. USA 90: 898-902 (1993)).

An episomal vector useful according to the disclosure is the BPV-I vector system described in Piirsoo et al., EMBO J., 15:1 (1996) and in WO 94/12629. The BPV-1 vector system described in Piirsoo et al. comprises a plasmid harboring the BPV-1 origin of replication (minimal origin plus extrachro osomal maintenance element) and optionally the E1 and E2 genes. The BPV-1E1 and E2 genes are required for stable maintenance of a BPV episomal vector. These factors ensure that the plasmid is replicated to a stable copy number of up to thirty copies per cell independent of cell cycle status. The gene construct therefore persists stably in both dividing and non-dividing cells. This allows the maintenance of the gene construct in cells such as hemopoietic stem cells and more committed precursor cells.

The BPV origin of replication has been located at the 31 end of the upstream regulatory region within a 60 base pair (bp) DNA fragment (nucleotides (nt) 7914-7927) which includes binding sites for the E1 and E2 replication factors. The minimal origin of replication of HPV has also been characterized and located in the URR fragment (nt 7022-7927) of HPV (see, for example, Chiang et al., Proc. Natl. Acad. Sci. USA 89:5799-5803 (1992)). As used herein, “E1” refers to the protein encoded by nucleotides (nt) 849-2663 of BPV subtype 1 or by nt 832-2779 of HPV of subtype 11, to equivalent E1 proteins of other papilloma viruses, or to functional fragments or mutants of a papilloma virus E1 protein, i.e., fragments or mutants of E1 which possess the replicating properties of E1.

As used herein, “E2H refers to the protein encoded by nt 2594-3837 of BPV subtype 1 or by nt 2723-3823 of HPV subtype 11, to equivalent E2 proteins of other papilloma viruses, or to functional fragments or mutants of a papilloma virus E2 protein, i.e., fragments or mutants of E2 which possess the replicating properties of E2. “Minichromosomal maintenance element” (MME) refers to the extrachromosomal maintenance element of the papilloma viral genome to which viral or human proteins essential for papilloma viral replication bind, which region is essential for stable episomal maintenance of the papilloma viral MO in a host cell, as described in Piirsoo et al. (supra). Preferably, the MME is a sequence containing multiple binding sites for the transcriptional activator E2. The MME in BPV is herein defined as the region of BPV located within the upstream regulatory region which includes a minimum of about six sequential E2 binding sites, and which gives optimum stable maintenance with about ten sequential E2 binding sites. E2 binding site 9 is an example sequence for this site, as described hereinbelow, wherein the sequential sites are separated by a spacer of about 4-10 nucleotides, and optimally 6 nucleotides. E1 and E2 can be provided to the plasmid either in cis or in trans, also as described in WO 94/12629 and in Piirsoo et al. (supra).

“E2 binding site” refers to the minimum sequence of papillomavirus double-stranded DNA to which the E2 protein binds. An E2 binding site may include the sequence 5* ACCGTTGCCGGT 3′ (SEQ ID NO: 208), which is high affinity E2 binding site 9 of the BPV-1 URR; alternatively, an E2 binding site may include permutations of binding site 9, which permutations are found within the URR, and fall within the generic E2 binding sequence 5′ ACCN6GGT 3′ (SEQ ID NO: 209). One or more transcriptional activator E2 binding sites are, in most papillomaviruses, located in the upstream regulatory region, as in BPV and HPV. A vector which also is useful according to the disclosure may include a region of BPV between 6959-7945/1-470 on the BPV genetic map (as described in WO 94/12629), which region includes an origin of replication, a first promoter operatively associated with a gene of interest, the BPV E1 gene operatively associated with a second promoter to drive transcription of the E1 gene; and the BPV E2 gene operatively associated with a third promoter to drive transcription of the E2 gene.

E1 and E2 from BPV will replicate vectors containing the BPV origin or the origin of many HPV subtypes (Chiang et al., supra). E1 and E2 from HPV will replicate vectors via the BPV origin and via the origin of many HPV subtypes (Chiang et al., supra). As with all vectors of the disclosure, the BPV-based episomal expression vectors of the disclosure must persist through 2-5 or more divisions of the host cell.

See also U.S. Pat. No. 7,790,446 and Abroi et al. (2004) “Analysis of chromatin attachment and partitioning functions of bovine papillomavirus type 1 E2 protein. Journal of Virology 78:2100-13 which have shown that the BPV1 E2 protein dependent MME and EBV EBNA1 dependent FR segregation/partitioning activities function independently from replication of the plasmids. The stable-maintenance function of EBNA1/FR and E2/MME can be used to ensure long-time episomal maintenance for cellular replication origins.

Papovavirus-Based, Self-Replicating, Episomal Expression Vectors. The vectors of the disclosure also may be derived from a human papovavirus BK genomic DNA molecule. For example, the BK viral genome can be digested with restriction enzymes EcoRI and BamHI to produce a 5 kilobase (kb) fragment that contains the BK viral origin of replication sequences that can confer stable maintenance on vectors (see, for example, De Benedetti and Rhoads, Nucleic Acids Res. 19:1925 (1991), as can a 3.2 kb fragment of the BK virus (Cooper and Miron, Human Gene Therapy 4:557 (1993)).

The Encoded Affimer constructs of the present disclosure can be provided as circular or linear nucleic acids. The circular and linear nucleic acids are capable of directing expression of the Affimer Agent coding sequence in an appropriate subject cell. The one or more nucleic acid systems for expressing an Affimer Agent may be chimeric, meaning that at least one of its components is heterologous with respect to at least one of its other components.

a. Viral Vectors

Exemplary viral gene therapy system that are readily adapted for use in the present disclosure include plasmid, adenovirus, adeno-associated virus (AAV), retrovirus, lentivirus, herpes simplex virus, vaccinia virus, poxvirus, reovirus, measles virus, Semliki Forest virus, and the like. Preferred viral vectors are based on non-cytopathic eukaryotic viruses in which non-essential genes have been replaced with the nucleic acid construct carrying the nucleic acid sequences encoding the epitopes and targeting sequences of interest.

To further illustrate, encoded affimers can be delivered in vivo using adenoviruses and adeno-associated (AAV) viruses, which are double-stranded DNA viruses that have already been approved for human use in gene therapy.

Adenovirus Vectors

One illustrative method for in vivo delivery of one or more nucleic acid sequences involves the use of an adenovirus (“AdV”) expression vector. AdVs are non-enveloped, double-stranded DNA viruses that neither integrate in the host genome nor replicate during cell division. AdV-mediated antibody gene transfer has shown therapeutic efficacy in a variety of different disease models advancing towards the clinic. Systemic mAb expression has mostly been pursued, via s.c. and especially i.v. and intramuscular AdV injection. See Wold et al. (2013) “Adenovirus vectors for gene therapy, vaccination and cancer gene therapy” Curr Gene Ther. 13(6):421-33; and Deal et al. “Engineering humoral immunity as prophylaxis or therapy” 2015 Curr Opin Immunol. 35:113-22. Other routes of delivery have focused on more local mAb production, such as via intranasal, intratracheal or intrapleural administration of the encoding AdV. The use of AdVs as oncolytic vectors is a popular approach particularly for generation of encoded antibodies at the site of tumors. Foreign genes delivered by current adenoviral gene delivery system are episomal, and therefore, have low genotoxicity to host cells. Therefore, gene therapy using adenoviral gene delivery systems may be considerably safe. The present disclosure specifically contemplates the delivery of Affimer Agents by expression of Encoded Affimer constructs delivered in the form of an adenoviral vector and delivery system.

Adenovirus has been usually employed as a gene delivery vector because of its mid-sized genome, ease of manipulation, high titer, wide target-cell range and high infectivity. Both ends of the viral genome contains 100-200 bp ITRs (inverted terminal repeats), which are cis elements necessary for viral DNA replication and packaging. The E1 region (E1A and E1B) of genome encodes proteins responsible for the regulation of transcription of the viral genome and a few cellular genes. The E2 region (E2A and E2B) encodes proteins responsible for viral DNA replication. Of adenoviral vectors developed so far, the replication incompetent adenovirus having the deleted E1 region is usually used and represent one exemplary choice of AdV for generating the Encoded Affimer constructs of the present disclosure. The deleted E3 region in adenoviral vectors may provide an insertion site for transgenes (Thimmappaya, B. et al., Cell, 31:543-551 (1982); and Riordan, J. R. et al., Science, 245:1066-1073 (1989)).

An “adenovirus expression vector” is meant to include those constructs containing adenovirus sequences sufficient to (a) support packaging of the construct and (b) to express a polynucleotide that encodes a polypeptide including an Affimer Agent such as a PD-L1 binding affimer (the Encoded Affimer sequence). In some embodiments, the sequence for an Encoded Affimer may be inserted into the DA promoter region. According to an exemplary embodiment, the recombinant adenovirus comprises deleted E1B and E3 region and the nucleotide sequence for an encoded affimer is inserted into the deleted E1B and E3 region.

Adeno-Associated Virus Vectors (AAV)

AAVs (or “rAAV” for recombinant AAV) are non-enveloped small, single-stranded DNA viruses capable of infecting both dividing and non-dividing cells. Similar to AdV, AAV-based vectors remain in an episomal state in the nucleus and display a limited risk of integration. In contrast to the generally limited durability of AdV-mediated gene transfer, transgene expression can persist for years following intramuscular recombinant AAV (rAAV) vector delivery.

Alipogene tiparvovec (Glybera™), an rAAV encoding the human lipoprotein lipase gene, was approved in 2012 as the first gene therapy product in Europe. Since then, various rAAV-based gene therapy products are currently under clinical evaluation. In the context of antibody gene transfer, a variety of reports have demonstrated in vivo production of an anti-human immune deficiency virus (HIV) mAb in mice following intramuscular injection of the mAb-encoding rAAV. The rAAV vector's potential for combination therapy has also been demonstrated, i.e. by expressing two mAbs. Similar to AdV, intramuscular and i.v. rAAV administration have been most often pursued. Reviewed in Deal et al. “Engineering humoral immunity as prophylaxis or therapy” 2015 Curr Opin Immunol. 35:113-22. A variety of additional delivery sites have also been demonstrated to achieve more local therapeutic effects, including intracranial, intranasal, intravitreal, intrathecal, intrapleural, and intraperitoneal routes. With the utility of rAAV demonstrated for antibody gene transfer, the present disclosure also specifically contemplates the use of rAAV systems for the delivery of Encoded Affimer sequences in vivo and the production of Affimer Agents in the body of a patient as a consequence to expression of the rAAV construct.

One important feature to AAV is that these gene transfer viruses are capable of infecting non-dividing cells and various types of cells, making them useful in constructing the Encoded Affimer delivery system of this disclosure. The detailed descriptions for use and preparation of exemplary AAV vectors are found in, for example, U.S. Pat. Nos. 5,139,941 and 4,797,368, as well as LaFace et al, Viology, 162:483486 (1988), Zhou et al., Exp. Hematol. (NY), 21:928-933 (1993), Walsh et al, J. Clin. Invest., 94:1440-1448 (1994) and Flotte et al., Gene Therapy, 2:29-37 (1995). AAV is a good choice of delivery vehicles due to its safety, i.e., genetically engineered (recombinant) does not integrate into the host genome. Likewise, AAV is not pathogenic and not associated with any disease. The removal of viral coding sequences minimizes immune reactions to viral gene expression, and therefore, recombinant AAV does not evoke an inflammatory response.

Typically, a recombinant AAV virus is made by co-transfecting a plasmid containing the gene of interest (i.e., the coding sequence for an Affimer Agent) flanked by the two AAV terminal repeats (McLaughlin et al., J. Virol., 62:1963-1973 (1988); Samulski et al., J. Virol., 63:3822-3828 (1989)) and an expression plasmid containing the wild type AAV coding sequences without the terminal repeats (McCarty et al., J. Virol., 65:2936-2945 (1991)). Typically, viral vectors containing an Encoded Affimer construct are assembled from polynucleotides encoding the affimer containing polypeptide, suitable regulatory elements and elements necessary for expression of the encoded affimer which mediate cell transduction. In some embodiments, adeno-associated viral (AAV) vectors are employed. In a more specific embodiment, the AAV vector is an AAV1, AAV6, or AAV8.

The AAV expression vector which harbors the Encoded Affimer sequence bounded by AAV ITRs, can be constructed by directly inserting the selected sequence(s) into an AAV genome which has had the major AAV open reading frames (“ORFs”) excised therefrom.

For eukaryotic cells, expression control sequences typically include a promoter, an enhancer, such as one derived from an immunoglobulin gene, SV40, cytomegalovirus, etc. (see above), and a polyadenylation sequence which may include splice donor and acceptor sites. The polyadenylation sequence generally is inserted following the transgene sequences and before the 3′ITR sequence.

Selection of these and other common vector and regulatory elements are conventional, and many such sequences are available. See, e.g., Sambrook et al., and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes of this disclosure. However, one of skill in the art may make a selection among these expression control sequences without departing from the scope of this disclosure. Suitable promoter/enhancer sequences may be selected by one of skill in the art using the guidance provided by this application. Such selection is a routine matter and is not a limitation of the molecule or construct.

Retrovirus Vectors

Non-cytopathic viruses useful in the context of delivery of Encoded Affimer constructs include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known to those of skill in the art.

In order to construct a retroviral vector, the Affimer Agent coding sequence is inserted into the viral genome in the place of certain viral sequences to produce a replication-defective virus. To produce virions, a packaging cell line containing the gag, pol and env genes but without the LTR (long terminal repeat) and psi (□) components is constructed (Mann et al., Cell, 33:153-159 (1983)). When a recombinant plasmid containing the cytokine gene, LTR and psi is introduced into this cell line, the psi sequence allows the RNA transcript of the recombinant plasmid to be packaged into viral particles, which are then secreted into the culture media (Nicolas and Rubinstein “Retroviral vectors,” In: Vectors: A survey of molecular cloning vectors and their uses, Rodriguez and Denhardt (eds.), Stoneham: Butterworth, 494-513 (1988)). The media containing the recombinant retroviruses is then collected, optionally concentrated and used for gene delivery system.

Successful gene transfer using such second-generation retroviral vectors has been reported. Kasahara et al. (Science, 266:1373-1376 (1994)) prepared variants of moloney murine leukemia virus in which the EPO (erythropoietin) sequence is inserted in the place of the envelope region, consequently, producing chimeric proteins having novel binding properties. Likely, the present gene delivery system can be constructed in accordance with the construction strategies for the second-generation retroviral vector.

In some embodiments, the retrovirus is a “gammaretroviruses”, which refers to a genus of the retroviridae family. Exemplary gammaretroviruses include mouse stem cell virus, murine leukemia virus, feline leukemia virus, feline sarcoma virus, and avian reticuloendotheliosis viruses.

In some embodiments, the retroviral vector for use in the present disclosure is a lentiviral vector, which refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Another class of widely used retroviral vectors that can be used for the delivery and expression of an Encoded Affimer include those based upon murine leukemia virus (MuLV), gibbon ape leukemia virus (GaLV) and combinations thereof (see, e.g., Buchscher et al., J. Virol. 66:2731-2739, 1992; Johann et al., J. Virol. 66: 1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700).

Still other retroviral vectors that can be also be used in the present disclosure include, e.g., vectors based on human foamy virus (HFV) or other viruses in the Spumavirus genera. Foamy viruses (FVes) are the largest retroviruses known today and are widespread among different mammals, including all non-human primate species, however are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, which provide a description of methods for efficiently introducing nucleic acids into human cells using such retroviral vectors. Other retroviral vectors include, for example, mouse mammary tumor virus vectors (e.g., Shackleford et al., Proc. Natl. Acad. Sci. U.S.A. 85:9655-9659, 1998), lentiviruses, and the like.

Additional retroviral viral delivery systems that can be readily adapted for delivery of a transgene encoding a PD-L1 Affimer Agent include, merely to illustrate Published PCT Applications WO/2010/045002, WO/2010/148203, WO/2011/126864, WO/2012/058673, WO/2014/066700, WO/2015/021077, WO/2015/148683, WO/2017/040815—the specifications and FIGS. of each of which are incorporated by reference herein.

In some embodiments, a retroviral vector contains all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail regarding retroviral vectors can be found in Boesen, et al., 1994, Biotherapy 6:291-302; Clowes, et ai, 1994, J. Clin. Invest. 93:644-651; Kiem, et al., 1994, Blood 83: 1467-1473; Salmons and Gunzberg, 1993, Human Gene Therapy 4: 129-141; Miller, et al., 1993, Meth. Enzymol. 217:581-599; and Grossman and Wilson, 1993, Curr. Opin. in Genetics and Devel. 3: 110-1 14.

In some embodiments, the retrovirus is a recombinant replication competent retrovirus comprising: a nucleic acid sequence encoding a retroviral GAG protein; a nucleic acid sequence encoding a retroviral POL protein; a nucleic acid sequence encoding a retroviral envelope; an oncoretroviral polynucleotide sequence comprising Long-Terminal Repeat (LTR) sequences at the 5′ and 3′ end of the oncoretroviral polynucleotide sequence; a cassette comprising an internal ribosome entry site (IRES) operably linked to a coding sequence for an Affimer Agent, such as for a PD-L1 Affimer Agent, wherein the cassette is positioned 5′ to the U3 region of the 3′ LTR and 3′ to the sequence encoding the retroviral envelope; and cis-acting sequences for reverse transcription, packaging and integration in a target cell.

In some embodiments, the retrovirus is a recombinant replication competent retrovirus comprising: a retroviral GAG protein; a retroviral POL protein; a retroviral envelope; a retroviral polynucleotide comprising Long-Terminal Repeat (LTR) sequences at the 3′ end of the retroviral polynucleotide sequence, a promoter sequence at the 5′ end of the retroviral polynucleotide, the promoter being suitable for expression in a mammalian cell, a gag nucleic acid domain, a pol nucleic acid domain and an env nucleic acid domain; a cassette comprising an Encoded Affimer sequence, wherein the cassette is positioned 5′ to the 3′ LTR and is operably linked and 3′ to the env nucleic acid domain encoding the retroviral envelope; and cis-acting sequences necessary for reverse transcription, packaging and integration in a target cell.

In some embodiments of the recombinant replication competent retrovirus, the envelope is chosen from one of amphotropic, polytropic, xenotropic, 10A1, GALV, Baboon endogenous virus, RD114, rhabdovirus, alphavirus, measles or influenza virus envelopes.

In some embodiments of the recombinant replication competent retrovirus, the retroviral polynucleotide sequence is engineered from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV).

In some embodiments of the recombinant replication competent retrovirus, retrovirus is a gammaretrovirus.

In some embodiments of the recombinant replication competent retrovirus, there is a second cassette comprising a coding sequence for a second therapeutic protein, such as another checkpoint inhibitor polypeptide, a co-stimulatory polypeptide and/or a immunostimulatory cytokine (merely as examples), e.g., downstream of the cassette. In certain instances, the second cassette can include an internal ribosome entry site (IRES) or a minipromoter or a polIII promoter operably linked to the coding sequence for the second therapeutic protein.

In some embodiments of the recombinant replication competent retrovirus, it is a nonlytic, amphotropic retroviral replicating vector which, preferably, selectively infects and replicates in the cells of the tumor microenvironment.

Other Viral Vectors as Expression Constructs

In the context of vectored intratumoral Encoded Affimer gene transfer, oncolytic viruses have a distinct advantage, as they can specifically target tumor cells, boost therapeutic Affimer Agent expression, and amplify antitumor therapeutic responses. Oncolytic viruses, which overlap with certain viral systems described above, promote anti-tumor responses through selective tumor cell killing and induction of systemic anti-tumor immunity. The mechanisms of action are not fully elucidated but are likely to depend on viral replication within transformed cells, induction of primary cell death, interaction with tumor cell anti-viral elements and initiation of innate and adaptive anti-tumor immunity. Reviewed in Kaufman et al. 2015 “Oncolytic viruses: a new class of immunotherapy drugs” Nat Rev Drug Discov. 14(9):642-62. Many of the oncolytic viruses that are currently in the clinic have a natural tropism for cell surface proteins that are aberrantly expressed by cancer cells. To date, AdV, poxviruses, coxsackieviruses, poliovirus, measles virus, Newcastle disease virus, reovirus, and others have entered into early□ phase clinical trials. In 2015, the FDA and EMA approved talimogene laherparepvec (T-VEC, Imlygic™), an oncolytic herpes virus armed with the gene for granulocyte-macrophage colony-stimulating factor (GM-CSF). The self-perpetuating nature of oncolytic viruses makes them an appealing platform for Encoded Affimer gene transfer of the present disclosure, as transgene products can be amplified along with viral replication, thereby maximizing therapeutic effect. Liu et al. 2008 “Oncolytic adenoviruses for cancer gene therapy” Methods Mol Biol. 433:243-58.

In the case of Affimer Agents that are large fusion proteins, i.e., which comprise other protein domains beyond a single affimer domain, local intratumoral expression can present an appealing strategy to overcome poor penetration in solid tumors if and where that might be an issue. Beckman et al. (2007) “Antibody constructs in cancer therapy: protein engineering strategies to improve exposure in solid tumors” Cancer 109(2):170-9; and Dronca et al. 2015 “Immunomodulatory antibody therapy of cancer: the closer, the better” Clin Cancer Res. 21(5):944-6. Likewise, intratumoral delivery of the Encoded Affimer construct and concomitant local expression of the Affimer Agent can create a better therapeutic index where dose-limiting toxicities might otherwise prevent reaching the effective intratumoral concentration for efficacy when the Affimer Agent is delivered (or expressed) systemically.

In the case of the PD-L1 Affimer Agents of the present disclosure, the immunomodulatory nature of these affimers are very relevant to the use of oncolytic viruses. Indeed, for oncolytic virus therapy, it is desirable to override immune checkpoint inhibitor networks and thereby create a pro-inflammatory environment within the cancer. Numerous clinical trials are currently underway to evaluate the combination of oncolytic viruses and conventional immunomodulatory mAb administration. Kaufman et al. 2015 “Oncolytic viruses: a new class of immunotherapy drugs” Nat Rev Drug Discov. 14(9):642-62; and Lichty et al. 2014 “Going viral with cancer immunotherapy” Nat Rev Cancer. 14(8):559-67. However, systemic treatment with checkpoint-blocking mAbs can lead to severe immune-related adverse effects, which may also be an issue for some embodiments of the subject PD-L1 Affimer Agents, highlighting the opportunity for local therapies, e.g. via Encoded Affimer-armed oncolytic viruses. Different studies have pursued this approach and can be readily adapted for use with the subject Encoded Affimers. Dias et al. armed a replication-deficient and -competent oncolytic AdV with an anti-human CTLA-4 mAb. Dias et al. 2012 “Targeted cancer immunotherapy with oncolytic adenovirus coding for a fully human monoclonal antibody specific for CTLA-4” Gene Ther. 19(10):988-98. Another system recently described (and that can be adapted for use with the Encoded Affimers of the present disclosure) involved armed oncolytic vaccinia viruses with anti-murine programmed cell death protein 1 (PD-1) Fab, scFv or full-length mAb. Reflecting virus replication, mAb levels in the tumor peaked 3-5 days after intratumoral injection at 9 or 30 μg/ml, depending on the tumor model. Serum mAb levels followed the same trend, albeit threefold or more lower, although mAb detection was lost after 5 days. Intratumorally expressed mAbs lasted longer compared to intratumoral injection of anti-PD-1 mAb protein, with follow-up limited to 11 days after injection. Fab and scFv expression were not reported. Anti-tumor responses of the virus armed with either the anti-PD-1 scFv or mAb were superior to the unarmed virus and as effective as the combination of the unarmed virus and systemic anti-PD-1 mAb protein injections. Kleinpeter et al. 2016 “Vectorization in an oncolytic vaccinia virus of an antibody, a Fab and a scFv against programmed cell death-1 (PD-1) allows their intratumoral delivery and an improved tumor-growth inhibition” Oncoimmunology. 5(10):e1220467 (online). Also recently, intratumoral administration of a combination of an oncolytic AdV and a helper-dependent AdV, armed with an anti-PD-L1 mini-antibody (a scFv CH2-CH3 fusion protein), improved the anti-tumor effect of chimeric antigen receptor (CAR) T cell therapy in mice. The benefits of locally produced anti-PD-L1 mini-antibody could not be achieved by anti-PD-L1 IgG infusion plus CAR T-cells and co-administration of an unarmed AdV. Tanoue et al 2017 “Armed oncolytic adenovirus expressing PD-L1 mini-body enhances anti-tumor effects of chimeric antigen receptor T-cells in solid tumors” Cancer Res. 77(8):2040-51. The use of that system, particularly in combination with CAR-T cell therapy, is also contemplated for use in delivering an Encoded Affimer to a target tumor.

Other viral vectors may be employed as a gene delivery system in the present disclosure. Vectors derived from viruses such as vaccinia virus (Puhlmann M. et al., Human Gene Therapy, 10:649-657 (1999); Ridgeway, “Mammalian expression vectors,” In: Vectors: A survey of molecular cloning vectors and their uses. Rodriguez and Denhardt, eds. Stoneham: Butterworth, 467-492 (1988); Baichwal and Sugden, “Vectors for gene transfer derived from animal DNA viruses: Transient and stable expression of transferred genes,” In: Kucherlapati R, ed. Gene transfer. New York: Plenum Press, 117-148 (1986) and Coupar et al., Gene, 68:1-10 (1988)), lentivirus (Wang G. et al., J. Clin. Invest., 104(11):R55-62 (1999)), herpes simplex virus (Chamber R., et al., Proc. Natl. Acad. Sci USA, 92:1411-1415 (1995)), poxvirus (GCE, NJL, Krupa M, Esteban M., The poxvirus vectors MVA and NYVAC as gene delivery systems for vaccination against infectious diseases and cancer Curr Gene Ther 8(2):97-120 (2008)), reovirus, measles virus, Semliki Forest virus, and polioviruses may be used in the present delivery systems for transferring the gene of interest into cells. They offer several attractive features for various mammalian cells. Also included are hepatitis B viruses.

b. Non-Viral Vectors

In 1990, Wolff et al. showed how injection of naked plasmid DNA (pDNA) into the skeletal muscle of mice led to the local expression of the encoded protein, kick-starting the field of DNA-based therapeutics. See Wolff et al. 1990 “Direct gene transfer into mouse muscle in vivo” Science. 247(4949 Pt 1):1465-8. The use of “pDNA” for delivering Encoded Affimers of the present disclosure waives the need for a virus as biological vector, and presents an appealing platform for Encoded Affimer gene transfer. Compared to viral vectors, pDNA is considered low-immunogenic (allowing e.g. repeated dosing), is cheaper to produce, ship, and store, and has a much longer shelf-life. After entry in the nucleus, pDNA remains in a non-replicating non-integrating episomal state, and is lost during the breakdown of the nuclear envelope at mitosis. pDNA has no defined restrictions regarding the size of the transgene compared to viral vectors, and its modular nature allows for straightforward molecular cloning, making them easy to manipulate and design for therapeutic use. Hardee et al. 2017 “Advances in non-viral DNA vectors for gene therapy” Genes. 8(2):65. Plasmids are used in about 17% of the ongoing or completed gene therapy clinical trials, and showed to be well-tolerated and safe.

The method of DNA administration can greatly impact transgene expression. In vivo DNA-mediated Encoded Affimer gene transfer can utilize such physical methods of transfection used for antibody gene transfer, such as electroporation or hydrodynamic injection. Electroporation presents the propagation of electrical fields within tissues, which induces a transient increase in cell membrane permeability. Electrotransfer of DNA is a multistep process, involving (i) electrophoretic migration of DNA towards the plasma membrane, (ii) DNA accumulation and interaction with the plasma membrane, and (iii) intracellular trafficking of the DNA to the nucleus, after which gene expression can commence. Heller L C. 2015 “Gene electrotransfer clinical trials” Adv Genet. 89:235-62. Intramuscular, intratumoral and intradermal administration have been evaluated in clinical trials and are also suitable target tissues for electroporation of Encoded Affimer constructs.

Hydrodynamic-based transfection utilizes the i.v. injection of high volumes of pDNA, driving DNA molecules out of the blood circulation and into tissue. Other potentially less invasive physical delivery methods include sonoporation and magnetofection. DNA uptake can also be improved by complexing the molecules with chemical delivery vehicles (e.g. cationic lipids or polymers and lipid nanoparticles). Such techniques can also be applied to in vivo DNA-mediated Encoded Affimer gene transfer.

In addition to the choice of delivery method, Encoded Affimer transgene expression can be improved by modifying the make-up of pDNA constructs. See, for example, Hardee et al. 2017 “Advances in non-viral DNA vectors for gene therapy” Genes 8(2):65; and Simcikova et al. 2015 “Towards effective non-viral gene delivery vector” Biotechnol Genet Eng Rev. 31(1-2):82-107. Conventional pDNA consists of a transcription unit and bacterial backbone. The transcription unit carries the Encoded Affimer sequence along with regulatory elements. The bacterial backbone includes elements like an antibiotic resistance gene, an origin of replication, unmethylated CpG motifs, and potentially cryptic expression signals. Some of these sequences are required for the production of plasmid DNA. However, in general, for therapeutic Encoded Affimer gene therapy the presence of a bacterial backbone will likely be counterproductive. However, there are a variety of different types of available minimal vectors that can be selected, including minicircle DNA (mcDNA) which already been used for antibody gene transfer and can be readily adapted for Encoded Affimer gene transfer. Minicircles are plasmid molecules devoid of bacterial sequences, generated via a process of recombination, restriction and/or purification. Simcikova et al. 2015 supra. Elimination of the bacterial backbone has shown higher transfection efficiency and prolonged transgene expression in a variety of tissues.

Also provided herein is a linear nucleic acid, or linear expression cassette (“LEC”), that is capable of being efficiently delivered to a subject via electroporation and expressing the Encoded Affimer sequence included therein. The LEC may be any linear DNA devoid of any phosphate backbone. The LEC may contain a promoter, an intron, a stop codon, and/or a polyadenylation signal. The expression of the Encoded Affimer coding sequence may be controlled by the promoter.

Plasmid Vectors

In some embodiments, the subject Encoded Affimers constructs are delivered as plasmid vectors. Plasmid vectors have been extensively described in the art and are well known to those of skill in the art. See e.g. Sambrook et al., 1989, cited above. In the last few years, plasmid vectors have been used as DNA vaccines for delivering antigen-encoding genes to cells in vivo. They are particularly advantageous for this because they reduced safety concerns relative to other vectors. These plasmids, however, having a promoter compatible with the host cell, can express a peptide epitope encoded by nucleic acid within the plasmid. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

To expand the application and efficiency of using plasmid DNA to deliver an Encoded Affimer construct to tissue in vivo, different approaches can be pursued based on principles producing higher mAb expression or overall efficacy in prior art reports. A first strategy simply relies on giving multiple or repeated pDNA doses. Kitaguchi et al. 2005 “Immune deficiency enhances expression of recombinant human antibody in mice after nonviral in vivo gene transfer” Int J Mol Med 16(4):683-8; and Yamazaki et al. 2011 “Passive immune-prophylaxis against influenza virus infection by the expression of neutralizing anti-hemagglutinin monoclonal antibodies from plasmids” Jpn J Infect Dis. 64(1):40-9. Another approach relates to the use of a delivery adjuvant. pDNA electrotransfer can be enhanced by pre-treating the muscle with hyaluronidase, an enzyme that transiently breaks down hyaluronic acid, decreasing the viscosity of the extracellular matrix and facilitating DNA diffusion. Yamazaki et al. 2011, supra; and McMahon et al. 2001 “Optimisation of electrotransfer of plasmid into skeletal muscle by pretreatment with hyaluronidase: increased expression with reduced muscle damage” Gene Ther. 8(16):1264-70. For antibody gene transfer, this led to an increase in mAb expression by approximately 3.5-fold, achieving plasma peak titers of 3.5 μg/ml with 30 μg pDNA, and would can be adapted by one skilled in the art for Encoded Affimer gene transfer. Still another strategy focuses on antibody or cassette engineering. Following codon-, RNA- and leader sequence-optimization, peak serum mAb or Fab titers have been attained with intramuscular electrotransfer of ‘optimized’ pDNA. See, for example, Flingai et al. 2015 “Protection against dengue disease by synthetic nucleic acid antibody prophylaxis/immunotherapy” Sci Rep. 5:12616.

The purpose of the plasmid is the efficient delivery of nucleic acid sequences to and expression of therapeutic Affimer Agents in a cell or tissue. In particular, the purpose of the plasmid may be to achieve high copy number, avoid potential causes of plasmid instability and provide a means for plasmid selection. As for expression, the nucleic acid cassette contains the necessary elements for expression of the Encoded Affimer within the cassette. Expression includes the efficient transcription of an inserted gene, nucleic acid sequence, or nucleic acid cassette with the plasmid. Thus, in some aspects, a plasmid is provided for expression of Encoded Affimer construct which includes an expression cassette comprising the coding sequence for the Affimer Agent; also referred to as a transcription unit. When a plasmid is placed in an environment suitable for epitope expression, the transcriptional unit will express the Affimer Agent and anything else encoded in the construct. The transcription unit includes a transcriptional control sequence, which is transcriptionally linked with a cellular immune response element coding sequence. Transcriptional control sequence may include promoter/enhancer sequences such as cytomegalovirus (CMV) promoter/enhancer sequences, such as described above. However, those skilled in the art will recognize that a variety of other promoter sequences suitable for expression in mammalian cells, including human patient cells, are known and can similarly be used in the constructs disclosed herein. The level of expression of the Affimer Agent will depend on the associated promoter and the presence and activation of an associated enhancer element.

In some embodiments, the Encoded Affimer sequence (encoding the desired Affimer Agent) can be cloned into an expression plasmid which contains the regulatory elements for transcription, translation, RNA stability and replication (i.e., including a transcriptional control sequence). Such expression plasmids are well known in the art and one of ordinary skill would be capable of designing an appropriate expression construct for producing a recombinant Affimer Agent in vivo.

Minicircle

Minicircle (mcDNA)-based antibody gene transfer can also be adapted for delivery of Encoded Affimers to tissues in vivo. Under certain circumstances, plasmid DNA used for non-viral gene delivery can cause unacceptable inflammatory responses. Where this happens, immunotoxic responses are largely due to the presence of unmethylated CpG motifs and their associated stimulatory sequences on plasmids following bacterial propagation of plasmid DNA. Simple methylation of DNA in vitro may be enough to reduce an inflammatory response, but can result in reduced gene expression. The removal of CpG islands by cloning out, or elimination of non-essential sequences has been a successful technique for reducing inflammatory responses. Yew et al. 2000 “Reduced inflammatory response to plasmid DNA vectors by elimination and inhibition of immunostimulatory CpG motifs” Mol Ther 1(3), 255-62.

Since bacterial DNA contains on average 4 times more CpG islands than mammalian DNA, a good solution is to eliminate entirely the bacterial control regions, such as the origin of replication and antibiotic resistance genes, from gene delivery vectors during the process of plasmid production. Thus, the “parent” plasmid is recombined into a “minicircle” which generally comprises the gene to be delivered (in this case, the Encoded Affimer coding sequence) and suitable control regions for its expression, and a miniplasmid which generally comprises the remainder of the parent plasmid.

Removal of bacterial sequences needs to be efficient, using the smallest possible excision site, whilst creating supercoiled DNA minicircles which consist solely of gene expression elements under appropriate—preferably mammalian—control regions. Some techniques for minicircle production use bacterial phage lambda (λ) integrase mediated recombination to produce minicircle DNA. See, for example, Darquet, et al. 1997 Gene Ther 4(12): 1341-9; Darquet et al. 1999 Gene Ther 6(2): 209-18; and Kreiss, et al. 1998 Appl Micbiol Biotechnol 49(5):560-7).

Therefore, embodiments of nucleic acid constructs described herein may be processed in the form of minicircle DNA. Minicircle DNA pertains to small (2-4 kb) circular plasmid derivatives that have been freed from all prokaryotic vector parts. Since minicircle DNA vectors contain no bacterial DNA sequences, they are less likely to be perceived as foreign and destroyed. As a result, these vectors can be expressed for longer periods of time compared to certain conventional plasmids. The smaller size of minicircles also extends their cloning capacity and facilitates their delivery into cells. Kits for producing minicircle DNA are known in the art and are commercially available (System Biosciences, Inc., Palo Alto, Calif.). Information on minicircle DNA is provided in Dietz et al., Vector Engineering and Delivery Molecular Therapy (2013); 21 8, 1526-1535 and Hou et al., Molecular Therapy—Methods & Clinical Development, Article number: 14062 (2015) doi:10.1038/mtm.2014.62. More information on Minicircles is provided in Chen Z Y, He C Y, Ehrhardt A, Kay M A. Mol Ther. 2003 September; 8(3):495-500 and Minicircle DNA vectors achieve sustained expression reflected by active chromatin and transcriptional level. Gracey Maniar L E, Maniar J M, Chen Z Y, Lu J, Fire A Z, Kay M A. Mol Ther. 2013 January; 21(1):131-8

As a nonlimiting example, a minicircle DNA vector may be produced as follows. An expression cassette, which comprises the Encoded Affimer coding sequence along with regulatory elements for its expression, is flanked by attachment sites for a recombinase. A sequence encoding the recombinase is located outside of the expression cassette and includes elements for inducible expression (such as, for example, an inducible promoter). Upon induction of recombinase expression, the vector DNA is recombined, resulting in two distinct circular DNA molecules. One of the circular DNA molecules is relatively small, forming a minicircle that comprises the expression cassette for the Encoded Affimer; this minicircle DNA vector is devoid of any bacterial DNA sequences. The second circular DNA sequence contains the remaining vector sequence, including the bacterial sequences and the sequence encoding the recombinase. The minicircle DNA containing the Encoded Affimer sequence can then be separately isolated and purified. In some embodiments, a minicircle DNA vector may be produced using plasmids similar to pBAD.ϕ.C31.hFIX and pBAD.ϕ.C31.RHB. See, e.g., Chen et al. (2003) Mol. Ther. 8:495-500.

Exemplary recombinases that may be used for creating a minicircle DNA vector include, but are not limited to, Streptomyces bacteriophage ϕ31 integrase, Cre recombinase, and the X integrase/DNA topoisomerase IV complex. Each of these recombinases catalyzes recombination between distinct sites. For example, ϕ31 integrase catalyzes recombination between corresponding attP and attB sites, Cre recombinase catalyzes recombination between loxP sites, and the λ integrase/DNA topoisomerase IV complex catalyzes recombination between bacteriophage λ attP and attB sites. In some embodiments, such as, for example, with ϕ31 integrase or with λ integrase in the absence of the λ is protein, the recombinase mediates an irreversible reaction to yield a unique population of circular products and thus high yields. In other embodiments, such as, for example, with Cre recombinase or with X integrase in the presence of the X protein, the recombinase mediates a reversible reaction to yield a mixture of circular products and thus lower yields. The reversible reaction by Cre recombinase can be manipulated by employing mutant loxP71 and loxP66 sites, which recombine with high efficiency to yield a functionally impaired P71/66 site on the minicircle molecule and a wild-type loxP site on the minicircle molecule, thereby shifting the equilibrium towards the production of the minicircle DNA product.

Published US Application 20170342424 also describes a system making use of a parent plasmid which is exposed to an enzyme which causes recombination at recombination sites, thereby forming a (i) minicircle including the Encoded Affimer sequence and (ii) a miniplasmid comprising the remainder of the parent plasmid. One recombination site is modified at the 5′ end such that its reaction with the enzyme is less efficient than the wild type site, and the other recombination site is modified at the 3′ end such that its reaction with the enzyme is less efficient than the wild type site, and the other recombination site is modified at the 3′ end such that its reaction with the enzyme is less efficient than the wild type site, both modified sites being located in the minicircle after recombination. This favors the formation of minicircle.

c. RNA-mediated Encoded Affimer Gene Transfer

Exemplary nucleic acids or polynucleotides for the encoded PD-L1 Affimer Agents of the present disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof.

mRNA presents an emerging platform for antibody gene transfer that can be adapted by those skilled in the art for delivery of Encoded Affimer constructs of the present disclosure. Although current results differ considerably, in certain instances the mRNA constructs appear to be able to rival viral vectors in terms of generated serum mAb titers. Levels were in therapeutically relevant ranges within hours after mRNA administration, a marked shift in speed compared to DNA. The use of lipid nanoparticles (LNP) for mRNA transfection, rather than the physical methods typically required for DNA, can provide significant advantages in some embodiments towards application range.

In their 1990 study, Wolff et al. (1990, supra) found that, in addition to pDNA, intramuscular injection of in vitro transcribed (IVT) mRNA also led to local expression of the encoded protein. mRNA was not pursued as actively as DNA at that time because of its low stability. Progress over the past years allowed mRNA to catch up with DNA and viral vectors as a tool for gene transfer. Reviewed in Sahin et al. (2014) “mRNA-based therapeutics: developing a new class of drugs” Nat Rev Drug Discov. 13(10):759-80. Conceptually, there are several differences with these expression platforms. mRNA does not need to enter into the nucleus to be functional. Once it reaches the cytoplasm, mRNA is translated instantly. mRNA-based therapeutics are expressed more transiently compared to DNA- or viral vector-mediated gene transfer, and do not pose the risk of insertional mutagenesis in the host genome. mRNA production is relatively simple and inexpensive. In terms of administration, mRNA uptake can be enhanced using electroporation. Broderick et al. 2017 “Enhanced delivery of DNA or RNA vaccines by electroporation” Methods Mol Biol. 2017; 1499:193-200. Most focus, however, has gone to non-physical transfection methods. Indeed, a variety of mRNA complexing formulations have been developed, including lipid nanoparticles (LNP), which have proven to be safe and very efficient mRNA carriers for administration in a variety of tissues and i.v. Pardi et al. 2015 “Expression kinetics of nucleoside-modified mRNA delivered in lipid nanoparticles to mice by various routes” J Control Release 217:345-51. In line with this progress, IVT mRNA has reached the stage of clinical evaluation.

Beissert et al. WO2017162266 “RNA Replicon for Versatile and Efficient Gene Expression” describes agents and methods suitable for efficient expression of affimers of the present disclosure, such as suitable for immunotherapeutic treatment for the prevention and therapy of tumors. For instance, the Affimer Agent coding sequence can be provided as an RNA replicon comprising a 5′ replication recognition sequence such as from an alphavirus 5′ replication recognition sequence. In some embodiments, the RNA replicon comprises a (modified) 5′ replication recognition sequence and an open reading frame encoding the Affimer Agent, in particular located downstream from the 5′ replication recognition sequence such as that the 5′ replication recognition sequence and the open reading frame do not overlap, e.g. the 5′ replication recognition sequence does not contain a functional initiation codon and in some embodiments does not contain any initiation codon. Most preferably, the initiation codon of the open reading frame encoding the Affimer Agent is in the 5′→3′ direction of the RNA replicon.

In some embodiments, to prevent immune activation, modified nucleosides can be incorporated into the in vitro-transcribed mRNA. In some embodiments, the IVT RNA can be 5′ capped, such an m7G5′ppp5′G2′-O-Met-capped IVT. Efficient translation of the modified mRNA can be ensured by removing double-stranded RNA. Moreover, the 5′ and 3′ UTRs and the poly(A) tail can be optimized for improved intracellular stability and translational efficiency. See, for example, Stadler et al. (2017) Nature Medicine 23:815-817 and Kariko et al. WO/2017/036889 “Method for Reducing Immunogenicity of RNA”.

In some embodiments, the mRNA that encodes the PD-L1 Affimer Agent may include at least one chemical modification described herein. As a non-limiting example, the chemical modification may be 1-methylpseudouridine, 5-methylcytosine or 1-methylpseudouridine and 5-methylcytosine. In some embodiments, linear polynucleotides encoding one or more PD-L1 Affimer Agents of the present disclosure which are made using only in vitro transcription (IVT) enzymatic synthesis methods are referred to as “IVT polynucleotides.” Methods of making IVT polynucleotides are known in the art and are described in PCT Application WO2013/151666, the contents of which are incorporated herein by reference in their entirety.

In another embodiment, the polynucleotides that encode the PD-L1 Affimer Agent of the present disclosure have portions or regions which differ in size and/or chemical modification pattern, chemical modification position, chemical modification percent or chemical modification population and combinations of the foregoing are known as “chimeric polynucleotides.” A “chimera” according to the present disclosure is an entity having two or more incongruous or heterogeneous parts or regions. As used herein a “part” or “region” of a polynucleotide is defined as any portion of the polynucleotide which is less than the entire length of the polynucleotide. Such constructs are taught in for example PCT Application WO2015/034928.

In yet another embodiment, the polynucleotides of the present disclosure that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP. Such constructs are taught in for example PCT Application WO2015/034925 and WO2015/034928, the contents of each of which are incorporated herein by reference in their entirety.

Exemplary mRNA (and other polynucleotides) that can be used to encode PD-L1 Affimer Agents of the present disclosure include those which can be adapted from the specifications and FIGS. of, for example, PCT Publications WO2017/049275, WO2016/118724, WO2016/118725, WO2016/011226, WO2015/196128, WO/2015/196130, WO/2015/196118, WO/2015/089511, with WO2015/105926 (the later titled “Polynucleotides for the In vivo Production Of Antibodies”), each of which is incorporated by reference herein.

Electroporation, as described below, is one exemplary method for introducing mRNA or other polynucleotides into a cell.

Lipid-containing nanoparticle compositions have proven effective as transport vehicles into cells and/or intracellular compartments for a variety of RNAs (and related polynucleotides described herein). These compositions generally include one or more “cationic” and/or ionizable lipids, phospholipids including polyunsaturated lipids, structural lipids (e.g., sterols), and lipids containing polyethylene glycol (PEG lipids). Cationic and/or ionizable lipids include, for example, amine-containing lipids that can be readily protonated.

d. Delivery of Encoded Affimer Constructs into Target Cells

The introduction into host cell of the gene delivery system can be performed through various methods known to those skilled in the art.

Where the present gene delivery system is constructed on the basis of viral vector construction, delivery can be performed as conventional infection methods known in the art.

Physical methods to enhance delivery both viral and non-viral Encoded Affimer constructs include electroporation (Neumann, E. et al., EMBO J., 1:841 (1982); and Tur-Kaspa et al., Mol. Cell Biol., 6:716-718 (1986)), gene bombardment (Yang et al., Proc. Natl. Acad. Sci., 87:9568-9572 (1990) where DNA is loaded onto (e.g., gold) particles and forced to achieve penetration of the DNA into the cells, sonoporation, magnetofection, hydrodynamic delivery and the like, all of which are known to those of skill in the art.

Electroporation

In the past several years, there has been a great advance in the plasmid DNA delivery technology that is utilized for in vivo production of proteins. This included codon optimization for expression in human cells, RNA optimization to improve mRNA stability as well as more efficient translation at the ribosomal level, the addition of specific leader sequences to enhance translation efficiency, the creation of synthetic inserts to further enhance production in vivo and the use of improved adaptive electroporation (EP) delivery protocols to improve in vivo delivery. EP assists in the delivery of plasmid DNA by generating an electrical field that allows the DNA to pass into the cell more efficiently. In vivo electroporation is a gene delivery technique that has been used successfully for efficient delivery of plasmid DNA to many different tissues. Kim et al. “Gene therapy using plasmid DNA-encoded anti-HER2 antibody for cancers that overexpress HER2” (2016) Cancer Gene Ther. 23(10): 341-347 teaches a vector and electroporation system for intramuscular injection and in vivo electroporation of the plasmids that results in high and sustained antibody expression in sera; the plasmid and electroporation system of Kim et al. can be readily adapted for the in vivo delivery of a plasmid for expressing an encoded PD-L1 binding affimer of the present disclosure.

Accordingly, in certain some embodiments of the present disclosure, the Encoded Affimer construct is introduced into target cells via electroporation.

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (VGX Pharmaceuticals, Blue Bell, Pa.) or Elgen electroporator (Genetronics, San Diego, Calif.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but in some embodiments is a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the Encoded Affimer constructs of the present disclosure, include those described in U.S. Pat. Nos. 7,245,963; 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

In some embodiments, the electroporation is carried using a minimally invasive electroporation device (“MID”). The device may comprise a hollow needle, DNA cassette, and fluid delivery means, wherein the device is adapted to actuate the fluid delivery means in use so as to concurrently (for example, automatically) inject the Encoded Affimer nucleic acid construct into body tissue during insertion of the needle into the body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the Encoded Affimer nucleic acid construct into tissue without the use of a needle. The MID may inject the Encoded Affimer nucleic acid construct as a small stream or jet with such force that the nucleic acid pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired Encoded Affimer nucleic acid construct in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the nucleic acid into the tissue. For example, if the tissue to be treated is a mucosa, skin or muscle, the agent is projected towards the mucosal or skin surface with sufficient force to cause the agent to penetrate through the stratum corneum and into dermal layers, or into underlying tissue and muscle, respectively. Needle-free injectors are well suited to deliver Encoded Affimer nucleic acid construct to all types of tissues, including into tumors (intratumoral delivery).

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering the Encoded Affimer nucleic acid construct to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the Encoded Affimer nucleic acid construct and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Plymouth Meeting, Pa.) device and system, which is a modular electrode system, that facilitates the introduction of a macromolecule, such as an Encoded Affimer nucleic acid construct, into cells of a selected tissue in a body. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The nucleic acid is then delivered via the hypodermic needle into the selected tissue. The programmable constant-current pulse controller is activated and constant-current electrical pulse is applied to the plurality of needle electrodes. The applied constant-current electrical pulse facilitates the introduction of the nucleic acid into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described Encoded Affimer nucleic acid construct herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present disclosure include tumor tissue, skin and other epithelial tissues, liver tissue and muscle tissue, merely as examples.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached the target tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present disclosure, both the volume of fluid injected and the size of electric field applied may be reduced while achieving a good fit between the electric field and the fluid.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes. The electrodes described in U.S. Pat. No. 7,245,963 and U.S. Patent Pub. 2005/005263 are, for example, 20 mm long and 21 gauge.

Use of in vivo electroporation enhances plasmid DNA uptake in tumor tissue, resulting in expression within the tumor, and delivers plasmids to muscle tissue, resulting in systemic expression of secreted proteins, such as cytokines (see, e.g., U.S. Pat. No. 8,026,223). Additional exemplary techniques, vectors and devices for electroporating PD-L1 Affimer Agent transgenes into cells in vivo include PCT Publications WO/2017/106795, WO/2016/161201, WO/2016/154473, WO/2016/112359 and WO/2014/066655.

Typically, the electric fields needed for in vivo cell electroporation are generally similar in magnitude to the fields required for cells in vitro. In some embodiments, the magnitude of the electric field range from approximately, 10 V/cm to about 1500 V/cm, 300 V/cm to 1500 V/cm, or 1000 V/cm to 1500 V/cm. Alternatively, lower field strengths (from about 10 V/cm to 100 V/cm, and more preferably from about 25 V/cm to 75 V/cm) the pulse length is long. For example, when the nominal electric field is about 25-75 V/cm, if is preferred that the pulse length is about 10 msec.

The pulse length can be about 10 s to about 100 ms. There can be any desired number of pulses, typically one to 100 pulses per second. The delay between pulses sets can be any desired time, such as one second. The waveform, electric field strength and pulse duration may also depend upon the type of cells and the type of molecules that are to enter the cells via electroporation.

Also encompassed are electroporation devices incorporating electrochemical impedance spectroscopy (“EIS”). Such devices provide real-time information on in vivo, in particular, intratumoral electroporation efficiency, allowing for the optimization of conditions. Examples of electroporation devices incorporating EIS can be found, e.g., in WO2016/161201, which is hereby incorporated by reference.

Uptake of the Encoded Affimer nucleic acid constructs of the present disclosure may also be enhanced by plasma electroporation also termed avalanche transfection. Briefly, microsecond discharges create cavitation microbubbles at electrode surface. The mechanical force created by the collapsing microbubbles combined with the magnetic field serve to increase transport efficiency across the cell membrane as compared with the diffusion mediated transport associated with conventional electroporation. The technique of plasma electroporation is described in U.S. Pat. Nos. 7,923,251 and 8,283,171. This technique may also be employed in vivo for the transformation of cells. Chaiberg, et al (2006) Investigative Ophthalmology & Visual Science 47:4083-4090; Chaiberg, et al U.S. Pat. No. 8,101,169 Issued Jan. 24, 2012.

Other alternative electroporation technologies are also contemplated. In vivo nucleic acid delivery can also be performed using cold plasma. Plasma is one of the four fundamental states of matter, the others being solid, liquid, and gas. Plasma is an electrically neutral medium of unbound positive and negative particles (i.e. the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo, et al. (1998) Phys. Plasma 5:2868-2870) and is accompanied by the dissociation of molecular bonds, if present.

Cold plasmas (i.e., non-thermal plasmas) are produced by the delivery of pulsed high voltage signals to a suitable electrode. Cold plasma devices may take the form of a gas jet device or a dielectric barrier discharge (DBD) device. Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, various other medical therapies and sterilization. As noted earlier, cold plasmas (i.e., non-thermal plasmas) are produced by the delivery of pulsed high voltage signals to a suitable electrode. Cold plasma devices may take the form of a gas jet device, a dielectric barrier discharge (DBD) device or multi-frequency harmonic-rich power supply.

Dielectric barrier discharge device, relies on a different process to generate the cold plasma. A dielectric barrier discharge (DBD) device contains at least one conductive electrode covered by a dielectric layer. The electrical return path is formed by the ground that can be provided by the target substrate undergoing the cold plasma treatment or by providing an in-built ground for the electrode. Energy for the dielectric barrier discharge device can be provided by a high voltage power supply, such as that mentioned above. More generally, energy is input to the dielectric barrier discharge device in the form of pulsed DC electrical voltage to form the plasma discharge. By virtue of the dielectric layer, the discharge is separated from the conductive electrode and electrode etching and gas heating is reduced. The pulsed DC electrical voltage can be varied in amplitude and frequency to achieve varying regimes of operation. Any device incorporating such a principle of cold plasma generation (e.g., a DBD electrode device) falls within the scope of various embodiments of the present disclosure.

Cold plasma has been employed to transfect cells with foreign nucleic acids. In particular, transfection of tumor cells (see, e.g., Connolly, et al. (2012) Human Vaccines & Immune-therapeutics 8: 1729-1733; and Connolly et al (2015) Bioelectrochemistry 103: 15-21).

In certain illustrative embodiments, the transgene construct encoding the PD-L1 Affimer Agent of the present disclosure is delivered using an electroporation device comprising: an applicator; a plurality of electrodes extending from the applicator, the electrodes being associated with a cover area; a power supply in electrical communication with the electrodes, the power supply configured to generate one or more electroporating signals to cells within the cover area; and a guide member coupled to the electrodes, wherein the guide member is configured to adjust the cover area of the electrodes. At least a portion of the electrodes can be positioned within the applicator in a conical arrangement. The one or more electroporating signals may be each associated with an electric field. The device may further comprise a potentiometer coupled to the power supply and electrodes. The potentiometer may be configured to maintain the electric field substantially within a predetermined range.

The one or more electroporating signals may be each associated with an electric field. The device may further comprise a potentiometer coupled to the power supply and the electrodes. The potentiometer may be configured to maintain the electric field within a predetermined range so as to substantially prevent permanent damage in the cells within the cover area and/or substantially minimize pain. For instance, potentiometer may be configured to maintain the electric field to about 1300 V/cm.

The power supply may provide a first electrical signal to a first electrode and a second electrical signal to a second electrode. The first and second electrical signals may combine to produce a wave having a beat frequency. The first and second electrical signals may each have at least one of a unipolar waveform and a bipolar waveform. The first electrical signal may have a first frequency and a first amplitude. The second electrical signal may have a second frequency and a second amplitude. The first frequency may be different from or the same as the second frequency. The first amplitude may be different from or the same as the second amplitude.

In some embodiments, the present disclosure provides a method for treating a subject having a tumor, the method comprising: injecting the tumor with an effective dose of plasmid coding for a PD-L1 Affimer Agent; and administering electroporation therapy to the tumor. In some embodiments, the electroporation therapy further comprises the administration of at least one voltage pulse of about 200 V/cm to about 1500 V/cm over a pulse width of about 100 microseconds to about 20 milliseconds.

In some embodiments, the plasmid (or a second electroporated plasmid) further encodes at least one immunostimulatory cytokine, such as selected from the group encoding IL-12, IL-15, and a combination of IL-12 and IL-15.

Transfection Enhancing Formulations Encoded Affimer nucleic acid constructs can also be encapsulated in liposomes, preferably cationic liposomes (Wong, T. K. et al., Gene, 10:87 (1980); Nicolau and Sene, Biochim. Biophys. Acta, 721:185-190 (1982); and Nicolau et al., Methods Enzymol., 149:157-176 (1987)) or polymersomes (synthetic liposomes) which can interact with the cell membrane and fuse or undergo endocytosis to effect nucleic acid transfer into the cell. The DNA also can be formed into complexes with polymers (polyplexes) or with dendrimers which can directly release their load into the cytoplasm of a cell.

Illustrative carriers useful in this regard include microparticles of poly(lactide-co-glycolide), polyacrylate, latex, starch, cellulose, dextran and the like. Other illustrative carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active agent contained within a sustained release formulation depends upon the site of implantation, the rate and expected duration of release and the nature of the condition to be treated or prevented.

Biodegradable microspheres (e.g., polylactate polyglycolate) may be employed as carriers for compositions. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which can have the added benefit when used intratumorally to deliver the coding sequence for a PD-L1 Affimer

Biodegradable polymeric nanoparticles facilitate nonviral nucleic acid transfer to cells. Small (approximately 200 nm), positively charged (approximately 10 mV) particles are formed by the self-assembly of cationic, hydrolytically degradable poly(beta-amino esters) and plasmid DNA.

Polynucleotides may also be administered to cells by direct microinjection, temporary cell permeabilizations (e.g., co-administration of repressor and/or activator with a cell permeabilizing agent), fusion to membrane translocating peptides, and the like.

Lipid-mediated nucleic acid delivery and expression of foreign nucleic acids, including mRNA, in vitro and in vivo has been very successful. Lipid based non-viral formulations provide an alternative to viral gene therapies. Current in vivo lipid delivery methods use subcutaneous, intradermal, intratumoral, or intracranial injection. Advances in lipid formulations have improved the efficiency of gene transfer in vivo (see PCT Application WO 98/07408). For instance, a lipid formulation composed of an equimolar ratio of 1,2-bis(oleoyloxy)-3-(trimethyl ammonio)propane (DOTAP) and cholesterol can significantly enhances systemic in vivo gene transfer. The DOTAP:cholesterol lipid formulation forms unique structure termed a “sandwich liposome”. This formulation is reported to “sandwich” DNA between an invaginated bi-layer or ‘vase’ structure. Beneficial characteristics of these lipid structures include a positive p, colloidal stabilization by cholesterol, two dimensional nucleic acid packing and increased serum stability.

Cationic liposome technology is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. Similarly, other polycations, such as poly-1-lysine and polyethylene-imine, complex with nucleic acids via charge interaction and aid in the condensation of DNA or RNA into nanoparticles, which are then substrates for endosome-mediated uptake. Several of these cationic-nucleic acid complex technologies have been developed as potential clinical products, including complexes with plasmid DNA (pDNA), oligodeoxynucleotides, and various forms of synthetic RNA, and be used as part of the delivery system for the Encoded Affimer nucleic acid constructs of the present disclosure.

The Encoded Affimer nucleic acid constructs disclosed herein may be associated with polycationic molecules that serve to enhance uptake into cells. Complexing the nucleic acid construct with polycationic molecules also helps in packaging the construct such their size is reduced, which is believed to assist with cellular uptake. Once in the endosome, the complex dissociates due to the lower pH, and the polycationic molecules can disrupt the endosome's membrane to facilitate DNA escape into the cytoplasm before it can be degraded. Preliminary data shows that the nucleic acid construct embodiments had enhanced uptake into SCs over DCs when complexed with the polycationic molecules polylysine or polyethyleneimine.

One example of polycationic molecules useful for complexing with nucleic acid constructs includes cell penetrating peptides (CPP), examples include polylysine (described above), polyarginine and Tat peptides. Cell penetrating peptides (CPP) are small peptides which can bind to DNA and, once released, penetrate cell membranes to facilitate escape of the DNA from the endosome to the cytoplasm. Another example of a CPP pertains to a 27 residue chimeric peptide, termed MPG, was shown some time ago to bind ss- and ds-oligonucleotides in a stable manner, resulting in a non-covalent complex that protected the nucleic acids from degradation by DNase and effectively delivered oligonucleotides to cells in vitro (Mahapatro A, et al., J Nanobiotechnol, 2011, 9:55). The complex formed small particles of approximately 150 nm to 1 um when different peptide:DNA ratios were examined, and the 10:1 and 5:1 ratios (150 nm and 1 um respectively). Another CPP pertains to a modified tetrapeptide [tetralysine containing guanidinocarbonylpyrrole (GCP) groups (TL-GCP)], which was reported to bind with high affinity to a 6.2 kb plasmid DNA resulting in a positive charged aggregate of 700-900 nm Li et al., Agnew Chem Int Ed Enl 2015; 54(10):2941-4). RNA can also be complexed by such polycationic molecules for in vivo delivery.

Other examples of polycationic molecules that may be complexed with the nucleic acid constructs described herein include polycationic polymers commercially available as JETPRIME® and In vivo JET (Polypus-transfection, S.A., Illkirch, France).

In some embodiments, the present disclosure contemplates a method of delivering an mRNA (or other polynucleotide)f encoding a PD-L1 Affimer Agent to a patient's cells by administering a nanoparticle composition comprising (i) a lipid component comprising a compound of formula (I), a phospholipid, a structural lipid, and a PEG lipid; and (ii) an mRNA (or other polynucleotide)f, said administering comprising contacting said mammalian cell with said nanoparticle composition, whereby said mRNA (or other polynucleotide)f is delivered to said cell.

In exemplary embodiments, the PEG lipid is selected from the group consisting of a PEG-modified phosphatidylethanolamine, a PEG-modified phosphatide acid, a PEG-modified ceramide, a PEG-modified dialkylamine, a PEG-modified diacylglycerol and a PEG-modified dialkylglycerol. In exemplary embodiments, the structural lipid is selected from the group consisting of cholesterol, fecosterol, sitosterol, ergosterol, campesterol, stigmasterol, brassicasterol, tomatidine, ursolic acid, and alpha-tocopherol. In some embodiments, the structural lipid is cholesterol.

In exemplary embodiments, the phospholipid includes a moiety selected from the group consisting of phosphatidyl choline, phosphatidyl ethanolamine, phosphatidyl glycerol, phosphatidyl serine, phosphatidic acid, 2-lysophosphatidyl choline, and a sphingomyelin. In some embodiments, the phospholipid includes one or more fatty acid moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid. In some embodiments, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (1 8:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine,1,2-dioleoyl-sn-glycero-3-phosphoethanola mine (DOPE), 1,2-diphytanoyl-sn-glycero-3-phosphoethanolamine (ME 1 6.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin In some embodiments, the phospholipid is DOPE or DSPC.

To further illustrate, the phospholipid can be DOPE and said the component can comprise about 35 mol % to about 45 mol % said compound, about 1 0 mol % to about 20 mol % DOPE, about 38.5 mol % to about 48.5 mol % structural lipid, and about 1.5 mol % PEG lipid. The lipid component can be about 40 mol % said compound, about 15 mol % phospholipid, about 43.5 mol % structural lipid, and about 1.5 mol % PEG lipid.

In some embodiments, the wt/wt ratio of lipid component to PD-L1 Affimer Agent encoding mRNA (or other polynucleotide) is from about 5:1 to about 50:1, or about 10:1 to about 40:1

In some embodiments, the mean size of said nanoparticle composition is from about 50 nm to about 150 nm, or from about 80 nm to about 120 nm.

In some embodiments, the polydispersity index of said nanoparticle composition is from about 0 to about 0.18, or from about 0.13 to about 0.17.

In some embodiments, the nanoparticle composition has a zeta potential of about −10 to about +20 mV.

In some embodiments, the nanoparticle composition further comprises a cationic and/or ionizable lipid selected from the group consisting of 3-(didodecylamino)-N1,N1,4-tridodecyl-1-piperazineethanamine (KL1 0), 14,25-ditridecyl-1 5, 1 8,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-1 9-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), and (2R)-2-({8-[(3P)-cholest-5-en-3-yloxy]octyl}oxy)-N,N-dimethyl-3-[(9Z,1 2Z)-octadeca-9, 12-dien-1-yl oxy]propan-1-amine (Octyl-CLinDMA (2R)).

### VI. Methods of Use and Pharmaceutical Compositions

The Affimer Agents of the disclosure are useful in a variety of applications including, but not limited to, therapeutic treatment methods, such as immunotherapy for cancer. In some embodiments, Affimer Agents described herein are useful for activating, promoting, increasing, and/or enhancing an immune response, inhibiting tumor growth, reducing tumor volume, inducing tumor regression, increasing tumor cell apoptosis, and/or reducing the tumorigenicity of a tumor. In some embodiments, the polypeptides or agents of the disclosure are also useful for immunotherapy against pathogens, such as viruses. In some embodiments, the Affimer Agents described herein are useful for inhibiting viral infection, reducing viral infection, increasing virally-infected cell apoptosis, and/or increasing killing of virus-infected cells. The methods of use may be in vitro, ex vivo, or in vivo methods.

The present disclosure provides methods for activating an immune response in a subject using an Affimer Agent. In some embodiments, the disclosure provides methods for promoting an immune response in a subject using an Affimer Agent described herein. In some embodiments, the disclosure provides methods for increasing an immune response in a subject using an Affimer Agent. In some embodiments, the disclosure provides methods for enhancing an immune response in a subject using an Affimer Agent. In some embodiments, the activating, promoting, increasing, and/or enhancing of an immune response comprises increasing cell-mediated immunity. In some embodiments, the activating, promoting, increasing, and/or enhancing of an immune response comprises increasing Th1-type responses. In some embodiments, the activating, promoting, increasing, and/or enhancing of an immune response comprises increasing T-cell activity. In some embodiments, the activating, promoting, increasing, and/or enhancing of an immune response comprises increasing CD4+ T-cell activity. In some embodiments, the activating, promoting, increasing, and/or enhancing of an immune response comprises increasing CD8+ T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CU activity and increasing NK cell activity. In some embodiments, the activating, promoting, increasing, and/or enhancing of an immune response comprises inhibiting or decreasing the suppressive activity of Treg cells. In some embodiments, the activating, promoting, increasing, and/or enhancing of an immune response comprises inhibiting or decreasing the suppressive activity of MDSCs. In some embodiments, the activating, promoting, increasing, and/or enhancing of an immune response comprises increasing the number of the percentage of memory T-cells. In some embodiments, the activating, promoting, increasing, and/or enhancing of an immune response comprises increasing long-term immune memory function. In some embodiments, the activating, promoting, increasing, and/or enhancing of an immune response comprises increasing long-term memory. In some embodiments, the activating, promoting, increasing, and/or enhancing of an immune response comprises no evidence of substantial side effects and/or immune-based toxicities. In some embodiments, the activating, promoting, increasing, and/or enhancing of an immune response comprises no evidence of cytokine release syndrome (CRS) or a cytokine storm. In some embodiments, the immune response is a result of antigenic stimulation. In some embodiments, the antigenic stimulation is a tumor cell. In some embodiments, the antigenic stimulation is cancer. In some embodiments, the antigenic stimulation is a pathogen. In some embodiments, the antigenic stimulation is a virally-infected cell.

In vivo and in vitro assays for determining whether an Affimer Agent activates, or inhibits an immune response are known in the art.

In some embodiments, a method of increasing an immune response in a subject comprises administering to the subject a therapeutically effective amount of an Affimer Agent described herein, wherein the an Affimer Agent binds human PD-L1. In some embodiments, a method of increasing an immune response in a subject comprises administering to the subject a therapeutically effective amount of an Affimer Agent described herein, wherein the Affimer Agent is a affimer-containing antibody or receptor trap fusion polypeptide including an affimer polypeptide that specifically binds to PD-L1. In some embodiments, a method of increasing an immune response in a subject comprises administering to the subject a therapeutically effective amount of an Encoded Affimer, wherein the Encoded Affimer, when expressed in the patient, produces a recombinant Affimer Agent polypeptide including an anti-PD-L1 affimer polypeptide.

In some embodiments of the methods described herein, a method of activating or enhancing a persistent or long-term immune response to a tumor comprises administering to a subject a therapeutically effective amount of an Affimer Agent which binds human PD-L1. In some embodiments, a method of activating or enhancing a persistent immune response to a tumor comprises administering to a subject a therapeutically effective amount of an Affimer Agent described herein, wherein the Affimer Agent is an affimer-containing antibody or receptor trap fusion polypeptide including an affimer polypeptide that specifically binds to PD-L1. In some embodiments, a method of activating or enhancing a persistent immune response to a tumor comprises administering to a subject a therapeutically effective amount of an Encoded Affimer, wherein the Encoded Affimer, when expressed in the patient, produces a recombinant Affimer Agent polypeptide including an anti-PD-L1 affimer polypeptide.

In some embodiments of the methods described herein, a method of inducing a persistent or long-term immunity which inhibits tumor relapse or tumor regrowth comprises administering to a subject a therapeutically effective amount of an Affimer Agent which binds human PD-L1. In some embodiments, a method of inducing a persistent immunity which inhibits tumor relapse or tumor regrowth comprises administering to a subject a therapeutically effective amount of an Affimer Agent described herein, wherein the Affimer Agent is a affimer-containing antibody or receptor trap fusion polypeptide including an affimer polypeptide that specifically binds to PD-L1. In some embodiments, a method of inducing a persistent immunity which inhibits tumor relapse or tumor regrowth comprises administering to a subject a therapeutically effective amount of an Encoded Affimer, wherein the Encoded Affimer, when expressed in the patient, produces a recombinant Affimer Agent polypeptide including an anti-PD-L1 affimer polypeptide.

In some embodiments of the methods described herein, a method of inhibiting tumor relapse or tumor regrowth comprises administering to a subject a therapeutically effective amount of an Affimer Agent which binds human PD-L1. In some embodiments, a method of inhibiting tumor relapse or tumor regrowth comprises administering to a subject a therapeutically effective amount of an Affimer Agent described herein, wherein the Affimer Agent is an affimer-containing antibody or receptor trap fusion polypeptide including an affimer polypeptide that specifically binds to PD-L1. In some embodiments, a method of inhibiting tumor relapse or tumor regrowth comprises administering to a subject a therapeutically effective amount of an Encoded Affimer, wherein the Encoded Affimer, when expressed in the patient, produces a recombinant Affimer Agent polypeptide including an anti-PD-L1 affimer polypeptide.

In some embodiments, the tumor expresses or overexpresses a tumor antigen that is targeted by an additional binding entity provided in the Affimer Agent along with the anti-PD-L1 affimer polypeptide, i.e., where the Affimer Agent is a bispecific or multispecific agent.

In some embodiments, the method of inhibiting growth of a tumor comprises administering to a subject a therapeutically effective amount of an Affimer Agent described herein. In some embodiments, the subject is a human. In some embodiments, the subject has a tumor, or the subject had a tumor which was removed.

In some embodiments, the tumor is a solid tumor. In some embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In some embodiments, the tumor is a colorectal tumor. In some embodiments, the tumor is an ovarian tumor. In some embodiments, the tumor is a lung tumor. In some embodiments, the tumor is a pancreatic tumor. In some embodiments, the tumor is a melanoma tumor. In some embodiments, the tumor is a bladder tumor.

To further illustrate, the subject Affimer Agents can be used to treat patients suffering from cancer, such as osteosarcoma, rhabdomyosarcoma, neuroblastoma, kidney cancer, leukemia, renal transitional cell cancer, bladder cancer, Wilm's cancer, ovarian cancer, pancreatic cancer, breast cancer (including triple negative breast cancer), prostate cancer, bone cancer, lung cancer (e.g., small cell or non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer, breast cancer or gastric cancer. In some embodiments of the disclosure, the cancer is metastatic cancer, e.g., of the varieties described above.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The present disclosure also provides pharmaceutical compositions comprising an Affimer Agent described herein and a pharmaceutically acceptable vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified Affimer Agent of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, an Affimer Agent described herein is lyophilized and/or stored in a lyophilized form. In some embodiments, a formulation comprising an Affimer Agent described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London).

The pharmaceutical compositions of the present disclosure can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The Affimer Agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In some embodiments, pharmaceutical formulations include an Affimer Agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, sustained-release preparations comprising Affimer Agents described herein can be produced. Suitable examples of sustained-release preparations include semi-permeable matrices of solid hydrophobic polymers containing an Affimer Agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In some embodiments, in addition to administering an Affimer Agent described herein, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the Affimer Agent. Pharmaceutical compositions comprising an Affimer Agent and the immune response stimulating agent(s) are also provided. In some embodiments, the immune response stimulating agent comprises 1, 2, 3, or more immune response stimulating agents.

In some embodiments, in addition to administering an Affimer Agent described herein, the method or treatment further comprises administering at least one additional therapeutic agent. An additional therapeutic agent can be administered prior to, concurrently with, and/or subsequently to, administration of the Affimer Agent. Pharmaceutical compositions comprising an Affimer Agent and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the Affimer Agent. Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, the combination of an Affimer Agent described herein and at least one additional therapeutic agent results in additive or synergistic results. In some embodiments, the combination therapy results in an increase in the therapeutic index of the Affimer Agent. In some embodiments, the combination therapy results in an increase in the therapeutic index of the additional therapeutic agent(s). In some embodiments, the combination therapy results in a decrease in the toxicity and/or side effects of the Affimer Agent. In some embodiments, the combination therapy results in a decrease in the toxicity and/or side effects of the additional therapeutic agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In some embodiments, the second therapeutic agent is an alkylating agent, an antimetabolite, an antimitotic, a topoisomerase inhibitor, or an angiogenesis inhibitor.

Therapeutic agents that may be administered in combination with the Affimer Agent described herein include chemotherapeutic agents. Thus, in some embodiments, the method or treatment involves the administration of an Affimer Agent of the present disclosure in combination with a chemotherapeutic agent or in combination with a cocktail of chemotherapeutic agents. Treatment with an Affimer Agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4.sup.th Edition, 2008, M. C. Perry, Editor, Lippincott, Williams & Wilkins, Philadelphia, Pa.

Chemotherapeutic agents useful in the present disclosure include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In some embodiments, the additional therapeutic agent is cisplatin. In some embodiments, the additional therapeutic agent is carboplatin.

In some embodiments of the methods described herein, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In some embodiments, the additional therapeutic agent is gemcitabine.

In some embodiments of the methods described herein, the chemotherapeutic agent is an antimitotic agent, including, but not limited to, agents that bind tubulin. In some embodiments, the agent is a taxane. In some embodiments, the agent is paclitaxel or docetaxel, or a pharmaceutically acceptable salt, acid, or derivative of paclitaxel or docetaxel. In some embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In some embodiments, the additional therapeutic agent is paclitaxel. In some embodiments, the additional therapeutic agent is nab-paclitaxel.

In some embodiments of the methods described herein, an additional therapeutic agent comprises an agent such as a small molecule. For example, treatment can involve the combined administration of an Affimer Agent of the present disclosure with a small molecule that acts as an inhibitor against tumor-associated antigens including, but not limited to, EGFR, HER2 (ErbB2), and/or VEGF. In some embodiments, an Affimer Agent of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In some embodiments of the methods described herein, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway. In some embodiments, the additional therapeutic agent is an inhibitor of the RSPO/LGR pathway.

In some embodiments of the methods described herein, an additional therapeutic agent comprises a biological molecule, such as an antibody. For example, treatment can involve the combined administration of an Affimer Agent of the present disclosure with antibodies against tumor-associated antigens including, but not limited to, antibodies that bind EGFR, HER2/ErbB2, and/or VEGF. In some embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In some embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits .beta.-catenin signaling. In some embodiments, the additional therapeutic agent is an antibody that is an angiogenesis inhibitor (e.g., an anti-VEGF or VEGF receptor antibody). In some embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In some embodiments of the methods described herein, the additional therapeutic agent is an antibody that modulates the immune response. In some embodiments, the additional therapeutic agent is an anti-PD-1 antibody, an anti-LAG-3 antibody, an anti-CTLA-4 antibody, an anti-TIM-3 antibody, or an anti-TIGIT antibody.

Furthermore, treatment with an Affimer Agent described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician. In some embodiments, the additional therapeutic agent is an immune response stimulating agent.

In some embodiments of the methods described herein, the Affimer Agent can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, IL-1, IL-2, IL-3, IL-4, IL-5, IL-6, IL-7, IL-12, IL-15, and IL-18.

In some embodiments of the methods described herein, the additional therapeutic agent is an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), 4-1BB ligand, anti-CD3 antibody, anti-CTLA-4 antibody, anti-TIGIT antibody, anti-PD-1 antibody, anti-LAG-3 antibody, and anti-TIM-3 antibody.

In some embodiments of the methods described herein, an immune response stimulating agent is selected from the group consisting of: a modulator of PD-1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

In some embodiments of the methods described herein, an immune response stimulating agent is selected from the group consisting of: a PD-1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and/or an IDO1 antagonist.

In some embodiments of the methods described herein, the PD-1 antagonist is an antibody that specifically binds PD-1. In some embodiments, the antibody that binds PD-1 is KEYTRUDA (MK-3475), pidilizumab (CT-011), nivolumab (OPDIVO, BMS-936558, MDX-1106), MEDI0680 (AMP-514), REGN2810, BGB-A317, PDR-001, or STI-A1110. In some embodiments, the antibody that binds PD-1 is described in PCT Publication WO 2014/179664, for example, an antibody identified as APE2058, APE1922, APE1923, APE1924, APE 1950, or APE1963, or an antibody containing the CDR regions of any of these antibodies. In other embodiments, the PD-1 antagonist is a fusion protein that includes PD-L2, for example, AMP-224. In other embodiments, the PD-1 antagonist is a peptide inhibitor, for example, AUNP-12.

In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the CTLA-4 antagonist a CTLA-4 fusion protein, for example, KAHR-102.

In some embodiments, the LAG3 antagonist is an antibody that specifically binds LAG3. In some embodiments, the antibody that binds LAG3 is IMP701, IMP731, BMS-986016, LAG525, and GSK2831781. In some embodiments, the LAG3 antagonist includes a soluble LAG3 receptor, for example, IMP321.

In some embodiments, the KIR antagonist is an antibody that specifically binds KIR. In some embodiments, the antibody that binds KIR is lirilumab.

In some embodiments, an immune response stimulating agent is selected from the group consisting of: a CD28 agonist, a 4-1BB agonist, an OX40 agonist, a CD27 agonist, a CD80 agonist, a CD86 agonist, a CD40 agonist, and a GITR agonist. p In some embodiments, the OX40 agonist includes OX40 ligand, or an OX40-binding portion thereof. For example, the OX40 agonist may be MEDI6383. In some embodiments, the OX40 agonist is an antibody that specifically binds OX40. In some embodiments, the antibody that binds OX40 is MEDI6469, MEDI0562, or MOXR0916 (RG7888). In some embodiments, the OX40 agonist is a vector (e.g., an expression vector or virus, such as an adenovirus) capable of expressing OX40 ligand. In some embodiments the OX40-expressing vector is Delta-24-RGDOX or DNX2401.

In some embodiments, the 4-1BB (CD137) agonist is a binding molecule, such as an anticalin. In some embodiments, the anticalin is PRS-343. In some embodiments, the 4-1BB agonist is an antibody that specifically binds 4-1BB. In some embodiments, antibody that binds 4-1BB is PF-2566 (PF-05082566) or urelumab (BMS-663513).

In some embodiments, the CD27 agonist is an antibody that specifically binds CD27. In some embodiments, the antibody that binds CD27 is varlilumab (CDX-1127).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518, MK-4166, or INBRX-110.

In some embodiments, immune response stimulating agents include, but are not limited to, cytokines such as chemokines, interferons, interleukins, lymphokines, and members of the tumor necrosis factor (TNF) family. In some embodiments, immune response stimulating agents include immunostimulatory oligonucleotides, such as CpG dinucleotides.

In some embodiments, an immune response stimulating agent includes, but is not limited to, anti-PD-1 antibodies, anti-PD-L2 antibodies, anti-CTLA-4 antibodies, anti-CD28 antibodies, anti-CD80 antibodies, anti-CD86 antibodies, anti-4-1BB antibodies, anti-OX40 antibodies, anti-KIR antibodies, anti-Tim-3 antibodies, anti-LAG3 antibodies, anti-CD27 antibodies, anti-CD40 antibodies, anti-GITR antibodies, anti-TIGIT antibodies, anti-CD20 antibodies, anti-CD96 antibodies, or anti-IDO1 antibodies.

In some embodiments, the Affimer Agents disclosed herein may be used alone, or in association with radiation therapy.

In some embodiments, the Affimer Agents disclosed herein may be used alone, or in association with targeted therapies. Examples of targeted therapies include: hormone therapies, signal transduction inhibitors (e.g., EGFR inhibitors, such as cetuximab (Erbitux) and erlotinib (Tarceva)); HER2 inhibitors (e.g., trastuzumab (Herceptin) and pertuzumab (Perjeta)); BCR-ABL inhibitors (such as imatinib (Gleevec) and dasatinib (Sprycel)); ALK inhibitors (such as crizotinib (Xalkori) and ceritinib (Zykadia)); BRAF inhibitors (such as vemurafenib (Zelboraf) and dabrafenib (Tafinlar)), gene expression modulators, apoptosis inducers (e.g., bortezomib (Velcade) and carfilzomib (Kyprolis)), angiogenesis inhibitors (e.g., bevacizumab (Avastin) and ramucirumab (Cyramza), monoclonal antibodies attached to toxins (e.g., brentuximab vedotin (Adcetris) and ado-trastuzumab emtansine (Kadcyla)).

In some embodiments, the Affimer Agents of the disclosure may be used in combination with an anti-cancer therapeutic agent or immunomodulatory drug such as an immunomodulatory receptor inhibitor, e.g., an antibody or antigen-binding fragment thereof that specifically binds to the receptor.

In some embodiments of the disclosure, an anti-PD-1 antibody or antigen-binding fragment thereof of the disclosure is administered in association with a Tim-3 pathway antagonist, for example, as part of a pharmaceutical composition.

In some embodiments of the disclosure, an anti-PD-1 antibody or antigen-binding fragment thereof of the disclosure is administered in association with a Vista pathway antagonist, for example, as part of a pharmaceutical composition.

In some embodiments of the disclosure, an anti-PD-1 antibody or antigen-binding fragment thereof of the disclosure is administered in association with a BTLA pathway antagonist, for example, as part of a pharmaceutical composition.

In some embodiments of the disclosure, an anti-PD-1 antibody or antigen-binding fragment thereof of the disclosure is administered in association with a LAG-3 pathway antagonist, for example, as part of a pharmaceutical composition.

In some embodiments of the disclosure, an anti-PD-1 antibody or antigen-binding fragment thereof of the disclosure is administered in association with a TIGIT pathway antagonist, for example, as part of a pharmaceutical composition.

In some embodiments of the disclosure, an anti-PD-1 antibody or antigen-binding fragment thereof of the disclosure is administered in association with an anti-PDL1 antibody

In some embodiments of the disclosure, an anti-PD-1 antibody or antigen-binding fragment thereof of the disclosure is administered in association with BMS-936559, MSB0010718C or MPDL3280A), for example, as part of a pharmaceutical composition.

In some embodiments of the disclosure, an anti-PD-1 antibody or antigen-binding fragment thereof of the disclosure is administered in association with an anti-CTLA4 antibody, for example, as part of a pharmaceutical composition.

In some embodiments of the disclosure, an anti-PD-1 antibody or antigen-binding fragment thereof of the disclosure is administered in association with an anti-CS1 antibody, for example, as part of a pharmaceutical composition.

In some embodiments of the disclosure, an anti-PD-1 antibody or antigen-binding fragment thereof of the disclosure is administered in association with an anti-KIR2DL1/2/3 antibody, for example, as part of a pharmaceutical composition.

In some embodiments of the disclosure, an anti-PD-1 antibody or antigen-binding fragment thereof of the disclosure is administered in association with an anti-CD137 antibody, for example, as part of a pharmaceutical composition.

In some embodiments of the disclosure, an anti-PD-1 antibody or antigen-binding fragment thereof of the disclosure is administered in association with an anti-GITR antibody, for example, as part of a pharmaceutical composition.

In some embodiments of the disclosure, an anti-PD-1 antibody or antigen-binding fragment thereof of the disclosure is administered in association with an anti-PD-L2 antibody, for example, as part of a pharmaceutical composition.

In some embodiments of the disclosure, an anti-PD-1 antibody or antigen-binding fragment thereof of the disclosure is administered in association with an anti-ILT1 antibody, for example, as part of a pharmaceutical composition.

In some embodiments of the disclosure, an anti-PD-1 antibody or antigen-binding fragment thereof of the disclosure is administered in association with an anti-ILT2 antibody, for example, as part of a pharmaceutical composition.

In some embodiments of the disclosure, an anti-PD-1 antibody or antigen-binding fragment thereof of the disclosure is administered in association with an anti-ILT3 antibody, for example, as part of a pharmaceutical composition.

In some embodiments of the disclosure, an anti-PD-1 antibody or antigen-binding fragment thereof of the disclosure is administered in association with an anti-ILT4 antibody, for example, as part of a pharmaceutical composition.

In some embodiments of the disclosure, an anti-PD-1 antibody or antigen-binding fragment thereof of the disclosure is administered in association with an anti-ILT5 antibody, for example, as part of a pharmaceutical composition.

In some embodiments of the disclosure, an anti-PD-1 antibody or antigen-binding fragment thereof of the disclosure is administered in association with an anti-ILT6 antibody, for example, as part of a pharmaceutical composition.

In some embodiments of the disclosure, an anti-PD-1 antibody or antigen-binding fragment thereof of the disclosure is administered in association with an anti-ILT7 antibody, for example, as part of a pharmaceutical composition.

In some embodiments of the disclosure, an anti-PD-1 antibody or antigen-binding fragment thereof of the disclosure is administered in association with an anti-ILT8 antibody, for example, as part of a pharmaceutical composition.

In some embodiments of the disclosure, an anti-PD-1 antibody or antigen-binding fragment thereof of the disclosure is administered in association with an anti-CD40 antibody, for example, as part of a pharmaceutical composition.

In some embodiments of the disclosure, an anti-PD-1 antibody or antigen-binding fragment thereof of the disclosure is administered in association with an anti-OX40 antibody, for example, as part of a pharmaceutical composition.

In some embodiments of the disclosure, an anti-PD-1 antibody or antigen-binding fragment thereof of the disclosure is administered in association with an anti-KIR2DL1 antibody, for example, as part of a pharmaceutical composition.

In some embodiments of the disclosure, an anti-PD-1 antibody or antigen-binding fragment thereof of the disclosure is administered in association with an anti-KIR2DL2/3 antibody, for example, as part of a pharmaceutical composition.

In some embodiments of the disclosure, an anti-PD-1 antibody or antigen-binding fragment thereof of the disclosure is administered in association with an anti-KIR2DL4 antibody, for example, as part of a pharmaceutical composition.

In some embodiments of the disclosure, an anti-PD-1 antibody or antigen-binding fragment thereof of the disclosure is administered in association with an anti-KIR2DL5A antibody, for example, as part of a pharmaceutical composition.

In some embodiments of the disclosure, an anti-PD-1 antibody or antigen-binding fragment thereof of the disclosure is administered in association with an anti-KIR2DL5B antibody, for example, as part of a pharmaceutical composition.

In some embodiments of the disclosure, an anti-PD-1 antibody or antigen-binding fragment thereof of the disclosure is administered in association with an anti-KIR3DL1 antibody, for example, as part of a pharmaceutical composition.

In some embodiments of the disclosure, an anti-PD-1 antibody or antigen-binding fragment thereof of the disclosure is administered in association with an anti-KIR3DL2 antibody, for example, as part of a pharmaceutical composition.

In some embodiments of the disclosure, an anti-PD-1 antibody or antigen-binding fragment thereof of the disclosure is administered in association with an anti-KIR3DL3 antibody, for example, as part of a pharmaceutical composition.

In some embodiments of the disclosure, an anti-PD-1 antibody or antigen-binding fragment thereof of the disclosure is administered in association with an anti-NKG2A antibody, for example, as part of a pharmaceutical composition.

In some embodiments of the disclosure, an anti-PD-1 antibody or antigen-binding fragment thereof of the disclosure is administered in association with an anti-NKG2C antibody, for example, as part of a pharmaceutical composition.

In some embodiments of the disclosure, an anti-PD-1 antibody or antigen-binding fragment thereof of the disclosure is administered in association with an anti-ICOS antibody, for example, as part of a pharmaceutical composition.

In some embodiments of the disclosure, an anti-PD-1 antibody or antigen-binding fragment thereof of the disclosure is administered in association with an anti-SIRP.alpha. antibody, for example, as part of a pharmaceutical composition.

In some embodiments of the disclosure, an anti-PD-1 antibody or antigen-binding fragment thereof of the disclosure is administered in association with an anti-CD47 antibody, for example, as part of a pharmaceutical composition.

In some embodiments of the disclosure, an anti-PD-1 antibody or antigen-binding fragment thereof of the disclosure is administered in association with an anti-4-1 BB antibody, for example, as part of a pharmaceutical composition.

In some embodiments of the disclosure, an anti-PD-1 antibody or antigen-binding fragment thereof of the disclosure is administered in association with an anti-IL-10 antibody, for example, as part of a pharmaceutical composition.

In some embodiments of the disclosure, an anti-PD-1 antibody or antigen-binding fragment thereof of the disclosure is administered in association with an anti-TSLP antibody, for example, as part of a pharmaceutical composition.

In some embodiments of the disclosure, an anti-PD-1 antibody or antigen-binding fragment thereof of the disclosure is administered in association with IL-10 or PEGylated IL-10, for example, as part of a pharmaceutical composition.

In some embodiments of the disclosure, an anti-PD-1 antibody or antigen-binding fragment thereof of the disclosure is administered in association with an anti-APRIL antibody, for example, as part of a pharmaceutical composition.

In some embodiments of the disclosure, an anti-PD-1 antibody or antigen-binding fragment thereof of the disclosure is administered in association with an anti-CD27 antibody, for example, as part of a pharmaceutical composition.

In some embodiments of the disclosure, an Affimer Agent of the disclosure is administered in with a STING agonist, for example, as part of a pharmaceutical composition. The cyclic-di-nucleotides (CDNs) cyclic-di-AMP (produced by Listeria monocytogenes and other bacteria) and its analogs cyclic-di-GMP and cyclic-GMP-AMP are recognized by the host cell as a pathogen associated molecular pattern (PAMP), which bind to the pathogen recognition receptor (PRR) known as Stimulator of INterferon Genes (STING). STING is an adaptor protein in the cytoplasm of host mammalian cells which activates the TANK binding kinase (TBK1)-IRF3 and the NF-.kappa.B signaling axis, resulting in the induction of IFN-.beta. and other gene products that strongly activate innate immunity. It is now recognized that STING is a component of the host cytosolic surveillance pathway, that senses infection with intracellular pathogens and in response induces the production of IFN-α, leading to the development of an adaptive protective pathogen-specific immune response consisting of both antigen-specific CD4+ and CD8+ T cells as well as pathogen-specific antibodies. U.S. Pat. Nos. 7,709,458 and 7,592,326; PCT Publication NOS: WO2007/054279, WO2014/093936, WO2014/179335, WO2014/189805, WO2015/185565, WO2016/096174, WO2016/145102, WO2017/027645, WO2017/027646, and WO2017/075477; and Yan et al., Bioorg. Med. Chem Lett. 18:5631-4, 2008.

In some embodiments of the disclosure, an Affimer Agent of the disclosure is administered in association with an Akt inhibitor. Exemplary AKT inhibitors include GDC0068 (also known as GDC-0068, ipatasertib and RG7440), MK-2206, perifosine (also known as KRX-0401), GSK690693, AT7867, triciribine, CCT128930, A-674563, PHT-427, Akti-1/2, afuresertib (also known as GSK2110183), AT13148, GSK2141795, BAY1125976, uprosertib (aka GSK2141795), Akt Inhibitor VIII (1,3-dihydro-1-[1-[[4-(6-phenyl-1H-imidazo[4,5-g]quinoxalin-7-yl)phenyl]m-ethyl]-4-piperidinyl]-2H-benzimidazol-2-one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f][-1,6]naphthyridin-3(2H)-one), uprosertib (N—((S)-1-amino-3-(3,4-difluorophenyl)propan-2-yl)-5-chloro-4-(4-chloro-1-methyl-1H-pyrazol-5-yl)furan-2-carboxamide), ipatasertib ((S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one)-, AZD 5363 (4-Piperidinecarboxamide, 4-amino-N-[(1S)-1-(4-chlorophenyl)-3-hydroxypropyl]-1-(7H-pyrrolo[2,3-d]pyrimidin-4-yl)), perifosine, GSK690693, GDC-0068, tricirbine, CCT128930, A-674563, PF-04691502, AT7867, miltefosine, PHT-427, honokiol, triciribine phosphate, and KP372-1A (10H-indeno[2,1-e]tetrazolo[1,5-b][1,2,4]triazin-10-one), Akt Inhibitor IX (CAS 98510-80-6). Additional Akt inhibitors include: ATP-competitive inhibitors, e.g. isoquinoline-5-sulfonamides (e.g., H-8, H-89, NL-71-101), azepane derivatives (e.g., (−)-balanol derivatives), aminofurazans (e.g., GSK690693), heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, A-674563, A-443654), phenylpyrazole derivatives (e.g., AT7867, AT13148), thiophenecarboxamide derivatives (e.g., Afuresertib (GSK2110183), 2-pyrimidyl-5-amidothiophene derivative (DC120), uprosertib (GSK2141795); Allosteric inhibitors, e.g., 2,3-diphenylquinoxaline analogues (e.g., 2,3-diphenylquinoxaline derivatives, triazolo[3,4-f][1,6]naphthyridin-3(2H)-one derivative (MK-2206)), alkylphospholipids (e.g., Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine), indole-3-carbinol analogues (e.g., indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (e.g., PH-316, PHT-427), thiourea derivatives (e.g., PIT-1, PIT-2, DM-PIT-1, N-[(1-methyl-1H-pyrazol-4-yl)carbonyl]-N′-(3-bromophenyl)-thiourea), purine derivatives (e.g., Triciribine (TCN, NSC 154020), triciribine mono-phosphate active analogue (TCN-P),4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3.alpha.- and 3.beta.-acetoxy-tirucallic acids, acetoxy-tirucallic acid; and irreversible inhibitors, e.g., natural products, antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives.

In some embodiments of the disclosure, an Affimer Agent of the disclosure is administered in association with a MEK inhibitor. Exemplary MEK inhibitors include AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib and PD318088.

In some embodiments of the disclosure, an Affimer Agent of the disclosure is administered in association with both an anthracycline such as doxorubicin and cyclophosphamide, including pegylated liposomal doxorubicin.

In some embodiments of the disclosure, an Affimer Agent of the disclosure is administered in association with both an anti-CD20 antibody and an anti-CD3 antibody, or a bispecific CD20/CD3 binder (including a CD20/CD3 BiTE).

In some embodiments of the disclosure, an Affimer Agent of the disclosure is administered in association with a CD73 inhibitor, a CD39 inhibitor or both. These inhibitors can be CD73 binders or CD39 binders (such as antibody, antibody fragments or antibody mimetics) that inhibit the ectonucleosidase activity. The inhibitor may be a small molecule inhibitor of the ectonucleosidase activity, such as 6-N,N-Diethyl-β-γ-dibromomethylene-D-adenosine-5′-triphosphate trisodium salt hydrate, PSB069, PSB 06126,

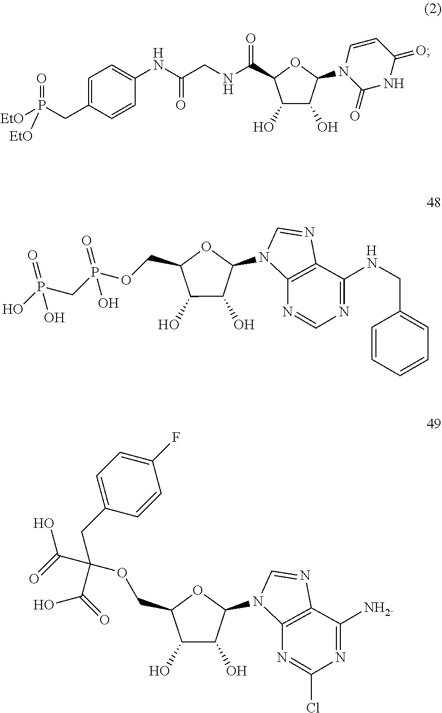

In some embodiments of the disclosure, an Affimer Agent of the disclosure is administered in association with an inhibitor poly ADP ribose polymerase (PARP). Exemplary PARP inhibitors include Olaparib, Niraparib, Rucaparib, Talazoparib, Veliparib, CEP9722, MK4827 and BGB-290.

In some embodiments of the disclosure, an Affimer Agent of the disclosure is administered in association with an oncolytic virus. An exemplary oncolytic virus is Talimogene Laherparepvec.

In some embodiments of the disclosure, an Affimer Agent of the disclosure is administered in association with an CSF-1 antagonist, such as an agent that binds to CSF-1 or CSF1R and inhibits the interaction of CSF-1 with CSF1R on macrophage. Exemplary CSF-1 antagonists include Emactuzumab and FPA008.

In some embodiments of the disclosure, an Affimer Agent of the disclosure is administered in association with an anti-CD38 antibody. Exemplary anti-CD39 antibodies include Daratumumab and Isatuximab.

In some embodiments of the disclosure, an Affimer Agent of the disclosure is administered in association with an anti-CD40 antibody. Exemplary anti-CD40 antibodies include Selicrelumab and Dacetuzumab.

In some embodiments of the disclosure, an Affimer Agent of the disclosure is administered in association with an inhibitor of anaplatic lymphoma kinase (ALK). Exemplary ALK inhibitors include Alectinib, Crizotinib and Ceritinib.

In some embodiments of the disclosure, an Affimer Agent of the disclosure is administered in association with multikinase inhibitor that inhibits one or more selected from the group consisting of the family members of VEGFR, PDGFR and FGFR, or an anti-angiogenesis inhibitor. Exemplary inhibitors include Axitinib, Cediranib, Linifanib, Motesanib, Nintedanib, Pazopanib, Ponatinib, Regorafenib, Sorafenib, Sunitinib, Tivozanib, Vatalanib, LY2874455, or SU5402.

In some embodiments of the disclosure, an Affimer Agent of the disclosure is administered in conjunction with one or more vaccines intended to stimulate an immune response to one or more predetermined antigens. The antigen(s) may be administered directly to the individual, or may be expressed within the individual from, for example, a tumor cell vaccine (e.g., GVAX) which may be autologous or allogenic, a dendritic cell vaccine, a DNA vaccine, an RNA vaccine, a viral-based vaccine, a bacterial or yeast vaccine (e.g., a Listeria monocytogenes or Saccharomyces cerevisiae), etc. See, e.g., Guo et al., Adv. Cancer Res. 2013; 119: 421-475; Obeid et al., Semin Oncol. 2015 August; 42(4): 549-561. The target antigen may also be a fragment or fusion polypeptide comprising an immunologically active portion of the antigens listed in the table.

In some embodiments of the disclosure, an Affimer Agent of the disclosure is administered in association with one or more antiemetics including, but not limited to: casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl by Pfizer; New York, N.Y.), hydroxyzine (sold as Atarax by Pfizer; New York, N.Y.), metoclopramide (sold as Reglan by AH Robins Co; Richmond, Va.), lorazepam (sold as Ativan by Wyeth; Madison, N.J.), alprazolam (sold as Xanax by Pfizer; New York, N.Y.), haloperidol (sold as Haldol by Ortho-McNeil; Raritan, N.J.), droperidol (Inapsine), dronabinol (sold as Marinol by Solvay Pharmaceuticals, Inc.; Marietta, Ga.), dexamethasone (sold as Decadron by Merck and Co.; Rahway, N.J.), methylprednisolone (sold as Medrol by Pfizer; New York, N.Y.), prochlorperazine (sold as Compazine by Glaxosmithkline; Research Triangle Park, N.C.), granisetron (sold as Kytril by Hoffmann-La Roche Inc.; Nutley, N.J.), ondansetron (sold as Zofran by Glaxosmithkline; Research Triangle Park, N.C.), dolasetron (sold as Anzemet by Sanofi-Aventis; New York, N.Y.), tropisetron (sold as Navoban by Novartis; East Hanover, N.J.).

Other side effects of cancer treatment include red and white blood cell deficiency. Accordingly, in some embodiments of the disclosure, an Affimer Agent is administered in association with an agent which treats or prevents such a deficiency, such as, e.g., filgrastim, PEG-filgrastim, erythropoietin, epoetin alfa or darbepoetin alfa.

In some embodiments of the disclosure, an Affimer Agent of the disclosure is administered in association with anti-cancer radiation therapy. For example, in some embodiments of the disclosure, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In some embodiments of the disclosure, the radiation therapy is proton beam therapy: a type of conformal therapy that bombards the diseased tissue with protons instead of X-rays. In some embodiments of the disclosure, the radiation therapy is conformal external beam radiation therapy: a procedure that uses advanced technology to tailor the radiation therapy to an individual's body structures. In some embodiments of the disclosure, the radiation therapy is brachytherapy: the temporary placement of radioactive materials within the body, usually employed to give an extra dose—or boost—of radiation to an area.

In some embodiments of the methods described herein, the treatment involves the administration of an Affimer Agent of the present disclosure in combination with anti-viral therapy. Treatment with an Affimer Agent can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of an Affimer Agent described herein and at least one additional therapeutic agent may be administered in any order or concurrently. In some embodiments, the Affimer Agent will be administered to patients that have previously undergone treatment with a second therapeutic agent. In certain other embodiments, the Affimer Agent and a second therapeutic agent will be administered substantially simultaneously or concurrently. For example, a subject may be given an Affimer Agent while undergoing a course of treatment with a second therapeutic agent (e.g., chemotherapy). In some embodiments, an Affimer Agent will be administered within 1 year of the treatment with a second therapeutic agent. In certain alternative embodiments, an Affimer Agent will be administered within 10, 8, 6, 4, or 2 months of any treatment with a second therapeutic agent. In certain other embodiments, an Affimer Agent will be administered within 4, 3, 2, or 1 weeks of any treatment with a second therapeutic agent. In some embodiments, an Affimer Agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of an Affimer Agent of the present disclosure depends on the type of disease to be treated, the severity and course of the disease, the responsiveness of the disease, whether the Affimer Agent is administered for therapeutic or preventative purposes, previous therapy, the patient's clinical history, and so on, all at the discretion of the treating physician. The Affimer Agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In some embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In some embodiments, the dosage of the Affimer Agent is from about 0.1 mg to about 20 mg/kg of body weight. In some embodiments, the dosage of the Affimer Agent is about 0.1 mg/kg of body weight. In some embodiments, the dosage of the Affimer Agent is about 0.25 mg/kg of body weight. In some embodiments, the dosage of the Affimer Agent is about 0.5 mg/kg of body weight. In some embodiments, the dosage of the Affimer Agent is about 1 mg/kg of body weight. In some embodiments, the dosage of the Affimer Agent is about 1.5 mg/kg of body weight. In some embodiments, the dosage of the Affimer Agent is about 2 mg/kg of body weight. In some embodiments, the dosage of the Affimer Agent is about 2.5 mg/kg of body weight. In some embodiments, the dosage of the Affimer Agent is about 5 mg/kg of body weight. In some embodiments, the dosage of the Affimer Agent is about 7.5 mg/kg of body weight. In some embodiments, the dosage of the Affimer Agent is about 10 mg/kg of body weight. In some embodiments, the dosage of the Affimer Agent is about 12.5 mg/kg of body weight. In some embodiments, the dosage of the Affimer Agent is about 15 mg/kg of body weight. In some embodiments, the dosage can be given once or more daily, weekly, monthly, or yearly. In some embodiments, the Affimer Agent is given once every week, once every two weeks, once every three weeks, or once every four weeks.

In some embodiments, an Affimer Agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the Affimer Agent is administered for 3, 4, 5, 6, 7, 8, or more cycles. For example, the Affimer Agent is administered every 2 weeks for 6 cycles, the Affimer Agent is administered every 3 weeks for 6 cycles, the Affimer Agent is administered every 2 weeks for 4 cycles, the Affimer Agent is administered every 3 weeks for 4 cycles, etc. Dosing schedules can be decided upon and subsequently modified by those skilled in the art.

Thus, the present disclosure provides methods of administering to a subject the polypeptides or agents described herein comprising using an intermittent dosing strategy for administering one or more agents, which may reduce side effects and/or toxicities associated with administration of an Affimer Agent, chemotherapeutic agent, etc. In some embodiments, a method for treating cancer in a human subject comprises administering to the subject a therapeutically effective dose of an Affimer Agent in combination with a therapeutically effective dose of a chemotherapeutic agent, wherein one or both of the agents are administered according to an intermittent dosing strategy. In some embodiments, the intermittent dosing strategy comprises administering an initial dose of an Affimer Agent to the subject, and administering subsequent doses of the Affimer Agent about once every 2 weeks. In some embodiments, the intermittent dosing strategy comprises administering an initial dose of an Affimer Agent to the subject, and administering subsequent doses of the Affimer Agent about once every 3 weeks. In some embodiments, the intermittent dosing strategy comprises administering an initial dose of an Affimer Agent to the subject, and administering subsequent doses of the Affimer Agent about once every 4 weeks. In some embodiments, the Affimer Agent is administered using an intermittent dosing strategy and the chemotherapeutic agent is administered weekly.

In some embodiments, the disclosure also provides methods for treating subjects using an Affimer Agent of the disclosure, wherein the subject suffers from a viral infection. In some embodiments, the viral infection is infection with a virus selected from the group consisting of human immunodeficiency virus (HIV), hepatitis virus (A, B, or C), herpes virus (e.g., VZV, HSV-I, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus or arboviral encephalitis virus.

In some embodiments, the disclosure provides methods for treating subjects using an Affimer Agent thereof of the disclosure, wherein the subject suffers from a bacterial infection. In some embodiments, the bacterial infection is infection with a bacterium selected from the group consisting of Chlamydia, rickettsial bacteria, mycobacteria, staphylococci, streptococci, pneumonococci, meningococci and gonococci, klebsiella, proteus, serratia, pseudomonas, Legionella, Corynebacterium diphtheriae, Salmonella, bacilli, Vibrio cholerae, Clostridium tetan, Clostridium botulinum, Bacillus anthricis, Yersinia pestis, Mycobacterium leprae, Mycobacterium lepromatosis, and Borriella.

In some embodiments, the disclosure provides methods for treating subjects using an Affimer Agent of the disclosure, wherein the subject suffers from a fungal infection. In some embodiments, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In some embodiments, the disclosure provides methods for treating subjects using an Affimer Agent of the disclosure, wherein the subject suffers from a parasitic infection. In some embodiments, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii and Nippostrongylus brasiliensis.

### EXAMPLES

### Example 1: Selection of PD-L1 Binding Affimers from Phage Display Library

A peptide of the present disclosure, for example, a PD-L1 binding component, may be identified by selection from a library of affimers with two random loops, for example, generally but not exclusively of the same length of 9 amino acids.

As indicated above, the PD-L1 binding peptides of the disclosure were identified by selection from a phage display library comprising random loop sequences nine amino acids in length displayed in a constant affimer framework backbone based upon the sequence for Stefin A. Such selection procedures are generally known. According to such procedures, suspensions of phage are incubated with target antigen (either biotinylated antigen captured on streptavidin beads or unbiotinylated antigen captured on a plate). Unbound phage are then washed away and, subsequently, bound phage are eluted either by incubating the antigen with low pH solution, followed by high pH solution. E. coli are then infected with released, pH neutralized phage solution and a preparation of first round phage is obtained. The cycle is performed repeatedly, for example, two or three times and, in order to enrich for targeting phage, the stringency conditions may be increased in the later rounds of selection, for example by increasing the number of wash steps, reducing the antigen concentration, and preselecting with blocked streptavidin beads or wells coated with blocking reagent.

Following selection by successive rounds of phage amplification, PD-L1 binding clones were identified by a soluble affimer ELISA. Briefly, affimer was overexpressed from the phagemid vector, the bacterial cell lysed and the lysate used in an ELISA, detecting affimer binding to PD-L1 immobilized on a plate with a conjugated antibody to the His6 tag on the affimer. Clones showing specific binding were sequenced to identify loop sequences.

To illustrate, selection of PD-L1 binding phage from the affimer library was carried out as described below using approximately 1×10¹² phage are added from a library of size approximately 6×10¹⁰ diversity.

Biotinylated antigen captured on M280 streptavidin or neutravidin beads (Thermo Scientific). Antigen was supplied in an Fc-cleaved format by R & D and was biotinylated in-house using the EZ Link Sulfo-NHS-LC Biotin kit (Pierce).

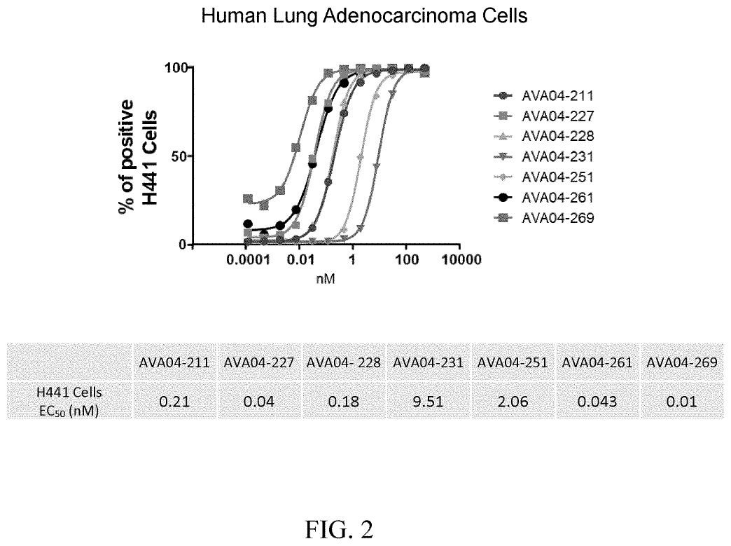

### Example 2: Binding Affinity of Anti-PD-L1 Affimer to Human Cancer Cell Line Expressing PD-L1 (FIG. 2)

The binding affinity of AVA04 Affimer polypeptides was determined using Flow cytometry on Human lung adenocarcinoma cell line. H441 cells expressing PD-L1 grown in RPMI-2640 (Sigma) containing 10% of FBS (Gibco) with Penicillin (100 U/ml, Hyclone) and Streptomycin (100 μg/ml, Hyclone) where detached from the tissue culture washed using DPBS. Cells were collected by centrifugation at 300 rpm for 5 min. The cells were resuspended in PBS and 50000 cells per wells were dispatched in a round bottom 96 well plate. Cells were washed with PBS. Affimer and controls were diluted in staining buffer (R&D) in duplicate and added on cells for staining for approximately 60 min at 4±1° c. Cells were washed and the secondary anti-Cystatin A (R&D) was diluted 1:15 in staining buffer (R&D) and added on cells for staining for approximately 40 min at 4±1° c. Cells were washed and the detection antibody A488 anti-Goat (Biolegend) was diluted 1:100 in staining buffer (R&D) and added on cells for staining for approximately 30 min at 4±1° c. Finally, cells were washed, live and dead cells were stained using L/D stain Zombie Aqua (Biolegend) diluted in staining buffer for 10 min at 4±1° c. Cells were washed and fixation buffer (R&D) was added to each well for 10 min at 4±1° C. then PBS with EDTA (Lonza) was added prior reading the plate on the flow cytometer (Guava 12 HT, Millipore). Dead cells were excluded and the fluorescent Green channel (488 nm/525/30) was acquired. Results were analyzed using Incyte and data were plotted using graphpad.

Examples of AVA04 Affimer polypeptides are provided in Tables 4A, 4B and 5.

TABLE 4A
Loop Sequences for Example PD-L1 Affimer
Polypeptides

| AVA04 | | SEQ ID | | SEQ ID |
|---|---|---|---|---|
| clone | loop 2 | NO: | loop 4 | NO: |
| 269 | KEYGPEEWW | 8 | GDYEQVLIH | 44 |
| 261 | HAYGPRDWD | 9 | PADHVLEEA | 45 |
| 211 | KDWGPSNWW | 21 | VDDKTLSKD | 57 |
| 227 | KDHGPIAWW | 10 | EDTNTDGAL | 46 |
| 228 | KPYGPRDWD | 7 | EPQLDTSPI | 43 |
| 251 | REGRQDWVL | 39 | WVPFPHQQL | 75 |
| 236 | KEHGPDSWW | 15 | QEKNQWVEE | 51 |
| 231 | NTWFPESFW | 18 | DDNQERQEH | 54 |
| 179 | YNHFPEYMW | 41 | PPRFPHEPF | 77 |
| 141 | AAHFPEHFW | 38 | QPADMSAEF | 74 |

TABLE 4B.
Full Amino Acis Sequences for Example PD-L1 Affimer Polypeptides

| | | SEQ ID |
|---|---|---|
| AVA04 clone | Full amino acid sequence | NO: |
| 269 | MIPRGLSEAKPATPEIQEIVDKVKPQLEEKTNETYGKLEAV | 119 |
| | QYKTQVLAKEYGPEEWWSTNYYIKVRAGDNKYMHLKVF | |
| | NGPGDYEQVLIHADRVLTGYQVDKNKDDELTGFAAAGH | |
| | HHHHH | |
| 261 | MIPRGLSEAKPATPEIQEIVDKVKPQLEEKTNETYGKLEAV | 120 |
| | QYKTQVLAHAYGPRDWDSTNYYIKVRAGDNKYMHLKVF | |
| | NGPPADHVLEEAADRVLTGYQVDKNKDDELTGFAAAGH | |
| | HHHHH | |
| 211 | MIPRGLSEAKPATPEIQEIVDKVKPQLEEKTNETYGKLEAV | 121 |
| | QYKTQVLAKDWGPSNWWSTNYYIKVRAGDNKYMHLKV | |
| | FNGPVDDKTLSKDADRVLTGYQVDKNKDDELTGFAAAG | |
| | HHHHHH | |
| 227 | MIPRGLSEAKPATPEIQEIVDKVKPQLEEKTNETYGKLEAV | 122 |
| | QYKTQVLAKDHGPIAWWSTNYYIKVRAGDNKYMHLKVF | |
| | NGPEDTNTDGALADRVLTGYQVDKNKDDELTGFAAAGH | |
| | HHHHH | |
| 228 | MIPRGLSEAKPATPEIQEIVDKVKPQLEEKTNETYGKLEAV | 123 |
| | QYKTQVLAKPYGPRDWDSTNYYIKVRAGDNKYMHLKVF | |
| | NGPEPQLDTSPIADRVLTGYQVDKNKDDELTGFAAAGHH | |
| | HHHH | |
| 251 | MIPRGLSEAKPATPEIQEIVDKVKPQLEEKTNETYGKLEAV | 124 |
| | QYKTQVLAREGRQDWVLSTNYYIKVRAGDNKYMHLKVF | |
| | NGPWVPFPHQQLADRVLTGYQVDKNKDDELTGFAAAGH | |
| | HHHHH | |
| 236 | MIPRGLSEAKPATPEIQEIVDKVKPQLEEKTNETYGKLEAV | 125 |
| | QYKTQVLAKEHGPDSWWSTNYYIKVRAGDNKYMHLKVF | |
| | NGPQEKNQWVEEADRVLTGYQVDKNKDDELTGFAAAGH | |
| | HHHHH | |
| 231 | MIPRGLSEAKPATPEIQEIVDKVKPQLEEKTNETYGKLEAV | 126 |
| | QYKTQVLANTWFPESFWSTNYYIKVRAGDNKYMHLKVF | |
| | NGPDDNQERQEHADRVLTGYQVDKNKDDELTGFAAAGH | |
| | HHHHH | |
| 179 | MIPRGLSEAKPATPEIQEIVDKVKPQLEEKTNETYGKLEAV | 127 |
| | QYKTQVLAYNHFPEYMWSTNYYIKVRAGDNKYMHLKVF | |
| | NGPPPRFPHEPFADRVLTGYQVDKNKDDELTGFAAAGHH | |
| | HHHH | |
| 141 | MIPRGLSEAKPATPEIQEIVDKVKPQLEEKTNETYGKLEAV | 128 |
| | QYKTQVLAAAHFPEHFWSTNYYIKVRAGDNKYMHLKVF | |
| | NGPQPADMSAEFADRVLTGYQVDKNKDDELTGFAAAGH | |
| | HHHHH | |

TABLE 5
Example PD-L1 Affimer Polypeptide Sequences

| Affimer | | |
|---|---|---|
| Format | Description | Amino Acid Sequence |
| AVA04- | human IgG4 Fc | IPRGLSEAKPATPEIQEIVDKVKPQLEEKTGETYGKL |
| 251 CG | fusion AAA(G4S)₄ | EAVQYKTQVLAREGRQDWVLSTNYYIKVRAGDNK |
| | | YMHLKVFNGPWVPFPHQQLADRVLTGYQVDKNKD |
| | | DELTGFAAAGGGGSGGGGSGGGGSGGGGSESKYGP |
| | | PCPPCPAPEFLGGPSVFLFPPKPKDTLMISRTPEVTCV |
| | | VVDVSQEDPEVQFNWYVDGVEVHNAKTKPREEQF |
| | | NSTYRVVSVLTVLHQDWLNGKEYKCKVSNKGLPSS |
| | | IEKTISKAKGQPREPQVYTLPPSQEEMTKNQVSLTCL |
| | | VKGFYPSDIAVEWESNGQPENNYKTTPPVLDSDGSF |
| | | FLYSRLTVDKSRWQEGNVFSCSVMHEALHNHYTQK |
| | | SLSLSLGK (SEQ ID NO: 129) |
| AVA04- | human IgG1 Fc | IPRGLSEAKPATPEIQEIVDKVKPQLEEKTGETYGKL |
| 251 V.2 | fusion AAA(G4S)₄ | EAVQYKTQVLAREGRQDWVLSTNYYIKVRAGDNK |
| | linker | YMHLKVFNGPWVPFPHQQLADRVLTGYQVDKNKD |
| | | DELTGFAAAGGGGSGGGGSGGGGSGGGGSEPKSCD |
| | | KTHTCPPCPAPELLGGPSVFLFPPKPKDTLMISRTPE |
| | | VTCVVVDVSHEDPEVKFNWYVDGVEVHNAKTKPR |
| | | EEQYNSTYRVVSVLTVLHQDWLNGKEYKCKVSNK |
| | | ALPAPIEKTISKAKGQPREPQVYTLPPSREEMTKNQV |
| | | SLTCLVKGFYPSDIAVEWESNGQPENNYKTTPPVLD |
| | | SDGSFFLYSKLTVDKSRWQQGNVFSCSVMHEALHN |
| | | HYTQKSLSLSPGK (SEQ ID NO: 130) |
| AVA04- | human IgG1 (L153A, | IPRGLSEAKPATPEIQEIVDKVKPQLEEKTGETYGKL |
| 251 CF | L154A mutations) Fc | EAVQYKTQVLAREGRQDWVLSTNYYIKVRAGDNK |
| | fusion AAA(G4S)₄ | YMHLKVFNGPWVPFPHQQLADRVLTGYQVDKNKD |
| | linker | DELTGFAAAGGGGSGGGGSGGGGSGGGGSEPKSCD |
| | | KTHTCPPCPAPEAAGGPSVFLFPPKPKDTLMISRTPE |
| | | VTCVVVDVSHEDPEVKFNWYVDGVEVHNAKTKPR |
| | | EEQYNSTYRVVSVLTVLHQDWLNGKEYKCKVSNK |
| | | ALPAPIEKTISKAKGQPREPQVYTLPPSREEMTKNQV |
| | | SLTCLVKGFYPSDIAVEWESNGQPENNYKTTPPVLD |
| | | SDGSFFLYSKLTVDKSRWQQGNVFSCSVMHEALHN |
| | | HYTQKSLSLSPGK (SEQ ID NO: 131) |
| AVA04- | human IgG1 Fc | IPRGLSEAKPATPEIQEIVDKVKPQLEEKTGETYGKL |
| 251 AZ | fusion AAA linker | EAVQYKTQVLAREGRQDWVLSTNYYIKVRAGDNK |
| | | YMHLKVFNGPWVPFPHQQLADRVLTGYQVDKNKD |
| | | DELTGFAAAEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQV |
| | | YTLPPSREEMTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK (SEQ ID |
| | | NO: 132) |
| AVA04- | Affimer-human IgG1 | IPRGLSEAKPATPEIQEIVDKVKPQLEEKTGETYGKL |
| 251 BS | Fc fusion-Affimer | EAVQYKTQVLAREGRQDWVLSTNYYIKVRAGDNK |
| | | YMHLKVFNGPWVPFPHQQLADRVLTGYQVDKNKD |
| | | DELTGFEPKSCDKTHTCPPCPAPELLGGPSVFLFPPK |
| | | PKDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYTL |
| | | PPSREEMTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGKIPRGLSEAKPA |
| | | TPEIQEIVDKVKPQLEEKTGETYGKLEAVQYKTQVL |
| | | AREGRQDWVLSTNYYIKVRAGDNKYMHLKVFNGP |
| | | WVPFPHQQLADRVLTGYQVDKNKDDELTGF (SEQ |
| | | ID NO: 133) |
| AVA04- | Affimer-AAA(G4S)₄- | IPRGLSEAKPATPEIQEIVDKVKPQLEEKTGETYGKL |
| 251 | Affimer LQA6(G4S)₄- | EAVQYKTQVLAREGRQDWVLSTNYYIKVRAGDNK |
| AG.3 | human IgG1 Fc fusion | YMHLKVFNGPWVPFPHQQLADRVLTGYQVDKNKD |
| | | DELTGFAAAGGGGSGGGGSGGGGSIPRGLSEAKPAT |
| | | PEIQEIVDKVKPQLEEKTGETYGKLEAVQYKTQVLA |
| | | REGRQDWVLSTNYYIKVRAGDNKYMHLKVFNGPW |
| | | VPFPHQQLADRVLTGYQVDKNKDDELTGFLQAAA |
| | | AAAGGGGSGGGGSGGGGSGGGGSEPKSCDKTHTCP |
| | | PCPAPELLGGPSVFLFPPKPKDTLMISRTPEVTCVVV |
| | | DVSHEDPEVKFNWYVDGVEVHNAKTKPREEQYNS |
| | | TYRVVSVLTVLHQDWLNGKEYKCKVSNKALPAPIE |
| | | KTISKAKGQPREPQVYTLPPSREEMTKNQVSLTCLV |
| | | KGFYPSDIAVEWESNGQPENNYKTTPPVLDSDGSFF |
| | | LYSKLTVDKSRWQQGNVFSCSVMHEALHNHYTQK |
| | | SLSLSPGK (SEQ ID NO: 134) |
| AVA04- | Affimer (G4S)₄ human | MIPRGLSEAKPATPEIQEIVDKVKPQLEEKTNETYGK |
| 261 BN | IgG1 hinge His | LEAVQYKTQVLAHAYGPRDWDSTNYYIKVRAGDN |
| | | KYMHLKVFNGPPADHVLEEAADRVLTGYQVDKNK |
| | | DDELTGFGGGGSGGGGSGGGGSGGGGSEPKSCDKT |
| | | HTCPPCPHHHHHH (SEQ ID NO: 135) |

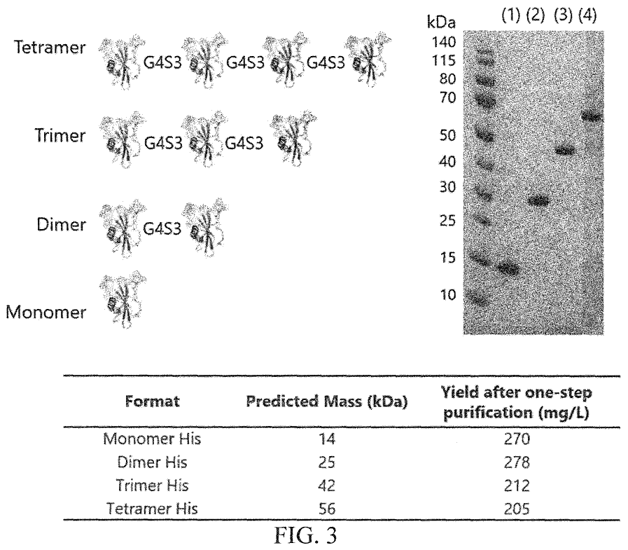

### Example 3: Expression of Affimer Multimers in E. coli (FIG. 3)

200 ng expression plasmid pD861 (Atum) was transformed into BL21 E. coli cells (Millipore) using the manufacturers protocol. Total transformed cell mixture was plated onto LB agar plates containing 50 ug/ml kanamycin (AppliChem) and incubated at 37° C. overnight.

Next, the lawn of transformed E. coli was transferred to a sterile flask of 1× broth media (Melford) & 50 ug/ml Kanamycin and incubated at 30° C. shaking at 250 rpm. Expression was induced with 10 mM Rhamnose (Alfa Aesar) once the cells reached an OD600 of 0.8-1.0 following which the culture was incubated for 5 hours at 37° C. Cells were harvested by centrifuging at 4,500 rpm for 1 h.

For culture volumes <500 ml E. coli cell pellet was lysed by resuspending in 1:10 NPI20 buffer (50 mM Sodium phosphate, 0.5M NaCl, 20 mM Imidazole (Sigma)) supplemented with 0.5 ml 10× BugBuster per gram of wet cell paste (Millipore), lysozyme (Applichem) and Benzonase (Millipore). Cells lysed for 1 hour at room temperature on a bottle roller. For culture volumes>500 ml, the cell pellet was resuspended in 1:10 supplemented NPI20 and sonicated for 2 minutes (10 seconds on/off cycles). Following lysing the solution was centrifuged at 20,000×g for 1 hour at 4° C.

Batch Bind Affinity Purification of His Tagged Protein from Clarified Supernatant Using Nickel Agarose Affinity Resin (Super-NiNTA500; Generon).

An appropriate volume of NiNTA resin (binding capacity 1 mL per 20 mg protein) was washed with 5 column volumes (CV) water to remove storage solution, followed by equilibration with 5 CV NPI20 buffer using gravity flow in a StEP™ column (Thompson). Resin was incubated with clarified E. coli solution for 1 hour at room temperature. Then, the solution was passed through a StEP™ column by gravity flow and the resin was washed with 5CV NPI20 buffer. Bound protein was eluted off the resin with 5 CV of NPI400 (50 mM Sodium phosphate, 0.5M NaCl, 0.4M Imidazole (Sigma)). Eluted protein was desalted into 1× PBS using Centripure columns (emp Biotech GmbH). The protein yield was estimated using Nanodrop (Thermo) A280 reading. Preparative SEC was performed using HiLoad 26/600 Superdex 200 pg (GE) run in PBS 1× at 2.6 ml/min flow rate on an AKTA Xpress (GE). Eluted protein samples were run on SDS-PAGE Bolt Bis Tris plus 4-12% gel (Thermo) in Novex™ 20× Bolt™ MES SDS running buffer (Thermo) at 200V with reducing sample buffer for 10 minutes at 95° C. Protein bands on gel were stained with Quick Coommassie (Generon). PageRuler prestained protein molecular weight marker (Thermo) was run on gel to estimate the molecular weight of the fusion proteins.

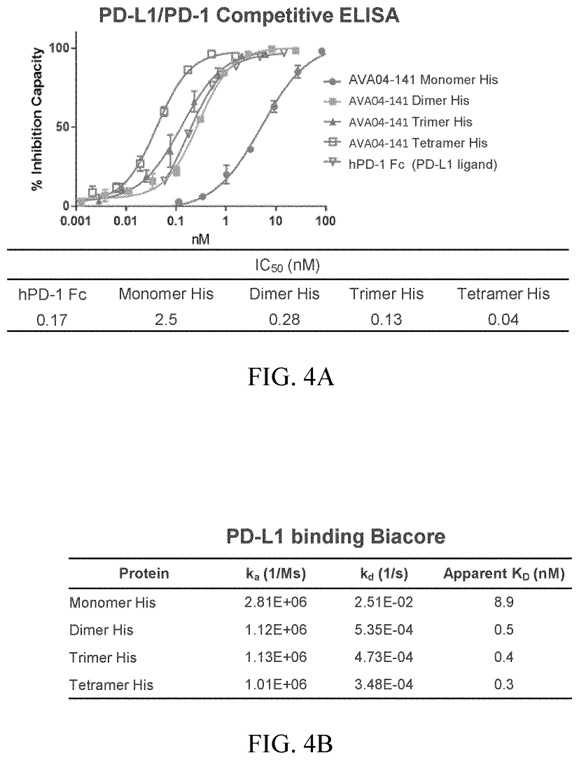

### Example 4: Competitive ELISA Assays and PDL-1 Binding Biacore (FIG. 4A and FIG. 4B) PD-L1/PD-1 Competitive ELISA (FIG. 4A)

The competitive inhibition of Affimer multimers was evaluated by enzyme linked immunosorbent assay (ELISA). hu PD-1-Fc (R&D Systems) was coated at 0.5 μg/ml on the plate. Plates were washed 2 times with 150 μl of washing buffer (PBS, Tween 20 0.1%) with a plate washer and saturated with Casein 5% (Sigma) in PBS for 90 minutes at room temperature (25±1° C.) Plates were washed as described previously. Affimer and controls (hu PD-1-Fc, R&D Systems, blank) were then diluted in duplicate, and preincubated with 30 ng/ml of huPD-L1-Fc (R&D Systems) for 30 minutes then loaded on the plate for 90 minutes at room temperature (25±1° C.) Plates were washed 3 times as described previously. Biotinylated polyclonal antibody anti hu PD-L1 (R&D Systems) was then diluted in dilution Buffer and incubated 90 minutes at room temperature (25±1° C.) Plates were washed 3 times as described previously and Streptavidin HRP was incubated for 30 minutes at room temperature (25±1° C.) Plates were washed and the substrate (TMB, Pierce Thermo-Scientific) was added in the plate for 10 minutes. The reaction was stopped using an acidic solution and plates were read at 450-630 nm. The IC 50 was then calculated using the interpolated non-linear four-parameters standard curve.

### PD-L1 Binding Biacore (FIG. 4B)

Biacore T200 kinetic analysis was performed on AVA04-141 in-line fusion (ILF) formats using running buffer HBS-EP+ (GE) and series S sensor CM5 chip immobilized with PD-L1-Fc (R&D Systems) in 10 mM Sodium acetate pH4.0 (GE) using amine coupling reagents (GE). The concentration titration of Affimer monomer was run as analyte with an association time of 150 seconds, followed by a dissociation time of 300 seconds at a flow rate of 30 μl/min. Affimer dimer, trimer and tetramer fusion proteins were run as analyte with an association time of 300 seconds, followed by a dissociation time of 600 seconds at a flow rate of 30 μl/min. PD-L1-Fc immobilized surface was regenerated with 5 mM NaOH (GE) for 20 seconds at 20 μl/min flow rate. The data blank was subtracted and fit to a 1:1 Langmuir binding model (BIAcore evaluation software; GE) to calculate an apparent KD value.

### Example 5: Characterization of Affimer Fc Fusions and Effector Function (ADCC) SEC-HPLC (FIG. 5A)

Suspension HEK cell (Expi293F cell line; Thermo) transfections were performed with expression vector pD2610v14 (Atum) using Expifectamine reagent (Thermo) following the manufacturers protocol. Supernatant was harvested 7 days post transfection by centrifuging at 10,000×g for 1 hour and filtered using 0.45 μm filter paper. Protein was purified using mabSelect Sure HiTrap columns on an AKTA Xpress (GE). Resin was washed with 5 column volumes (CV) water, equilibrated with 5 CV PBS 1×. Then, supernatant was run through at a flow rate of 5 ml/min followed by a wash with 10 CV PBS 1×. Bound protein was eluted in 5 CV 0.1M glycine pH 2.8, buffer exchanging into PBS 1× using Centripure desalting columns (empBiotech GmbH). After, a preparative SEC was performed as described in Example 3. Analytical SEC was carried out using a MAbPac SEC-1 column (Thermo) run on an Ultimate 3000 HPLC (Thermo) at 0.8 ml/min in PBS 1×.

Biacore Kinetics (FIG. 5B)

Biacore kinetic analysis was performed as described in Example 4 with an association time of 700 seconds and a dissociation time of 1200 seconds. Surface regeneration was performed with 3.5 nM NaOH (GE) for 20 seconds at a flow rate of 20 μl/min for low concentrations. For high concentrations time was increased to 30 seconds.

ADCC Reporter Bioassay (FIG. 5C)

The ADCC Reporter Bioassay is a bioluminescent reporter assay from Promega Corp. for quantifying biological activity on pathway activation by therapeutic antibody drugs in an ADCC mechanism of action (MOA) assay. AVA04-251 formatted on a functional IgG1 Fc was able to show ADCC function with a EC50 of ≥1 nM.

Briefly, target cells H441 cells (expressing huPD-L1) were seeded in a 96 well plate at a density 20 000 cells per well in 100 ul and incubated in a humidified CO2 incubator for 20 h. The next day, samples and control were diluted in ADCC buffer (Promega) in duplicate. In the meantime, effector cells were thawed (Jurkat FCgRIIIa reporter NEAT cells, Promega) and 630 ul of the initial cell suspension were diluted in 3.6 ml of ADCC buffer. 95 ul of medium was removed from plates and 25 ul of ADCC buffer, 25 ul of sample dilution or control and 25 ul of effector cells were added in each well. Plates were incubated 6 hours in a humidified CO2 incubator. Then plates were equilibrated at room temperature and 75 ul of BioG Lo Luciferase Assay reagent (Promega) was added in each reaction wells and incubated between 5-10 minutes at room temperature. Luminescence was then measured using a plate reader (Clariostar, BMG). Results were platted using graphpad as fold of induction=f(log concentration).

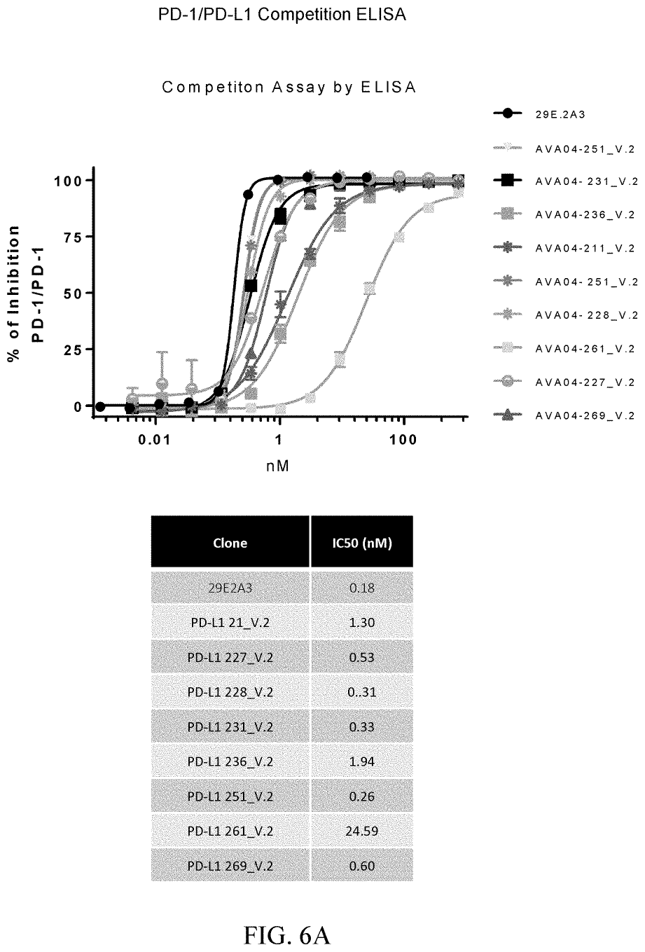

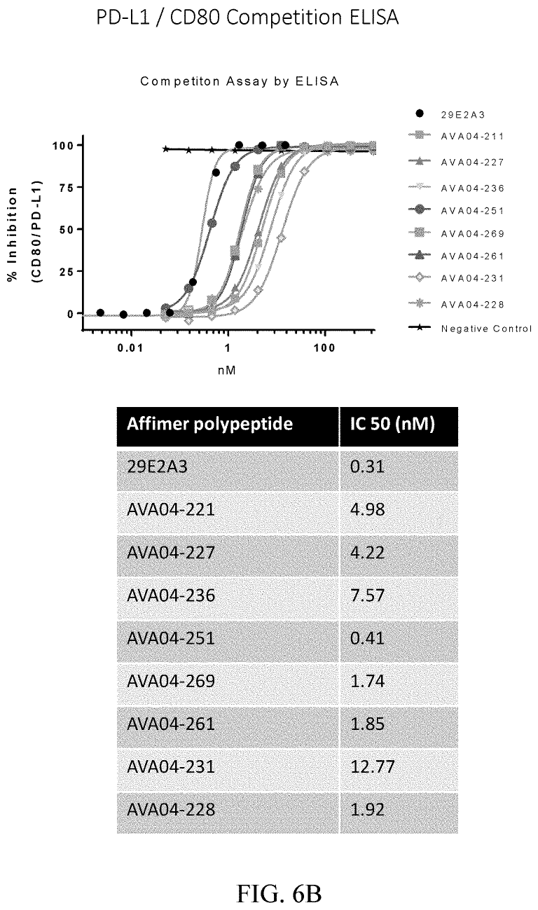

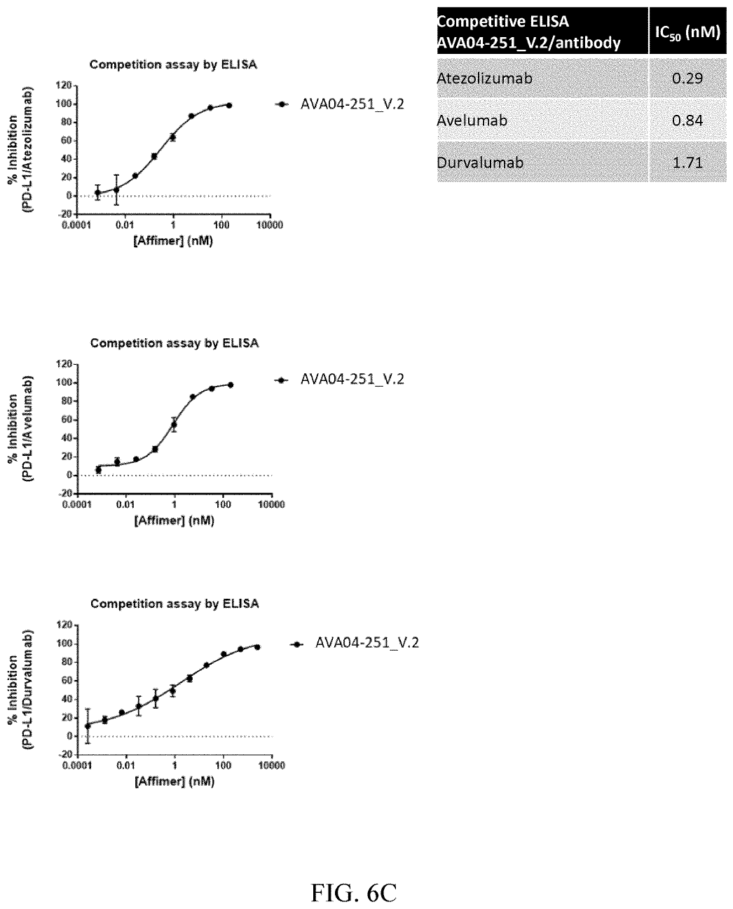

### Example 6: Competitive Elisa Assays (FIG. 6A-6C)

### PD-L1/PD-1 Competition ELISA (FIG. 6A)

Various Affimer polypeptides formatted on _V.2 were tested in a competitive ELISA as described FIG. 4A. All the formatted Affimers tested were competitive with an IC50 ranging from 0.26 to 24.6 nM. The control Antibody (29E2A3, Biolegend) was competitive with an IC50 comparable to the formatted Affimers (IC50=0.18 nM).

PD-L1/CD80 Competition ELISA (FIG. 6B)

Various Affimer polypeptides formatted on _V.2 were tested in a competitive ELISA.

All the formatted Affimers tested were competitive with CD80 with an IC50 ranging from 0.41 to 12.77 nM. The control Antibody (29E2A3, Biolegend) was competitive with an IC50 comparable to the formatted Affimers (IC50=0.31 nM).

Briefly the competitive inhibition was evaluated by enzyme linked immunosorbent assay (ELISA). hu CD80-Fc (R&D Systems) was coated at 1 ug/ml on the plate. Plates were washed 2 times with 150 μl of washing buffer (PBS, Tween 20 0.1%) with a plate washer and saturated with Casein 5% (Sigma) in PBS for 90 minutes at room temperature (25±1° C.) Plates were washed as described previously. Affimer and controls (mAb anti huPD-L1 29E.2A3, Biolegend; hu PD-L1-Fc, R&D Systems, blank) were then diluted in duplicate, and preincubated with 1.6 nM of huPD-L1-Fc (R&D Systems) for 30 minutes then loaded on the plate for 90 minutes at room temperature (25±1° C.) Plates were washed 3 times as described previously. Biotinylated polyclonal antibody anti hu PD-L1 (R&D Systems) was then diluted in dilution Buffer and incubated 90 minutes at room temperature (25±1° C.) Plates were washed 3 times as described previously and Streptavidin HRP was incubated for 30 minutes at room temperature (25±1° C.) Plates were washed and the substrate (TMB, Pierce Thermo-Scientific) was added in the plate for 10 minutes. The reaction was stopped using an acidic solution and plates were read at 450-630 nm. The IC 50 was then calculated using the interpolated non-linear four-parameters standard curve.

Benchmark Antibody Competition ELISA (FIG. 6C)

AVA04-25_V.2 was tested in a competitive ELISA to Avelumab, Atezolizumab and Durvalumab. AVA04-251_V.2 was competitive with each benchmark antibodies with an IC50 ranging from 0.29 to 1.71 nM.

Briefly, the competitive inhibition with benchmark antibodies was evaluated by enzyme linked immunosorbent assay (ELISA). Avelumab, Atezolizumab and Durvalumab were coated at 0.5 ug/ml on the plate. Plates were washed 2 times with 150 μl of washing buffer (PBS, Tween 20 0.1%) with a plate washer and saturated with Casein 5% (Sigma) in PBS for 90 minutes at room temperature (25±1° C.) Plates were washed as described previously. Affimer and controls (benchmark antibody, blank) were then diluted in duplicate, and preincubated with 0.07 nM of huPD-L1-Fc (R&D Systems) for 60 minutes then loaded on the plate for 90 minutes at room temperature (25±1° C.) Plates were washed 3 times as described previously. Biotinylated polyclonal antibody anti hu PD-L1 (R&D Systems) was then diluted in dilution Buffer and incubated 90 minutes at room temperature (25±1° C.) Plates were washed 3 times as described previously and Streptavidin HRP was incubated for 30 minutes at room temperature (25±1° C.) Plates were washed and the substrate (TMB, Pierce Thermo-Scientific) was added in the plate for 10 minutes. The reaction was stopped using an acidic solution and plates were read at 450-630 nm. The IC 50 was then calculated using the interpolated non-linear four-parameters standard curve.

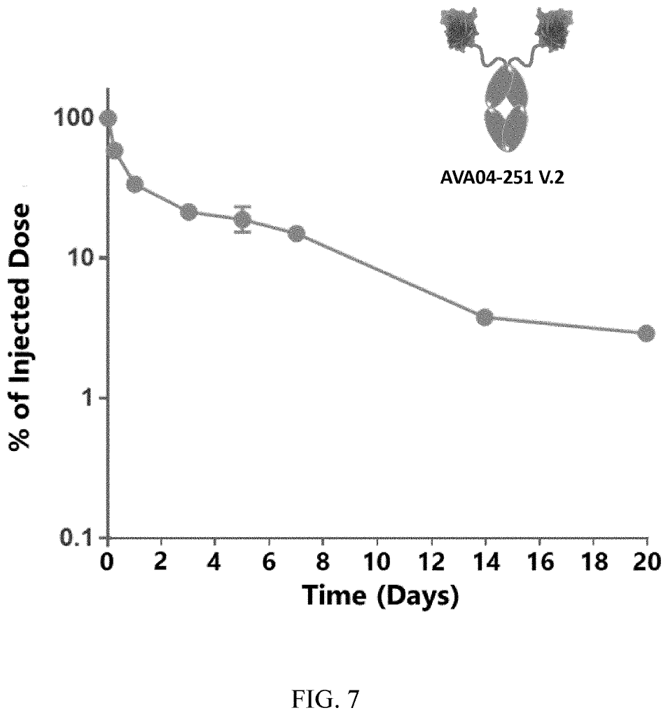

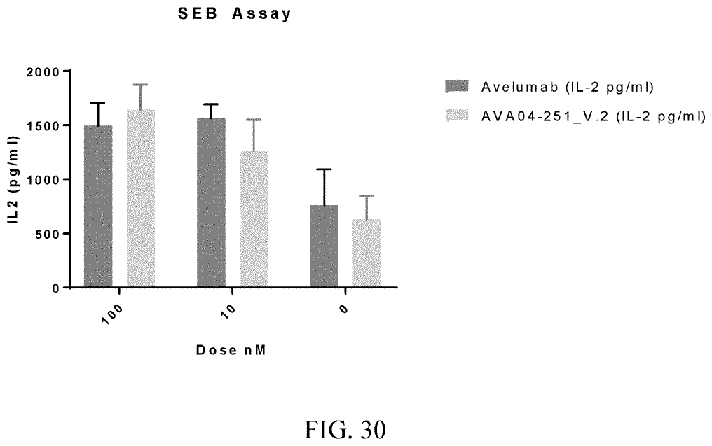

### Example 7: Pharmacokinetic Study of Affimer Half-Life Extended (FIG. 7) and Tissue Distribution (FIG. 30)

AVA04-251_V.2 was injected intravenously (IV) to C57BL/6 mice at 10 mg/kg. 6 mice were injected and 9 time points were collected (0 h, 0.25 h, 6 h, 24 h, 72 h, 120, 168 h, 336 h and 480 h). The serum samples for each time point were pooled and analyzed by sandwich ELISA using the molecules injected as reference standard. Results were expressed as percentage of the initial dose at 15 mins.

AVA04-251_V.2 used in this pharmacokinetic study was produced from Expi293F using Expifectamine transfections as described in Example 5. LAL assay was performed using Endosafe Nexgen-PTS™ reader (Charles River) to confirm endotoxin levels in protein samples as 0.01 EU/mg. Mammalian host cell proteins were quantified using HEK293 HCP 2nd generation ELISA (Cygnus) using the manufacturers protocol. The sample contained 5 ug/ml HCP per mg of protein.

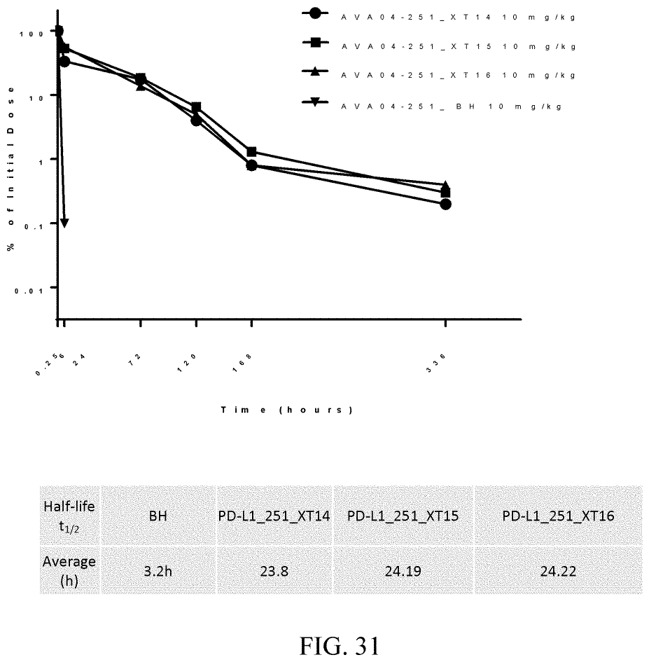

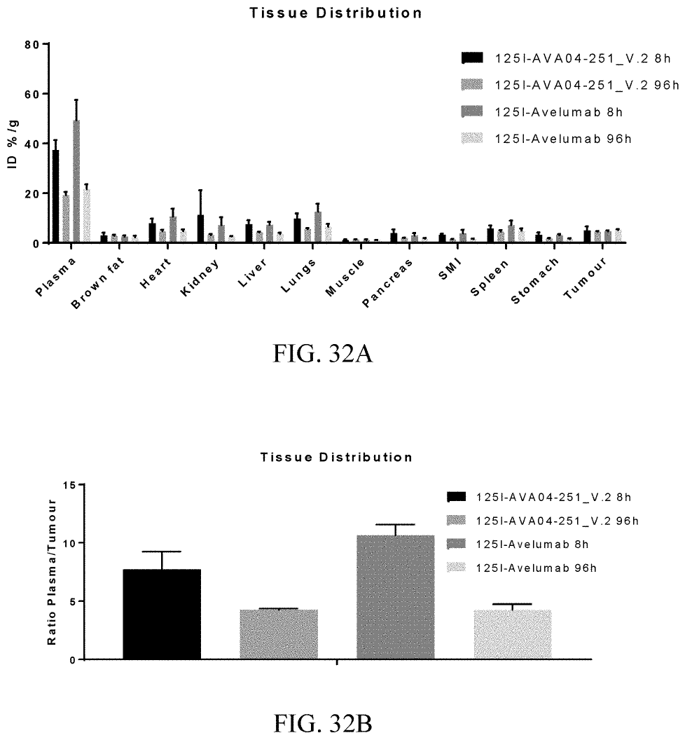

The tissue distribution of total radioactivity following intravenous administration of 125I-Affimer and 125I-Avelumab to humanized NOG mice bearing orthotopic MDA-MB-231 tumor cells was evaluated (FIG. 32A). Mice were randomized on their tumor size and injected IV with Avelumab or AVA04-251_V.2 (n=5 per group and time point). Mice were sacrificed at 8 and 96 h. Blood and organs were taken and removed and weight. The total radioactivity contained in the biological samples were determined using a RIASTAR A5410 multidetector gamma counter (Packard) designed for the detection and quantitative measurement of gamma radiations. Aliquots of weighed specimens were introduced into 5-mL polypropylene tubes. Then, the tubes were loaded into a counting tray for direct gamma detection. FIG. 31B shows the ratio plasma/tumor.

Overall, AVA04-251_V.2 has a similar tissue distribution to Avelumab.

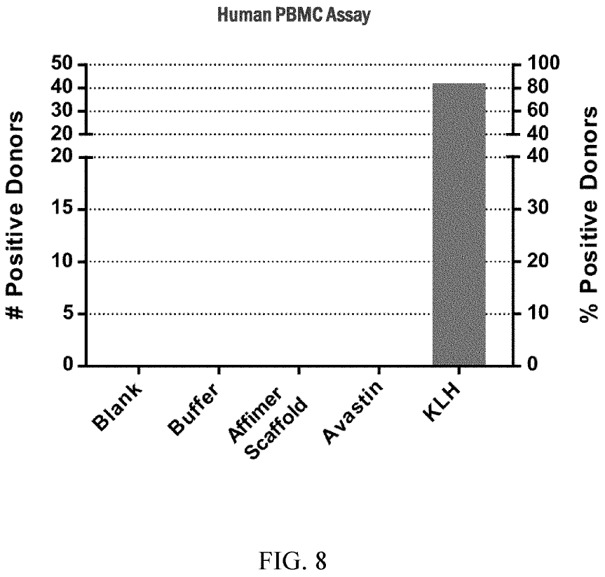

### Example 9: Immunogenicity Testing by human PBMC Stimulation assay (FIG. 8)

Affimer parent HEK cell transfection and mabSelectSure purification was performed as described in Example 5, with the addition of cation exchange purification step using an SP high performance column (GE) and using a 20 mM sodium acetate pH4 buffer. A 10CV wash of running buffer and 0.1% triton 114× (Sigma) was used to remove negatively charged endotoxin. Bound protein was eluted with 20 mM sodium acetate pH4 & 2M NaCl. Preparative SEC was performed as described in Example 3. LAL assay was performed using Endosafe Nexgen-PTS™ reader (Charles River) to confirm endotoxin levels in protein samples as 0.04 EU/mg. Host cell proteins were quantified using a HEK293 HCP quantification kit (Cygnus) using the manufacturers protocol. Samples contained 30 ng/ml HCP per mg of protein.

PBMC, isolated from each of the healthy donors, were retrieved from cryogenic storage and thawed in serum free cell culture media and were seeded into tissue-culture treated 96-well round bottom microplates. All test products were diluted in serum free cell culture medium and added to the wells at a final concentration of 50 μg/ml, Stefin A, was tested at 25 μg/ml. The cells were kept in culture for 7 days. CD4+ T cell proliferation was assessed by measuring 5-Ethynyl-2′-deoxyuridine (EdU) incorporation. On day 6 PBMC cultures were pulsed with EdU for approx. 16 hours. On day 7 the cells were fluorescently stained for T cell surface markers (CD3, CD4 and CD8), fixed and permeabilized, and the incorporated EdU was stained with a fluorescent azide. Stimulation index were calculated as a ratio against Buffer. SI>2 was considered as a positive response. % of responder was plotted for each molecule tested.

Overall, only the positive control KLH was able to stimulate T cell proliferation after 7 days of treatment.

### Example 10: Formatting Affimers at Various Sites on an Fc (FIG. 9)

Expi293F transfections and one stage mabSelect Sure purification was performed as described in Example 5 on AVA04-236-A(EAAAK)6 (SEQ ID NO: 196) hlgG1 Fc (AR), AVA04-236-A(EAAAK)6 (SEQ ID NO: 196) hlgG1 Fc C terminal Affimer (AQ), and AVA04-236 dimer on hlgG1 Fc (AG). Analytical SEC was carried out using a MAbPac SEC-1 (Thermo) and was run on an Ultimate 3000 HPLC (Thermo) at 0.8 ml/min in PBS 1× mobile phase. Results for AVA04-236-A(EAAAK)6 (SEQ ID NO: 196) hlgG1 Fc, AVA04-236-A(EAAAK)6 (SEQ ID NO: 196) hlgG1 Fc C terminal Affimer, and AVA04-236 dimer on hlgG1 Fc were 96%, 97%, and 92%, respectively.

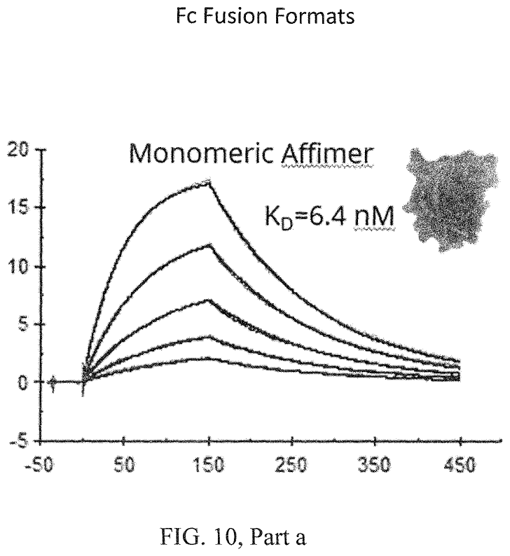

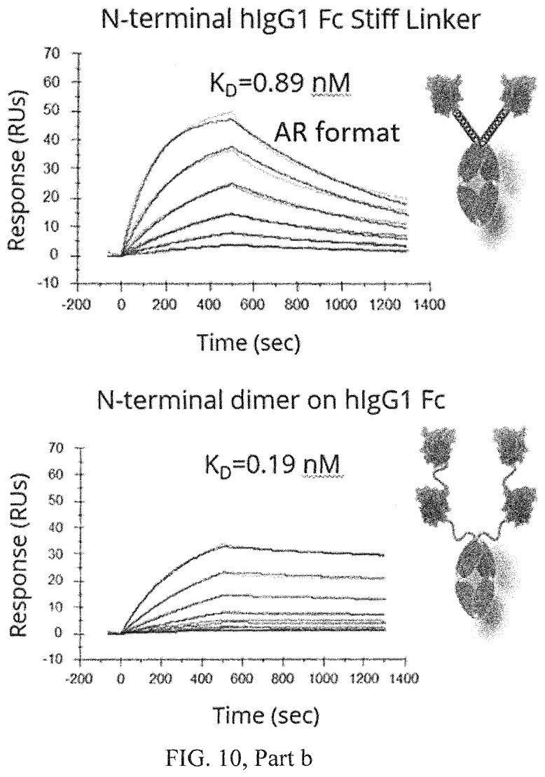

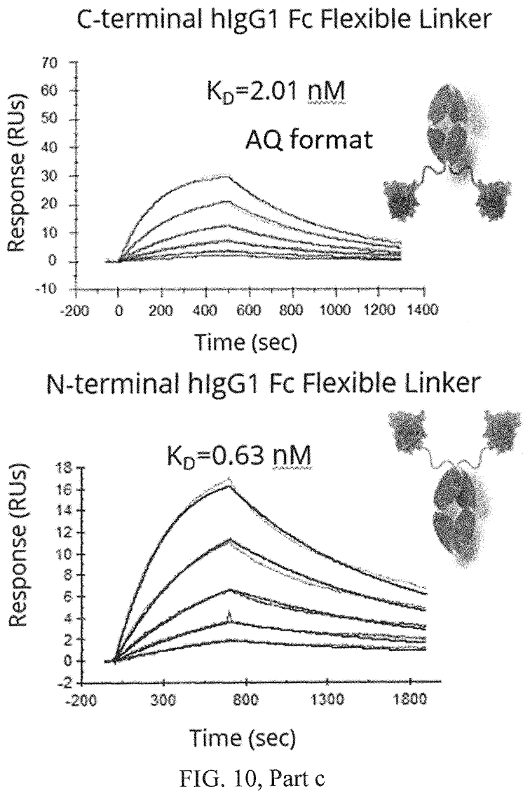

### Example 11: Kinetic Analyses (FIG. 10)

Biacore kinetic analysis performed as described in Example 4 on a monomeric Affimer, N-terminal hlgG1 Fc stiff linker (AR), N-terminal dimer hlgG1 Fc (AG), C-terminal hlgG1 flexible linker (AQ), and N-terminal hlgG1 flexible linker (V.2). Analysis used an association time of 500 seconds and a dissociation time of 800 seconds. Regeneration was done with 3 mM NaOH (GE) for 20 seconds at 20 ul/min. K_{D} results for monomeric Affimer, N-terminal hlgG1 Fc stiff linker, N-terminal dimer hlgG1 Fc, C-terminal hlgG1 flexible linker, and N-terminal hlgG1 flexible linker were 6.4 nM, 0.89 nM, 0.19 nM, 2.01 nM, and 0.63 nM, respectively.

### Example 12: Ipilimumab (Biosimilar) Fusion Analyses (FIGS. 11A-11B)

Expi293F transfections were performed on Ipilimumab and Ipilimumab-AVVA04-141 with a 1:1 ratio of heavy:light chain vectors pD2610v14 (Atum). Affinity chromatography and analytical SEC HPLC was carried out as described in Example 5. Preparative SEC and reducing and non-reducing SDS-PAGE QC was run as described in Example 3.

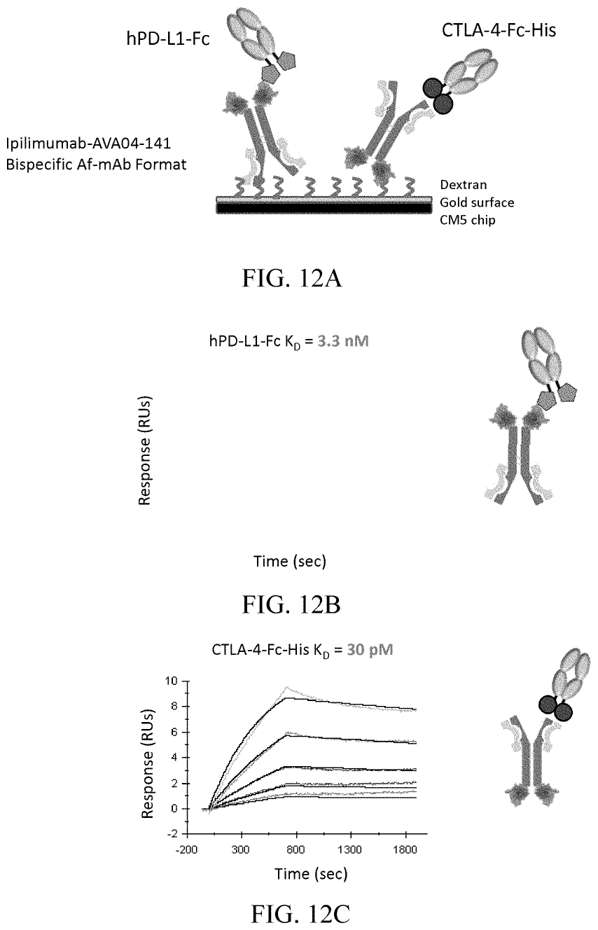

### Example 13: Kinetic Analyses (FIGS. 12A-12C)

Biacore kinetic analysis carried was out using protocol described in Example 4. Ipilimumab-141 fusion protein was immobilized on the CM5 chip surface using amine coupling in 10 mM sodium acetate pH4 buffer (GE). PD-L1-Fc or CTLA4-Fc antigens (R&D Systems) were titrated for an association time of 300 seconds and dissociation time of 600 seconds for PD-L1-Fc, and 700 seconds association and 1200 seconds dissociation times for CTLA4-Fc. Regeneration was done using 3 mM NaOH (GE) for 20 seconds at 20 μl/min.

### Example 14: Bispecific Bevacizumab (Biosimilar)-PD-L1 Affimer Characterization (FIGS. 13A-13C) and Half-Life Determination by Pharmacokinetic Analysis (FIG. 13D)

Bevacizumab-AVA04-251 Bispecific Af-mAbs was produced as described in Example 5 using transfection ratio of 1:1 VH:Vk plasmids pD2610v14 (Atum). Bevacizumab-AVA04-251 Bispecific Af-mAbs with rigid or flexible linkers was immobilized on CM5 chip surface using amine coupling with 10 mM sodium acetate pH5.0 (GE). hPDL1-Fc (R&D Systems) was run as analyte with an association time of 300 seconds and a dissociation time of 600 seconds, regenerating with 3 mM NaOH (GE) for 20 seconds at 20 μl/min. (FIG. 13B)

Bevacizumab-AVA04-251 Bispecific Af-mAb or Bevacizumab (biosimilar, anti-VEGF, Invivogen) was immobilized on CM5 chip surface using amine coupling with 10 mM sodium acetate pH4.5 and 5.5 respectively (GE). hVEGF (Peprotech) was run as analyte and analyzed as single cycle kinetics, ×5 concentrations were run with an association time of 700 seconds and a dissociation time 1200 seconds, regenerating with 3.5 mM NaOH (GE) for 20 seconds at 20 μl/min. The data blank was subtracted and analyzed with a 1:1 Langmuir binding model BIAcore evaluation software; GE) to calculate apparent KD values.

FIG. 13D: Pharmacokinetic study in C57/B16 mice: As described FIG. 7, mice were injected intravenously (IV) at 10 mg/kg. 6 mice were injected and 9 time points were collected (0 h, 0.25 h, 6 h, 24 h, 72 h, 120, 168 h and 336 h). The serum samples for each time point were pooled and analyzed by sandwich ELISA using the molecules injected as reference standard. Results were expressed as percentage of the initial dose at 15 mins. Affimer Af-mAb half-life was estimated in the beta phase around 127 hours) comparable to the Fc formatted affimer (AVA04-251_AQ.2).

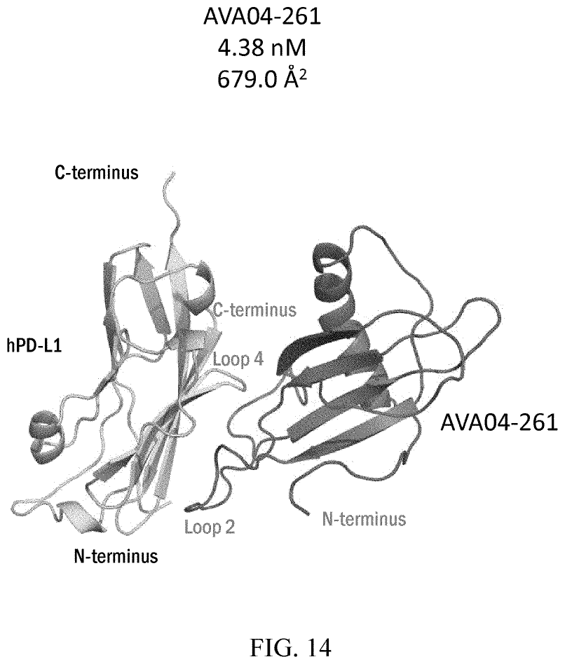

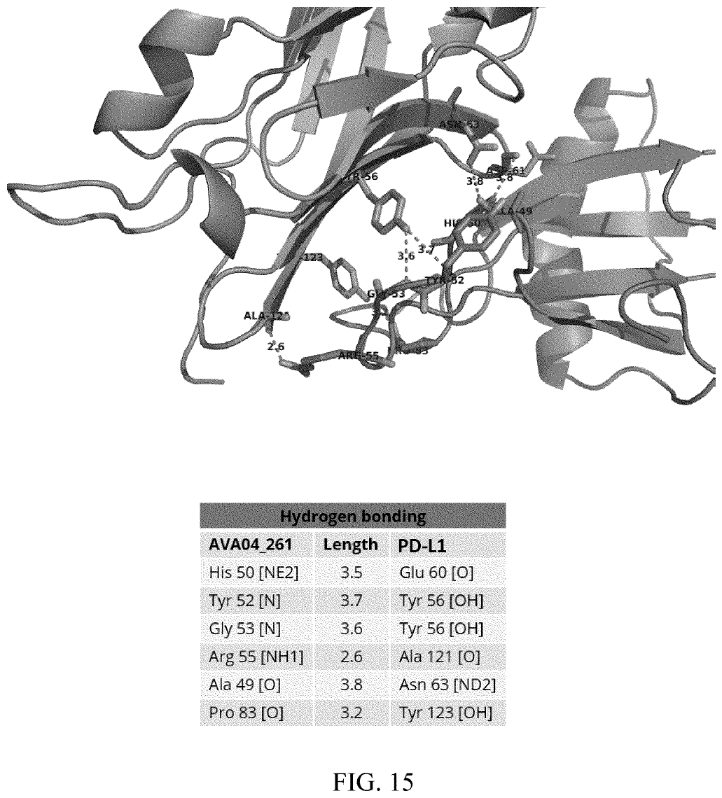

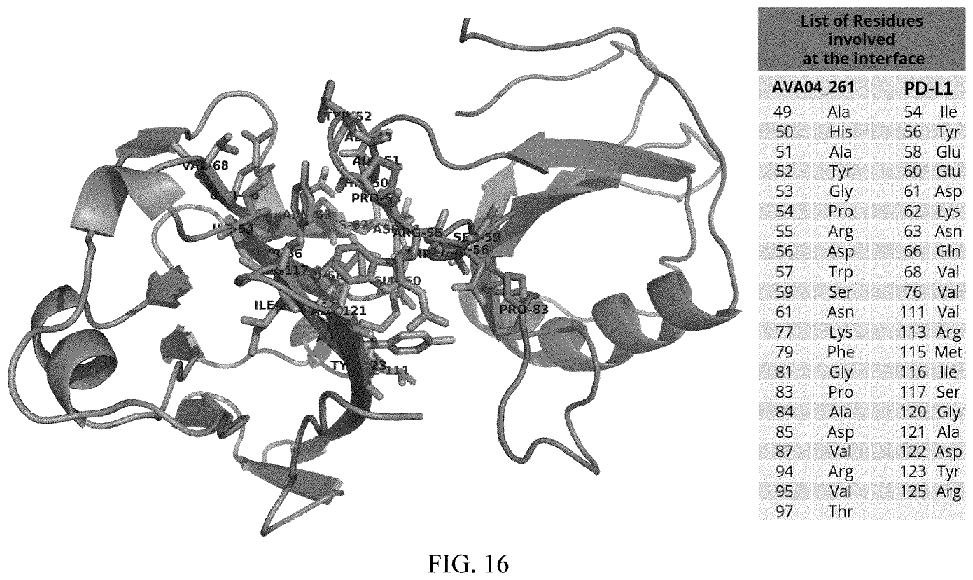

### Example 15: Protein Production for Crystallography (FIGS. 14-16)

The hPD-L1 binding domain (N-terminal IgV domain 18-134) was expressed as inclusion bodies in E. coli. Cell pellet was solubilized in tris buffered saline (TBS) and supplemented with 8 M urea for 16 h at 4° C. His tagged antigen was purified using NiNTA as described in FIG. 3, with elution buffer TBS, 8 M urea and 400 mM imidazole. Protein was refolded by drop wise addition of ˜100 mg protein to 1 L refolding buffer; 100 mM Tris pH 8 supplemented with 0.5 M arginine. 0.25 mM glutathione (reduced and oxidized) and protease inhibitor tablets (EDTA free; Roche) then stirred over night at 4° C. The protein was then concentrated by tangential flow filtration (TTF) and buffer exchanged into TBS & 10% glycerol. The protein was purified using Hiload 26/600 Superdex 75 pg using AKTA Xpress (GE) run at 2.6 ml/min in TBS & 10% glycerol running buffer. AVA04-261 was expressed and purified as described in Example 3 using TBS running buffer. Eluted protein was stored at −20° C. at 2 mg/ml followed by mixing of AVA04-261 with hPD-L1 antigen in equimolar quantities. Then the preparative SEC was run as described above. Eluted fractions were concentrated to 68 mg/ml.

To perform the crystallography, several commercial screens were set up with sitting drop vapor diffusion method using 0.1 μl protein and 0.1 μl reservoir solution. They were left at room temperature overnight and obtained hexagonal crystals from many different buffers. Diffraction data was collected on crystals obtained in 25% Jeffamine SD-2001, 100 mM MES pH 5.5 and 100 mM Sodium Malonate dibasic. Crystals were flash-cooled in liquid nitrogen in mother liquor. Data was collected at Diamond light source, UK. The AVA04-261/hPD-L1 binding domain protein complex structure was solved using the CCP4 suite using molecular replacement with models derived from pdb structures 3KSE, 4N6T and 5C3T, diffraction was collected to 2.09 Å. The main interaction surface was hydrophobic with loop 2 of AVA04-261 interacting with hD-L1 antigen. The hPD-L1 binding surface was very similar to literature examples of proteins binding PD-L1 (1. Lee, J. et al. (2016) Nature Communications 7, 13354, 2.Zak, K. et al. (2016) Oncotarget, 7, 30323, 3.Zhang F. et al (2017) Cell Discovery, 3, 17004.)

### Example 16: Half-Life Extended Anti-Human PD-L1 Dimer Affimer in-Line Fusions (ILF): Schematic Representation and XT Nomenclature with Rigid or Flexible Genetic Linkers (FIGS. 17A-17B)

Affimer ILF was produced as detailed in Example 3. Following preparative SEC, protein was run on SEC-HPLC as described in Example 3, >95% purity for all fusion proteins. See FIG. 17A.

Biacore kinetic analysis demonstrated both anti-human PD-L1 and anti-human serum albumin Affimers whilst genetically fused were able to engage target proteins, AVA04 Affimers bind human PD-L1 within 2-6 fold affinity and the HSA Affimer binds HSA with 5 fold affinity (experiments were carried out as described in Example 4). Biacore T200 kinetic analysis was performed for serum albumin using running buffer HBS-EP+(GE) and series S sensor CM5 chip was immobilized on surface Fc2 with human serum albumin (Sigma; A37812) in 10 mM Sodium acetate pH 5.0 (GE) using amine coupling reagents (GE). A concentration titration of Affimer monomers were run as analyte at a flow rate of 30 μl/min. Kinetic data was blank subtracted and fit to a 1:1 Langmuir binding model (BIAcore evaluation software; GE) to calculate KD values. See FIG. 17B.

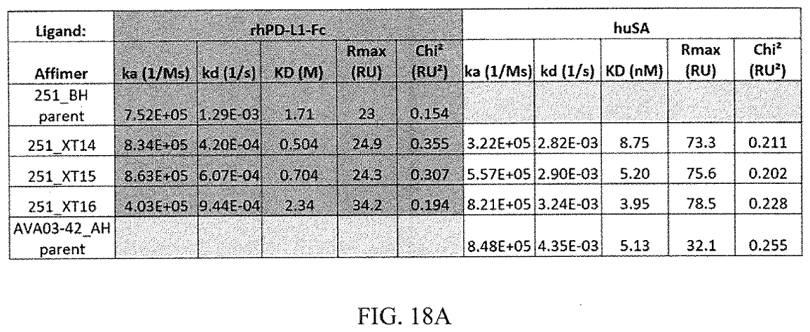

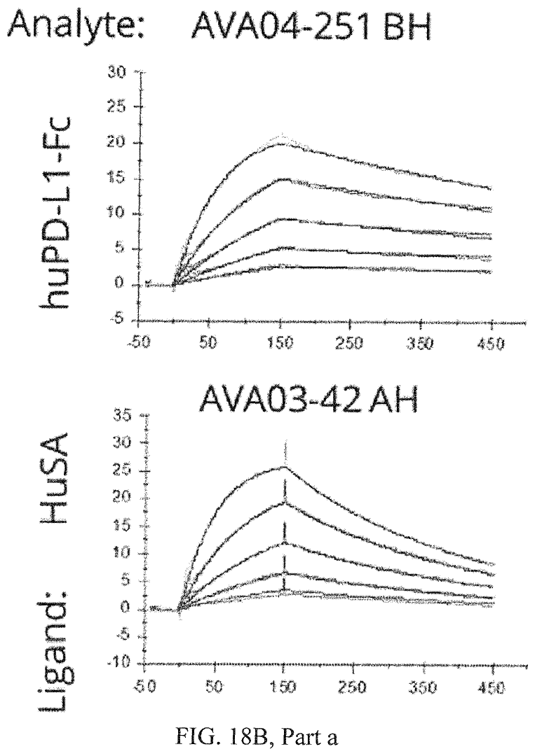

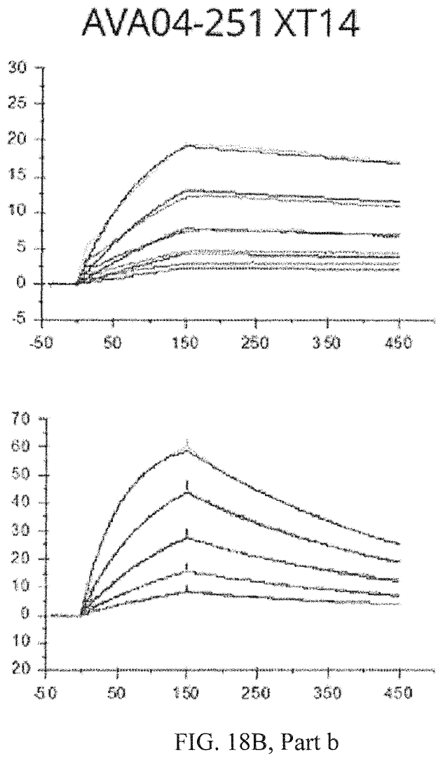

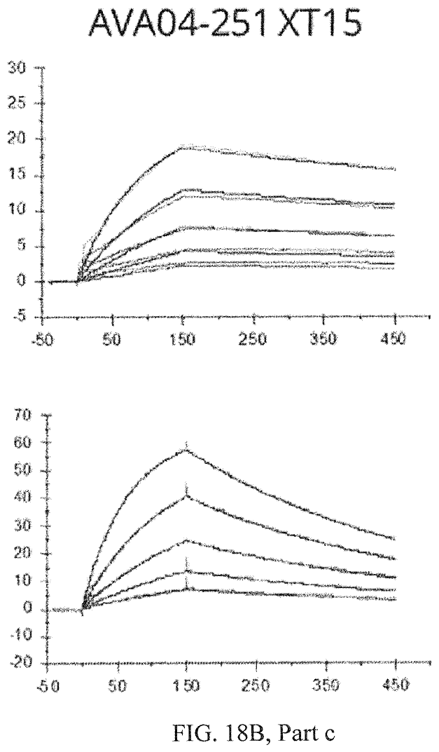

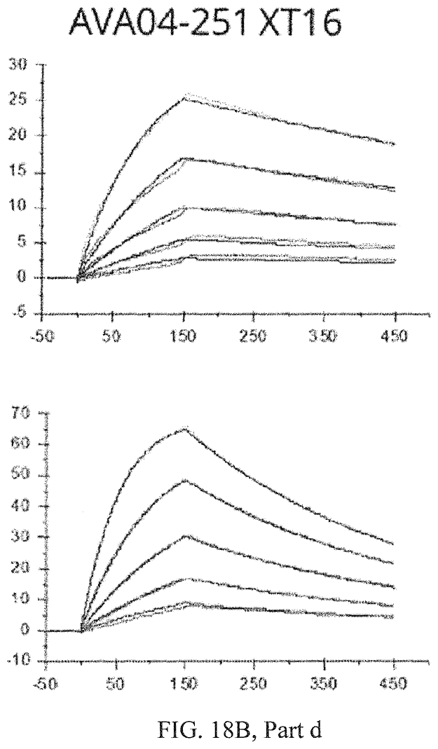

### Example 17: ILF Trimers with Half-Life Extension (FIG. 18A, B and FIG. 31)

ILF AVA04-251 trimers with half-life extension affimer in position A, B or C: Biacore kinetic analysis showed Affimers whilst genetically fused were able to engage target proteins. AVA04 Affimer dimer bound to human PD-L1 within 2-fold of the 251 ILF dimer (format BH) and the other Affimer engaged HSA. Biacore was carried out as described in Example 4 for PD-L1 and Example 4 for HSA binding.

FIG. 31: Pharmacokinetic of ILF Affimer XT: The ILF AVA04-251 trimers with half-life extension have been tested in a pharmacokinetic study in C57/B16 mice. As described FIG. 7, mice were injected intravenously (IV) at 10 mg/kg. 6 mice were injected and 9 time points were collected (0 h, 0.25 h, 6 h, 24 h, 72 h, 120, 168 h and 336 h). The serum samples for each time point were pooled and analyzed by sandwich ELISA using the molecules injected as reference standard. Results were expressed as percentage of the initial dose at 15 mins. Affimer ILF without half-life extension (BH) had a fast clearance (t_{1/2} 3.2 h) but ILF AVA04-251 XT format and half-life were estimated in the beta phase ranging from 23.8-24.2 hours).

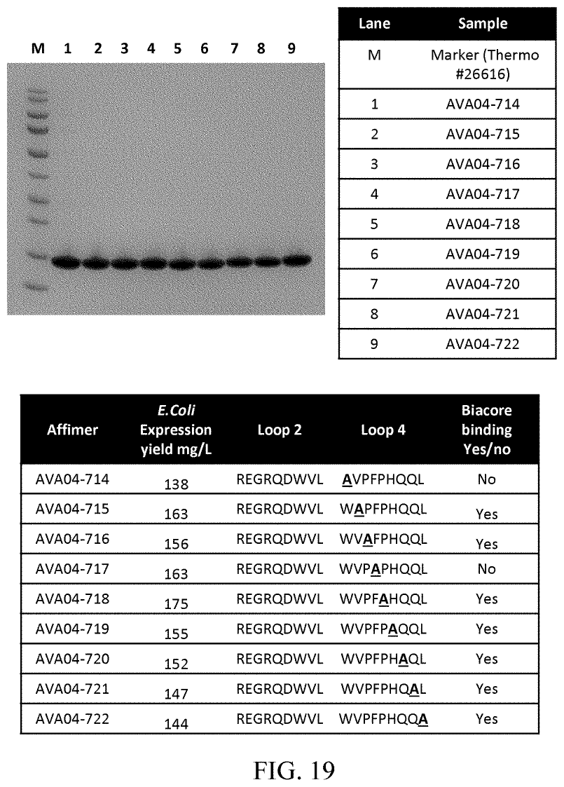

### Example 18: Analysis of Target Binding Sequence (FIG. 19)

AVA04-251 alanine scanning across ×9 amino acid positions in loop 4. Reducing SDS-PAGE run of 1 μl of purified variant proteins, E. coli production yield approximately 150 mg/L for all variants. hPD-L1 Biacore kinetic analysis was run at 50 nM concentration, identifying position 1 and 4 of loop 4 as essential for target binding.

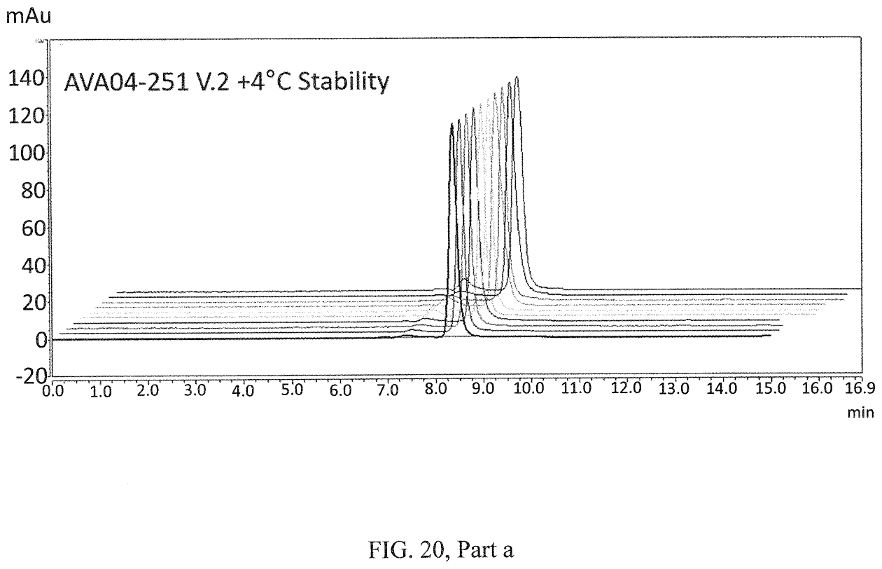

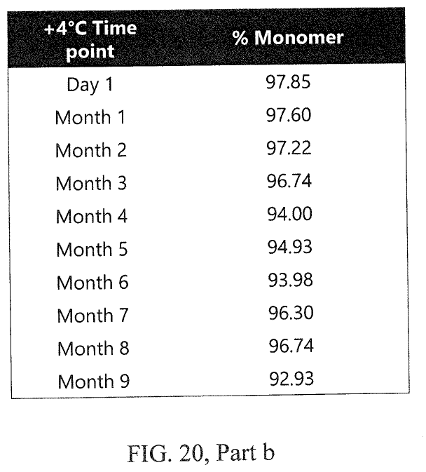

### Example 19: Stability (FIG. 20)

To test the stability of AVA04-251 V.2, SEC-HPLC analysis was performed on a Yarra-3000 (Phenomenex) column run in PBS 1× buffer at 1 ml/min each month for nine months. Samples were stored at +4° C. Monomeric purity decreased by 3-5%

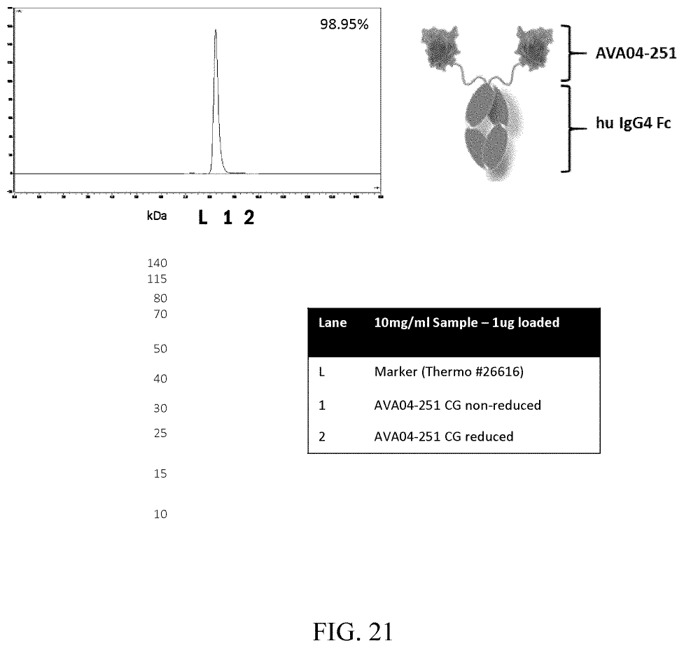

### Example 20: (FIG. 21)

AVA04-251 CG, anti-PDL1 Affimer was attached IgG4 Fc fusion protein with hinge S228P mutation and (G4S)4 (SEQ ID NO: 197) linker. The protein was produced from transient Expi293F cells as described in Example 5. The fusion protein was purified using PrismA (GE) protein A affinity resin eluted in 100 mM sodium citrate pH3.5 buffer. Then preparative SEC was performed as described in Example 3. Purity was assessed using a SEC-HPLC Yarra-3000 column run on an Ultimate 3000 HPLC (Thermo) at 1 ml/min in PBS 1× buffer. The final protein was >98% pure.

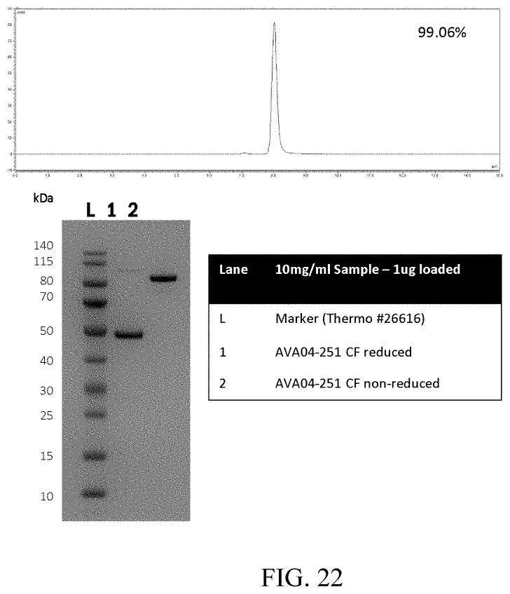

### Example 21: (FIG. 22)

AVA04-251 CF was a IgG1 Fc fusion with L153A, L154A Fc CH1 mutations for reducing ADCC. The protein was produced from transient Expi293F cells and purified as described in Example 5. Purity was assessed using a SEC-HPLC Yarra-3000 column run on an Ultimate 3000 HPLC (Thermo) at 1 ml/min in PBS 1× buffer. The final protein was >99% pure.

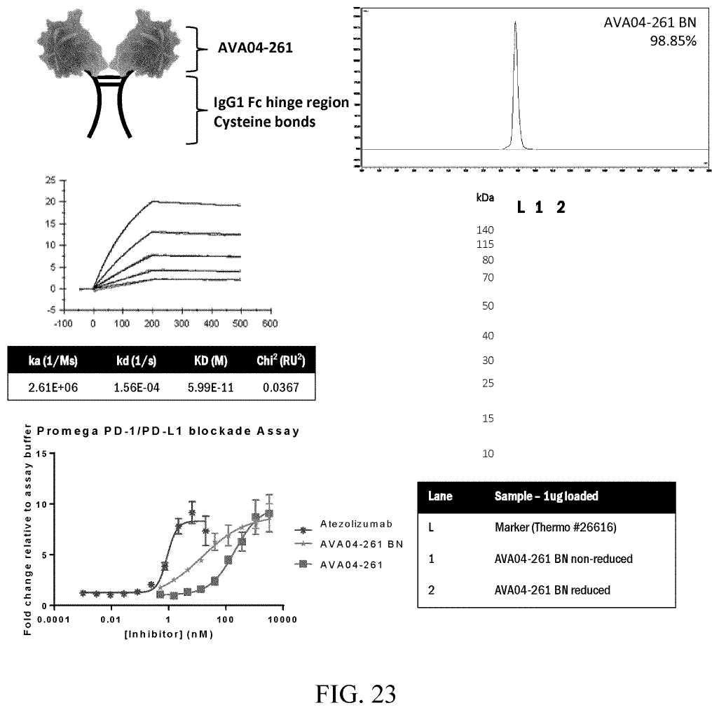

### Example 22: (FIG. 23)

AVA04-261 Affimer was genetically fused to the IgG1 hinge region of Fc and produced from E. coli (BN format). The protein was purified as described in Example 3. SDS-PAGE was run to show that dimeric species were dimerized through cysteines in the hinge under non-reducing conditions and reduced to monomeric species in reducing buffer. PD-1 PD-L1 blockade Bioassay (Promega) assay was run according to manufacturer instructions in duplicate and showed the AVA04-261 BN format had avidity when compared to monomeric AVA04-261. Biacore of the AVA04-261 BN format resulted in 59.9 pM KD.

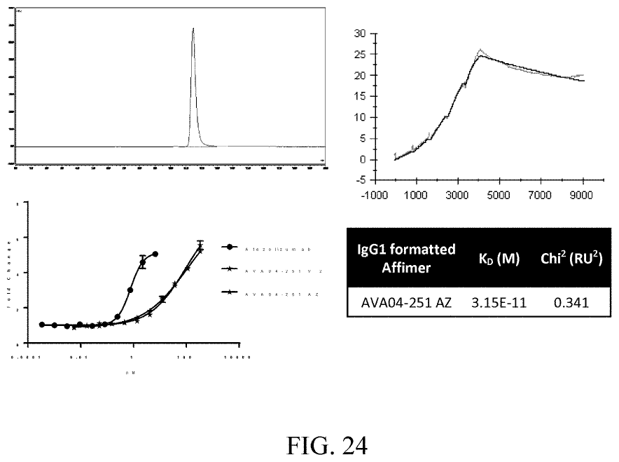

### Example 23: (FIG. 24)

In the AVA04-251 AZ human IgG1 Fc fusion protein, the Affimer was directly fused to Fc hinge region. The protein was produced from transient Expi293F cells and purified as described in Example 5. Purity was assessed using a SEC-HPLC Yarra-3000 column run on an Ultimate 3000 HPLC (Thermo) at 1 ml/min in PBS 1× buffer. The final protein was >99% pure. The Biacore single cycle kinetics data blank was subtracted and fitted to a 1:1 binding model and demonstrated a KD of 31.5 pM. PD-1 PD-L1 blockade Bioassay (Promega) demonstrated the same activity as V.2 format of (G4S)4 (SEQ ID NO: 197) linker between Fc hinge and Affimer.

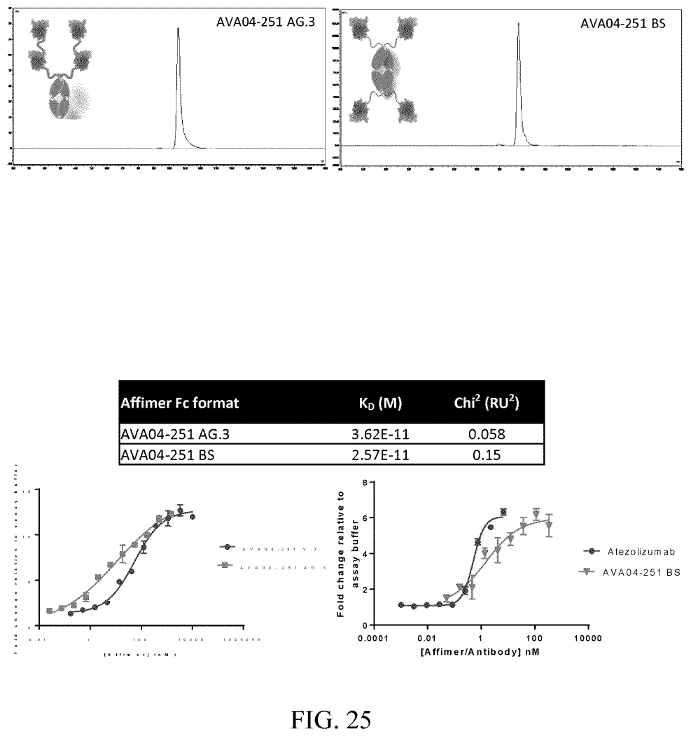

### Example 24: (FIG. 25)

Human IgG1 Fc fusions with four anti-human PD-L1 affimers were fused at the N or C terminus of the Fc. AVA04-251 AG.3, AVA04-251 BS formats were produced from Expi293F cells and purified using mabselect sure resin. Then, preparative SEC was performed as described in FIG. 5A. In a PD-1 PD-L1 blockade Bioassay (Promega) both formats showed blockade of PD-L1 binding to PD-1. The Biacore single cycle kinetics data blank was subtracted and fitted to a 1:1 binding model to demonstrate a KD of 36.2 pM and 25.7 pM for AVA04-251 AG.3 and AVA04-251 BS, respectively.

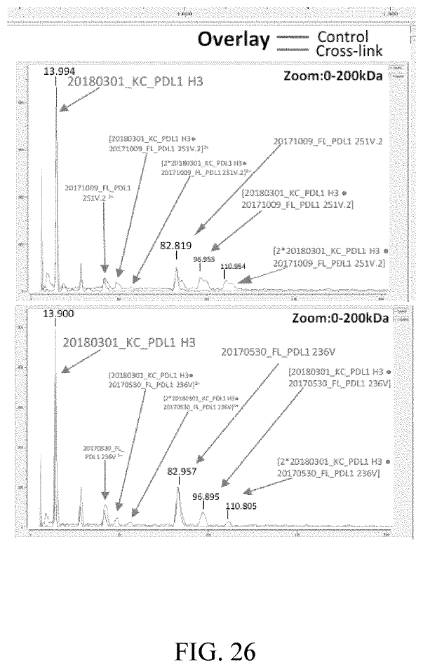

### Example 25: (FIG. 26)

Cross linking mass spectrometry of PD-L1 binding domain (14 KDa) with Fc fusion AVA04-251 V.2 or AVA04-236 V (82 kDa) was performed to analyze non-covalent binding complexes. Affimer Fc fusions were mixed with excess human PD-L1 domain using a specially developed cross-linking mixture (Bich, C et al. Anal. Chem., 2010, 82(1), pp 172-179). The solution was mixed at +4° C. and left overnight. The High-Mass detection system characterized the interaction in the High-Mass range of 0 to 1500 kDa. MS parameters, both linear and positive mode, ion Source 1: 20 kVlon Source 2: 17 kV. With lens 12 kVPulse, ion Extraction 400 nsHM4, gain voltage: 3.14 kV and acceleration voltage of 20 kV. Using High-Mass MALDI mass spectrometry and chemical cross-linking detected AVA04-Fc fusion protein alone, species binding one (+14 kDa) and two PD-L1 (+28 kDa) proteins confirming the presence of a stoichiometry of 1:1 and 2:1 species.

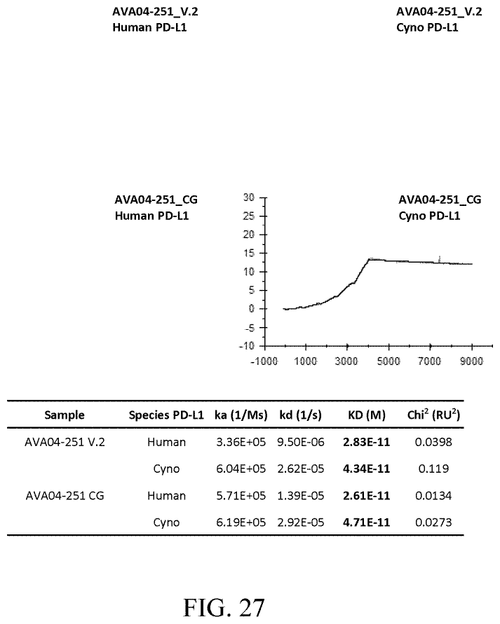

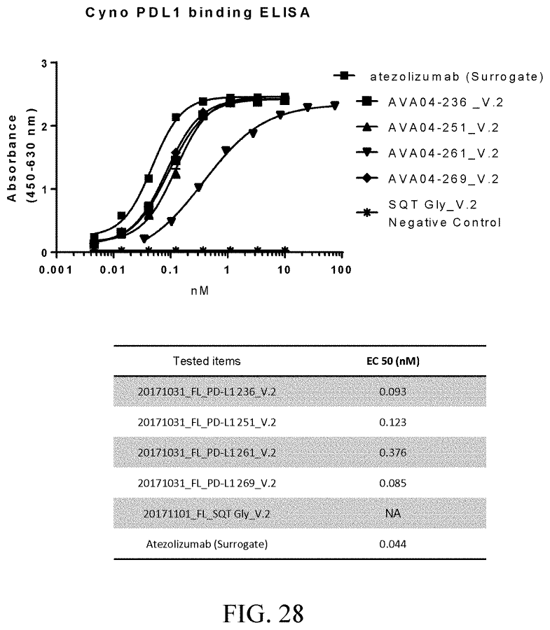

### Example 28: Cynomolgus Cross Reactivity (FIGS. 27 and 28)

Cross reactivity of AVA04-251 V.2 and CG formats exemplified using Biacore performed as described in FIG. 4 (FIG. 27). Affinity to human and cynomolgus PD-L1 Fc antigen (R&D Systems) is within 2-fold, in the range of 26-47 pM.

Cross reactivity of various _V.2 formatted Affimer by ELISA (FIG. 28). All formatted Affimers tested were binding to cynomolgus PD-L1 with an EC50 ranging from 0.085 to 0.375 nM. The benchmark antibody was detected at EC50=0.044 nM similar to the best formatted Affimer.

Briefly, the cross reactivity was evaluated by enzyme linked immunosorbent assay (ELISA). Cynomolgus PD-L1 His tag (Sino Biological) was coated at 2 ug/ml on the plate. Plates were washed 2 times with 150 μl of washing buffer (PBS, Tween 20 0.1%) with a plate washer and saturated with Casein 5% (Sigma) in PBS for 90 minutes at room temperature (25±1° C.) Plates were washed as described previously. Affimer and controls (Atezolizumab and Blank) were then diluted in duplicate, and incubated for 90 minutes at room temperature (25±1° C.) Plates were washed 3 times as described previously. Polyclonal antibody HRP anti hu Fc (Abcam) was then diluted in dilution Buffer and incubated 90 minutes at room temperature (25±1° C.) Plates were washed 3 times as described previously and the substrate (TMB, Pierce Thermo-Scientific) was added in the plate for 10 minutes. The reaction was stopped using an acidic solution and plates were read at 450-630 nm. The EC 50 was then calculated using the interpolated non-linear four-parameters standard curve.

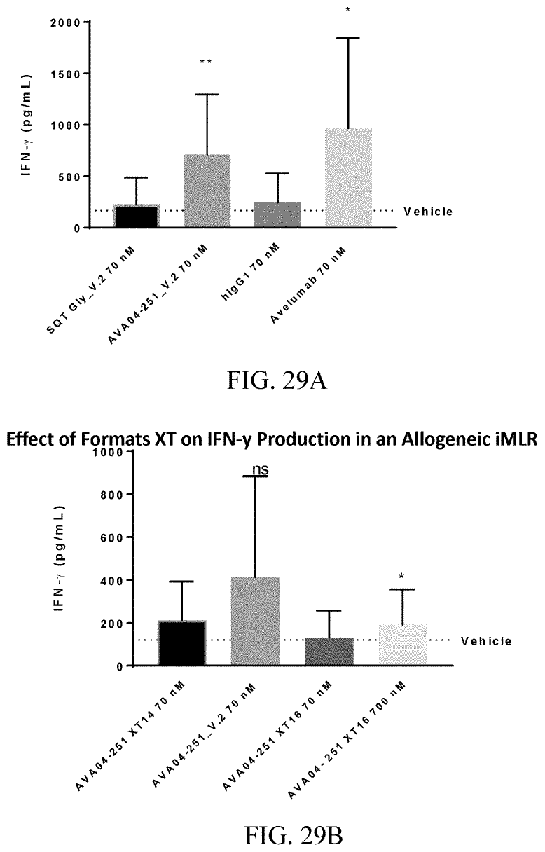

### Example 29: Mixed Lymphocyte Reaction (FIGS. 29A and 29B)

hFc1 formatted affimer were tested in a MLR assay (FIG. 29A). Briefly, dendritic cells (DC) derived from monocytes were prepared from CD14+ monocytes cultured for 7 days. Immature DCs were used on day 7 and cultured together with allogeneic T cells (negative isolation) and reference substance or vehicle control. Cells were cultured for 4 days and IFN-γ was measured in supernatants at the end of the culture period by ELISA. Data are presented as mean+/−S.E.M. pg/ml (left) or normalised to vehicle control (right), (n=6). **p<0.01, ***p<0.001, ****p<0.0001, using two-way ANOVA with Dunnett's post-test comparing test substances to the respective concentration SQT Gly V.2 control. Dotted line represents mean vehicle (RPMI-10) value. AVA04-251_V.2 did increase by >2 fold the level of IFNγ in a comparable manner as the benchmark antibody (avelumab).

XT formatted affimer were tested in a MLR assay as previously described (FIG. 29A) AVA04-251 XT14 and XT16 did increase the level of IFNγ as ILF format (FIG. 29B).

### Example 30: Staphylococcal Enterotoxin B Stimulation of PBMCs (FIG. 30)

PBMCs from healthy human donors (N=5) were cultured for 96 h with AVA04-251_V.2 or control antibody at the initiation of the assay together at low concentration (10 and 100 nM) with a fixed concentration of staphylococcal enterotoxin B (SEB; Toxin Technology). IL-2 levels in culture supernatants were measured by HTRF analysis (Cisbio) and compared with the basal level without test items, SEB alone. AVA04-251_V.2 increased interleukin-2 (IL-2) production in an in a dose dependant manner.

### Example 31: Tissue Distribution of AVA04-251 Fc in Humanized NOG Mice Bearing Orthotopic MDA-MB-231 Tumor Cells (FIG. 32A)

Mice (5/group) were injected with 125I-AVA04 Fc or 125I-Avelumab at 10 mg/kg (500 μCi/kg) via IV route. Mice were sacrificed at 8 h or 96 h and organs were removed and weighed. The total radioactivity contained in the biological samples were measured using a RIASTAR A5410 (Packard). Results are presented for each organ as percentage of initial dose (ID %). The ration Plasma over Tumor (FIG. 32B) shows the accumulation in the Tumor is times the level in plasma after 96 h and equivalent to Avelumab.

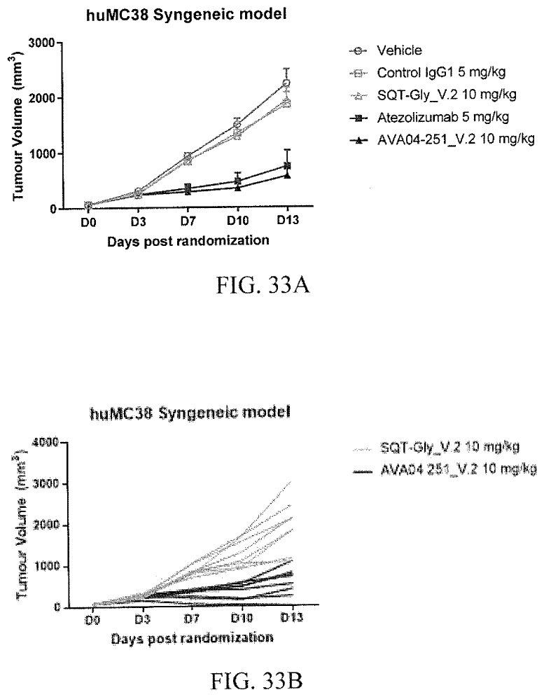

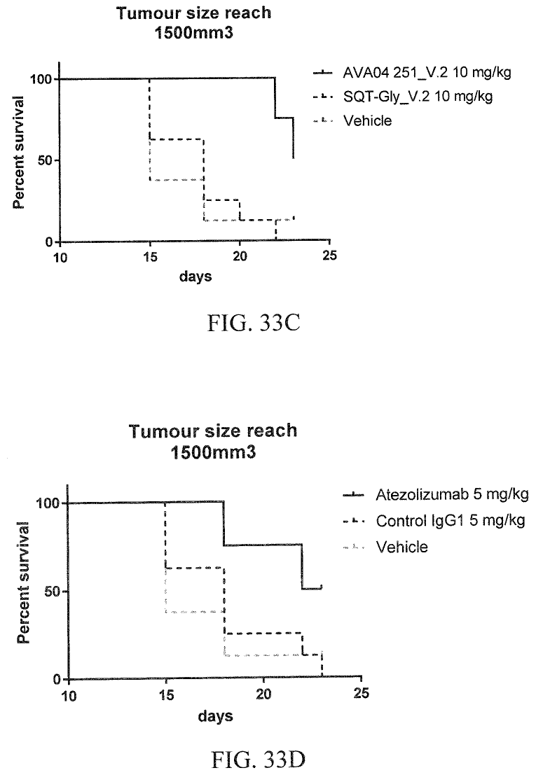

### Example 32: Tumor Growth Inhibition Following Treatment with AVA04-251_V.2 in a Humanised PD-L1 MC38 in C57/B16 Mice Syngeneic Model (FIG. 33A-33D)

Mice (n=8) were inoculated subcutaneously in the right flank region with humanized PD-L1 MC38 Tumor cell line (which have mouse PD-L1 extracellular part replaced by hPD-L1). Products and controls were injected once Tumor were ≥80 mm³. Treatment were given 2 times a week for 3 weeks at a dose of 10 mg/kg (AVA04-251_V.2 and its control SQT V.2) or 5 mg/kg (Control antibody Atezolizumab and isotype control). Overall, Tumor growth inhibition was shown for both treatments (FIG. 33A). All mice treated with AVA04-251_V.2 had a reduced Tumor size compared to the control (FIG. 33B) The survival curve at 1500 mm³ showed 50% of the AVA04-25_V.2 injected mice were still alive at 22 days after randomization (FIG. 33C). Also, the survival curve for the control antibody (Atezolizumab) (FIG. 33D) showed that 50% of the mice were alive at D22.

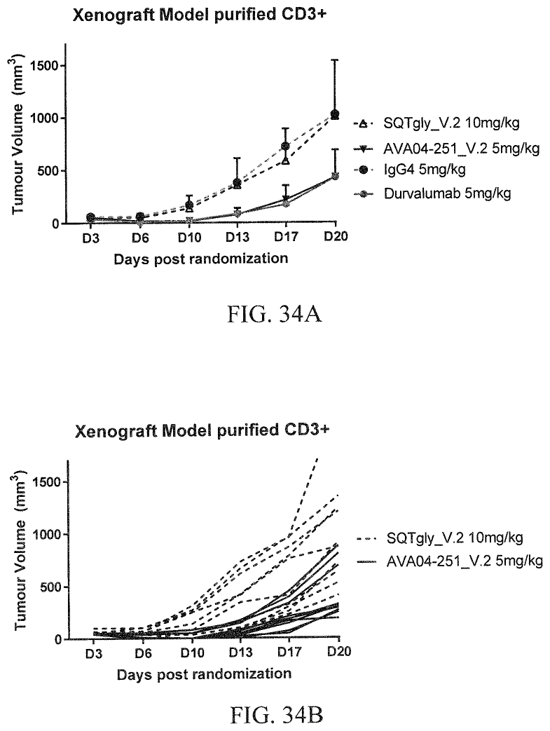

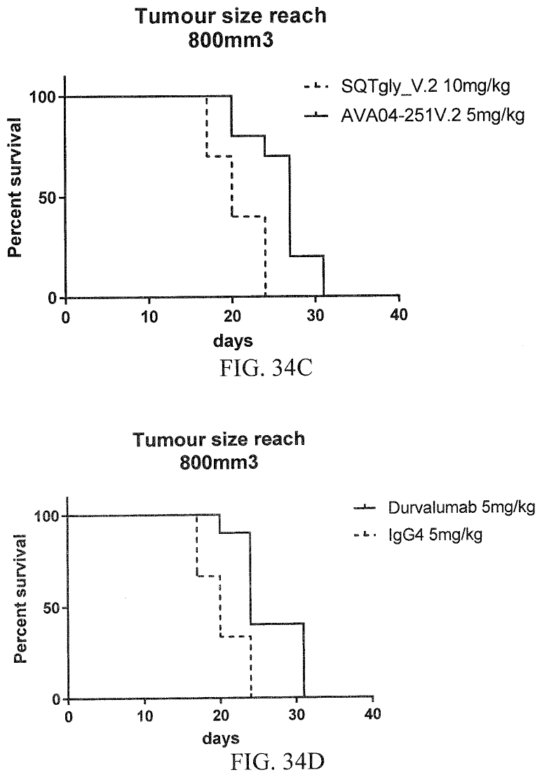

### Example 33. Tumor Growth Inhibition in Group Treated with AVA04-251_V.2 in a In Vivo Efficacy Study in the Treatment of Subcutaneous A375 Human Melanoma Humanised Model in NCG Mice. (FIG. 34A-34D)

PBMCs were isolated from two healthy donors. Total T cells were isolated and expanded on A375 cells for two rounds for 7 to 10 days in complete medium supplemented with IL-2. Mice (n=10) were inoculated subcutaneously at the right flank region with A375 tumor cell and activated T cells in 0.2 ml PBS for tumor development. The treatments started 1 h post the cell inoculation. AVA04-251_V.2 and were given 2 times a week for 3 weeks. Overall, Tumor growth inhibition was shown for both treatments when compare to controls (FIG. 34A). More than 70% of mice treated with AVA04-251_V.2 had a reduced Tumor size compared to the control (FIG. 34B) The survival curve at 800 mm³ showed that all mice treated with AVA04-25_V.2 reached 800 mm3 after 32 days after randomization where it was at 24 days for control groups (FIG. 33C). Also, the survival curve for the control antibody (Atezolizumab) (FIG. 34D). showed all the mice in the group treated with the control antibody reach a Tumor size of 800 mm³ after 32 days after randomization equally.

### Example 34. Activity of Mouse Surrogate AVA04-182 V.2 in a Mouse Allogenic Mixed Lymphocyte Reaction (MLR) Assay

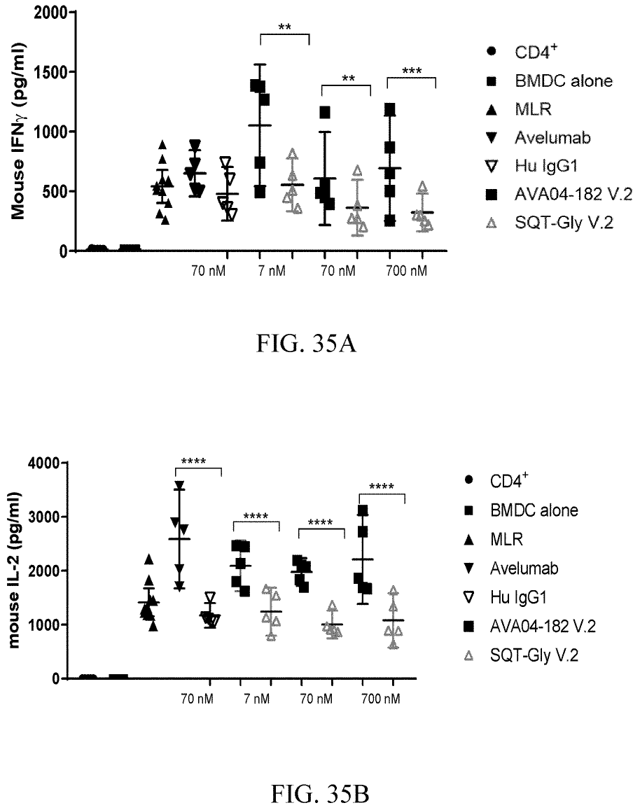

The mouse surrogate Affimer AVA04-182 V.2 that antagonizes mouse PD-L1 demonstrated the mechanism of action using bone marrow dendritic cells (BMDCs) prepared from C57/Black6 mouse. BMDCs were harvested and mixed with CD4+ T cells isolated from spleen of Balb/C mice. BMDC and CD4+ T cells were co-cultured for 4 days with each of the test molecules at 70 nM for Avelumab and the isotype control mAb and 7, 70 and 700 nM for AVA04-182 V.2 and SQT Gly V.2. Mouse IFN-γ (FIG. 35A) and IL-2 (FIG. 35B) levels in supernatants were measured by ELISA.

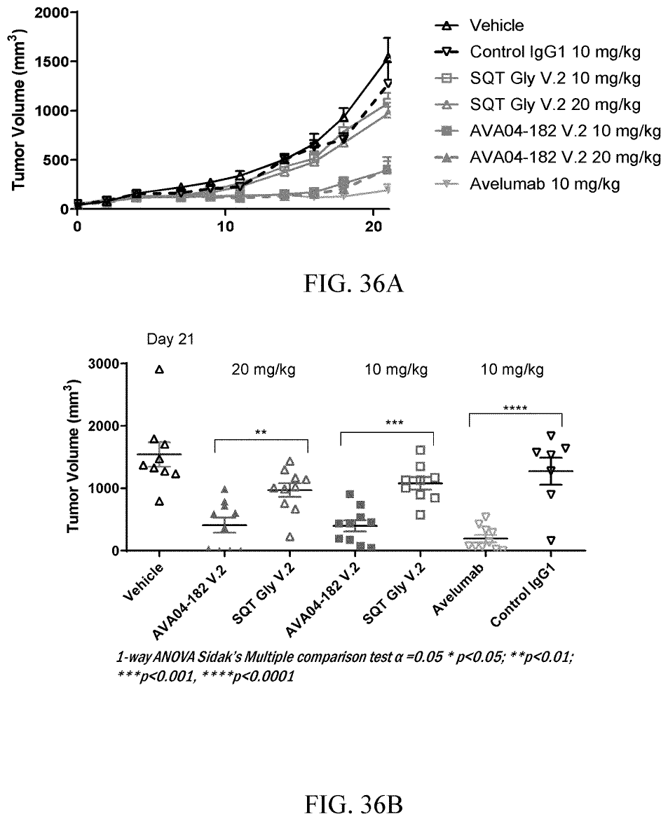

### Example 35. Tumor Growth Inhibition Using the Mouse Surogate Affimer AVA04-182 V.2 which Antagonises Mouse PD-L1. In Vivo MB49 Mouse Bladder Carcinoma Syngeneic Model using AVA04-182 V.2 and Avelumab (FIG. 36A-36B)

The MB49 Tumor cell line was inoculated subcutaneously in the right flank of the mice. Treatments were initiated when the Tumor reached 50 mm3 and were administered via IP route, bi-weekly for 3 weeks (n=10). The molecules tested were isotype mab control hIgG1 (10 mg/kg), the Affimer control SQT-Gly V.2 (10 and 20 mg/kg), the mouse PD-L1 antagonist AVA04-182 V.2 (10 and 20 mg/kg), Avelumab (10 mg/kg) and the vehicle control (PBS). The growth curves of the Tumors were measured and plotted over the course of the experiment (FIG. 36A). It was shown that AVA04-182 V.2 was able to significantly reduce the Tumor growth rate at both 10 and 20 mg/kg compared to the vehicle and Affimer control (SQT-GlyV.2) that was statistically significant (FIG. 36B).

What is claimed is:

1. A protein comprising a PD-L1 binding affimer polypeptide sequence which binds to PD-L1 with a Kd of 1×10⁻⁶M or less and inhibits interaction of the PD-L1 to which it is bound with PD-1.

2. The protein of claim 1, wherein the PD-L1 binding affimer polypeptide has an amino acid sequence represented in general formula (I)
FR1-(Xaa)ₙ-FR2-(Xaa)ₘ-FR3 (I)
wherein
FR1 is a polypeptide sequence represented by MIPGGLSEAK PATPEIQEIV DKVKPQLEEK TNETYGKLEA VQYKTQVLA (SEQ ID NO: 1) or a polypeptide sequence having at least 70% homology thereto;
FR2 is a polypeptide sequence represented by GTNYYIKVRA GDNKYMHLKV FKSL (SEQ ID NO: 2) or a polypeptide sequence having at least 70% homology thereto;
FR3 is a polypeptide sequence represented by EDLVLTGYQV DKNKDDELTG F (SEQ ID NO: 3) or a polypeptide sequence having at least 70% homology thereto; and
Xaa, individually for each occurrence, is an amino acid residue; and
n and m are each, independently, an integer from 3 to 20.

3. The protein of claim 2, wherein the PD-L1 binding affimer polypeptide has an amino acid sequence represented in the general formula:
(SEQ ID NO: 4)
MIP-Xaa1-GLSEAKPATPEIQEIVDKVKPQLEEKTNETYGKLEAVQYKTQVLA-
(Xaa)ₙ-Xaa2-TNYYIKVRAGDNKYMHLKVF-Xaa3-Xaa4-Xaa5-(Xaa)ₘ-Xaa6-D-Xaa7-
VLTGYQVDKNKDDELTGF
wherein
Xaa, individually for each occurrence, is an amino acid residue;
n and m are each, independently, an integer from 3 to 20;
Xaa1 is Gly, Ala, Val, Arg, Lys, Asp, or Glu;
Xaa2 is Gly, Ala, Val, Ser or Thr;
Xaa3 is Arg, Lys, Asn, Gln, Ser, Thr;
Xaa4 is Gly, Ala, Val, Ser or Thr;
Xaa5 is Ala, Val, Ile, Leu, Gly or Pro;
Xaa6 is Gly, Ala, Val, Asp or Glu; and
Xaa5 is Ala, Val, Ile, Leu, Arg or Lys.

4. The protein of claim 2, wherein the PD-L1 binding affimer polypeptide has an amino acid sequence represented in the general formula:
(SEQ ID NO: 5)
MIPRGLSEAKPATPEIQEIVDKVKPQLEEKTNETYGKLEAVQYKT
QVLA-(Xaa)ₙ-STNYYIKVRAGDNKYMHLKVFNGP-(Xaa)ₘ-ADR
VLTGYQVDKNKDDELTGF
wherein
Xaa, individually for each occurrence, is an amino acid residue; and
n and m are each, independently, an integer from 3 to 20.

5. The protein of claim 2, 3 or 4, wherein (Xaa)ₙ is an amino acid sequence represented in the general formula (II)
aa1-aa2-aa3-Gly-Pro-aa4-aa5-Trp-aa6- (II)
wherein
aa1 represents an amino acid residue with a basic sidechain;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain;
aa3 represents an amino acid residue with an aromatic or basic sidechain;
aa4 represents an amino acid residue with a neutral polar or non-polar sidechain or a charged (acidic or basic) sidechain; preferably a neutral polar sidechain or a charged (acidic or basic) sidechain;
aa5 represents an amino acid residue with a neutral polar or a charged (acidic or basic) or a small aliphatic or an aromatic sidechain; preferably a neutral polar sidechain or a charged sidechain; and
aa6 represents an amino acid residue with an aromatic or acid sidechain.

6. The protein of claim 2, 3 or 4, wherein (Xaa)ₙ is an amino acid sequence represented in the general formula (III)
aa1-aa2-aa3-Phe-Pro-aa4-aa5-Phe-Trp- (III)
wherein
aa1 represents an amino acid residue with a basic sidechain or aromatic sidechain;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain;
aa3 represents an amino acid residue with an aromatic or basic sidechain, preferably Phe, Tyr, Trp, Lys, Arg or His, more preferably Phe, Tyr, Trp or His, and even more preferably Tyr, Trp or His;
aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu; and
aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg.

7. The protein of any of claim 2, 3 or 4, wherein (Xaa)ₙ is an amino acid sequence selected from SEQ ID NOS: 6 to 41, or an amino acid sequence having at least 80% homology thereto.

8. The protein of any of claim 2, 3 or 4, wherein (Xaa)ₙ is an amino acid sequence selected from SEQ ID NOS: 6 to 41, or an amino acid sequence having at least 80% identity thereto.

9. The protein of any of claims 2-8, wherein (Xaa)ₘ is an amino acid sequence represented in the general formula (IV)
-aa7-aa8-aa9-aa10-aa11-aa12-aa13-aa14-aa15- (IV)
wherein
aa7 represents an amino acid residue with neutral polar or non-polar sidechain or an acidic sidechain;
aa8 represents an amino acid residue, preferably an amino acid residue with a neutral polar or non-polar sidechain or a charged (acidic or basic) sidechain or aromatic sidechain, more preferably a charged (acidic or basic) sidechain;
aa9 represents an amino acid residue, preferably an amino acid residue with a neutral polar or non-polar sidechain or a charged (acidic or basic) sidechain or aromatic sidechain, more preferably a neutral polar side chain or an acid side chain;
aa10 represents an amino acid residue, preferably an amino acid residue with a neutral polar or non-polar sidechain or a charged (acidic or basic) sidechain or aromatic sidechain, more preferably a neutral polar side chain or a basic or acid side chain;
aa11 represents an amino acid residue, preferably an amino acid residue with a neutral polar sidechain or a charged (acidic or basic) sidechain or a nonpolar aliphatic sidechain or an aromatic sidechain, more preferably a neutral polar side chain or a basic or acid side chain;
aa12 represents an amino acid residue, preferably an amino acid residue with a neutral polar sidechain or a charged (acidic or basic) sidechain or a nonpolar aliphatic sidechain or an aromatic sidechain, more preferably an acid side chain;
aa13 represents an amino acid residue, preferably an amino acid residue with a neutral polar sidechain or a charged (acidic or basic) sidechain or a nonpolar aliphatic sidechain or an aromatic sidechain, more preferably an acid side chain;
aa14 represents an amino acid residue, preferably an amino acid residue with a neutral polar sidechain or a charged (acidic or basic) sidechain; and
aa15 represents an amino acid residue, preferably an amino acid residue with a neutral polar or neutral non-polar sidechain or a charged (acidic or basic) sidechain.

10. The protein of any of claims 2-8, wherein (Xaa)ₘ is an amino acid sequence selected from SEQ ID NOS: 42 to 77, or an amino acid sequence having at least 80% homology thereto.

11. The protein of any of claims 2-8, wherein (Xaa)ₘ is an amino acid sequence selected from SEQ ID NOS: 42 to 77, or an amino acid sequence having at least 80% identity thereto.

12. The protein of any of the preceding claims, wherein the PD-L1 binding affimer polypeptide has an amino acid sequence selected from SEQ ID NOS: 78 to 86, or an amino acid sequence having at least 70% homology thereto.

13. The protein of any of the preceding claims, wherein the PD-L1 binding affimer polypeptide has an amino acid sequence selected from SEQ ID NOS: 78 to 86, or an amino acid sequence having at least 70% identity thereto.

14. The protein of any of the preceding claims, wherein the PD-L1 binding affimer polypeptide has an amino acid sequence that can be encoded by a nucleic acid having a coding sequence corresponding to nucleotides 1-336 of one of SEQ ID NOS: 87 to 94, or a coding sequence at least 70% identical thereto.

15. The protein of any of the preceding claims, wherein the PD-L1 binding affimer polypeptide has an amino acid sequence that can be encoded by a nucleic acid comprising a coding sequence that hybridizes to any one of SEQ ID NOS: 87 to 94 under stringent conditions of 6× sodium chloride/sodium citrate (SSC) at 45° C. followed by a wash in 0.2×SSC at 65° C.

16. The protein of any of the preceding claims, binding the protein binds PD-L1 is competitive with PD-L1 binding by anti-PD-L1 antibodies Atezolizumab, Avelumab and/or Durvalumab.

17. The protein of any of the preceding claims, wherein the PD-L1 binding affimer polypeptide forms a crystal structure with PD-L1 comprising an interface involving at least 10 residues of PD-L1 selected from Ile-54, Tyr-56, Glu-58, Glu-60, Asp-61, Lys-62, Asn-63, Gln 66, Val-68, Val-76, Val-111, Arg-113, Met-115, Ile-116, Ser-117, Gly-120, Ala-121, Asp-122, Tyr-123, and Arg-125.

18. The protein of any of the preceding claims, binding to PD-L1 (a) increases T-cell proliferation in a mixed lymphocyte reaction (MLR) assay; (b) increases interferon-γ production in an MLR assay; and/or (c) increases interleukin-2 (IL-2) secretion in an MLR assay.

19. The protein of any of the preceding claims, which is a fusion protein comprising one or more additional amino acid sequences selected from the group consisting of: secretion signal sequences, peptide linker sequences, affinity tags, transmembrane domains, cell surface retention sequence, substrate recognition sequences for post-translational modifications, multimerization domains to create multimeric structures of the protein aggregating through protein-protein interactions, half-life extending polypeptide moieties, polypeptide sequences for altering tissue localization and antigen binding site of an antibody, and one or more additional affimer polypeptide sequences binding the PD-L1 or a different target.

20. The protein of claim 19, which is a fusion protein comprising a half-life extending polypeptide moieties selected from the group consisting of an Fc domain or portion thereof, an albumin protein or portion thereof, an albumin-binding polypeptide moiety, transferrin or portion thereof, a transferrin-binding polypeptide moiety, fibronectin or portion thereof, or a fibronectin-binding polypeptide moiety.

21. The protein of claim 20, wherein the Fc domain or a portion thereof retains FcRn binding.

22. The protein of claim 20, wherein the Fc domain or a portion thereof is from IgA, IgD, IgE, IgG, and IgM or a subclass (isotype) thereof such as IgG1, IgG2, IgG3, IgG4, IgA1 or IgA2.

23. The protein of claim 20, wherein the Fc domain or a portion thereof retains effector function selected from C1q binding, complement dependent cytotoxicity (CDC), antibody-dependent cell-mediated cytotoxicity (ADCC); phagocytosis; down regulation of B cell receptor, or a combination thereof.

24. The protein of claim 20, wherein the half-life extending polypeptide moiety increases the serum half-life of the protein by at least 5-fold relative to its absence from the protein.

25. The protein of claim 22, comprising an amino acid sequence of SEQ ID NO: 112 or SEQ ID NO: 113 or a sequence having at least 70% homology thereto.

26. A recombinant antibody comprising one or more V_{H} and/or V_{L} chains forming one or more antigen binding sites that bind to a target antigen, wherein at least one of the V_{H} and/or V_{L} chains is a fusion protein also including at least one PD-L1 binding affimer polypeptide sequences that binds PD-L1 with a Kd of 1×10⁻⁶M or less and inhibits interaction of PD-1 with the PD-L1 to which it is bound.

27. The recombinant antibody of claim 26, wherein the V_{H} chain includes an Fc domain.

28. The recombinant antibody of claim 26, wherein the target antigen is an immune checkpoint.

29. The recombinant antibody of claim 26, wherein the target antigen is a immune costimulatory receptor and the chimeric antibody agonizes the costimulatory receptor on binding.

30. The recombinant antibody of claim 26, wherein the target antigen is an angiogenic factor or a receptor therefore and the chimeric antibody antagonizes the angiogenic factor or receptor therefore.

31. The recombinant antibody of claim 26, wherein the target antigen is a tumor antigen.

32. The recombinant antibody of claim 26, wherein the target antigen is a soluble immunosuppressive factor or a receptor therefore, and the chimeric antibody inhibits the immunosuppressive activity of the immunosuppressive factor to act as an immunostimulatory signal.

33. The recombinant antibody of claim 26, wherein the target antigen is selected from the group consisting of PD-1, PD-L2, CTLA-4, NKG2A, KIR, LAG-3, TIM-3, CD96, VISTA, TIGIT, CD28, ICOS, CD137, OX40, GITR, CD27, CD30, HVEM, DNAM-1 or CD28H, CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX40L, PVR, SIRP□, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DL5A, KIR2DL5B, KIR3DL1, KIR3DL2, KIR3DL3, NKG2A, NKG2C, NKG2E or TSLP.

34. A recombinant Affimer-Ipilimumab antibody fusion protein comprising
(i) an affimer-heavy chain fusion protein (wherein the secretion signal sequence MPLLLLLPLLWAGALA (SEQ ID NO: 136) is optionally removed) with an amino acid sequence of SEQ ID NO: 112 or a sequence having at least 70% homology thereto, and
(ii) a light chain protein (wherein the secretion signal sequence MPLLLLLPLLWAGALA (SEQ ID NO: 136) is optionally removed) with an amino acid sequence of SEQ ID NO: 113 or a sequence having at least 70% homology thereto.

35. A recombinant Affimer-Bevacizumab antibody fusion protein comprising
(i) an affimer-heavy chain fusion protein (wherein the secretion signal sequence MPLLLLLPLLWAGALA (SEQ ID NO: 136) is optionally removed) with an amino acid sequence of SEQ ID NO: 116 or 118 or a sequence having at least 70% homology thereto, and
(ii) a light chain protein (wherein the secretion signal sequence MPLLLLLPLLWAGALA (SEQ ID NO: 136) is optionally removed) with an amino acid sequence of SEQ ID NO: 116 or a sequence having at least 70% homology thereto.

36. A recombinant receptor trap fusion protein comprising (i) a ligand binding domain of a receptor, and (ii) a PD-L1 binding affimer polypeptide sequence(s) which binds to PD-L1 with a Kd of 1×10⁻⁶M or less and inhibits interaction of PD-1 to the PD-L1 to which it is bound.

37. The recombinant receptor trap fusion protein of claim 36, wherein the binding domain binds to PGE2, TGF-β, VEGF, CCL2, IDO, CSF1, IL-10, IL-13, IL-23, or adenosine.

38. The recombinant receptor trap fusion protein of claim 36, further including an multimerization domain that induces multimerization of the recombinant receptor trap fusion protein.

39. A recombinant receptor ligand fusion protein comprising (i) a polypeptide ligand sequence that binds to an agonizes or antagonizes its cognate receptor, and (ii) a PD-L1 binding affimer polypeptide sequence(s) which binds to PD-L1 with a Kd of 1×10⁻⁶M or less and inhibits interaction of PD-1 to the PD-L1 to which it is bound.

40. The recombinant receptor ligand fusion protein of claim 39, wherein the polypeptide ligand is a ligand for a co-stimulatory receptor and agonizes the co-stimulatory receptor upon binding.

41. The recombinant receptor ligand fusion protein of claim 40, wherein the polypeptide ligand is selected from B7.1, 4-1BBL, OX40L, GITRL or LIGHT.

42. The recombinant receptor ligand fusion protein of claim 39, further including an multimerization domain that induces multimerization of the recombinant receptor ligand fusion protein.

43. The recombinant receptor ligand fusion protein of claim 39, wherein the polypeptide ligand is an immunostimulatory cytokine that promotes antitumor immunity.

44. The recombinant receptor ligand fusion protein of claim 43, wherein the polypeptide ligand is selected from IFN-α2, IL-2, IL-15, IL-21, and IL-12.

45. A multispecific T-cell engaging fusion protein comprising (i) a CD3 binding polypeptide binds to CD3 on the surface of T-cells, and (ii) a PD-L1 binding affimer polypeptide sequence(s) which binds to PD-L1 with a Kd of 1×10⁻⁶M or less and inhibits interaction of PD-1 to the PD-L1 to which it is bound.

46. A chimeric receptor fusion protein comprising (i) an extracellular portion including a PD-L1 binding affimer polypeptide sequence(s) which binds to PD-L1 with a Kd of 1×10⁻⁶M or less and inhibits interaction of PD-1 to the PD-L1 to which it is bound; (ii) a transmembrane domain; and (c) a cytoplasmic domain comprising a 4-1BB signaling domain and a CDR signaling domain, and optional a costimulatory signaling region.

47. A nucleic acid comprising a coding sequence encoding a protein of any of claims 1-46.

48. The nucleic acid of claim 47, wherein the coding sequence is operably linked to one or more transcriptional regulatory sequences, such as a promoter and/or enhancer.

49. The nucleic acid of claim 47, including one or more origins of replication, minichromosome maintenance elements (MME) and/or nuclear localization elements.

50. The nucleic acid of claim 47, including a polyadenylation signal sequence which is operably linked and transcribed with the coding sequence.

51. The nucleic acid of claim 47, wherein the coding sequence includes one or more intronic sequences

52. The nucleic acid of claim 47, including one or more ribosome binding sites which are transcribed with the coding sequence.

53. The nucleic acid of claim 47, which is DNA.

54. The nucleic acid of claim 47, which is RNA.

55. A viral vector including the nucleic acid of claim 47.

56. Plasmid DNA, Plasmid Vector or Minicircle including the nucleic acid of claim 47.

57. An antibody or antigen binding fragment thereof further comprising a PD-L1 binding affimer polypeptide conjugated thereto.

58. A soluble receptor or ligand binding domain thereof further comprising a PD-L1 binding affimer polypeptide conjugated thereto.

59. A growth factor, cytokine or chemokine biologically active polypeptide fragment thereof further comprising a PD-L1 binding affimer polypeptide conjugated thereto.

60. A costimulatory agonist polypeptide further comprising a PD-L1 binding affimer polypeptide conjugated thereto.

61. A checkpoint inhibitory polypeptide further comprising a PD-L1 binding affimer polypeptide conjugated thereto.

62. An Affimer Agent comprising a PD-L1 binding affimer polypeptide and a detectable label, a toxin or one or more therapeutic agents conjugated thereto.

63. A pharmaceutical preparation suitable for therapeutic use in a human patient, comprising (i) a recombinant protein of any of claims 1-25, a recombinant antibody of any of claims 26-35, a recombinant receptor trap fusion protein of any of claims 36-38, a recombinant receptor ligand fusion of any of claims 39-44, a multispecific T-cell engaging fusion protein of claim 45, a chimeric receptor fusion protein of claim 46, an antibody of claim 57, a soluble receptor of claim 58, a growth factor, cytokine or chemokine of claim 59, a costimulatory agonist polypeptide of claim 60, a checkpoint inhibitory polypeptide of claim 61 or an Affimer Agent of claim 62, and (ii) one or more pharmaceutically acceptable excipients, buffers, salts or the like.

64. A pharmaceutical preparation suitable for therapeutic gene delivery in a human patient, comprising a nucleic acid of any of claims 47-54, a viral vector of claim 55 or plasmid DNA, plasmid Vector or minicircle of claim 56, and (ii) one or more pharmaceutically acceptable excipients, buffers, salts, transfection enhancers, electroporation enhancers or the like.

65. A method comprising administering to a subject the protein, recombinant antibody, or nucleic acid of any one of the foregoing claims.

66. The method of claim 65, wherein the subject comprises cancer cells that express PD-L1, optionally wherein the cancer cells are melanoma cells.

67. The method of claim 65 or 66, wherein the protein, recombinant antibody, or nucleic acid is administered in an effective amount to elicit a mixed lymphocyte reaction.

68. The method of claim 67, wherein the protein, recombinant antibody, or nucleic acid is administered in an effective amount to increase IFNγ production by T cells in the subject by at least 2-fold, relative to a vehicle-only control.

69. The method of any one of claims 66-68, wherein the subject has a tumor comprising the cancer cells that express PD-L1, and the level of PD-L1 binding affimer polypeptide accumulation in the tumor is at least 5 times the level in plasma at 96 hours post-administration.

70. The method of any one of claims 66-68, wherein the subject has a tumor comprising the cancer cells that express PD-L1, and the protein, recombinant antibody, or nucleic acid is administered in an effective amount to inhibit growth of the tumor in the subject by at least 10%.

71. The method of any one of claims 65-70, wherein the subject has a melanoma.


SEQUENCE LISTING (209 sequences)

SEQ ID NO: 1 - protein, 49 aa, Artificial Sequence
  Synthetic polypeptide
MIPGGLSEAKPATPEIQEIVDKVKPQLEEKTNETYGKLEAVQYKTQVLA

SEQ ID NO: 2 - protein, 24 aa, Artificial Sequence
  Synthetic polypeptide
GTNYYIKVRAGDNKYMHLKVFKSL

SEQ ID NO: 3 - protein, 21 aa, Artificial Sequence
  Synthetic polypeptide
EDLVLTGYQVDKNKDDELTGF

SEQ ID NO: 4 - protein, 134 aa, Artificial Sequence
  Synthetic polypeptide
  misc_feature (4)..(4): Xaa is Gly, Ala, Val, Arg, Lys, Asp, or Glu
  misc_feature (50)..(69): Xaa can be any naturally occurring amino acid
  misc_feature (53)..(69): May be absent
  misc_feature (70)..(70): Xaa is Gly, Ala, Val, Ser or Thr
  misc_feature (91)..(91): Xaa is Arg, Lys, Asn, Gln, Ser, Thr
  misc_feature (92)..(92): Xaa is Gly, Ala, Val, Ser or Thr
  misc_feature (93)..(93): Xaa is Ala, Val, Ile, Leu, Gly or Pro
  misc_feature (94)..(113): Xaa can be any naturally occurring amino acid
  misc_feature (97)..(113): May be absent
  misc_feature (114)..(114): Xaa is Gly, Ala, Val, Asp or Glu
  misc_feature (116)..(116): Xaa is Ala, Val, Ile, Leu, Arg or Lys
MIPXGLSEAKPATPEIQEIVDKVKPQLEEKTNETYGKLEAVQYKTQVLAXXXXXXXXXXXXXXXXXXXXXTNYYIKVRAGDNKYMHLKVFXXXXXXXXXXXXXXXXXXXXXXXXDXVLTGYQVDKNKDDELTGF

SEQ ID NO: 5 - protein, 134 aa, Artificial Sequence
  Synthetic polypeptide
  misc_feature (50)..(69): Xaa can be any naturally occurring amino acid
  misc_feature (53)..(69): May be absent
  misc_feature (94)..(113): Xaa can be any naturally occurring amino acid
  misc_feature (97)..(113): Xaa can be any naturally occurring amino acid
MIPRGLSEAKPATPEIQEIVDKVKPQLEEKTNETYGKLEAVQYKTQVLAXXXXXXXXXXXXXXXXXXXXSTNYYIKVRAGDNKYMHLKVFNGPXXXXXXXXXXXXXXXXXXXXADRVLTGYQVDKNKDDELTGF

SEQ ID NO: 6 - protein, 9 aa, Artificial Sequence
  Synthetic polypeptide
KAWGPKQWW

SEQ ID NO: 7 - protein, 9 aa, Artificial Sequence
  Synthetic polypeptide
KPYGPRDWD

SEQ ID NO: 8 - protein, 9 aa, Artificial Sequence
  Synthetic polypeptide
KEYGPEEWW

SEQ ID NO: 9 - protein, 9 aa, Artificial Sequence
  Synthetic polypeptide
HAYGPRDWD

SEQ ID NO: 10 - protein, 9 aa, Artificial Sequence
  Synthetic polypeptide
KDHGPIAWW

SEQ ID NO: 11 - protein, 9 aa, Artificial Sequence
  Synthetic polypeptide
NKHFHQRFW

SEQ ID NO: 12 - protein, 9 aa, Artificial Sequence
  Synthetic polypeptide
NKHFPIHFW

SEQ ID NO: 13 - protein, 9 aa, Artificial Sequence
  Synthetic polypeptide
HEFGPAEWD

SEQ ID NO: 14 - protein, 9 aa, Artificial Sequence
  Synthetic polypeptide
NAHFPQSFW

SEQ ID NO: 15 - protein, 9 aa, Artificial Sequence
  Synthetic polypeptide
KEHGPDSWW

SEQ ID NO: 16 - protein, 9 aa, Artificial Sequence
  Synthetic polypeptide
NQHFPHSFW

SEQ ID NO: 17 - protein, 9 aa, Artificial Sequence
  Synthetic polypeptide
NAHFGPRFW

SEQ ID NO: 18 - protein, 9 aa, Artificial Sequence
  Synthetic polypeptide
NTWFPESFW

SEQ ID NO: 19 - protein, 9 aa, Artificial Sequence
  Synthetic polypeptide
NQHFPQSFW

SEQ ID NO: 20 - protein, 9 aa, Artificial Sequence
  Synthetic polypeptide
KQYGPDDWW

SEQ ID NO: 21 - protein, 9 aa, Artificial Sequence
  Synthetic polypeptide
KDWGPSNWW

SEQ ID NO: 22 - protein, 9 aa, Artificial Sequence
  Synthetic polypeptide
KQFGPKDWW

SEQ ID NO: 23 - protein, 9 aa, Artificial Sequence
  Synthetic polypeptide
NHHFPKRFW

SEQ ID NO: 24 - protein, 8 aa, Artificial Sequence
  Synthetic polypeptide
YRHFPQWH

SEQ ID NO: 25 - protein, 9 aa, Artificial Sequence
  Synthetic polypeptide
NIHFPPNFW

SEQ ID NO: 26 - protein, 8 aa, Artificial Sequence
  Synthetic polypeptide
YTHFPQWT

SEQ ID NO: 27 - protein, 9 aa, Artificial Sequence
  Synthetic polypeptide
NDHFPHTFW

SEQ ID NO: 28 - protein, 9 aa, Artificial Sequence
  Synthetic polypeptide
NQHFPSYFW

SEQ ID NO: 29 - protein, 9 aa, Artificial Sequence
  Synthetic polypeptide
NQYFPPHFW

SEQ ID NO: 30 - protein, 9 aa, Artificial Sequence
  Synthetic polypeptide
KKHFPASFW

SEQ ID NO: 31 - protein, 9 aa, Artificial Sequence
  Synthetic polypeptide
KKFFPKHFW

SEQ ID NO: 32 - protein, 9 aa, Artificial Sequence
  Synthetic polypeptide
KLHFPRSFW

SEQ ID NO: 33 - protein, 9 aa, Artificial Sequence
  Synthetic polypeptide
YKHFPPNFW

SEQ ID NO: 34 - protein, 9 aa, Artificial Sequence
  Synthetic polypeptide
EEHFPFQFW

SEQ ID NO: 35 - protein, 9 aa, Artificial Sequence
  Synthetic polypeptide
KPHFPDNFW

SEQ ID NO: 36 - protein, 9 aa, Artificial Sequence
  Synthetic polypeptide
YQYFPDQFN

SEQ ID NO: 37 - protein, 9 aa, Artificial Sequence
  Synthetic polypeptide
VQWFPRSFW

SEQ ID NO: 38 - protein, 9 aa, Artificial Sequence
  Synthetic polypeptide
AAHFPEHFW

SEQ ID NO: 39 - protein, 9 aa, Artificial Sequence
  Synthetic polypeptide
REGRQDWVL

SEQ ID NO: 40 - protein, 9 aa, Artificial Sequence
  Synthetic polypeptide
WVPFPHQQL

SEQ ID NO: 41 - protein, 9 aa, Artificial Sequence
  Synthetic polypeptide
YNHFPEYMW

SEQ ID NO: 42 - protein, 5 aa, Artificial Sequence
  Synthetic polypeptide
GRTIQ

SEQ ID NO: 43 - protein, 9 aa, Artificial Sequence
  Synthetic polypeptide
EPQLDTSPI

SEQ ID NO: 44 - protein, 9 aa, Artificial Sequence
  Synthetic polypeptide
GDYEQVLIH

SEQ ID NO: 45 - protein, 9 aa, Artificial Sequence
  Synthetic polypeptide
PADHVLEEA

SEQ ID NO: 46 - protein, 9 aa, Artificial Sequence
  Synthetic polypeptide
EDTNTDGAL

SEQ ID NO: 47 - protein, 9 aa, Artificial Sequence
  Synthetic polypeptide
GQSWDQRRQ

SEQ ID NO: 48 - protein, 9 aa, Artificial Sequence
  Synthetic polypeptide
SKSPIDLPF

SEQ ID NO: 49 - protein, 9 aa, Artificial Sequence
  Synthetic polypeptide
DPQDVYLNQ

SEQ ID NO: 50 - protein, 9 aa, Artificial Sequence
  Synthetic polypeptide
GSLHSFGST

SEQ ID NO: 51 - protein, 9 aa, Artificial Sequence
  Synthetic polypeptide
QEKNQWVEE

SEQ ID NO: 52 - protein, 9 aa, Artificial Sequence
  Synthetic polypeptide
QKNYEEDPH

SEQ ID NO: 53 - protein, 9 aa, Artificial Sequence
  Synthetic polypeptide
WDGHKRFAD

SEQ ID NO: 54 - protein, 9 aa, Artificial Sequence
  Synthetic polypeptide
DDNQERQEH

SEQ ID NO: 55 - protein, 9 aa, Artificial Sequence
  Synthetic polypeptide
AVTQEDQAV

SEQ ID NO: 56 - protein, 9 aa, Artificial Sequence
  Synthetic polypeptide
EVDWKYQDH

SEQ ID NO: 57 - protein, 9 aa, Artificial Sequence
  Synthetic polypeptide
VDDKTLSKD

SEQ ID NO: 58 - protein, 9 aa, Artificial Sequence
  Synthetic polypeptide
QGQGKDPSQ

SEQ ID NO: 59 - protein, 9 aa, Artificial Sequence
  Synthetic polypeptide
GHQSEVQHS

SEQ ID NO: 60 - protein, 9 aa, Artificial Sequence
  Synthetic polypeptide
TGTSIWNQD

SEQ ID NO: 61 - protein, 11 aa, Artificial Sequence
  Synthetic polypeptide
GVHDSLQGYDA

SEQ ID NO: 62 - protein, 9 aa, Artificial Sequence
  Synthetic polypeptide
QKGQKIDKF

SEQ ID NO: 63 - protein, 9 aa, Artificial Sequence
  Synthetic polypeptide
DDELHDTRH

SEQ ID NO: 64 - protein, 9 aa, Artificial Sequence
  Synthetic polypeptide
ATTGDEWDR

SEQ ID NO: 65 - protein, 9 aa, Artificial Sequence
  Synthetic polypeptide
SHPHSNHTS

SEQ ID NO: 66 - protein, 9 aa, Artificial Sequence
  Synthetic polypeptide
WRTDYKYEE

SEQ ID NO: 67 - protein, 9 aa, Artificial Sequence
  Synthetic polypeptide
NDPHDSVPH

SEQ ID NO: 68 - protein, 9 aa, Artificial Sequence
  Synthetic polypeptide
GQQRENEQE

SEQ ID NO: 69 - protein, 9 aa, Artificial Sequence
  Synthetic polypeptide
GERQQDDAN

SEQ ID NO: 70 - protein, 9 aa, Artificial Sequence
  Synthetic polypeptide
AYREGSQWT

SEQ ID NO: 71 - protein, 9 aa, Artificial Sequence
  Synthetic polypeptide
EFYDHGIIQ

SEQ ID NO: 72 - protein, 9 aa, Artificial Sequence
  Synthetic polypeptide
ENEATRDQH

SEQ ID NO: 73 - protein, 9 aa, Artificial Sequence
  Synthetic polypeptide
GYDHEDNRG

SEQ ID NO: 74 - protein, 9 aa, Artificial Sequence
  Synthetic polypeptide
QPADMSAEF

SEQ ID NO: 75 - protein, 9 aa, Artificial Sequence
  Synthetic polypeptide
WVPFPHQQL

SEQ ID NO: 76 - protein, 9 aa, Artificial Sequence
  Synthetic polypeptide
REGRQDWVL

SEQ ID NO: 77 - protein, 9 aa, Artificial Sequence
  Synthetic polypeptide
PPRFPHEPF

SEQ ID NO: 78 - protein, 112 aa, Artificial Sequence
  Synthetic polypeptide
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGF

SEQ ID NO: 79 - protein, 112 aa, Artificial Sequence
  Synthetic polypeptide
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGF

SEQ ID NO: 80 - protein, 112 aa, Artificial Sequence
  Synthetic polypeptide
MIPRGLSEAKPATPEIQEIVDKVKPQLEEKTNETYGKLEAVQYKTQVLAKDHGPIAWWSTNYYIKVRAGDNKYMHLKVFNGPEDTNTDGALADRVLTGYQVDKNKDDELTGF

SEQ ID NO: 81 - protein, 112 aa, Artificial Sequence
  Synthetic polypeptide
MIPRGLSEAKPATPEIQEIVDKVKPQLEEKTNETYGKLEAVQYKTQVLAKDWGPSNWWSTNYYIKVRAGDNKYMHLKVFNGPVDDKTLSKDADRVLTGYQVDKNKDDELTGF

SEQ ID NO: 82 - protein, 112 aa, Artificial Sequence
  Synthetic polypeptide
MIPRGLSEAKPATPEIQEIVDKVKPQLEEKTNETYGKLEAVQYKTQVLANTWFPESFWSTNYYIKVRAGDNKYMHLKVFNGPDDNQERQEHADRVLTGYQVDKNKDDELTGF

SEQ ID NO: 83 - protein, 112 aa, Artificial Sequence
  Synthetic polypeptide
MIPRGLSEAKPATPEIQEIVDKVKPQLEEKTNETYGKLEAVQYKTQVLAKPYGPRDWDSTNYYIKVRAGDNKYMHLKVFNGPEPQLDTSPIADRVLTGYQVDKNKDDELTGF

SEQ ID NO: 84 - protein, 112 aa, Artificial Sequence
  Synthetic polypeptide
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGF

SEQ ID NO: 85 - protein, 112 aa, Artificial Sequence
  Synthetic polypeptide
MIPRGLSEAKPATPEIQEIVDKVKPQLEEKTNETYGKLEAVQYKTQVLAAAHFPEHFWSTNYYIKVRAGDNKYMHLKVFNGPQPADMSAEFADRVLTGYQVDKNKDDELTGF

SEQ ID NO: 86 - protein, 112 aa, Artificial Sequence
  Synthetic polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 87 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCAAAAGATTGGGGTCCATCTAACTGGTGGTCCACCAACTATTACATTAAGGTTCGTGCCGGTGACAATAAGTATATGCACCTGAAAGTGTTCAACGGCCCGGTTGATGATAAAACCCTGTCTAAAGATGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 88 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCAAAAGATCATGGTCCAATCGCATGGTGGTCCACCAACTATTACATTAAGGTTCGTGCCGGTGACAATAAGTATATGCACCTGAAAGTGTTCAACGGCCCGGAAGATACCAACACCGATGGTGCACTGGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 89 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCAAAACCATACGGTCCACGTGATTGGGATTCCACCAACTATTACATTAAGGTTCGTGCCGGTGACAATAAGTATATGCACCTGAAAGTGTTCAACGGCCCGGAACCACAGCTGGATACCTCTCCAATCGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 90 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCAAACACCTGGTTTCCAGAATCTTTTTGGTCCACCAACTATTACATTAAGGTTCGTGCCGGTGACAATAAGTATATGCACCTGAAAGTGTTCAACGGCCCGGATGATAACCAGGAACGTCAGGAACATGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 91 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCACGTGAAGGTCGTCAGGATTGGGTTCTGTCCACCAACTATTACATTAAGGTTCGTGCCGGTGACAATAAGTATATGCACCTGAAAGTGTTCAACGGCCCGTGGGTTCCATTTCCACATCAGCAGCTGGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 92 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCACATGCATACGGTCCACGTGATTGGGATTCCACCAACTATTACATTAAGGTTCGTGCCGGTGACAATAAGTATATGCACCTGAAAGTGTTCAACGGCCCGCCAGCAGATCATGTTCTGGAAGAAGCAGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 93 - protein, 369 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCAAAAGAATACGGTCCAGAAGAATGGTGGTCCACCAACTATTACATTAAGGTTCGTGCCGGTGACAATAAGTATATGCACCTGAAAGTGTTCAACGGCCCGGGTGATTACGAACAGGTTCTGATCCATGCGGACCGTGTTCTGACCGGTTACCAGGTTGACAAGAACAAAGATGACGAGCTGACGGGTTTCGCGGCCGCGGGTCATCACCACCACCACCATTAG

SEQ ID NO: 94 - protein, 371 aa, Artificial Sequence
  Synthetic polypeptide
ATGATCCCGCGTGGCCTGTCTGAAGCTAAACCAGCAACTCCGGAAATTCAAGAGATCGTCGATAAGGTGAAACCGCAGCTGGAAGAGAAAACGAACGAAACCTACGGTAAGCTGGAAGCGGTCCAGTACAAAACCCAAGTGCTAGCAGCTGCTCATTTCCCGGAACATTTCTGGTCCACCAACTATTACATTAAGGTTCGTGCCGGTGACAATAAGTATATGCACCTGAAAGTGTTCAACGGCCCGCAGCCGGCTGATATGTCTGCTGAATTCGCGGACCGTGTTCTGACCGGTTACCAGGTTGACAAGAACAAAGATGACGAGCTGACGGGTTTCCTGCAGGCGGCCGCGCACCACCACCACCACCACTG

SEQ ID NO: 95 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 227 aa, Artificial Sequence; identical to SEQ ID NO: 95
  Synthetic polypeptide

SEQ ID NO: 97 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 98 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 227 aa, Artificial Sequence
  Synthetic polypeptide
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 232 aa, Artificial Sequence
  Synthetic polypeptide
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 226 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 30 aa, Artificial Sequence
  Synthetic polypeptide
GGGGSGGGGSGGGGSGGGGSGGGGSGGGGS

SEQ ID NO: 107 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 233 aa, Artificial Sequence
  Synthetic polypeptide
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 233 aa, Artificial Sequence
  Synthetic polypeptide
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 233 aa, Artificial Sequence
  Synthetic polypeptide
SEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 382 aa, Artificial Sequence
  Synthetic polypeptide
MPLLLLLPLLWAGALAIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 382 aa, Artificial Sequence
  Synthetic polypeptide
MPLLLLLPLLWAGALAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAA

SEQ ID NO: 113 - protein, 585 aa, Artificial Sequence [sequence omitted]

SEQ ID NO: 114 - protein, 590 aa, Artificial Sequence
  Synthetic polypeptide
MPLLLLLPLLWAGALAQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIPRGLSEAKPATPEIQEIVDKVKPQLEEKTNETYGKLEAVQYKTQVLAAAHFPEHFWSTNYYIKVRAGDNKYMHLKVFNGPQPADMSAEFADRVLTGYQVDKNKDDELTGF

SEQ ID NO: 115 - protein, 231 aa, Artificial Sequence
  Synthetic polypeptide
MPLLLLLPLLWAGALAEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 594 aa, Artificial Sequence
  Synthetic polypeptide
MPLLLLLPLLWAGALAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGF

SEQ ID NO: 117 - protein, 230 aa, Artificial Sequence
  Synthetic polypeptide
MPLLLLLPLLWAGALADIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 596 aa, Artificial Sequence
  Synthetic polypeptide
MPLLLLLPLLWAGALAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAEAAAKEAAAKEAAAKIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGF

SEQ ID NO: 119 - protein, 122 aa, Artificial Sequence
  Synthetic polypeptide
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFAAAGHHHHHH

SEQ ID NO: 120 - protein, 122 aa, Artificial Sequence
  Synthetic polypeptide
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFAAAGHHHHHH

SEQ ID NO: 121 - protein, 122 aa, Artificial Sequence
  Synthetic polypeptide
MIPRGLSEAKPATPEIQEIVDKVKPQLEEKTNETYGKLEAVQYKTQVLAKDWGPSNWWSTNYYIKVRAGDNKYMHLKVFNGPVDDKTLSKDADRVLTGYQVDKNKDDELTGFAAAGHHHHHH

SEQ ID NO: 122 - protein, 122 aa, Artificial Sequence
  Synthetic polypeptide
MIPRGLSEAKPATPEIQEIVDKVKPQLEEKTNETYGKLEAVQYKTQVLAKDHGPIAWWSTNYYIKVRAGDNKYMHLKVFNGPEDTNTDGALADRVLTGYQVDKNKDDELTGFAAAGHHHHHH

SEQ ID NO: 123 - protein, 122 aa, Artificial Sequence
  Synthetic polypeptide
MIPRGLSEAKPATPEIQEIVDKVKPQLEEKTNETYGKLEAVQYKTQVLAKPYGPRDWDSTNYYIKVRAGDNKYMHLKVFNGPEPQLDTSPIADRVLTGYQVDKNKDDELTGFAAAGHHHHHH

SEQ ID NO: 124 - protein, 122 aa, Artificial Sequence
  Synthetic polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAAAGHHHHHH

SEQ ID NO: 125 - protein, 122 aa, Artificial Sequence
  Synthetic polypeptide
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFAAAGHHHHHH

SEQ ID NO: 126 - protein, 122 aa, Artificial Sequence
  Synthetic polypeptide
MIPRGLSEAKPATPEIQEIVDKVKPQLEEKTNETYGKLEAVQYKTQVLANTWFPESFWSTNYYIKVRAGDNKYMHLKVFNGPDDNQERQEHADRVLTGYQVDKNKDDELTGFAAAGHHHHHH

SEQ ID NO: 127 - protein, 122 aa, Artificial Sequence
  Synthetic polypeptide
MIPRGLSEAKPATPEIQEIVDKVKPQLEEKTNETYGKLEAVQYKTQVLAYNHFPEYMWSTNYYIKVRAGDNKYMHLKVFNGPPPRFPHEPFADRVLTGYQVDKNKDDELTGFAAAGHHHHHH

SEQ ID NO: 128 - protein, 122 aa, Artificial Sequence
  Synthetic polypeptide
MIPRGLSEAKPATPEIQEIVDKVKPQLEEKTNETYGKLEAVQYKTQVLAAAHFPEHFWSTNYYIKVRAGDNKYMHLKVFNGPQPADMSAEFADRVLTGYQVDKNKDDELTGFAAAGHHHHHH

SEQ ID NO: 129 - protein, 363 aa, Artificial Sequence
  Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 366 aa, Artificial Sequence
  Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 366 aa, Artificial Sequence
  Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 346 aa, Artificial Sequence
  Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 454 aa, Artificial Sequence
  Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGF

SEQ ID NO: 134 - protein, 500 aa, Artificial Sequence
  Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFLQAAAAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 153 aa, Artificial Sequence
  Synthetic polypeptide
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFGGGGSGGGGSGGGGSGGGGSEPKSCDKTHTCPPCPHHHHHH

SEQ ID NO: 136 - protein, 16 aa, Artificial Sequence
  Synthetic polypeptide
MPLLLLLPLLWAGALA

SEQ ID NO: 137 - protein, 18 aa, Artificial Sequence
  Synthetic polypeptide
MKWVTFISLLFLFSSAYS

SEQ ID NO: 138 - protein, 22 aa, Artificial Sequence
  Synthetic polypeptide
MDMRAPAGIFGFLLVLFPGYRS

SEQ ID NO: 139 - protein, 19 aa, Artificial Sequence
  Synthetic polypeptide
MTRLTVLALLAGLLASSRA

SEQ ID NO: 140 - protein, 19 aa, Artificial Sequence
  Synthetic polypeptide
MELGLSWIFLLAILKGVQC

SEQ ID NO: 141 - protein, 19 aa, Artificial Sequence
  Synthetic polypeptide
MELGLRWVFLVAILEGVQC

SEQ ID NO: 142 - protein, 19 aa, Artificial Sequence
  Synthetic polypeptide
MKHLWFFLLLVAAPRWVLS

SEQ ID NO: 143 - protein, 19 aa, Artificial Sequence
  Synthetic polypeptide
MDWTWRILFLVAAATGAHS

SEQ ID NO: 144 - protein, 19 aa, Artificial Sequence
  Synthetic polypeptide
MDWTWRFLFVVAAATGVQS

SEQ ID NO: 145 - protein, 19 aa, Artificial Sequence
  Synthetic polypeptide
MEFGLSWLFLVAILKGVQC

SEQ ID NO: 146 - protein, 19 aa, Artificial Sequence
  Synthetic polypeptide
MEFGLSWVFLVALFRGVQC

SEQ ID NO: 147 - protein, 26 aa, Artificial Sequence
  Synthetic polypeptide
MDLLHKNMKHLWFFLLLVAAPRWVLS

SEQ ID NO: 148 - protein, 22 aa, Artificial Sequence
  Synthetic polypeptide
MDMRVPAQLLGLLLLWLSGARC

SEQ ID NO: 149 - protein, 22 aa, Artificial Sequence
  Synthetic polypeptide
MKYLLPTAAAGLLLLAAQPAMA

SEQ ID NO: 150 - protein, 17 aa, Artificial Sequence
  Synthetic polypeptide
MGVKVLFALICIAVAEA

SEQ ID NO: 151 - protein, 19 aa, Artificial Sequence
  Synthetic polypeptide
DKTHTCPPCPAPELLGGSS

SEQ ID NO: 152 - protein, 18 aa, Artificial Sequence
  Synthetic polypeptide
MAFLWLLSCWALLGTTFG

SEQ ID NO: 153 - protein, 14 aa, Artificial Sequence
  Synthetic polypeptide
MQLLSCIALILALV

SEQ ID NO: 154 - protein, 15 aa, Artificial Sequence
  Synthetic polypeptide
MNLLLILTFVAAAVA

SEQ ID NO: 155 - protein, 16 aa, Artificial Sequence
  Synthetic polypeptide
MPLLLLLPLLWAGALA

SEQ ID NO: 156 - protein, 30 aa, Artificial Sequence
  Synthetic polypeptide
MDSKGSSQKGSRLLLLLVVSNLLLCQGVVS

SEQ ID NO: 157 - protein, 23 aa, Artificial Sequence
  Synthetic polypeptide
MDAMKRGLCCVLLLCGAVFVSPS

SEQ ID NO: 158 - protein, 16 aa, Artificial Sequence
  Synthetic polypeptide
MLLLLLLLLLLALALA

SEQ ID NO: 159 - protein, 21 aa, Artificial Sequence
  Synthetic polypeptide
MWWRLWWLLLLLLLLWPMVWA

SEQ ID NO: 160 - protein, 18 aa, Artificial Sequence
  Synthetic polypeptide
KESGSVSSEQLAQFRSLD

SEQ ID NO: 161 - protein, 14 aa, Artificial Sequence
  Synthetic polypeptide
EGKSSGSGSESKST

SEQ ID NO: 162 - protein, 12 aa, Artificial Sequence
  Synthetic polypeptide
GSAGSAAGSGEF

SEQ ID NO: 163 - protein, 46 aa, Artificial Sequence
  Synthetic polypeptide
AEAAAKEAAAKEAAAKEAAAKALEAEAAAKEAAAKEAAAKEAAAKA

SEQ ID NO: 164 - protein, 5 aa, Artificial Sequence
  Synthetic polypeptide
PAPAP

SEQ ID NO: 165 - protein, 12 aa, Artificial Sequence
  Synthetic polypeptide
AEAAAKEAAAKA

SEQ ID NO: 166 - protein, 7 aa, Artificial Sequence
  Synthetic polypeptide
GGGSGGG

SEQ ID NO: 167 - protein, 8 aa, Artificial Sequence
  Synthetic polypeptide
ESGGGGVT

SEQ ID NO: 168 - protein, 9 aa, Artificial Sequence
  Synthetic polypeptide
LESGGGGVT

SEQ ID NO: 169 - protein, 6 aa, Artificial Sequence
  Synthetic polypeptide
GRAQVT

SEQ ID NO: 170 - protein, 6 aa, Artificial Sequence
  Synthetic polypeptide
WRAQVT

SEQ ID NO: 171 - protein, 8 aa, Artificial Sequence
  Synthetic polypeptide
ARGRAQVT

SEQ ID NO: 172 - protein, 33 aa, Artificial Sequence
  Synthetic polypeptide
SGTTSGTTRLLSGHTCFTLTGLLGTLVTMGLLT

SEQ ID NO: 173 - protein, 26 aa, Artificial Sequence
  Synthetic polypeptide
SGTSPGLSAGATVGIMIGVLVGVALI

SEQ ID NO: 174 - protein, 24 aa, Artificial Sequence
  Synthetic polypeptide
SAPVLSAVATVGITIGVLARVALI

SEQ ID NO: 175 - protein, 24 aa, Artificial Sequence
  Synthetic polypeptide
SSPDLSAGTAVSIMIGVLAGMALI

SEQ ID NO: 176 - protein, 25 aa, Artificial Sequence
  Synthetic polypeptide
TLGGNSASYTFVSLLFSAVTLLLLC

SEQ ID NO: 177 - protein, 26 aa, Artificial Sequence
  Synthetic polypeptide
SGTSPGLSAGATVGIMIGVLVGVALI

SEQ ID NO: 178 - protein, 16 aa, Artificial Sequence
  Synthetic polypeptide
DKTHTCPPCPAPELLG

SEQ ID NO: 179 - protein, 16 aa, Artificial Sequence
  Synthetic polypeptide
GSTHTCPPCPAPELLG

SEQ ID NO: 180 - protein, 21 aa, Artificial Sequence
  Synthetic polypeptide
EPKSCDKTHTCPPCPAPELLG

SEQ ID NO: 181 - protein, 24 aa, Artificial Sequence
  Synthetic polypeptide
EPKSCDKTHTCPPCPAPELLGGPS

SEQ ID NO: 182 - protein, 24 aa, Artificial Sequence
  Synthetic polypeptide
EPKSSDKTHTCPPCPAPELLGGPS

SEQ ID NO: 183 - protein, 24 aa, Artificial Sequence
  Synthetic polypeptide
EPKSSDKTHTCPPCPAPELLGGSS

SEQ ID NO: 184 - protein, 24 aa, Artificial Sequence
  Synthetic polypeptide
EPKSSGSTHTCPPCPAPELLGGSS

SEQ ID NO: 185 - protein, 19 aa, Artificial Sequence
  Synthetic polypeptide
DKTHTCPPCPAPELLGGPS

SEQ ID NO: 186 - protein, 19 aa, Artificial Sequence
  Synthetic polypeptide
DKTHTCPPCPAPELLGGSS

SEQ ID NO: 187 - protein, 9 aa, Artificial Sequence
  Synthetic polypeptide
AVPFPHQQL

SEQ ID NO: 188 - protein, 9 aa, Artificial Sequence
  Synthetic polypeptide
WAPFPHQQL

SEQ ID NO: 189 - protein, 9 aa, Artificial Sequence
  Synthetic polypeptide
WVAFPHQQL

SEQ ID NO: 190 - protein, 9 aa, Artificial Sequence
  Synthetic polypeptide
WVPAPHQQL

SEQ ID NO: 191 - protein, 9 aa, Artificial Sequence
  Synthetic polypeptide
WVPFAHQQL

SEQ ID NO: 192 - protein, 9 aa, Artificial Sequence
  Synthetic polypeptide
WVPFPAQQL

SEQ ID NO: 193 - protein, 9 aa, Artificial Sequence
  Synthetic polypeptide
WVPFPHAQL

SEQ ID NO: 194 - protein, 9 aa, Artificial Sequence
  Synthetic polypeptide
WVPFPHQAL

SEQ ID NO: 195 - protein, 9 aa, Artificial Sequence
  Synthetic polypeptide
WVPFPHQQA

SEQ ID NO: 196 - protein, 31 aa, Artificial Sequence
  Synthetic polypeptide
AEAAAKEAAAKEAAAKEAAAKEAAAKEAAAK

SEQ ID NO: 197 - protein, 20 aa, Artificial Sequence
  Synthetic polypeptide
GGGGSGGGGSGGGGSGGGGS

SEQ ID NO: 198 - protein, 30 aa, Artificial Sequence; identical to SEQ ID NO: 106
  Synthetic polypeptide
  misc_feature (6)..(30): May be absent

SEQ ID NO: 199 - protein, 8 aa, Artificial Sequence
  Synthetic polypeptide
GGGGGGGG

SEQ ID NO: 200 - protein, 6 aa, Artificial Sequence
  Synthetic polypeptide
GGGGGG

SEQ ID NO: 201 - protein, 30 aa, Artificial Sequence
  Synthetic polypeptide
  misc_feature (6)..(30): May be absent
EAAAKEAAAKEAAAKEAAAKEAAAKEAAAK

SEQ ID NO: 202 - protein, 68 aa, Artificial Sequence
  Synthetic polypeptide
  misc_feature (21)..(68): May be absent
APAPAPAPAPAPAPAPAPAPAPAPAPAPAPAPAPAPAPAPAPAPAPAPAPAPAPAPAPAPAPAPAPAP

SEQ ID NO: 203 - protein, 4 aa, Artificial Sequence
  Synthetic polypeptide
GGSG

SEQ ID NO: 204 - protein, 4 aa, Artificial Sequence
  Synthetic polypeptide
GSGS

SEQ ID NO: 205 - protein, 4 aa, Artificial Sequence
  Synthetic polypeptide
GGGS

SEQ ID NO: 206 - protein, 22 aa, Artificial Sequence
  Synthetic polypeptide
  misc_feature (5)..(22): May be absent
SGGSGGSGGSGGSGGSGGSGGS

SEQ ID NO: 207 - protein, 6 aa, Artificial Sequence
  Synthetic polypeptide
RGVFRR

SEQ ID NO: 208 - DNA, 12 bp, Artificial Sequence
  Synthetic polynucleotide
accgttgccggt

SEQ ID NO: 209 - DNA, 12 bp, Artificial Sequence
  Synthetic polynucleotide
  misc_feature (4)..(9): n is a, c, g, or t
accnnnnnnggt